(19) United States
(12) Patent Application Publication
(10) Pub. No.: US 2009/0048269 A1
(43) Pub. Date: Feb. 19, 2009

(54) CHEMICAL COMPOUNDS-821

(75) Inventors: Maurice Raymond Finlay, Macclesfield (GB); Frederick Woolf Goldberg, Macclesfield (GB); Craig Anthony Roberts, Macclesfield (GB); Nicola Jane Roberts, Macclesfield (GB); David Alan Rudge, Macclesfield (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/190,008

(22) Filed: Aug. 12, 2008

Related U.S. Application Data
(60) Provisional application No. 60/955,486, filed on Aug. 13, 2007.

(51) Int. Cl. A61K 31/497; C07D 401/12; C07D 401/14; A61K 31/444

(52) U.S. Cl. 514/255.05; 546/261; 514/335; 544/405

(57) ABSTRACT

The invention relates to chemical compounds of the formula (I),

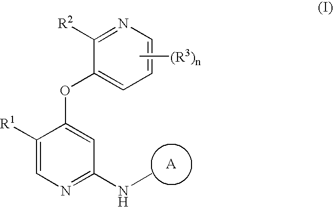

or pharmaceutically acceptable salts thereof, which possess ALK5 (TGFβR1) inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments for use in the production of an anti-cancer effect in a warm-blooded animal such as man.

This application claims the benefit under 35 U.S.C. §119(e) for U.S. Provisional Application No. 60/955486 filed on 13 Aug. 2007.

The invention relates to chemical compounds, or pharmaceutically acceptable salts thereof, which possess ALK5 (TGFβR1) inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments for use in the production of an anti-cancer effect in a warm-blooded animal such as man.

The transforming growth factor family of ligands, including TGFβ, activins, inhibins, nodal and bone morphogenic proteins, play a key role in controlling cellular functions such as differentiation, migration, proliferation, adhesion and development (Miyazono, K et al, 2001, J. Cell Physiol., 187, 265-276). This family of ligands signal through heteromeric complexes of type I (e.g. activin receptor like kinases or ALKs) and type II serine/threonine kinase (e.g. TGFβRII) receptors. Upon ligand binding primarily to a common coreceptor, [e.g. βglycan (Roberts et al, 1980, Proc. Natl. Acad. Sci USA, 77, 3494-3498)], type II receptors and type I receptors are phosphorylated and hence activated to enable downstream signalling events to occur. In mammals, seven type I receptors have been identified (ALK1 through to ALK7), with ALK4, 5, and 7 showing a high degree of structural similarity. Signalling specificity is determined by the ligand, receptor complex and the cellular context. For TGFβ signalling, all three ligands (TGFβ1, TGFβ2, TGFβ3) are known to signal through TGFβRII and TGFβRI (ALK5) by effecting phosphorylation of a glycine/serine or GS domain (Huse, M et al, 2001, Molecular Cell, 8, 671-682) on ALK5. This enables binding to downstream substrates and activation of TGFβ pathways. These can be divided into Smad-dependent and Smad-independent pathways, the former being the most clearly understood. Activated ALK5 phosphorylates and specifically activates Smad2 and Smad3 which subsequently form heterotrimeric complexes with Smad4. The Smad complex then translocates to the nucleus where it associates with various cell type specific transcription factors to regulate TGFβ-dependent gene expression (Massague, 1998, Ann. Rev. Biochem. Med., 67, 773-).

Due to the diversity of biological effects, inhibitors of the TGFβ superfamily pathway possess enormous potential for several clinical applications. These include cancer (Siegel, P. M. et al, 2003, Nat. Rev. Cancer, 3, 807-820, Li, M. O. et al, 2006, Ann. Rev. Immunol., 24, 99-146), fibrosis (Border et al, 1994, N. Engl. J. Med., 331 (19), 1286-1292, McCaffrey, T et al, 1995, J. Clin. Invest., 96(6), 2667-2675), osteoporosis and muscular disorders (Iyer, S et al, 2005, Cancer Biol. Ther., 4, 261, Lee, S. J. and McPherron, A. C., 2001, Proc. Natl. Acad. Sci. USA, 98, 9306). In cancer TGFβ signalling appears to play a dual role in cancer progression (Pardal, K and Moustakas, A, 2007, Biochemica et Biophysica Acta, 1775, 21-62). Antiproliferative and apoptotic responses to TGFβ in normal epithelial, neuronal and haematopoietic cells effectively limit the growth of these cell lineages yet in advanced and late stage tumours, TGFβ elicits a tumour promotion effect. Notably, this results in an increase in tumour cell invasiveness and metastasis, the mechanism behind which is not completely understood. Accordingly, TGFβ can induce epithelial to mesenchymal transition, a hallmark of cancer invasion. It follows that the TGFβpathway offers an opportunity for therapeutic intervention in late stage and metastatic cancer disease.

Therefore, in one aspect of the invention, there is provided a compound of formula (I):

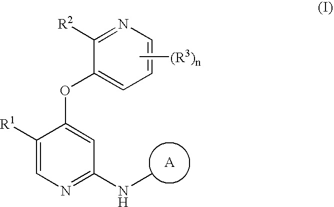

wherein:

R¹ is hydrogen, halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;

R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on is carbon by one or more R¹²;

R² is hydrogen, halo, cyano, nitro, mercapto, sulfo, hydroxy, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N,N—(C₁₋₆alkyl)₂amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;

R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;

n is 0 to 3; wherein the values of R³ may be the same or different;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, nitro, mercapto, sulfo, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R⁴³;

R⁴⁵ and R⁴⁶ are independently selected from a direct bond, —C(O)—, —C(═NH)—, —N(R¹⁰¹)—C(═NH)—, —N(R⁴⁹)C(O)—, —N(R⁵⁰)SO₂—, —O—C(O)— and —S(O)ₐ— wherein a is 1 or 2;

R⁴³ and R⁴⁷ are independently halo, cyano, nitro, mercapto, sulfo, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N-(C₁₋₆alkyl)₂sulfamoyl, N—[(C₁-₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R²² and R³² are independently selected from C₁₋₆alkyl, C₃-₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R⁴⁴, R⁴⁸ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴, R⁴³ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷ )N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷—, carbocyclyl-R⁸⁸— and (C₁₋₆alkyl)-S(O)ₐ— wherein a is 0 to 2; and wherein if said heterocyclyl contains an —NH— moiety that nitrogen may be optionally substituted by R⁸⁹;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ and R⁸⁹ are each independently selected from C₁₋₆alkyl, C₁₋₆alkanoyl; C₁₋₆alkylsulfonyl;

R¹⁹ and R²⁰ are independently selected from a direct bond, —CH(R⁶¹)—, —CH(OR⁶²)—, —C(R⁶³)═C(R⁶⁴)—, ethynylene, —O—, —C(O)—, —N(R⁶⁶)C(O)—, —N(R⁶⁹)SO₂— and —S(O)ₐ— wherein a is 0 to 2;

R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R¹², R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, carbamimidoyl, carbamimidoylamino, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴, R⁵, R⁶, R⁷, R⁸, R⁹, R²³, R²⁴, R²⁵, R²⁶, R²⁷, R²⁸, R³⁵, R³⁶, R³⁷, R³⁸, R³⁹, R⁴⁰, R^{49, R}⁵⁰, R⁵¹, R⁵², R⁵³, R⁵⁴, R⁵⁵, R⁵⁶, R⁶¹, R⁶², R⁶³, R⁶⁴, R⁶⁶, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl;

or a pharmaceutically acceptable salt thereof,

wherein the compound of formula (I) is other than:
3-(2-{[4-(1-acetylpiperidin-4-yl)-1,3-thiazol-2-yl]amino}pyridin-4-yl)-oxypyridine-4-carbonitrile;
N-(4-methyl-1,3-thiazol-2-yl)-4-pyridin-3-yloxypyridin-2-amine;
ethyl 5-{5-bromo-2-[(3-{1-[(2-methylpropan-2-yl)oxycarbonyl]piperidin-4-yl}-1,2,4-thiadiazol-5-yl)amino]pyridin-4-yl}oxy-4,6-dimethylpyridine-3-carboxylate;
5-{5-bromo-2-[(3-piperidin-4-yl-1,2,4-thiadiazol-5-yl)amino]pyridin-4-yl]oxy-N-(2-dimethylaminoethyl}-4,6-dimethylpyridine-3-carboxamide;
tert-butyl 4-[5-({5-bromo-4-[5-(2-dimethylaminoethylcarbamoyl)-2,4-dimethylpyridin-3-yl]oxypyridin-2-yl}amino)-1,2,4-thiadiazol-3-yl]piperidine-1-carboxylate; or
5-{5-bromo-2-[(3-{1-[(2-methylpropan-2-yl)oxycarbonyl]piperidin-4-yl}-1,2,4-thiadiazol-5-yl)amino]pyridin-4-yl}oxy-4,6-dimethylpyridine-3-carboxylic acid;
or a pharmaceutically acceptable salt thereof

A “heterocyclyl” is a saturated, partially saturated or fully unsaturated, mono or bicyclic ring system containing 4-12 ring atoms of which at least one ring atom is chosen from nitrogen, sulfur or oxygen, which may unless otherwise specified, be carbon or nitrogen linked, wherein a ring —CH₂— can optionally be replaced by a —C(O)—, a ring sulfur is may be optionally oxidised to form the S-oxides and a ring nitrogen may be optionally oxidised to form the N-oxide.

Examples of a heterocyclyl include morpholinyl, piperidinyl, pyridyl, pyranyl, pyrrolyl, pyrazolyl, isothiazolyl, indolyl, indolinyl, benzo[b]furanyl, 1H-indazolyl, benzimidazolyl, benzthiazolyl, isoquinolinyl, cinnolinyl, quinoxalinyl, 1,8-naphthyridinyl, pteridinyl, quinolyl, isoquinolyl, 1H-pyrrolo[2,3-b]pyridinyl, thienyl, furyl, 1,3-benzodioxolyl, thiadiazolyl, piperazinyl, thiazolidinyl, pyrrolidinyl, thiomorpholinyl, pyrrolinyl, homopiperazinyl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

Further examples of “heterocyclyl” include oxazolyl, tetrahydrofuranyl, triazolyl, azetidinyl, 2-oxoimidazolidinyl, azepanyl, 1,1-dioxothiomorpholinyl, azabicyclo[3.2.1]octanyl and 1,3-thiazolyl.

In one aspect of the invention a heterocyclyl is a saturated, partially saturated or fully unsaturated, mono or bicyclic ring system containing 4-12 ring atoms of which 1-4 ring atoms are chosen from nitrogen, sulfur or oxygen, which may unless otherwise specified, said heterocyclyl may be carbon or nitrogen linked, wherein a ring —CH₂— can optionally be replaced by a —C(O)—, a ring sulfur may be optionally oxidised to form the S-oxides and a ring nitrogen may be optionally oxidised to form the N-oxide.

In a further aspect of the invention a heterocyclyl is a saturated, partially saturated or fully unsaturated, mono or bicyclic ring system containing 4-12 ring atoms of which 1-2 ring atoms are chosen from nitrogen, sulfur or oxygen, which may unless otherwise specified, said heterocyclyl may be carbon or nitrogen linked, wherein a ring —CH₂— can optionally be replaced by a —C(O)—, a ring sulfur may be optionally oxidised to form the S-oxides and a ring nitrogen may be optionally oxidised to form the N-oxide.

In one aspect of the invention a “heterocyclyl” is:

a fully unsaturated monocyclic ring containing 5-6 ring atoms, or

a fully unsaturated or partially saturated bicyclic ring system containing 8-10 ring atoms,

of which at least one ring atom is chosen from nitrogen, sulfur or oxygen, which may unless otherwise specified, be carbon or nitrogen linked, wherein a ring —CH₂— is can optionally be replaced by a —C(O)—.

In one aspect of the invention a “heterocyclyl” is:

a fully unsaturated monocyclic ring containing 5-6 ring atoms, or

a fully unsaturated or partially saturated bicyclic ring system containing 8-10 ring atoms,

of which 1-4 ring atoms are chosen from nitrogen, sulfur or oxygen, which may unless otherwise specified, be carbon or nitrogen linked, wherein a ring —CH₂— can optionally be replaced by a —C(O)—.

In one aspect of the invention a “heterocyclyl” is:

a fully unsaturated monocyclic ring containing 5-6 ring atoms, or

a fully unsaturated or partially saturated bicyclic ring system containing 8-10 ring atoms,

of which 1-2 ring atoms are chosen from nitrogen, sulfur or oxygen, which may unless otherwise specified, be carbon or nitrogen linked, wherein a ring —CH₂— can optionally be replaced by a —C(O)—.
In a further aspect of the invention a “heterocyclyl” is:

a fully unsaturated monocyclic ring containing 5-6 ring atoms, or

a fully unsaturated bicyclic ring system containing 8 to 10 ring atoms,

of which at least one ring atom is chosen from nitrogen, sulfur or oxygen, which may unless otherwise specified, be carbon or nitrogen linked.
In another aspect of the invention a “heterocyclyl” is:

a saturated monocyclic or bicyclic ring system containing 4-12 ring atoms of which at least one ring atom is chosen from nitrogen, sulfur or oxygen, which unless other wise specified, be carbon or nitrogen linked, wherein a ring —CH₂— can optionally be replaced by a —C(O)—, a ring sulfur may be optionally oxidised to form the S-oxides and a ring nitrogen may be optionally oxidised to form the N-oxide.

In a further embodiment a “heterocyclyl” is selected from pyridinyl, pyrrolidinyl, oxazolyl, piperazinyl, morpholinyl, furanyl, tetrahydrofuranyl, quinolyl, pyrimidinyl, triazolyl, tetrahydropyranyl, azetidinyl, imidazolyl, pyrazinyl, piperidinyl, pyrazolyl, pyrrolyl, 2-oxoimidazolidinyl, azepanyl, 1,1-dioxothiomorpholinyl, 2-oxopyrrolidinyl, azabicyclo[3.2.1]octanyl, 1,3-thiazolyl and thienyl.

A “carbocyclyl” is a saturated, partially unsaturated or fully unsaturated ring system containing 3-12 carbon atoms; wherein a —CH₂— can optionally be replaced by a —C(O)—.

In one aspect, a “carbocyclyl” is a saturated, partially saturated or fully unsaturated, mono or bicyclic ring containing 3-12 atoms; wherein a —CH₂— can optionally be replaced by a —C(O)—.

In one aspect of the invention a “carbocyclyl” is a monocyclic ring containing 3-6 ring atoms, or a bicyclic ring system containing 9 or 10 ring atoms.

Examples of “carbocyclyl” include cyclopropyl, cyclobutyl, 1-oxocyclopentenyl, cyclopentyl, cyclopentenyl, cyclohexyl, cyclohexenyl, phenyl, naphthyl, tetralinyl, indanyl and 1-oxoindanyl.

A further example of “carbocyclyl” is adamantyl.

In one aspect of the invention a “carbocyclyl” is:

phenyl, or

a fully unsaturated or partially saturated bicyclic ring system containing 9 or 10 ring atoms that contains a benzene ring within the ring system.

Examples of a fully unsaturated or partially saturated bicyclic ring system containing 9 or 10 ring atoms that contains a benzene ring within the ring system are naphthyl, tetralinyl and indanyl.

In another aspect of the invention a “carbocyclyl” is:

a saturated or partially saturated monocyclic ring containing 3-6 ring atoms.

In one embodiment a “carbocyclyl” is selected from phenyl, cyclopropyl, adamantyl, cyclohexyl and cyclobutyl.

A “heterocyclic group” is a saturated, partially saturated or fully unsaturated, mono or bicyclic ring system containing 4-12 ring atoms of which at least one ring atom is chosen from nitrogen, sulfur or oxygen, which is linked via a carbon, wherein a ring —CH₂— can optionally be replaced by a —C(O)—, a ring sulfur may be optionally oxidised to form the S-oxides and a ring nitrogen may be optionally oxidised to form the N-oxide.

Examples of a heterocyclic group are morpholinyl, piperidinyl, pyridyl, pyranyl, pyrrolyl, pyrazolyl, isothiazolyl, indolyl, indolinyl, benzo[b]furanyl, 1H-indazolyl, benzimidazolyl, benzthiazolyl, isoquinolinyl, cinnolinyl, quinoxalinyl, 1,8-naphthyridinyl, pteridinyl, quinolyl, isoquinolyl, 1H-pyrrolo[2,3-b]pyridinyl, thienyl, furyl, 1,3-benzodioxolyl, thiadiazolyl, piperazinyl, thiazolidinyl, pyrrolidinyl, thiomorpholinyl, pyrrolinyl, homopiperazinyl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

A further example of “heterocyclic group” is 2,2-dioxo-1,3-dihydro-2-benzothiophen-5-yl.

In one embodiment a heterocyclic group is a saturated, partially saturated or fully unsaturated, mono or bicyclic ring system containing 4-12 ring atoms of which 1-4 ring atoms are chosen from nitrogen, sulfur or oxygen, which is linked via a carbon, wherein a ring —CH₂— can optionally be replaced by a —C(O)—, a ring sulfur may be optionally oxidised to form the S-oxides and a ring nitrogen may be optionally oxidised to form the N-oxide.

In a further embodiment a heterocyclic group is a saturated, partially saturated or fully unsaturated, mono or bicyclic ring system containing 4-12 ring atoms of which 1-2 ring atoms are chosen from nitrogen, sulfur or oxygen, which is linked via a carbon, wherein a ring —CH₂— can optionally be replaced by a —C(O)—, a ring sulfur may be optionally oxidised to form the S-oxides and a ring nitrogen may be optionally oxidised to form the N-oxide.

In one aspect of the invention a “heterocyclic group” is:

a fully unsaturated or partially saturated monocyclic ring containing 5-6 ring atoms, or

a fully unsaturated or partially saturated bicyclic ring system containing 8-10 ring atoms,

of which at least one ring atom is chosen from nitrogen, sulfur or oxygen, which is linked via a carbon, wherein a ring —CH₂— can optionally be replaced by a —C(O)—, and a ring sulfur may be optionally oxidised to form the S-oxides and a ring nitrogen may be optionally oxidised to form the N-oxide.

In a further aspect of the invention a “heterocyclic group” is:

a fully unsaturated monocyclic ring containing 5-6 ring atoms, or

a fully unsaturated bicyclic ring system containing 8 to 10 ring atoms, of which at least one ring atom is chosen from nitrogen, sulfur or oxygen, which is linked via a carbon.

In a further aspect of the invention a “heterocyclic group” is:

a fully unsaturated monocyclic ring containing 5-6 ring atoms, or

a fully unsaturated bicyclic ring system containing 8 to 10 ring atoms, of which 1-4 ring atoms are chosen from nitrogen, sulfur or oxygen, which is linked via a carbon.

In a further aspect of the invention a “heterocyclic group” is:

a fully unsaturated monocyclic ring containing 5-6 ring atoms, or

a fully unsaturated bicyclic ring system containing 8 to 10 ring atoms, of which 1-2 ring atoms are chosen from nitrogen, sulfur or oxygen, which is linked via a carbon.

Examples of “heterocyclic group” are 3-isoxazolyl, 2-pyridyl, 3-pyridiyl, 4-pyridyl, 2-pyrazinyl, 1,2-dihydropyrazol-3-yl, 3-pyrazolyl, 2-pyrimidinyl, 4-pyrimidinyl, 1,2,4-thiadiazolyl, indolyl, 1H-indazolyl, 1,3-denzodioxol-5-yl, benzimidazole, 1,3-dihydro-2H-indol-2-one and 1,3-benzothiazolyl.

Examples of “heterocyclic group” are pyridyl, pyrazolyl and indolyl.

In one embodiment a “heterocyclic group” is selected from pyrazolyl, pyridinyl, 2,2-dioxo-1,3-dihydro-2-benzothiophen-5-yl and indolyl.

A “carbocyclic group” is a saturated, partially saturated or fully unsaturated, mono- or bicyclic ring containing 4-12 atoms; wherein a —CH₂— can optionally be replaced by a —C(O)— group.

In one aspect of the invention a “carbocyclic group” is a monocyclic ring containing 4-6 ring atoms, or a bicyclic ring system containing 9 or 10 ring atoms.

Examples of “carbocyclic group” include cyclobutyl, 1-oxocyclopentenyl, cyclopentyl, cyclopentenyl, cyclohexyl, cyclohexenyl, phenyl, naphthyl, tetralinyl, indanyl and 1-oxoindanyl.

In one aspect of the invention a “carbocyclic group” is:
phenyl, or
a fully unsaturated or partially saturated bicyclic ring system containing 9 or 10 ring atoms that comprises a benzene ring within the ring system,
wherein a —CH₂— can optionally be replaced by a —C(O)— group.

In one embodiment when Ring A comprises a carbocyclic group that is fully unsaturated or partially saturated bicyclic ring system containing 9 or 10 ring atoms wherein a —CH₂— can optionally be replaced by a —C(O)—, wherein one ring of the bicyclic ring is a benzene ring, then the benzene ring of Ring A is directly attached to the 2-aminopyridyl nitrogen of formula (I).

In one embodiment a “carbocyclic group” is selected from phenyl, indenyl, azulenyl, naphthyl, tetralinyl, indanyl and 1-oxoindanyl.

In one aspect a “carbocyclic group” is phenyl. The term “halo” refers to fluoro, chloro, bromo and iodo.

Where a group may be optionally substituted by “one or more” R^{x}, it is to be understood that the selection is to be made from all of the substituents listed for R^{x} and that when two or more substituents are chosen these may be the same or different.

In this specification, the term “alkyl” includes both straight and branched chain alkyl groups. References to individual alkyl groups such as “propyl” are specific for the straight chain version only and references to individual branched chain alkyl groups such as “isopropyl” are specific for the branched chain version only. This convention applies to other radicals described within this specification such as alkenyl radicals, alkynyl radicals, alkoxy radicals and alkanoyl radicals.

For example, “C₁₋₆alkyl” includes C₁₋₄alkyl, C₁₋₃alkyl, methyl, ethyl, propyl, isopropyl and t-butyl.

Examples of “C₁₋₃alkyl” are methyl, ethyl, propyl and isopropyl.

In this specification ”C₂₋₆alkenyl” includes C₂₋₃alkenyl, butenyl, isobutenyl, 1,5-hexadien3-yl. Examples of “C₂₋₃alkenyl” are vinyl, prop-2-en-1-yl and prop-1-en-2-yl.

Examples of the term “C₂₋₆alkynyl” include C₂₋₃alkynyl, butynyl, propynyl and ethynyl.

Examples of the term “C₁₋₆alkoxy” include C₁₋₃alkoxy, t-butyloxy, isopropoxy, butoxy, ethoxy and methoxy.

Examples of the term “C₁₋₆alkoxy” include C₁₋₃alkoxy, methoxy, ethoxy, propoxy and isopropoxy.

Examples of the term “(C₁₋₆alkyl)-S(O)ₐ— wherein a is 0 to 2” include “(C₁₋₆alkyl)-S—”, “(C₁₋₃alkyl)-S(O)ₐ— wherein a is 0 to 2”, “(C₁₋₃alkyl)-S(O)₂—”, isopropylsulfanyl, propylsulfonyl, mesyl and ethylsulfanyl, butanesulfinyl and isopentylsulfinyl.

Examples of the term “C₁₋₆alkoxycarbonyl” include C₁₋₃alkoxycarbonyl, methoxycarbonyl, ethoxycarbonyl, isopropoxycarbonyl and isopentoxycarbonyl.

Examples of the term “C₁₋₆alkanoyl” include C₁₋₃alkanoyl, formyl, acetyl and propionyl.

Examples of the term “C₁₋₆alkanoyloxy” include C₁₋₃alkanoyloxy, acetyloxy and propionyloxy.

Examples of the term “N—(C₁₋₆alkyl)amino” include N—(C₁₋₃alkyl)amino, methylamino, isopropylamino and isohexylamino.

Examples of the term “N,N—(C₁₋₆alkyl)₂amino” include N,N—(C₁₋₃alkyl)₂amino, N,N-dimethylamino, N-isopropyl-N-methylamino and N-pentyl-N-ethylamino.

Examples of the term “N—(C₁₋₆alkanoyl)-N—(Rⁿ)amino” wherein Rⁿ can be hydrogen, C₁₋₃alkyl or cyclopropyl, include N—(C₁₋₃alkanoyl)-N—(Rⁿ)amino, N-propionoyl-N—(Rⁿ)amino, N-propionoylamino, N-acetyl-N-methylamino and N-acetyl-N-cyclopropylamino.

Examples of the term “N—(C₁₋₆alkanoyl)-N—(Rⁿ)-sulfamoyl” are N-acetylsulfamoyl, N-propanoylsulfamoyl. One example of the term “N—(C₁₋₆alkanoyl)-N—(Rⁿ)-sulfamoyl” is N-acetylsulfamoyl.

Examples of the term “N—(C₁₋₆alkoxycarbonyl)-N—(Rⁿ)amino” wherein Rⁿ can be hydrogen, C₁₋₃alkyl or cyclopropyl, include N—(C₁₋₃alkoxycarbonyl)-N—(Rⁿ)amino, N—(C₁₋₆alkoxycarbonyl)-N-amino, N-isopentoxycarbonyl-N-ethylamino, N-propoxycarbonyl-N-cyclopropylamino and N-methoxycarbonylamino.

Examples of “N—(C₁₋₆alkyl)carbamoyl” include N—(C₁₋₃alkyl)carbamoyl, N-isopentylaminocarbonyl, N-methylaminocarbonyl and N-ethylaminocarbonyl.

Examples of “N,N—(C₁₋₆alkyl)₂carbamoyl” include N,N—(C₁₋₃alkyl)₂carbamoyl, N-isopentyl-N-ethylaminocarbonyl, N,N-dimethylaminocarbonyl and N-methyl-N-ethylaminocarbonyl.

Examples of “N—(C₁₋₆alkyl)sulfamoyl” include N—(C₁₋₃alkyl)sulfamoyl, N-isopentylsulfamoyl, N-methylsulfamoyl and N-ethylsulfamoyl.

Examples of “N,N—(C₁₋₆alkyl)₂sulfamoyl” include N,N—(C₁₋₃alkyl)₂sulfamoyl, N-isopentyl-N-ethylsulfamoyl, N,N-dimethylsulfamoyl and N-methyl-N-ethylsulfamoyl.

Examples of “N—[(C₁₋₆alkyl)sulfonyl]-N—(Rⁿ)amino” wherein Rⁿ can be hydrogen, C₁₋₃alkyl or cyclopropyl, include N—[(C₁₋₃alkyl)sulfonyl]-N—(Rⁿ)amino, N—[(C₁₋₆alkyl)sulfonyl]amino, N-(isopentylsulfonyl)-N-(cyclopropyl)amino, N-mesyl-N-ethylamino and N-(isopropylsulfonyl)amino.

Examples of “N—(C₁₋₆alkyl)carbamimidoyl” include N—(C₁₋₃alkyl)carbamimidoyl, N-methylcarbamimidoyl, N-ethylcarbamimidoyl N-isopropylcarbamimidoyl and N-isohexylcarbamimidoyl.

Examples of “N,N—(C₁₋₆alkyl)₂carbamimidoyl” include N,N—(C₁₋₃alkyl)₂carbamimidoyl N,N-dimethylcarbamimidoyl, N-methyl-N-isopropylcarbamimidoyl and N-ethyl-N-pentylcarbamimidoyl.

Examples of “N—[N′,N′—(Rⁿ)(R^{m})carbamimidoyl]-N—(R^{q})amino” wherein Rⁿ, R^{m}, and R^{q} can each represent hydrogen, C₁₋₃alkyl or cyclopropyl include: carbamimidoylamino, N—(N′-methylcarbamimidoyl)amino, N—(N′,N′-diethylcarbamimidoyl)-N-cyclopropylamino and N-(carbamimidoyl)-N-ethylamino.

Examples of “(Rⁿ)(R^{m})N—S(O)₂—N(R^{q})—” wherein Rⁿ, R^{m}, and R^{q} can each represent hydrogen, C₁₋₃alkyl or cyclopropyl include: (ethyl)(methyl)N—SO₂—N(methyl)-, otherwise known as: 3-ethyl-1,3-dimethyl-2,2-dioxido-2λ⁶-diazathianyl; and (cyclopropyl)NH—SO₂—NH—, otherwise known as:
3-cyclopropyl-2,2-dioxido-2λ⁶-diazathianyl.

One example of “(Rⁿ)(R^{m})N—S(O)₂—N(R^{q})—” is (methyl)(methyl)N—SO₂—NH—, otherwise known as 3,3-dimethyl-2,2-dioxido-2λ⁶-diazathianyl.

Examples of “3,3-(Rⁿ)(R^{m})-1-(R^{q})ureido” wherein Rⁿ, R^{m}, and R^{q} can each represent hydrogen, C₁₋₃alkyl or cyclopropyl, include 3-propyl-1-methylureido, 3,3-dimethylureido, 1-cyclopropylureido, 3-cyclopropyl-3-methyl-1-ethylureido and ureido.

For the avoidance of doubt, using the definition of R²¹ as an example:
R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;
wherein: R²³, R²⁴, R²⁵, R²⁶, R²⁷ and R²⁸ are independently selected from hydrogen, C₁₋₃alkyl and cyclopropyl;
and: R²⁹ and R³⁰ are independently selected from a direct bond, —O—, —N(R⁷⁰)—, —C(O)—, —N(R⁷¹)C(O)—, —C(O)N(R⁷²)—, —SO₂N(R⁷³)—, —N(R⁷⁴)SO₂— and —S(O)ₐ— wherein a is 0 to 2;
   it is intended that the optional R³¹ substituent(s) may for example be attached to the carbon(s) of any of the groups listed, including the carbons of the R²³, R²⁴, R²⁵, R²⁶, R²⁷ or R²⁸ substituents, and also, that the optional R³¹ substituent(s) may for example be attached to the carbon(s) of any of the R²⁹ or R³⁰ linker units, which means in effect that the R³¹ substituent(s) may be substituted on the carbon(s) of any of the R⁷⁰, R⁷¹, R⁷², R⁷³ or R⁷⁴ linker subunits. This convention applies similarly to other group definitions in this specification.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess ALK5 inhibitory activity. The invention further relates to any and all tautomeric forms of the compounds of the formula (I) that possess ALK5 inhibitory activity.

It is also to be understood that certain compounds of the formula (I) can exist in solvated as well as unsolvated forms such as, for example, hydrated forms. It is to be understood that the invention encompasses all such solvated forms which possess ALK5 inhibitory activity.

Some values of variable groups are as follows. Such values may be used where appropriate with any of the definitions, claims or embodiments defined hereinbefore or hereinafter.

In a further aspect there is provided a compound of formula (I) wherein the compound of formula (I) is a compound of formula (IA):

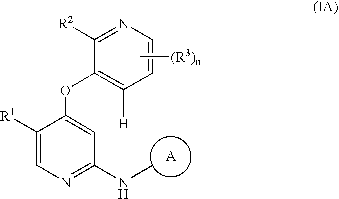

wherein

In a further aspect there is provided a compound of formula (I), wherein the compound of formula (I) is a compound of formula (IB):

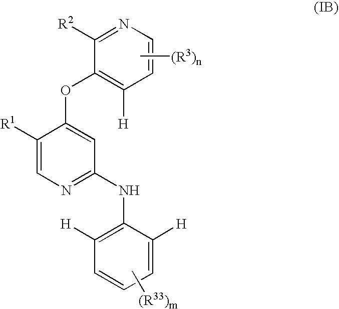

wherein:

m is 0 to 3; wherein the values of R³³ may be the same of different; and the values of R², n, R¹, R³ and R³³ are as defined herein.

In a further embodiment there is provided a compound of formula (I) which is a compound of formula (IB) wherein:

m is 1 to 3; wherein the values of R³³ may be the same of different.

In a further embodiment there is provided a compound of formula (I) which is a compound of formula (IB) wherein:

m is 1 or 2; wherein the values of R³³ may be the same of different.

In a further embodiment there is provided a compound of formula (I) which is a compound of formula (IB) wherein m is 1.

For the avoidance of doubt, it is to be understood that for a compound of formula (IA), the R³ substituent can be substituted on an “available carbon” of the relevant pyridine ring, and that a carbon is not an “available carbon” where a hydrogen substituent is explicitly shown. Therefore, in the compound of formula (IA), there are only two positions of the relevant pyridine ring where R³ may be substituted. The same convention applies to the explicitly shown hydrogen atoms on the phenyl ring of formula (IB).

In one embodiment R¹ is hydrogen.

R² is halo, carboxy or a group selected from C₁₋₆alkyl, C₁₋₆alkoxycarbonyl, N—(C₁₋₆alkyl)carbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;

R²¹ is halo, cyano, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, carbocyclyl-, heterocyclyl- and (C₁₋₆alkyl)-S(O)ₐ— wherein a is 0 to 2; and

R²² is C₁₋₆alkyl.

In a further embodiment:

R² is halo, carboxy or a group selected from C₁₋₆alkyl, C₁₋₆alkoxycarbonyl, N—(C₁₋₆alkyl)carbamoyl, carbocyclyl- and heterocyclyl-; wherein said group may be independently optionally substituted on carbon by one or more R²¹; and wherein if said heterocyclyl contains an —NH— moiety that nitrogen may be optionally substituted by R²²;

R²¹ is halo, cyano, carbamoyl or a group selected from C₁₋₆alkyl, C₁₋₆alkoxy, N—(C₁₋₆alkanoyl)amino, carbocyclyl- and heterocyclyl; and

R²² is C₁₋₆alkyl.

In a further embodiment:

R² is hydrogen halo, carboxy or a group selected from C₁₋₆alkyl, C₁₋₆alkoxycarbonyl, N—(C₁₋₆alkyl)carbamoyl, carbocyclyl- and heterocyclyl-; wherein said group may be independently optionally substituted on carbon by one or more R²¹; and wherein if said heterocyclyl contains an —NH— moiety that nitrogen may be optionally substituted by R²²;

R²¹ is halo, cyano, carbamoyl or a group selected from C₁₋₆alkyl, C₁₋₆alkoxy, N—(C₁₋₆alkanoyl)amino, carbocyclyl- and heterocyclyl; and

R²² is C₁₋₆alkyl.

In a further embodiment R² is chloro, carboxy or a group selected from methyl, propyl, methoxycarbonyl, methylcarbamoyl, ethylcarbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;

R²¹ is fluoro, chloro, cyano, carbamoyl or a group selected from methyl, methoxy, acetylamino, cyclopropyl, phenyl and pyrrolyl; and

R²² is methyl.

In a further embodiment R² is pyridin-2-yl, pyridin-3-yl, pyrimidin-5-yl, 6-methylpyridin-2-yl, 1-methyl-1H-pyrazol-4-yl, methyl, propyl, 4,5-dimethyl-1,3-thiazol-2-yl, chloro, phenyl, 2-cyanophenyl, 4-(acetylamino)phenyl, 4-fluorophenyl, 3-chlorophenyl, 3-fluorophenyl, 3-methylphenyl, 3-methoxyphenyl, 4-methoxyphenyl, 4-methylthien-3-yl, pyrazin-2-yl, methoxycarbonyl, carboxy, N-(cyclopropylmethyl)carbamoyl, N-(1H-pyrrol-2-ylmethyl)carbamoyl, N-(phenylethyl)carbamoyl, N-benzylcarbamoyl, N-methylcarbamoyl, furan-2-yl, pyrrolidin-1-yl, tetrahydrofuran-2-yl, 6-methoxypyridin-3-yl, quinolin-4-yl or 2-carbamoylphenyl.

In a further embodiment of the invention R² is hydrogen, pyridin-2-yl, pyridin-3-yl, pyrimidin-5-yl, 6-methylpyridin-2-yl, 1-methyl-1H-pyrazol-4-yl, methyl, propyl, 4,5-dimethyl-1,3-thiazol-2-yl, chloro, phenyl, 2-cyanophenyl, 4-(acetylamino)phenyl, 4-fluorophenyl, 3-chlorophenyl, 3-fluorophenyl, 3-methylphenyl, 3-methoxyphenyl, 4-methoxyphenyl, 4-methylthien-3-yl, pyrazin-2-yl, methoxycarbonyl, carboxy, N-(cyclopropylmethyl)carbamoyl, N-(1H-pyrrol-2-ylmethyl)carbamoyl, N-(phenylethyl)carbamoyl, N-benzylcarbamoyl, N-methylcarbamoyl, furan-2-yl, pyrrolidin-1-yl, tetrahydrofuran-2-yl, 6-methoxypyridin-3-yl, quinolin-4-yl or 2-carbamoylphenyl.

In a further embodiment R² is hydrogen, halo or a group selected from C₁₋₆alkyl, carbocyclyl- and heterocyclyl- wherein said group may be independently optionally substituted on carbon by one or more R²¹; and wherein if said heterocyclyl contains an —NH— moiety that nitrogen may be optionally substituted by R²²;

R²¹ is halo, cyano or a group selected from C₁₋₆alkyl, C₁₋₆alkoxy and N—(C₁₋₆alkanoyl)amino;

R²² is C₁₋₆alkyl.

In a further embodiment R² is hydrogen, chloro or a group selected from methyl, phenyl, pyridinyl, thiazolyl, thienyl, pyrazinyl and pyrazolyl; wherein said group may be optionally substituted on carbon by one or more R²¹; and wherein if said heterocyclyl contains an —NH— moiety that nitrogen may be optionally substituted by R²²;

R²¹ is chloro, fluoro, cyano, or a group selected from methyl, methoxy and acetylamino; and

R²² is methyl.

In a further embodiment R² is hydrogen, chloro or a group selected from methyl, phenyl, pyridinyl, thiazolyl, thienyl, pyrazinyl and pyrazolyl; wherein said group may be optionally substituted on carbon by one or more R²¹; and wherein if said pyrazolyl contains an —NH— moiety that nitrogen may be optionally substituted by R²²;

R²¹ is chloro, fluoro, cyano, or a group selected from methyl, methoxy and acetylamino; and

R²² is methyl.

In a further embodiment R² is hydrogen, chloro, methyl, phenyl, 2-cyanophenyl, 4-fluorophenyl, 3-fluorophenyl, 3-chlorophenyl, 3-methylphenyl, 3-methoxyphenyl, 4-acetylaminophenyl, pyrazin-2-yl, 4-methylthien-3-yl, 6-methylpyridin-2-yl, 4,5-dimethylthiazol-2-yl, 1-methylpyrazol-4-yl or pyridin-2-yl.

In a further embodiment R² is a group selected from C₁₋₆alkyl and heterocyclyl.

In a further embodiment R² is methyl, propyl or pyridin-2-yl.

In a further embodiment R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2.

In a further embodiment n is 1.

In a further embodiment n is 2 wherein the values of R³ may be the same or different.

In a further embodiment R³ is independently C₁₋₃alkyl.

In a further embodiment n is 0 to 2; wherein the values of R³ may be the same or different.

In a further embodiment R³ are independently methyl or ethyl.

In a further embodiment R³ is independently C₁₋₃alkyl and n is 2 wherein the values of R³ may be the same or different.

In a further embodiment R³ is C₁₋₃alkyl and n is 1 or 2 wherein the values of R³ may be the same or different.

In a further embodiment R³ is methyl or ethyl and n is 1 or 2 wherein the values of R³ may be the same or different.

In a further embodiment R³ is C₁₋₃alkyl and n is 1.

In a further embodiment R³ is methyl and n is 2.

In a further embodiment R³ is methyl and n is 1.

In one embodiment Ring A is phenyl or a heterocyclic group, wherein the heterocyclic group is a fully unsaturated monocyclic ring containing 5 or 6 ring atoms of which at least one ring atom is chosen from nitrogen, sulfur or oxygen, which is linked via a carbon; wherein said phenyl or heterocyclic group may be optionally substituted on one or more carbons by R³³.

In a further embodiment Ring A is a phenyl or pyridinyl, wherein said phenyl or pyridinyl may be optionally substituted on carbon by one or more R³³.

In a further embodiment Ring A is phenyl which is substituted by one or more R³³.

In a further embodiment Ring A is phenyl which is substituted by one R³³.

R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹ )amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl is contains an —NH— moiety that nitrogen may be optionally substituted by R⁶⁰;

R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N'S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷— and carbocyclyl-R⁸⁸—;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ is selected from C₁₋₆alkyl, C₁₋₆alkanoyl and C₁₋₆alkylsulfonyl;

R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino;

R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl.

In a further embodiment:

R³³ is independently halo, cyano, hydroxy, carboxy, carbamoyl, sulfamoyl or a group selected from C₁₋₆alkyl, C₁₋₆alkoxy, C₁₋₆alkoxycarbonyl, N—(C₁₋₆alkanoyl)-N—(R³⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, carbocyclyl-R⁴¹—, heterocyclyl-R⁴²— and (C₁₋₆alkyl)-S(O)₂—; wherein said group may be optionally substituted on carbon by one or more R⁴³; and wherein if said heterocyclyl contains an —NH— moiety that nitrogen may be optionally substituted by R⁴⁴;

R⁴³ is halo, cyano, hydroxy, amino, carbamoyl, or a group selected from C₁₋₆alkyl, C₂₋₆alkynyl, C₁₋₆alkoxy, N—(C₁₋₆alkyl)amino, N—(C₁₋₆alkanoyl)-N—(R⁵¹)amino, N—(C₁₋₆alkoxycarbonyl)-N—(R⁵²)amino, N—(C₁₋₆alkyl)sulfamoyl, (R⁷⁹)(R⁸⁰)N—S(O)₂—N(R⁸¹)—, heterocyclyl-R⁵⁸— and (C₁₋₆alkyl)-S(O)₂—; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from C₁₋₆alkyl, C₁₋₆alkanoyl, C₁₋₆alkylsulfonyl, C₁₋₆alkoxycarbonyl, N—(C₁₋₆alkyl)carbamoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, C₁₋₆alkoxy, amino, carbamoyl, N—(C₁₋₆alkyl)carbamoyl, N—(C₁₋₆alkoxycarbonyl)-N—(R⁸⁶)-amino, heterocyclyl-R⁸⁷—, carbocyclyl-R⁸⁸—;

R⁸², R⁸³, R⁸⁷ and R⁸⁸ are each independently selected from a direct bond, —C(O)—, —N(R⁹⁰)C(O)— and —S(O)₂—;

R⁸⁵ is C₁₋₆alkyl;

R⁴¹, R⁴² and R⁵⁸ are independently selected from a direct bond, —C(O)—, —N(R⁷¹)C(O)—, —N(R⁷⁴)SO₂— and —S(O)ₐ— wherein a is 2;

R⁵⁹ is fluoro,

R³⁵, R³⁷, R⁵¹, R⁵², R⁷¹, R⁷⁴, R⁷⁵, R⁷⁹, R⁸⁰, R⁸¹, R⁸⁶ and R⁹⁰ are independently selected from hydrogen, C₁₋₃alkyl and cyclopropyl.

In a further embodiment:

R³³ is independently fluoro, chloro, cyano, hydroxy, carboxy, carbamoyl, sulfamoyl or a group selected from methyl, methoxy, ethoxy, propoxy, methoxycarbonyl, ethoxycarbonyl, N-acetyl-N—(R³⁵)amino, methylcarbamoyl, ethylcarbamoyl, propylcarbamoyl, isopropylcarbamoyl, dimethylcarbamoyl, methylsulfamoyl, ethylsulfamoyl, propylsulfamoyl, isopropylsulfamoyl, pentylsulfamoyl, N-ethyl-N-methyl-sulfamoyl, N,N-diethylsulfamoyl, N,N-dimethylsulfamoyl, N-(acetyl)sulfamoyl, N-(methylsulfonyl)amino, cyclopropyl-R⁴¹—, cyclobutyl-R⁴¹—, phenyl-R⁴¹—, morpholinyl-R⁴²—, piperazinyl-R⁴²— piperdinyl-R⁴²—, tetrahydropyranyl-R⁴²—, azetidinyl-R⁴²—, pyrrolidinyl-R⁴²—, oxazolyl-R⁴²—, azepanyl-R⁴²—, pyridinyl-R⁴²—, 2-oxopyrrolidinyl-R⁴²— and methylsulfonyl; wherein said group may be optionally substituted on carbon by one or more R⁴³; and wherein if said heterocyclyl contains an —NH— moiety that nitrogen may be optionally substituted by R⁴⁴;

R⁴³ is chloro, cyano, hydroxy, amino, carbamoyl, or a group selected from methyl, ethynyl, methoxy, isopropylamino, acetylamino, methylsulfamoyl, N-(tert-butoxycarbonyl)amino, 3,3-dimethyl-2,2-dioxido-2λ⁶-diazathianyl, pyrazinyl-R⁵⁸—, piperazinyl-R⁵⁸—, morpholinyl-R⁵⁸—, pyridinyl-R⁵⁸—, 1,1-dioxidothiomorpholinyl-R⁵⁸—, piperidinyl-R⁵⁸—, imidazolyl-R⁵⁸—, pyrazolyl-R⁵⁸—, pyrrolidinyl-R⁵⁸—, pyrrolyl-R^{58—, 8}-oxa-3-azabicyclo[3.2.1]octanyl-R⁵⁸— and methylsulfonyl; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from methyl, ethyl, ethanoyl, propanoyl, methylsulfonyl, tert-butoxycarbonyl, pentylcarbamoyl, adamantyl-R⁸²—, cyclohexyl-R⁸²—, phenyl-R⁸²—, cyclopropyl-R⁸²—, pyridinyl-R⁸³—, pyrrolyl-R⁸³—, and tetrahydropyranyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from chloro, fluoro, methoxy, amino, carbamoyl, methylcarbamoyl, N-(tert-butoxycarbonyl)amino, triazolyl-R⁸⁷—, 2-oxoimidazolidinyl-R⁸⁷— and cyclopropyl-R⁸⁸—;

R⁸², R⁸³, R⁸⁷ and R⁸⁸ are each independently selected from a direct bond, —C(O)—, —NH—C(O)—, and —S(O)₂—;

R⁸⁵ is methyl;

R⁴¹, R⁴² and R⁵⁸ are each independently selected from a direct bond, —C(O)—, —N(R⁷¹)C(O)—, —NH—SO₂— and —S(O)₂—;

R⁵⁹ is fluoro,

R³⁵ is hydrogen or methyl; and

R⁷¹ is hydrogen or cyclopropyl.

In a further embodiment, R³³ is independently methoxy, fluoro, morpholin-4-yl, chloro, methylsulfonyl, acetylamino, methyl, 4-methylpiperazin-1-yl, cyclopropyl(1-methylpiperidin-4-yl)carbamoyl, cyclopropyl(oxan-4-yl)carbamoyl, azetidin-1-ylcarbonyl, dimethylcarbamoyl, (1H-imidazol-2-ylmethyl)carbamoyl, (pyrazin-2-ylmethyl)-carbamoyl, (3-acetamidopyrrolidin-1-yl)carbonyl, [3-(methylsulfonyl)pyrrolidin-1-yl]carbonyl, (2-amino-2-oxoethyl)carbamoyl, (3-carbamoylpiperidin-1-yl)carbonyl, [4-(2-amino-2-oxoethyl)piperazin-1-yl]carbonyl, (1-methylpiperidin-4-yl)carbamoyl, (4-methylpiperazin-1-yl)carbonyl, cyclopropylcarbamoyl, cyanomethyl, 2-hydroxy-3-[(1-methylethyl)amino]propoxy, (methylsulfonyl)amino, 1,3-oxazol-5-yl, (4-methylpiperazin-1-yl)methyl, 2-morpholin-4-ylethoxy, (methylsulfamoyl)methyl, carbamoyl, acetyl(methyl)amino, hydroxymethyl, sulfamoyl, cyano, methoxy, (pyridin-2-ylmethyl)carbamoyl, (pyridin-4-ylmethyl)carbamoyl, 3-methylpiperidin-1-ylcarbonyl, azepan-1-ylcarbonyl, 1,1-dimethylprop-2-yn-1-yl, pyridin-2-ylsulfamoyl, acetylsulfamoyl, methylsulfamoyl, 3-methoxypropylsulfamoyl, 2-methoxyethylsulfamoyl, phenylsulfamoyl, (2-hydroxyethyl)(methyl)sulfamoyl, cyclobutylsulfamoyl, pentylsulfamoyl, piperidin-1-ylsulfonyl, diethylsulfamoyl, dimethylsulfamoyl, (2-methoxy-1-methylethyl)sulfamoyl, morpholin-4-ylsulfonyl, hydroxy, 2-morpholin-4-ylethyl)sulfamoyl, [2-(1,1-dioxidothiomorpholin-4-yl)ethyl]sulfamoyl, [2-(4-methylpiperazin-1-yl)ethyl]sulfamoyl, (2-piperidin-1-ylethyl)sulfamoyl, [2-( 1H-imidazol-1-yl)ethyl]sulfamoyl, [2-(1H-pyrazol-1-yl)ethyl]sulfamoyl, {2-[(3S)-3-fluoropyrrolidin-1-yl]ethyl}sulfamoyl, [(3R)-3-(methylsulfonyl)pyrrolidin-1-yl]carbonyl, [(3S)-3-(methylsulfonyl)pyrrolidin-1-yl]carbonyl, [2-(1H-pyrazol-1-yl)ethyl]carbamoyl, [2-(2-oxopyrrolidin-1-yl)ethyl]carbamoyl, {2-[(dimethylsulfamoyl)amino]ethyl}carbamoyl, methoxycarbonyl, [4-(methylsulfonyl)piperazin-1-yl]methyl, {4-[2-(methylamino)-2-oxoethyl]piperazin-1-yl}methyl, [4-(adamantylcarbamoyl)piperazin-1-yl]methyl, [4-(cyclohexylcarbamoyl)piperazin-1-yl]methyl, {4-[(4-methoxyphenyl)carbamoyl]-piperazin-1-yl}methyl, {4-[(3-chloro-4-fluorophenyl)carbamoyl]piperazin-1-yl}methyl, [4-(pentylcarbamoyl)piperazin-1-yl]methyl, (4-{[2-(1H-1,2,4-triazol-1-yl)pyridin-3-yl]sulfonyl}piperazin-1-yl)methyl, [4-(cyclopropylcarbonyl)piperazin-1-yl]methyl, {4-[N-(tert-butoxycarbonyl)-o-alanyl]piperazin-1-yl}methyl, {4-[(1-methyl-1H-pyrrol-3-yl)carbonyl]piperazin-1-yl}methyl, [4-(tetrahydro-2H-pyran-4-ylcarbonyl)piperazin-1-yl]methyl, [4-(3-amino-3-oxopropanoyl)piperazin-1-yl]methyl, {4-[2-(cyclopropylamino)-2-oxoethyl]piperazin-1-yl}methyl, [4-(3-aminopropanoyl)piperazin-1-yl]methyl, [4-(2-amino-1-methyl-2-oxoethyl)piperazin-1-yl]methyl, {4-[2-(2-oxoimidazolidin-1-yl)ethyl]piperazin-1-yl}methyl, 2-[(tert-butoxycarbonyl)amino]ethoxy, {3-[(tert-butoxycarbonyl)amino]propyl}carbamoyl, {3-[(tert-butoxycarbonyl)amino]ethyl}carbamoyl, (3-aminopropyl)carbamoyl, (3-aminoethyl)carbamoyl, carboxy, (2-chloroethyl)sulfamoyl, ethoxycarbonyl, [4-(tert-butoxycarbonyl)piperazin-1-yl]methyl, chloromethyl, [2-(1,1-dioxidothiomorpholin-4-yl)ethyl]carbamoyl, [2-(1H-pyrrol-1-yl)ethyl]carbamoyl, (2-piperidin-1-ylethyl)carbamoyl, [2-(8-oxa-3-azabicyclo [3.2.1]oct-3-yl)ethyl]carbamoyl, [2-(4,4-difluoropiperidin-1-yl)ethyl]carbamoyl or (2-morpholin-4-ylethyl)carbamoyl.

In a further embodiment R³³ is selected from carbamoyl, sulfamoyl, hydroxymethyl, cyanomethyl, hydroxy, methoxy, piperazin-1-ylmethyl, 4-methylsulfonylpiperazin-1-ylmethyl, 3-methylsulfonylpyrrolidin-1-ylcarbonyl, carbamoylmethylcarbamoyl, 4-(cyclopropylcarbamoylmethyl)piperazin-1-ylmethyl, [4-(2-amino-1-methyl-2-oxoethyl)piperazin-1-yl]methyl and methylsulfonylamino.

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)-, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl and C₁₋₆alkanoyl;

R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, is N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷ )N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷— and carbocyclyl-R⁸⁸—;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ is selected from C₁₋₆alkyl, C₁₋₆alkanoyl and C₁₋₆alkylsulfonyl;

R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino;

R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl.

In one embodiment Ring A is phenyl or a heterocyclic group, wherein the heterocyclic group is a fully unsaturated monocyclic ring containing 5 or 6 ring atoms of which at least one ring atom is chosen from nitrogen, sulfur or oxygen, which is linked via a carbon; wherein said phenyl or heterocyclic group may be optionally substituted on one or more carbons by R³³; and if said heterocyclic group contains an —NH— moiety that nitrogen may be optionally substituted by R³⁴;

R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl and C₁₋₆alkanoyl;

R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and is R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R ¹⁰⁶)carbamimidoyl]-N—(R¹⁰⁷)amino, heterocyclyl-R⁸⁷— and carbocyclyl-R⁸⁸—;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ is selected from C₁₋₆alkyl, C₁₋₆alkanoyl and C₁₋₆alkylsulfonyl;

R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino;

R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl.

In a further embodiment Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, hydroxy, carboxy, carbamoyl, sulfamoyl or a group selected from C₁₋₆alkyl, C₁₋₆alkoxy, C₁₋₆alkoxycarbonyl, N—(C₁₋₆alkanoyl)-N—(R³⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, carbocyclyl-R⁴¹—, heterocyclyl-R⁴²— and (C₁₋₆alkyl)-S(O)₂—; wherein said group may be optionally substituted on carbon by one or more R⁴³; and wherein if said heterocyclyl contains an —NH— moiety that nitrogen may be optionally substituted by R⁴⁴;

R³⁴ is C₁₋₆alkyl;

R⁴³ is halo, cyano, hydroxy, amino, carbamoyl, or a group selected from C₁₋₆alkyl, C₂₋₆alkynyl, C₁₋₆alkoxy, N—(C₁₋₆alkyl)amino, N—(C₁₋₆alkanoyl)-N—(R⁵¹)amino, N—(C₁₋₆alkoxycarbonyl)-N—(R⁵²)amino, N—(C₁₋₆alkyl)sulfamoyl, (R⁷⁹)(R⁸⁰)N—S(O)₂—N(R⁸¹)—, heterocyclyl-R⁵⁸— and (C₁₋₆alkyl)-S(O)₂—; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from C₁₋₆alkyl, C₁₋₆alkanoyl, C₁₋₆alkylsulfonyl, C₁₋₆alkoxycarbonyl, N—(C₁₋₆alkyl)carbamoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, C₁₋₆alkoxy, amino, carbamoyl, N—(C₁₋₆alkyl)carbamoyl, N—(C₁₋₆alkoxycarbonyl)-N—(R⁸⁶)-amino, heterocyclyl-R⁸⁷—, carbocyclyl-R⁸⁸—;

R⁸², R⁸³, R⁸⁷ and R⁸⁸ are each independently selected from a direct bond, —C(O)—, —N(R⁹⁰)C(O)— and —S(O)₂—;

R⁸⁵ is C₁₋₆alkyl;

R⁴¹, R⁴² and R⁵⁸ are independently selected from a direct bond, —C(O)—, —N(R⁷¹)C(O)—, —N(R⁷⁴)SO₂— and —S(O)ₐ— wherein a is 2;

R⁵⁹ is fluoro,

R³⁵, R³⁷, R⁵¹, R⁵², R⁷¹, R⁷⁴, R⁷⁵, R⁷⁹, R⁸⁰, R⁸¹, R⁸⁶ and R⁹⁰ are independently selected from hydrogen, C₁₋₃alkyl and cyclopropyl.

In a further embodiment of the invention Ring A is phenyl or a heterocyclic group selected from pyridinyl, pyrazolyl, indolyl and 2,2-dioxido-1,3-dihydro-2-benzothienyl; wherein said phenyl and heterocyclic group may be optionally substituted on one or more carbons by R³³; and wherein if said heterocyclic group contains an —NH— moiety that is nitrogen may be optionally substituted by R³⁴;

R³³ is independently fluoro, chloro, cyano, hydroxy, carboxy, carbamoyl, sulfamoyl or a group selected from methyl, methoxy, ethoxy, propoxy, methoxycarbonyl, ethoxycarbonyl, N-acetyl-N—(R³⁵)amino, methylcarbamoyl, ethylcarbamoyl, propylcarbamoyl, isopropylcarbamoyl, dimethylcarbamoyl, methylsulfamoyl, ethylsulfamoyl, propylsulfamoyl, isopropylsulfamoyl, pentylsulfamoyl, N-ethyl-N-methyl-sulfamoyl, N,N-diethylsulfamoyl, N,N-dimethylsulfamoyl, N-(acetyl)sulfamoyl, N-(methylsulfonyl)amino, cyclopropyl-R⁴¹—, cyclobutyl-R⁴¹—, phenyl-R⁴¹—, morpholinyl-R⁴²—, piperazinyl-R⁴²— piperdinyl-R⁴²—, tetrahydropyranyl-R⁴²—, azetidinyl-R⁴²—, pyrrolidinyl-R⁴²—, oxazolyl-R⁴²—, azepanyl-R⁴²—, pyridinyl-R⁴²—, 2-oxopyrrolidinyl-R⁴²— and methylsulfonyl; wherein said group may be optionally substituted on carbon by one or more R⁴³; and wherein if said heterocyclyl contains an —NH— moiety that nitrogen may be optionally substituted by R⁴⁴;

R³⁴ is methyl or ethyl;

R⁴³ is chloro, cyano, hydroxy, amino, carbamoyl, or a group selected from methyl, ethynyl, methoxy, isopropylamino, acetylamino, methylsulfamoyl, N-(tert-butoxycarbonyl)amino, 3,3-dimethyl-2,2-dioxido-2λ⁶-diazathianyl, pyrazinyl-R⁵⁸—, piperazinyl-R⁵⁸—, morpholinyl-R⁵⁸—, pyridinyl-R⁵⁸—, 1,1-dioxidothiomorpholinyl-R⁵⁸—, piperidinyl-R⁵⁸—, imidazolyl-R⁵⁸—, pyrazolyl-R⁵⁸—, pyrrolidinyl-R⁵⁸—, pyrrolyl-R⁵⁸—, 8-oxa-3-azabicyclo[3.2.1]octanyl-R⁵⁸— and methylsulfonyl; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from methyl, ethyl, ethanoyl, propanoyl, methylsulfonyl, tert-butoxycarbonyl, pentylcarbamoyl, adamantyl-R⁸²—, cyclohexyl-R⁸²—, phenyl-R⁸²—, cyclopropyl-R⁸²—, pyridinyl-R⁸³—, pyrrolyl-R⁸³—, and tetrahydropyranyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from chloro, fluoro, methoxy, amino, carbamoyl, methylcarbamoyl, N-(tert-butoxycarbonyl)amino, triazolyl-R⁸⁷—, 2-oxoimidazolidinyl-R⁸⁷— and cyclopropyl-R⁸⁸—;

R⁸², R⁸³, R⁸⁷ and R⁸⁸ are each independently selected from a direct bond, —C(O)—, —NH—C(O)—, and —S(O)₂—;

R⁸⁵ is methyl;

R⁴¹, R⁴² and R⁵⁸ are each independently selected from a direct bond, —C(O)—, —N(R⁷¹)C(O)—, —NH-SO₂— and —S(O)₂—;

R⁵⁹ is fluoro,

R³⁵ is hydrogen or methyl; and

R⁷¹ is hydrogen or cyclopropyl.

In a further embodiment Ring A is phenyl or a heterocyclic group selected from pyridinyl, pyrazolyl, indolyl and 2,2-dioxido-1,3-dihydro-2-benzothienyl; wherein said phenyl and heterocyclic group may be optionally substituted on one or more carbons by R³³; and wherein if said heterocyclic group contains an —NH— moiety that nitrogen may be optionally substituted by R³⁴;

R³³ is independently methoxy, fluoro, morpholin-4-yl, chloro, methylsulfonyl, acetylamino, methyl, 4-methylpiperazin-1-yl, cyclopropyl(1-methylpiperidin-4-yl)carbamoyl, cyclopropyl(oxan-4-yl)carbamoyl, azetidin-1-ylcarbonyl, dimethylcarbamoyl, (1H-imidazol-2-ylmethyl)carbamoyl, (pyrazin-2-ylmethyl)-carbamoyl, (3-acetamidopyrrolidin-1-yl)carbonyl, [3-(methylsulfonyl)pyrrolidin-1-yl]carbonyl, (2-amino-2-oxoethyl)carbamoyl, (3-carbamoylpiperidin-1-yl)carbonyl, [4-(2-amino-2-oxoethyl)piperazin-1-yl]carbonyl, (1-methylpiperidin-4-yl)carbamoyl, (4-methylpiperazin-1-yl)carbonyl, cyclopropylcarbamoyl, cyanomethyl, 2-hydroxy-3-[(1-methylethyl)amino]propoxy, (methylsulfonyl)amino, 1,3-oxazol-5-yl, (4-methylpiperazin-1-yl)methyl, 2-morpholin-4-ylethoxy, (methylsulfamoyl)methyl, carbamoyl, acetyl(methyl)amino, hydroxymethyl, sulfamoyl, cyano, methoxy, (pyridin-2-ylmethyl)carbamoyl, (pyridin-4-ylmethyl)carbamoyl, 3-methylpiperidin-1-ylcarbonyl, azepan-1-ylcarbonyl, 1,1-dimethylprop-2-yn-1-yl, pyridin-2-ylsulfamoyl, acetylsulfamoyl, methylsulfamoyl, 3-methoxypropylsulfamoyl, 2-methoxyethylsulfamoyl, phenylsulfamoyl, (2-hydroxyethyl)(methyl)sulfamoyl, cyclobutylsulfamoyl, pentylsulfamoyl, piperidin-1-ylsulfonyl, diethylsulfamoyl, dimethylsulfamoyl, (2-methoxy-1-methylethyl)sulfamoyl, morpholin-4-ylsulfonyl, hydroxy, 2-morpholin-4-ylethyl)sulfamoyl, [2-(1,1-dioxidothiomorpholin-4-yl)ethyl]sulfamoyl, [2-(4-methylpiperazin-1-yl)ethyl]sulfamoyl, (2-piperidin-1-ylethyl)sulfamoyl, [2-(1H-imidazol-1-yl)ethyl]sulfamoyl, [2-(1H-pyrazol-1-yl)ethyl]sulfamoyl, {2-[(3S)-3-fluoropyrrolidin-1-yl]ethyl}sulfamoyl, [(3R)-3-(methylsulfonyl)pyrrolidin-1-yl]carbonyl, [(3S)-3-(methylsulfonyl)pyrrolidin-1-yl]carbonyl, [2-(1H-pyrazol-1-yl)ethyl]carbamoyl, [2-(2-oxopyrrolidin-1-yl)ethyl]carbamoyl, {2-[(dimethylsulfamoyl)amino]ethyl}carbamoyl, methoxycarbonyl, [4-(methylsulfonyl)piperazin-1-yl]methyl, {4-[2-(methylamino)-2-oxoethyl]piperazin-1-yl}methyl, [4-(adamantylcarbamoyl)piperazin-1-yl]methyl, [4-(cyclohexylcarbamoyl)piperazin-1-yl]methyl, {4-[(4-methoxyphenyl)carbamoyl]-piperazin-1-yl}methyl, {4-[(3-chloro-4-fluorophenyl)carbamoyl]piperazin-1-yl}methyl, [4-(pentylcarbamoyl)piperazin-1-yl]methyl, (4-{[2-(1H-1,2,4-triazol-1-yl)pyridin-3-yl]sulfonyl}piperazin-1-yl)methyl, [4-(cyclopropylcarbonyl)piperazin-1-yl]methyl, {4-[N-(tert-butoxycarbonyl)-β-alanyl]piperazin-1-yl}methyl, {4-[(1-methyl-1H-pyrrol-3-yl)carbonyl]piperazin-1-yl}methyl, [4-(tetrahydro-2H-pyran-4-ylcarbonyl)piperazin-1-yl]methyl, [4-(3-amino-3-oxopropanoyl)piperazin-1-yl]methyl, {4-[2-(cyclopropylamino)-2-oxoethyl]piperazin-1-yl}methyl, [4-(3-aminopropanoyl)piperazin-1-yl]methyl, [4-(2-amino-1-methyl-2-oxoethyl)piperazin-1-yl]methyl, {4-[2-(2-oxoimidazolidin-1-yl)ethyl]piperazin-1-yl}methyl, 2-[(tert-butoxycarbonyl)amino]ethoxy, {3-[(tert-butoxycarbonyl)amino]propyl}carbamoyl, {3-[(tert-butoxycarbonyl)amino]ethyl}carbamoyl, (3-aminopropyl)carbamoyl, (3-aminoethyl)carbamoyl, carboxy, (2-chloroethyl)sulfamoyl, ethoxycarbonyl, [4-(tert-butoxycarbonyl)piperazin-1-yl]methyl, chloromethyl, [2-(1,1-dioxidothiomorpholin-4-yl)ethyl]carbamoyl, [2-(1H-pyrrol-1-yl)ethyl]carbamoyl, (2-piperidin-1-ylethyl)carbamoyl, [2-(8-oxa-3-azabicyclo [3.2.1]oct-3-yl)ethyl]carbamoyl, [2-(4,4-difluoropiperidin-1-yl)ethyl]carbamoyl or (2-morpholin-4-ylethyl)carbamoyl; and

R³⁴ is methyl or ethyl.

In a further embodiment Ring A is phenyl or a pyridinyl, wherein said phenyl or pyridinyl may be optionally substituted on one or more carbons by R³³;

R³³ is independently hydroxy, carbamoyl, sulfamoyl or a group selected from C₁₋₆alkyl, C₁₋₆alkoxy, N—(C₁₋₆alkyl)carbamoyl, N—[(C₁₋₆alkyl)sulfonyl]amino, heterocyclyl-C(O)—; wherein said group may be optionally substituted on carbon by one or more R⁴³;

R⁴³ is independently cyano, hydroxy, carbamoyl, C₁₋₆alkylsulfonyl or heterocyclyl; wherein if said heterocyclyl contains an —NH— moiety that nitrogen may be optionally substituted by R⁶⁰;

R⁶⁰ is independently selected from C₁₋₆alkyl and C₁₋₆alkylsulfonyl, wherein R⁶⁰ is optionally independently substituted on carbon by one or more R⁸⁴;

R⁸⁴ is selected carbamoyl and carbocyclyl-NH—C(O)—.

In a further embodiment Ring A is phenyl or a pyridinyl, wherein said phenyl or pyridinyl may be optionally substituted on one or more carbons by R³³;

R³³ is independently hydroxy, carbamoyl, sulfamoyl or a group selected from methyl, methoxy, methylcarbamoyl, methylsulfonylamino and pyrrolidin-1-ylcarbonyl; wherein said group may be optionally substituted on carbon by one or more R⁴³;

R⁴³ is independently cyano, hydroxy, carbamoyl, methylsulfonyl or piperazin-1-yl; wherein if said piperazin-1-yl contains an —NH— moiety that nitrogen may be optionally substituted by R⁶⁰;

R⁶⁰ is independently selected from methyl, ethyl, and methylsulfonyl, wherein R⁶⁰ is optionally independently substituted on carbon by one or more R⁸⁴;

R⁸⁴ is selected carbamoyl and cyclopropylcarbamoyl In a further embodiment Ring A is selected from 4-sulfamoylphenyl, pyridin-2-yl, 4-(hydroxymethyl)phenyl, 4(cyanomethyl)phenyl, 4-carbamoylphenyl, 3-hydroxyphenyl, 3,4,5-trimethoxyphenyl, 3-(piperazin-1-ylmethyl)phenyl, 3-[4-(methylsulfonyl)piperazin-1-ylmethyl]phenyl, 3-{[3-(methylsulfonyl)pyrrolidin-1-yl]carbonyl}phenyl, 3-[(2-amino-2-oxoethyl)carbamoyl]phenyl, 3-({4-[2-(cyclopropylamino)-2-oxoethyl]piperazin-1-yl}methyl)phenyl, 3-{[4-(2-amino-1-methyl-2-oxoethyl)piperazin-1-yl]methyl}phenyl, 3-sulfamoylphenyl and 3-(methylsulfonylamino)phenyl.

In a further embodiment Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ are independently halo, cyano, sulfamoyl or a group selected from C₁₋₆alkyl, C₁₋₆alkoxy, N—(C₁₋₆alkanoyl)amino, N,N—(C₁₋₆alkyl)₂carbamoyl, heterocyclyl- and (C₁₋₆alkyl)-S(O)ₐ— wherein a is 0 to 2; wherein said group may be optionally substituted on carbon by one or more R⁴³ and wherein if said heterocyclyl contains an —NH— moiety that nitrogen may be optionally substituted by R⁴⁴;

R³⁴ is C₁₋₆alkyl;

R⁴³ is hydroxy; and

R⁴⁴ is C₁₋₆alkyl.

In a further embodiment Ring A is a carbocyclic group selected from phenyl, or a heterocyclic group selected from pyrazolyl, indolyl and pyridyl, wherein said heterocyclic or carbocyclic groups may be optionally substituted on one or more carbons by R³³;

R³³ is independently chloro, fluoro, cyano, sulfamoyl, or a group selected from methyl, methoxy, mesyl, N-acetylamino, N,N-dimethylcarbamoyl, morpholinyl, and piperazinyl; wherein said piperazinyl may be optionally substituted on nitrogen by R⁴⁴; and

R⁴⁴ is methyl.

In a further embodiment Ring A is selected from 3,4,5-trimethoxyphenyl, 4-fluorophenyl, 2-morpholinophenyl, 3-chlorophenyl, 4-mesylphenyl, 2-pyridyl, 3-pyridyl, 3-acetamido-4-methylphenyl, 2-methoxy-5-mesylphenyl, 3-methoxyphenyl, 2-methoxyphenyl, 4-(4-methylpiperazino)phenyl, 3,5-dimorpholinophenyl, 3-morpholinophenyl, 4-morpholinophenyl, phenyl, 4-methoxyphenyl, 4-sulfamoylphenyl, 4-(N,N-dimethylcarbamoyl)phenyl, 3-fluorophenyl, 4-chlorophenyl, 2,5-difluorophenyl, 2-ethylpyrazol-3-yl, 4-hydroxymethylphenyl, 4-cyanophenyl, indol-5-yl, 4-pyridyl, 2,4-difluorophenyl, 3-hydroxymethylphenyl, 1-methylpyrazol-3-yl, indol-6-yl and 3-cyanophenyl.

In a further aspect there is provided a compound of formula (I) wherein the compound of formula (I) is a compound of formula (IA):

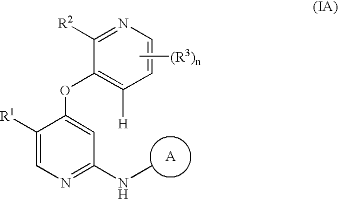

wherein:

R¹ is hydrogen, halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;

R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;

R² is halo, cyano, nitro, mercapto, sulfo, hydroxy, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N,N—(C₁₋₆alkyl)₂amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;

n is 0 to 2; wherein the values of R³ may be the same or different;

and when n is 1 or 2, R² is hydrogen or a value of R² as defined above;

R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, nitro, mercapto, sulfo, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₋₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R⁴⁸;

R⁴⁵ and R⁴⁶ are independently selected from a direct bond, —C(O)—, —C(═NH)—, —N(R¹⁰¹)—C(═NH)—, —N(R⁴⁹)C(O)—, —N(R⁵⁰)SO₂—, —O—C(O)— and —S(O)ₐ— wherein a is 1 or 2;

R⁴³ and R⁴⁷ are independently halo, cyano, nitro, mercapto, sulfo, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′-(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R²² and R³² are independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R⁴⁴, R⁴⁸ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, is N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴, R⁴⁸ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷—, carbocyclyl-R⁸⁸— and (C₁₋₆alkyl)-S(O)ₐ— wherein a is 0 to 2; and wherein if said heterocyclyl contains an —NH— moiety that nitrogen may be optionally substituted by R⁸⁹;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ and R⁸⁹ are each independently selected from C₁₋₆alkyl, C₁₋₆alkanoyl; C₁₋₆alkylsulfonyl;

R¹⁹ and R²⁰ are independently selected from a direct bond, —CH(R⁶¹)—, —CH(OR⁶²)—, —C(R⁶³)═C(R⁶⁴)—, ethynylene, —O—, —C(O)—, —N(R⁶⁶)C(O)—, —N(R⁶⁹)SO₂— and —S(O)ₐ— wherein a is 0 to 2;

R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R¹², R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, carbamimidoyl, carbamimidoylamino, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴, R⁵, R⁶, R⁷, R⁸, R⁹, R²³, R²⁴, R²⁵, R²⁶, R²⁷, R²⁸, R³⁵, R³⁶, R³⁷, R³⁸, R³⁹, R⁴⁰, R⁴⁹, R⁵⁰, R⁵¹, R⁵², R⁵³, R⁵⁴, R⁵⁵, R⁵⁶, R⁶¹, R⁶², R⁶³, R⁶⁴, R⁶⁶, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl;

or a pharmaceutically acceptable salt thereof

Therefore, in a further embodiment there is provided a compound of formula (I) (as depicted above), wherein:

R¹ is hydrogen;

R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2;

R² is halo, cyano, nitro, mercapto, sulfo, hydroxy, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N,N—(C₁₋₆alkyl)₂amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;

n is 0 to 2; wherein the values of R³ may be the same or different;

and when n is 1 or 2, R² is hydrogen or a value of R² as defined above;

is R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, nitro, mercapto, sulfo, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₋₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R⁴⁸;

R⁴⁵ and R⁴⁶ are independently selected from a direct bond, —C(O)—, —C(═NH)—, —N(R¹⁰¹)—C(═NH)—, —N(R⁴⁹)C(O)—, —N(R⁵⁰)SO₂—, —O—C(O)— and —S(O)ₐ— wherein a is 1 or 2;

R⁴³ and R⁴⁷ are independently halo, cyano, nitro, mercapto, sulfo, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴ )amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R²² and R³² are independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R⁴⁴, R⁴⁸ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴, R⁴⁸ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷ )N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷—, carbocyclyl-R⁸⁸— and (C₁₋₆alkyl)-S(O)ₐ— wherein a is 0 to 2; and wherein if said heterocyclyl contains an —NH— moiety that nitrogen may be optionally substituted by R⁸⁹;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ and R⁸⁹ are each independently selected from C₁₋₆alkyl, C₁₋₆alkanoyl; C₁₋₆alkylsulfonyl;

R¹⁹ and R²⁰ are independently selected from a direct bond, —CH(R⁶¹)—, —CH(OR⁶²)—, —C(R⁶³)═C(R⁶⁴)—, ethynylene, —O—, —C(O)—, —C(═NH)—, —N(R¹¹⁰)—C(═NH)—, —C(═NH)—N(R¹¹¹)—, —N(R⁶⁶)C(O)—, —N(R⁶⁹)SO₂— and —S(O)ₐ— wherein a is 0 to 2;

R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, carbamimidoyl, carbamimidoylamino, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴, R⁵, R⁶, R⁷, R⁸, R⁹, R²³, R²⁴, R²⁵, R²⁶, R²⁷, R²⁸, R³⁵, R³⁶, R³⁷, R³⁸, R³⁹, R⁴⁰, R⁴⁹, R⁵⁰, R⁵¹, R⁵², R⁵³, R⁵⁴, R⁵⁵, R⁵⁶, R⁶¹, R⁶², R⁶³, R⁶⁴, R⁶⁶, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl;

or a pharmaceutically acceptable salt thereof

In a further embodiment there is provided a compound of formula (I) wherein the compound of formula (I) is a compound of formula (IA) (as depicted above) wherein:

R¹ is hydrogen;

R³ is C₁₋₃alkyl;

n is 1 or 2 wherein the values of R³ may be the same or different;

R² is halo, carboxy or a group selected from C₁₋₆alkyl, C₁₋₆alkoxycarbonyl, N—(C₁₋₆alkyl)carbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;

R²¹ is halo, cyano, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, carbocyclyl-, heterocyclyl- and (C₁₋₆alkyl)-S(O)ₐ— wherein a is 0 to 2; and

R²² is C₁₋₆alkyl;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, is N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl and C₁₋₆alkanoyl;

R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R60;

R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷ )N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷— and carbocyclyl-R⁸⁸—;

R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ is selected from C₁₋₆alkyl, C₁₋₆alkanoyl and C₁₋₆alkylsulfonyl;

R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino;

R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹ , R¹¹⁰ and R¹¹¹ are independently selected from hydrogen, CI-₃alkyl and cyclopropyl;

or a pharmaceutically acceptable salt thereof.

Therefore, in a further embodiment there is provided a compound of formula (I) wherein the compound of formula (I) is a compound of formula (IA), as depicted above, wherein:

R¹ is hydrogen;

R² is halo, carboxy or a group selected from C₁₋₆alkyl, C₁₋₆alkoxycarbonyl, N—(C₁₋₆alkyl)carbamoyl, carbocyclyl- and heterocyclyl-; wherein said group may be independently optionally substituted on carbon by one or more R²¹; and wherein if said heterocyclyl contains an —NH— moiety that nitrogen may be optionally substituted by R²²;

R²¹ is halo, cyano, carbamoyl or a group selected from C₁₋₆alkyl, C₁₋₆alkoxy, N—(C₁₋₆alkanoyl)amino, carbocyclyl- and heterocyclyl; and

R²² is C₁₋₆alkyl;

n is 0 to 2; wherein the values of R³ may be the same or different;

R³ is independently C₁₋₃alkyl;

Ring A is a carbocyclic group or a heterocyclic group, wherein said heterocyclic is group or carbocyclic group may be optionally substituted on one or more carbons by R³³; and wherein if said heterocyclic group contains an —NH— moiety that nitrogen may be optionally substituted by R³⁴;

R³³ is independently halo, cyano, hydroxy, carboxy, carbamoyl, sulfamoyl or a group selected from C₁₋₆alkyl, C₁₋₆alkoxy, C₁₋₆alkoxycarbonyl, N—(C₁₋₆alkanoyl)-N—(R³⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, carbocyclyl-R⁴¹—, heterocyclyl-R⁴²— and (C₁₋₆alkyl)-S(O)₂—; wherein said group may be optionally substituted on carbon by one or more R⁴³; and wherein if said heterocyclyl contains an —NH— moiety that nitrogen may be optionally substituted by R⁴⁴;

R³⁴ is C₁₋₆alkyl;

R⁴³ is halo, cyano, hydroxy, amino, carbamoyl, or a group selected from C₁₋₆alkyl, C₂₋₆alkynyl, C₁₋₆alkoxy, N—(C₁₋₆alkyl)amino, N—(C₁₋₆alkanoyl)-N—(R⁵¹)amino, N—(C₁₋₆alkoxycarbonyl)-N—(R⁵²)amino, N—(C₁₋₆alkyl)sulfamoyl, (R⁷⁹)(R⁸⁰)N—S(O)₂—N(R⁸¹)—, heterocyclyl-R⁵⁸— and (C₁₋₆alkyl)-S(O)₂—; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from C₁₋₆alkyl, C₁₋₆alkanoyl, C₁₋₆alkylsulfonyl, C₁₋₆alkoxycarbonyl, N—(C₁₋₆alkyl)carbamoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, C₁₋₆alkoxy, amino, carbamoyl, N—(C₁₋₆alkyl)carbamoyl, N—(C₁₋₆alkoxycarbonyl)-N—(R⁸⁶)-amino, heterocyclyl-R⁸⁷—, carbocyclyl-R⁸⁸—;

R⁸², R⁸³, R⁸⁷ and R⁸⁸ are each independently selected from a direct bond, —C(O)—, —N(R⁹⁰)C(O)— and —SO₂—;

R⁸⁵ is C₁₋₆alkyl;

R⁴¹, R⁴² and R⁵⁸ are independently selected from a direct bond, —C(O)—, —N(R⁷¹)C(O)—, —N(R⁷⁴)SO₂— and —SO₂—;

R⁵⁹ is fluoro,

R³⁵, R³⁷, R⁵¹, R⁵², R⁷¹, R⁷⁴, R⁷⁵, R⁷⁹, R⁸⁰, R⁸¹, R⁸⁶ and R⁹⁰ are independently selected from hydrogen, C₁₋₃alkyl and cyclopropyl;

or a pharmaceutically acceptable salt thereof.

Therefore, in a further embodiment there is provided a compound of formula (I) wherein the compound of formula (I) is a compound of formula (IA), as depicted above, wherein:

R¹ is hydrogen;

R³ is methyl or ethyl and n is 1 or 2 wherein the values of R³ may be the same or different;

R² is hydrogen, chloro or a group selected from methyl, phenyl, pyridinyl, thiazolyl, thienyl, pyrazinyl and pyrazolyl; wherein said group may be optionally substituted on carbon by one or more R²¹; and wherein if said pyrazolyl contains an —NH— moiety that nitrogen may be optionally substituted by R²²;

R²¹ is chloro, fluoro, cyano, or a group selected from methyl, methoxy and acetylamino; and

R²² is methyl;

Ring A is phenyl or a heterocyclic group selected from pyridinyl, pyrazolyl, indolyl and 2,2-dioxido-1,3-dihydro-2-benzothienyl; wherein said phenyl and heterocyclic group may be optionally substituted on one or more carbons by R³³; and wherein if said heterocyclic group contains an —NH— moiety that nitrogen may be optionally substituted by R³⁴;

R³³ is independently fluoro, chloro, cyano, hydroxy, carboxy, carbamoyl, sulfamoyl or a group selected from methyl, methoxy, ethoxy, propoxy, methoxycarbonyl, ethoxycarbonyl, N-acetyl-N—(R³⁵)amino, methylcarbamoyl, ethylcarbamoyl, propylcarbamoyl, isopropylcarbamoyl, dimethylcarbamoyl, methylsulfamoyl, ethylsulfamoyl, propylsulfamoyl, isopropylsulfamoyl, pentylsulfamoyl, N-ethyl-N-methyl-sulfamoyl, N,N-diethylsulfamoyl, N,N-dimethylsulfamoyl, N-(acetyl)sulfamoyl, N-(methylsulfonyl)amino, cyclopropyl-R⁴¹—, cyclobutyl-R⁴¹—, phenyl-R⁴¹—, morpholinyl-R⁴²—, piperazinyl-R⁴²— piperdinyl-R⁴²—, tetrahydropyranyl-R⁴²—, azetidinyl-R⁴²—, pyrrolidinyl-R⁴²—, oxazolyl-R⁴²—, azepanyl-R⁴²—, pyridinyl-R⁴²—, 2-oxopyrrolidinyl-R⁴²— and methylsulfonyl; wherein said group may be optionally substituted on carbon by one or more R⁴³; and wherein if said heterocyclyl contains an —NH— moiety that nitrogen may be optionally substituted by R⁴⁴;

R³⁴ is methyl or ethyl;

R⁴³ is chloro, cyano, hydroxy, amino, carbamoyl, or a group selected from methyl, ethynyl, methoxy, isopropylamino, acetylamino, methylsulfamoyl, N-(tert-butoxycarbonyl)amino, 3,3-dimethyl-2,2-dioxido-2λ⁶-diazathianyl, pyrazinyl-R⁵⁸—, piperazinyl-R⁵⁸—, morpholinyl-R⁵⁸—, pyridinyl-R⁵⁸—, 1,1-dioxidothiomorpholinyl-R⁵⁸—, piperidinyl-R⁵⁸—, imidazolyl-R⁵⁸—, pyrazolyl-R⁵⁸—, pyrrolidinyl-R⁵⁸—, pyrrolyl-R⁵⁸—, 8-oxa-3-azabicyclo[3.2.1]octanyl-R⁵⁸— and methylsulfonyl; wherein said group may each be optionally independently substituted on carbon by one or more R⁵⁹; and wherein if said heterocyclyl contains an —NH— moiety that nitrogen may be optionally substituted by R⁶⁰;

R⁴⁴ and R⁶⁰ are independently selected from methyl, ethyl, ethanoyl, propanoyl, methylsulfonyl, tert-butoxycarbonyl, pentylcarbamoyl, adamantyl-R⁸²—, cyclohexyl-R⁸²—, phenyl-R⁸²—, cyclopropyl-R⁸²—, pyridinyl-R⁸³—, pyrrolyl-R⁸³—, and tetrahydropyranyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from chloro, fluoro, methoxy, amino, carbamoyl, methylcarbamoyl, N-(tert-butoxycarbonyl)amino, triazolyl-R⁸⁷—, 2-oxoimidazolidinyl-R⁸⁷— and cyclopropyl-R⁸⁸—;

R⁸², R⁸³, R⁸⁷ and R⁸⁸ are each independently selected from a direct bond, —C(O)—, —NH—C(O)—, and —S(O)₂—;

R⁸⁵ is methyl;

R⁴¹, R⁴² and R⁵⁸ are each independently selected from a direct bond, —C(O)—, —N(R⁷¹)C(O)—, —NH—SO₂— and —S(O)₂—;

R⁵⁹ is fluoro,

R³⁵ is hydrogen or methyl; and

R⁷¹ is hydrogen or cyclopropyl;

or a pharmaceutically acceptable salt thereof.

In a further embodiment there is provided a compound of formula (I), wherein the compound of formula (I) is a compound of formula (IB) (as shown above); wherein:

m is 1 to 3; wherein the values of R³³ may be the same of different;

R¹ is hydrogen;

R² is halo, carboxy or a group selected from C₁₋₆alkyl, C₁₋₆alkoxycarbonyl, N—(C₁₋₆alkyl)carbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;

R²¹ is halo, cyano, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, carbocyclyl-, heterocyclyl- and (C₁₋₆alkyl)-S(O)ₐ— wherein a is 0 to 2;

R²² is C₁₋₆alkyl;

R³ is independently C₁₋₃alkyl;

n is 0 to 2; wherein the values of R³ may be the same or different;

R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹ )amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R^{60;}

R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;

R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷ )N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′-(R¹⁰⁵)(R¹⁰⁶)carbamimidoyl]-N—(R¹⁰⁷)amino, heterocyclyl-R⁸⁷— and carbocyclyl-R⁸⁸—;

R⁸², R⁸³, R⁸⁷ and R⁸⁸ are each independently selected from a direct bond, —C(O)—, —C(═NH)—, —N(R¹¹⁰)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;

R⁸⁵ is selected from C₁₋₆alkyl, C₁₋₆alkanoyl and C₁₋₆alkylsulfonyl;

R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino;

R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl

or a pharmaceutically acceptable salt thereof.

In a further embodiment there is provided a compound of formula (I) wherein the compound of formula (I) is a compound of formula (IB), as depicted above, wherein:

m is 1 or 2; wherein the values of R³³ may be the same of different;

and the values of R², n, R¹, R³ and R³³ are as defined hereinbefore;

or a pharmaceutically acceptable salt thereof.

Therefore in a further aspect, there is provided a compound of formula (I) wherein the compound of formula (I) is a compound of formula (IB), as depicted above, wherein:

m is 0 to 3; wherein the values of R³³ may be the same or different;

n is 0 to 2; wherein the values of R³ may be the same or different;

R¹ is hydrogen;

R³ is independently methyl or ethyl;

R² is hydrogen, pyridin-2-yl, pyridin-3-yl, pyrimidin-5-yl, 6-methylpyridin-2-yl, 1-methyl-1H-pyrazol-4-yl, methyl, propyl, 4,5-dimethyl-1,3-thiazol-2-yl, chloro, phenyl, 2-cyanophenyl, 4-(acetylamino)phenyl, 4-fluorophenyl, 3-chlorophenyl, 3-fluorophenyl, 3-methylphenyl, 3-methoxyphenyl, 4-methoxyphenyl, 4-methylthien-3-yl, pyrazin-2-yl, methoxycarbonyl, carboxy, N-(cyclopropylmethyl)carbamoyl, N-(1H-pyrrol-2-ylmethyl)carbamoyl, N-(phenylethyl)carbamoyl, N-benzylcarbamoyl, N-methylcarbamoyl, furan-2-yl, pyrrolidin-1-yl, tetrahydrofuran-2-yl, 6-methoxypyridin-3-yl, quinolin-4-yl or 2-carbamoylphenyl;

R³³ is independently methoxy, fluoro, morpholin-4-yl, chloro, methylsulfonyl, acetylamino, methyl, 4-methylpiperazin-1-yl, cyclopropyl(1-methylpiperidin-4-yl)carbamoyl, cyclopropyl(oxan-4-yl)carbamoyl, azetidin-1-ylcarbonyl, dimethylcarbamoyl, (1H-imidazol-2-ylmethyl)carbamoyl, (pyrazin-2-ylmethyl)-carbamoyl, (3-acetamidopyrrolidin-1-yl)carbonyl, [3-(methylsulfonyl)pyrrolidin-1-yl]carbonyl, (2-amino-2-oxoethyl)carbamoyl, (3-carbamoylpiperidin-1-yl)carbonyl, [4-(2-amino-2-oxoethyl)piperazin-1-yl]carbonyl, (1-methylpiperidin-4-yl)carbamoyl, (4-methylpiperazin-1-yl)carbonyl, cyclopropylcarbamoyl, cyanomethyl, 2-hydroxy-3-[(1-methylethyl)amino]propoxy, (methylsulfonyl)amino, 1,3-oxazol-5-yl, (4-methylpiperazin-1-yl)methyl, 2-morpholin-4-ylethoxy, (methylsulfamoyl)methyl, carbamoyl, acetyl(methyl)amino, hydroxymethyl, sulfamoyl, cyano, methoxy, (pyridin-2-ylmethyl)carbamoyl, (pyridin-4-ylmethyl)carbamoyl, 3-methylpiperidin-1-ylcarbonyl, azepan-1-ylcarbonyl, 1,1-dimethylprop-2-yn-1-yl, pyridin-2-ylsulfamoyl, acetylsulfamoyl, methylsulfamoyl, 3-methoxypropylsulfamoyl, 2-methoxyethylsulfamoyl, phenylsulfamoyl, (2-hydroxyethyl)(methyl)sulfamoyl, cyclobutylsulfamoyl, pentylsulfamoyl, piperidin-1-ylsulfonyl, diethylsulfamoyl, dimethylsulfamoyl, (2-methoxy-1-methylethyl)sulfamoyl, morpholin-4-ylsulfonyl, hydroxy, 2-morpholin-4-ylethyl)sulfamoyl, [2-(1,1-dioxidothiomorpholin-4-yl)ethyl]sulfamoyl, [2-(4-methylpiperazin-1-yl)ethyl]sulfamoyl, (2-piperidin-1-ylethyl)sulfamoyl, [2-(1H-imidazol-1-yl)ethyl]sulfamoyl, [2-(1H-pyrazol-1-yl)ethyl]sulfamoyl, {2-[(3S)-3-fluoropyrrolidin-1-yl]ethyl}sulfamoyl, [(3R)-3-(methylsulfonyl)pyrrolidin-1-yl]carbonyl, [(3S)-3-(methylsulfonyl)pyrrolidin-1-yl]carbonyl, [2-(1H-pyrazol-1-yl)ethyl]carbamoyl, [2-(2-oxopyrrolidin-1-yl)ethyl]carbamoyl, {2-[(dimethylsulfamoyl)amino]ethyl}carbamoyl, methoxycarbonyl, [4-(methylsulfonyl)piperazin-1-yl]methyl, {4-[2-(methylamino)-2-oxoethyl]piperazin-1-yl}methyl, [4-(adamantylcarbamoyl)piperazin-1-yl]methyl, [4-(cyclohexylcarbamoyl)piperazin-1-yl]methyl, {4-[(4-methoxyphenyl)carbamoyl]-piperazin-1-yl}methyl, {4-[(3-chloro-4-fluorophenyl)carbamoyl]piperazin-1-yl}methyl, [4-(pentylcarbamoyl)piperazin-1-yl]methyl, (4-{[2-(1H-1,2,4-triazol-1-yl)pyridin-3-yl]sulfonyl}piperazin-1-yl)methyl, [4-(cyclopropylcarbonyl)piperazin-1-yl]methyl, {4-[N-(tert-butoxycarbonyl)-o-alanyl]piperazin-1-yl}methyl, {4-[(1-methyl-1H-pyrrol-3-yl)carbonyl]piperazin-1-yl}methyl, [4-(tetrahydro-2H-pyran-4-ylcarbonyl)piperazin-1-yl]methyl, [4-(3-amino-3-oxopropanoyl)piperazin-1-yl]methyl, {4-[2-(cyclopropylamino)-2-oxoethyl]piperazin-1-yl}methyl, [4-(3-aminopropanoyl)piperazin-1-yl]methyl, [4-(2-amino-1-methyl-2-oxoethyl)piperazin-1-yl]methyl, {4-[2-(2-oxoimidazolidin-1-yl)ethyl]piperazin-1-yl}methyl, 2-[(tert-butoxycarbonyl)amino]ethoxy, {3-[(tert-butoxycarbonyl)amino]propyl}carbamoyl, {3-[(tert-butoxycarbonyl)amino]ethyl}carbamoyl, (3-aminopropyl)carbamoyl, (3-aminoethyl)carbamoyl, carboxy, (2-chloroethyl)sulfamoyl, ethoxycarbonyl, [4-(tert-butoxycarbonyl)piperazin-1-yl]methyl, chloromethyl, [2-(1,1-dioxidothiomorpholin-4-yl)ethyl]carbamoyl, [2-(1H-pyrrol-1-yl)ethyl]carbamoyl, (2-piperidin-1-ylethyl)carbamoyl, [2-(8-oxa-3-azabicyclo[3.2.1]oct-3-yl)ethyl]carbamoyl, [2-(4,4-difluoropiperidin-1-yl)ethyl]carbamoyl or (2-morpholin-4-ylethyl)carbamoyl; or a pharmaceutically acceptable salt thereof.

In one aspect there is provided a compound of formula (I):

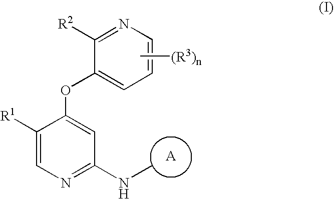

wherein:

R¹ and R³ are independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹ and (C₁₋₃alkyl)-S(O)ₐ— wherein a is 0 to 2; wherein said group may be independently optionally substituted on carbon by one or more R¹²;

R² is hydrogen, halo, cyano, nitro, mercapto, sulfo, hydroxy, carboxy, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R¹³)amino, N—(C₁₋₆alkoxycarbonyl)-N—(R¹⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R¹⁵)amino, 3,3-(R¹⁶)(R¹⁷)-1-(R¹³)ureido, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;

R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;

n is 0 to 3; wherein the values of R³ may be the same or different;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, nitro, mercapto, sulfo, hydroxy, carboxy, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R³⁷)amino, 3,3-(R³⁸)(R³⁹)-1-(R⁴⁰)ureido, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₋₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R^{48;}

R⁴⁵ and R⁴⁶ are independently selected from a direct bond, —C(O)—, —N(R⁴⁹)C(O)—, —N(R⁵⁰)SO₂—, —O—C(O)— and —S(O)ₐ— wherein a is 1 or 2;

R⁴³ and R⁴⁷ are independently halo, cyano, nitro, mercapto, sulfo, hydroxy, amino, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R²², R³², R⁴⁴, R⁴⁸ and R⁶⁰ are independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R¹⁹ and R²⁰ are independently selected from a direct bond, —CH(R⁶¹)—, —CH(OR⁶²)—, —C(R⁶³)═C(R⁶⁴)—, ethynylene, —O—, —N(R⁶⁵)—, —C(O)—, —N(R⁶⁶)C(O)—, —C(O)N(R⁶⁷)—, —SO₂N(R⁶⁸)—, —N(R⁶⁹)SO₂— and —S(O)ₐ— wherein a is 0 to 2;

R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R¹², R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴, R⁵, R⁶, R⁷, R⁸, R⁹, R¹³, R¹⁴, R¹⁵, R¹⁶, R¹⁷, R¹⁸, R²³, R²⁴, R²⁵, R²⁶, R²⁷, R²⁸, R³⁵, R³⁶, R³⁷, R³⁸, R³⁹, R⁴⁰, R⁴⁹, R⁵⁰, R⁵¹, R⁵², R⁵³, R⁵⁴, R⁵⁵, R⁵⁶, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³ and R⁷⁴ are independently selected from hydrogen, C₁₋₃alkyl and cyclopropyl;

or a pharmaceutically acceptable salt thereof.

In one embodiment of the invention, there is provided a compound of formula (I) as shown hereinbefore wherein:

R¹ is hydrogen, halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;

R³ are independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;

R² is hydrogen, halo, cyano, nitro, mercapto, sulfo, hydroxy, carboxy, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R¹³)amino, N—(C₁₋₆alkoxycarbonyl)-N—(R¹⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R¹⁵)amino, 3,3-(R¹⁶)(R¹⁷)-1-(R¹⁸)ureido, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;

R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;

n is 0 to 3; wherein the values of R³ may be the same or different;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;

R³³ is independently halo, cyano, nitro, mercapto, sulfo, hydroxy, carboxy, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R³⁷)amino, 3,3-(R³⁸)(R³⁹)-1-(R⁴⁰)ureido, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;

R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₋₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R^{48;}

R⁴⁵ and R⁴⁶ are independently selected from a direct bond, —C(O)—, —N(R⁴⁹)C(O)—, —N(R⁵⁰)SO₂—, —O—C(O)— and —S(O)ₐ— wherein a is 1 or 2;

R⁴³ and R⁴⁷ are independently halo, cyano, nitro, mercapto, sulfo, hydroxy, amino, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;

R²², R³², R⁴⁴, R⁴⁸ and R⁶⁰ are independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R¹⁹ and R²⁰ are independently selected from a direct bond, —CH(R⁶¹)—, —CH(OR⁶²)—, —C(R⁶³)═C(R⁶⁴)—, ethynylene, —O—, —N(R⁶⁵)—, —C(O)—, —N(R⁶⁶)C(O)—, —C(O)N(R⁶⁷)—, —SO₂N(R⁶⁸)—, —N(R⁶⁹)SO₂— and —S(O)ₐ— wherein a is 0 to 2;

R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —N(R⁷¹)C(O)—, —C(O)N(R⁷²)—, —SO₂N(R⁷³)—, —N(R⁷⁴)SO₂— and —S(O)ₐ— wherein a is 0 to 2;

R¹², R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴, R⁵, R⁶, R⁷, R⁸, R⁹, R¹³, R¹⁴, R¹⁵, R¹⁶, R¹⁷, R¹⁸, R²³, R²⁴, R²⁵, R²⁶, R²⁷, R²⁸, R³⁵, R³⁶, R³⁷, R³⁸, R³⁹, R⁴⁰, R⁴⁹, R⁵⁰, R⁵¹, R⁵², R⁵³, R⁵⁴, R⁵⁵, R⁵⁶, R⁶¹, R⁶², R⁶³, R⁶⁴, R⁶⁵, R⁶⁶, R⁶⁷ , R⁶⁸, R⁶⁹, R⁷⁰, R⁷¹, R⁷², R⁷³, and R⁷⁴ are independently selected from hydrogen, C₁₋₃alkyl and cyclopropyl;

or a pharmaceutically acceptable salt thereof.

In one embodiment there is provided a subset of compounds of formula (I) wherein:

R² is a group selected from C₁₋₆alkyl, carbocyclyl- and heterocyclyl- wherein said group may be independently optionally substituted on carbon by one or more R²¹; and wherein if said heterocyclyl contains an —NH— moiety that nitrogen may be optionally substituted by R²²;

R²¹ is halo, cyano or a group selected from C₁₋₆alkyl, C₁₋₆alkoxy and N—(C₁₋₆alkanoyl)amino; and

R²² is C₁₋₆alkyl.

Therefore, in a further embodiment of the invention, there is provided a compound of formula (I) wherein:

R¹ is hydrogen;

R² is hydrogen, halo or a group selected from C₁₋₆alkyl, carbocyclyl- and heterocyclyl- wherein said group may be independently optionally substituted on carbon by one or more R²¹; and wherein if said heterocyclyl contains an —NH— moiety that nitrogen may be optionally substituted by a group selected from R²²;

R²¹ is halo, cyano or a group selected from C₁₋₆alkyl, C₁₋₆alkoxy and N—(C₁₋₆alkanoyl)amino;

R²² is C₁₋₆alkyl;

R³ are independently C₁₋₃alkyl;

n is 0 to 2; wherein the values of R³ may be the same or different;

Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by a group selected from R³⁴;

R³³ is independently halo, cyano, sulfamoyl or a group selected from C₁₋₆alkyl, C₁₋₆alkoxy, N—(C₁₋₆alkanoyl)amino, N,N—(C₁₋₆alkyl)₂carbamoyl, heterocyclyl- and (C₁₋₆alkyl)-S(O)ₐ— wherein a is 0 to 2; wherein said group may be optionally substituted on carbon by one or more R⁴³ and wherein if said heterocyclyl contains an —NH— moiety that nitrogen may be optionally substituted by R⁴⁴;

R³⁴ is C₁₋₆alkyl;

R⁴³ is hydroxy; and

R⁴⁴ is C 1₆alkyl;

or a pharmaceutically acceptable salt thereof.

Furthermore, in a further embodiment of the invention there is provided a compound of formula (I) wherein:

R¹ is hydrogen;

R² is hydrogen, chloro, methyl, phenyl, 2-cyanophenyl, 4-fluorophenyl, 3-fluorophenyl, 3-chlorophenyl, 3-methylphenyl, 3-methoxyphenyl, 4-acetylaminophenyl, pyrazin-2-yl, 4-methylthien-3-yl, 6-methylpyridin-2-yl, 4,5-dimethylthiazol-2-yl, 1-methylpyrazol-4-yl or pyridin-2-yl;

R³ is independently methyl or ethyl;

n is 0 to 2; wherein the values of R³ may be the same or different; and

Ring A is selected from 3,4,5-trimethoxyphenyl, 4-fluorophenyl, 2-morpholinophenyl, 3-chlorophenyl, 4-mesylphenyl, 2-pyridyl, 3-pyridyl, 3-acetamido-4-methylphenyl, 2-methoxy-5-mesylphenyl, 3-methoxyphenyl, 2-methoxyphenyl, 4-(4-methylpiperazino)phenyl, 3,5-dimorpholinophenyl, 3-morpholinophenyl, 4-morpholinophenyl, phenyl, 4-methoxyphenyl, 4-sulfamoylphenyl, 4-(N,N-dimethylcarbamoyl)phenyl, 3-fluorophenyl, 4-chlorophenyl, 2,5-difluorophenyl, 2-ethylpyrazol-3-yl, 4-hydroxymethylphenyl, 4-cyanophenyl, indol-5-yl, 4-pyridyl, 2,4-difluorophenyl, 3-hydroxymethylphenyl, 1-methylpyrazol-3-yl, indol-6-yl and 3-cyanophenyl;

or a pharmaceutically acceptable salt thereof.

Therefore, in a further aspect there is provided a compound of formula (I) or a pharmaceutically acceptable salt thereof, wherein the compound of formula (I) is a compound of formula (IA), as depicted above, wherein:

R¹ is hydrogen;

R³ are each independently C₁₋₃alkyl;

R² is a group selected from C₁₋₆alkyl and heterocyclyl-

n is 1 or 2; wherein the values of R³ may be the same or different;

Ring A is phenyl or a pyridinyl, wherein said phenyl or pyridinyl may be optionally substituted on one or more carbons by R³³;

R³³ is independently hydroxy, carbamoyl, sulfamoyl or a group selected from C₁₋₆alkyl, C₁₋₆alkoxy, N—(C₁₋₆alkyl)carbamoyl, N—[(C₁₋₆alkyl)sulfonyl]amino, heterocyclyl-C(O)—; wherein said group may be optionally substituted on carbon by one or more R⁴³;

R⁴³ is independently cyano, hydroxy, carbamoyl, C₁₋₆alkylsulfonyl or heterocyclyl; wherein if said heterocyclyl contains an —NH— moiety that nitrogen may be optionally substituted by R⁶⁰;

R⁶⁰ is independently selected from C₁₋₆alkyl and C₁₋₆alkylsulfonyl, wherein R⁶⁰ is optionally independently substituted on carbon by one or more R⁸⁴;

R⁸⁴ is selected carbamoyl and carbocyclyl-NH—C(O)—.

In a further embodiment there is provided a compound of formula (I) or a pharmaceutically acceptable salt thereof, wherein the compound of formula (I) is a compound of formula (IA), as depicted above, wherein:

R¹ is hydrogen;

R³ is methyl or ethyl; and n is 1 or 2; wherein the values of n are the same or different;

R² is methyl, propyl or pyridin-2-yl;

Ring A is phenyl or a pyridinyl, wherein said phenyl or pyridinyl may be optionally substituted on one or more carbons by R³³;

R³³ is independently hydroxy, carbamoyl, sulfamoyl or a group selected from methyl, methoxy, methylcarbamoyl, methylsulfonylamino and pyrrolidin-1-ylcarbonyl; wherein said group may be optionally substituted on carbon by one or more R⁴³;

R⁴³ is independently cyano, hydroxy, carbamoyl, methylsulfonyl or piperazin-1-yl; wherein if said piperazin-1-yl contains an —NH— moiety that nitrogen may be optionally substituted by R⁶⁰;

R⁶⁰ is independently selected from methyl, ethyl and methylsulfonyl, wherein R⁶⁰ is optionally independently substituted on carbon by one or more R⁸⁴;

R⁸⁴ is selected carbamoyl and cyclopropylcarbamoyl.

In a further embodiment there is provided a compound of formula (I) or a pharmaceutically acceptable salt thereof, wherein the compound of formula (I) is a compound of formula (IB), as depicted above, wherein:

m is 1 to 3; wherein the values of R³³ are the same or different;

R¹ is hydrogen;

R³ is methyl or ethyl; and n is 1 or 2; wherein the values of n are the same or different;

R² is methyl, propyl or pyridin-2-yl;

R³³ is selected from carbamoyl, sulfamoyl, hydroxymethyl, cyanomethyl, hydroxy, methoxy, piperazin-1-ylmethyl, 4-methylsulfonylpiperazin-1-ylmethyl, 3-methylsulfonylpyrrolidin-1-ylcarbonyl, carbamoylmethylcarbamoyl, 4-(cyclopropylcarbamoylmethyl)piperazin-1-ylmethyl, [4-(2-amino-1-methyl-2-oxoethyl)piperazin-1-yl]methyl and methylsulfonylamino.

In a further embodiment there is provided a compound of formula (I) or a pharmaceutically acceptable salt thereof wherein the compound of formula (I) is a compound of formula (IA), as depicted above, wherein:

R¹ is hydrogen;

R³ is methyl or ethyl; and n is 1 or 2; wherein the values of n may be the same or different;

R² is methyl, propyl or pyridin-2-yl;

Ring A is selected from 4-sulfamoylphenyl, pyridin-2-yl, 4-(hydroxymethyl)phenyl, 4(cyanomethyl)phenyl, 4-carbamoylphenyl, 3-hydroxyphenyl, 3,4,5-trimethoxyphenyl, 3-(piperazin-1-ylmethyl)phenyl, 3-[4-(methylsulfonyl)piperazin-1-ylmethyl]phenyl, 3-{[3-(methylsulfonyl)pyrrolidin-1-yl]carbonyl}phenyl, 3-[(2-amino-2-oxoethyl)carbamoyl]phenyl, 3-( {4-[2-(cyclopropylamino)-2-oxoethyl]piperazin-1-yl}methyl)phenyl, 3-{[4-(2-amino-1-methyl-2-oxoethyl)piperazin-1-yl]methyl}phenyl, 3-sulfamoylphenyl and 3-(methylsulfonylamino)phenyl.

In another aspect of the invention, compounds of the invention are any one of the Examples or a pharmaceutically acceptable salt thereof.

In another embodiment there is provided a compound of formula (I) selected from: 4-[2-(6-methylpyridin-2-yl)pyridin-3-yl]oxy-N-(3,4,5-trimethoxyphenyl)pyridin-2-amine, 4-[5,6-dimethyl-2-(6-methylpyridin-2-yl)pyridin-3-yl]oxy-N-(3,4,5-trimethoxyphenyl)pyridin-2-amine, 4-[6-ethyl-2-(6-methylpyridin-2-yl)pyridin-3-yl]oxy-N-(3,4,5-trimethoxyphenyl)pyridin-2-amine, {4-[2-(4,5-dimethyl-thiazol-2-yl)-5,6-dimethyl-pyridin-3-yloxy]-pyridin-2-yl}-(3,4,5-trimethoxyphenyl)-amine, 4-[6-methyl-2-(1-methylpyrazol-4-yl)pyridin-3-yl]oxy-N-(3,4,5-trimethoxy-phenyl)pyridin-2-amine, 4-pyridin-3-yloxy-N-(3,4,5-trimethoxyphenyl)pyridin-2-amine, 4-(2,6-dimethylpyridin-3-yl)oxy-N-(3,4,5-trimethoxyphenyl)-pyridin-2-amine, 4-(6-methylpyridin-3-yl)oxy-N-(3,4,5-trimethoxyphenyl)-pyridin-2-amine, 4-(2-methylpyridin-3-yl)oxy-N-(3,4,5-trimethoxyphenyl)-pyridin-2-amine, 4-(5,6-dimethyl-2-pyridin-2-yl-pyridin-3-yl)oxy-N-(4-fluorophenyl)pyridin-2-amine, 4-(5,6-dimethyl-2-pyridin-2-yl-pyridin-3-yl)oxy-N-(2-morpholin-4-ylphenyl)pyridin-2-amine, N-(3-chlorophenyl)-4-(5,6-dimethyl-2-pyridin-2-yl-pyridin-3-yl)-oxy-pyridin-2-amine, 4-(5,6-dimethyl-2-pyridin-2-yl-pyridin-3-yl)oxy-N-(4-methylsulfonyl-phenyl)-pyridin-2-amine, 4-(5,6-dimethyl-2-pyridin-2-yl-pyridin-3-yl)oxy-N-pyridin-2-yl-pyridin-2-amine, 4-(5,6-dimethyl-2-pyridin-2-yl-pyridin-3-yl)oxy-N-pyridin-3-ylpyridin-2-amine, N-(5-{[4-(5,6-dimethyl-2-pyridin-2-yl-pyridin-3-yl)oxypyridin-2-yl]amino}-2-methylphenyl)acetamide, 4-(5,6-dimethyl-2-pyridin-2-yl-pyridin-3-yl)oxy-N-(2-methoxy-5-methylsulfonylphenyl)pyridin-2-amine, 4-(5,6-dimethyl-2-pyridin-2-yl-pyridin-3-yl)oxy-N-(3-methoxyphenyl)-pyridin-2-amine, 4-(5,6-dimethyl-2-pyridin-2-yl-pyridin-3-yl)oxy-N-(2-methoxyphenyl)-pyridin-2-amine, 4-(5,6-dimethyl-2-pyridin-2-ylpyridin-3-yl)oxy-N-[4-(4-methylpiperazin-1-yl)phenyl]pyridin-2-amine, 4-(5,6-dimethyl-2-pyridin-2-yl-pyridin-3-yl)oxy-N-(3,5-dimorpholin-4-ylphenyl)pyridin-2-amine, 4-(5,6-dimethyl-2-pyridin-2-yl-pyridin-3-yl)oxy-N-(3-morpholin-4-ylphenyl)pyridin-2-amine, 4-(5,6-dimethyl-2-pyridin-2-yl-pyridin-3-yl)oxy-N-(4-morpholin-4-ylphenyl)pyridin-2-amine, 4-(5,6-dimethyl-2-pyridin-2-yl-pyridin-3-yl)oxy-N-phenyl-pyridin-2-amine, 4-(5,6-dimethyl-2-pyridin-2-yl-pyridin-3-yl)oxy-N-(4-methoxyphenyl)-pyridin-2-amine, 4-{[4-(5,6-dimethyl-2-pyridin-2-yl-pyridin-3-yl)oxypyridin-2-yl]-amino}benzenesulfonamide, 4-{[4-(5,6-dimethyl-2-pyridin-2-yl-pyridin-3-yl)oxypyridin-2-yl]-amino}-N,N-dimethyl-benzamide, 4-(5,6-dimethyl-2-pyridin-2-yl-pyridin-3-yl)oxy-N-(3,4,5-tri-methoxy-phenyl)pyridin-2-amine, N-(3-fluorophenyl)-4-(5,6-dimethyl-2-pyridin-2-yl-pyridin-3-yl)-oxy-pyridin-2-amine, N-(4-chlorophenyl)-4-(5,6-dimethyl-2-pyridin-2-yl-pyridin-3-yl)oxy-pyridin-2-amine, N-(2,5-difluorophenyl)-4-(5,6-dimethyl-2-pyridin-2-yl-pyridin-3-yl)-oxy-pyridin-2-amine, 4-(5,6-dimethyl-2-pyridin-2-yl-pyridin-3-yl)oxy-N-(2-ethylpyrazol-3-yl)-pyridin-2-amine, (4-{[4-(5,6-dimethyl-2-pyridin-2-yl-pyridin-3-yl)-oxypyridin-2-yl]amino}phenyl)methanol, 4-{[4-(5,6-dimethyl-2-pyridin-2-yl-pyridin-3-yl)-oxypyridin-2-yl]amino}benzonitrile, N-[4-(5,6-dimethyl-2-pyridin-2-yl-pyridin-3-yl)-oxypyridin-2-yl]-1H-indol-5-amine, 4-(5,6-dimethyl-2-pyridin-2-yl-pyridin-3-yl)oxy-N-pyridin-4-yl-pyridin-2-amine, N-(2,4-difluorophenyl)-4-(5,6-dimethyl-2-pyridin-2-yl-pyridin-3-yl)oxy-pyridin-2-amine, (3-{[4-(5,6-dimethyl-2-pyridin-2-yl-pyridin-3-yl)-oxypyridin-2-yl]amino}phenyl)-methanol, 4-(5,6-dimethyl-2-pyridin-2-yl-pyridin-3-yl)oxy-N-(1-methylpyrazol-3-yl)-pyridin-2-amine; N-[4-(5,6-dimethyl-2-pyridin-2-yl-pyridin-3-yl)-oxypyridin-2-yl]-1H-indol-6-amine, 3-{[4-(5,6-dimethyl-2-pyridin-2-yl-pyridin-3-yl)oxypyridin-2-yl]-amino}benzonitrile, 4-({4-[(2,6-dimethylpyridin-3-yl)oxy]pyridin-2-yl}amino)-benzenesulfonamide, 4-{[4-(6-methyl-2-pyridin-2-yl-pyridin-3-yl)oxypyridin-2-yl]-amino}benzenesulfonamide, 4-{[4-(6-ethyl-2-pyridin-2-yl-pyridin-3-yl)oxypyridin-2-yl]-amino}benzene-sulfonamide, 4-(6-methyl-2-phenyl-pyridin-3-yl)oxy-N-(3,4,5-trimethoxyphenyl)pyridin-2-amine, 4-[6-methyl-2-(4-methylthiophen-3-yl)pyridin-3-yl]oxy-N-(3,4,5-trimethoxyphenyl)pyridin-2-amine, 4-(6-methyl-2-pyridin-2-yl-pyridin-3-yl)oxy-N-(3,4,5-trimethoxy-phenyl)pyridin-2-amine, 4-(6-methyl-2-pyrazin-2-yl-pyridin-3-yl)oxy-N-(3,4,5-trimethoxy-phenyl)pyridin-2-amine, 4-(2-phenylpyridin-3-yl)oxy-N-(3,4,5-trimethoxyphenyl)pyridin-2-amine, 2-(3-{2-[(3,4,5-trimethoxyphenyl)amino]pyridin-4-yl}-oxypyridin-2-yl)benzonitrile, N-[4-(3-{2-[(3 ,4,5-trimethoxyphenyl)amino]pyridin-4-yl}-oxypyridin-2-yl)phenyl]acetamide, 4-[2-(4-fluorophenyl)pyridin-3-yl]oxy-N-(3,4,5-trimethoxy-phenyl)pyridin-2-amine, 4-(2-chloropyridin-3-yl)oxy-N-(3,4,5-trimethoxyphenyl)pyridin-2-amine, 4-(2-chloro-6-methyl-pyridin-3-yl)oxy-N-(3,4,5-trimethoxyphenyl)-pyridin-2-amine, 4-[2-(3-chlorophenyl)-6-methyl-pyridin-3-yl]oxy-N-(3,4,5-trimethoxyphenyl)-pyridin-2-amine, 4-[2-(3-fluorophenyl)-6-methyl-pyridin-3-yl]oxy-N-(3,4,5-trimethoxy-phenyl)pyridin-2-amine, 4-[6-methyl-2-(3-methylphenyl)pyridin-3-yl]oxy-N-(3,4,5-trimethoxyphenyl)-pyridin-2-amine, 4-[2-(3-methoxyphenyl)-6-methyl-pyridin-3-yl]oxy-N-(3,4,5-trimethoxyphenyl)pyridin-2-amine, N-cyclopropyl-3-{[4-(2,6-dimethylpyridin-3-yl)oxypyridin-2-yl]-amino}-N-(1-methylpiperidin-4-yl)benzamide, N-cyclopropyl-3-{[4-(2,6-dimethylpyridin-3-yl)oxypyridin-2-yl]-amino}-N-(oxan-4-yl)benzamide, azetidin-1-yl-(3-{[4-(2,6-dimethylpyridin-3-yl)oxypyridin-2-yl]-amino}phenyl)methanone, 3-{[4-(2,6-dimethylpyridin-3-yl)oxypyridin-2-yl]amino}-N,N-dimethylbenzamide, 3-{[4-(2,6-dimethylpyridin-3-yl)oxypyridin-2-yl]amino}-N-(1H-imidazol-2-ylmethyl)benzamide, 3-{[4-(2,6-dimethylpyridin-3-yl)oxypyridin-2-yl]amino}-N-(pyrazin-2-ylmethyl)benzamide, N-[1-(3-{[4-(2,6-dimethylpyridin-3-yl)oxypyridin-2-yl]-amino}benzoyl)pyrrolidin-3-yl]acetamide, (3-{[4-(2,6-dimethylpyridin-3-yl)oxypyridin-2-yl]amino}phenyl)-(3-methylsulfonylpyrrolidin-1-yl)methanone, N-(2-amino-2-oxoethyl)-3-{[4-(2,6-dimethylpyridin-3-yl)-oxypyridin-2-yl]amino}benzamide, 1-(3-{[4-(2,6-dimethylpyridin-3-yl)oxypyridin-2-yl]amino}-benzoyl)piperidine-3-carboxamide, 2-[4-(3-{[4-(2,6-dimethylpyridin-3-yl)oxypyridin-2-yl]amino}-benzoyl)piperazin-1-yl]acetamide, 3-{[4-(2,6-dimethylpyridin-3-yl)oxypyridin-2-yl]amino}-N-( 1-methylpiperidin-4-yl)benzamide, [3-{[4-(2,6-dimethylpyridin-3-yl)oxypyridin-2-yl]amino}phenyl]-(4-methylpiperazin-1-yl)methanone, N-cyclopropyl-3-{[4-(5,6-dimethyl-2-pyridin-2-ylpyridin-3-yl)-oxypyridin-2-yl]amino}benzamide, N-(3-{[4-(5,6-dimethyl-2-pyridin-2-ylpyridin-3-yl)oxypyridin-2-yl]-amino}-4-methoxyphenyl)acetamide, 2-(4-{[4-(5,6-dimethyl-2-pyridin-2-ylpyridin-3-yl)oxypyridin-2-yl]-amino}phenyl)acetonitrile, 1-(4-{[4-(5,6-dimethyl-2-pyridin-2-ylpyridin-3-yl)oxypyridin-2-yl]-amino}phenoxy)-3-(propan-2-ylamino)propan-2-ol, N-(3-{[4-(5,6-dimethyl-2-pyridin-2-ylpyridin-3-yl)oxypyridin-2-yl]amino}phenyl)methanesulfonamide, 4-(5,6-dimethyl-2-pyridin-2-ylpyridin-3-yl)oxy-N-[3-(1,3-oxazol-5-yl)phenyl]pyridin-2-amine, 4-(5,6-dimethyl-2-pyridin-2-ylpyridin-3-yl)oxy-N-{3-[(4-methyl-piperazin-1-yl)methyl]phenyl}pyridin-2-amine, 4-(5,6-dimethyl-2-pyridin-2-ylpyridin-3-yl)oxy-N-[3-(2-morpholin-4-ylethoxy)phenyl]pyridin-2-amine, 1-(4-{[4-(5,6-dimethyl-2-pyridin-2-ylpyridin-3-yl)oxypyridin-2-yl]-amino}phenyl)-N-methylmethanesulfonamide, 4-{[4-(5,6-dimethyl-2-pyridin-2-ylpyridin-3-yl)oxypyridin-2-yl]amino}benzamide, methyl 3-(2-anilinopyridin-4-yl)oxy-6-methylpyridine-2-carboxylate, N-(4-{[4-(5,6-dimethyl-2-pyridin-2-ylpyridin-3-yl)oxypyridin-2-yl]amino}phenyl)-N-methylacetamide, 3-{[4-(5,6-dimethyl-2-pyridin-2-ylpyridin-3-yl)oxypyridin-2-yl]-amino}-5-methane-sulfonamidobenzamide, sodium 3-(2-anilinopyridin-4-yl)oxy-6-methylpyridine-2-carboxylate, N-(4-{[4-(5,6-dimethyl-2-pyridin-2-ylpyridin-3-yl)oxypyridin-2-yl]-amino}phenyl)acetamide, 3-{[4-(5,6-dimethyl-2-pyridin-2-ylpyridin-3-yl)oxypyridin-2-yl]amino}-5-(hydroxymethyl)benzenesulfonamide, 4-[(2,6-dimethylpyridin-3-yl)oxy]-N-(1-ethyl-1H-pyrazol-5-yl)pyridin-2-amine, 4-{[4-(2,6-dimethylpyridin-3-yl)oxypyridin-2-yl]amino}benzonitrile, 4-(2,6-dimethylpyridin-3-yl)oxy-N-(4-methoxyphenyl)pyridin-2-amine, 2-(4-{[4-(2,6-dimethylpyridin-3-yl)oxypyridin-2-yl]amino}phenyl)-acetonitrile, 4-{[4-(2,6-dimethylpyridin-3-yl)oxypyridin-2-yl]amino}benzamide, 3-(2-anilinopyridin-4-yl)oxy-N-(cyclopropylmethyl)-6-methyl-pyridine-2-carboxamide, 3-(2-anilinopyridin-4-yl)oxy-6-methyl-N-(1H-pyrrol-2-ylmethyl)-pyridine-2-carboxamide, 3-(2-anilinopyridin-4-yl)oxy-6-methyl-N-phenethylpyridine-2-carboxamide, 3-(2-anilinopyridin-4-yl)oxy-N-benzyl-6-methylpyridine-2-carboxamide, 3-(2-anilinopyridin-4-yl)oxy-N,6-dimethylpyridine-2-carboxamide, azetidin-1-yl-(3-{[4-(5,6-dimethyl-2-pyridin-2-ylpyridin-3-yl)-oxypyridin-2-yl]amino}phenyl)methanone, 3-{[4-(5,6-dimethyl-2-pyridin-2-ylpyridin-3-yl)oxypyridin-2-yl]-amino}-N-(pyridin-2-ylmethyl)benzamide, 3-{[4-(5,6-dimethyl-2-pyridin-2-ylpyridin-3-yl)oxypyridin-2-yl]-amino}-N-(pyridin-4-ylmethyl)benzamide, (3-{[4-(5,6-dimethyl-2-pyridin-2-ylpyridin-3-yl)oxypyridin-2-yl]amino}phenyl)-(3-methylpiperidin-1-yl)methanone, azepan-1-yl-(3-{[4-(5,6-dimethyl-2-pyridin-2-ylpyridin-3-yl)oxypyridin-2-yl]amino}phenyl)methanone, 3-{[4-(5,6-dimethyl-2-pyridin-2-ylpyridin-3-yl)oxypyridin-2-yl]-amino}-N-(2-methylbut-3-yn-2-yl)benzamide, 3-{[4-(5,6-dimethyl-2-pyridin-2-ylpyridin-3-yl)oxypyridin-2-yl]-amino}benzenesulfonamide, 3-{[4-(2,6-dimethylpyridin-3-yl)oxypyridin-2-yl]amino}benzene-sulfonamide, 4-[4-(2,6-dimethylpyridin-3-yloxy)pyridin-2-ylamino]-N-(pyridin-2-yl)benzenesulfonamide, N-{4-[4-(2,6-dimethylpyridin-3-yloxy)pyridin-2-ylamino]phenylsulfonyl}acetamide, 4-{[4-(2,6-dimethylpyridin-3-yl)oxypyridin-2-yl]amino}-N-methylbenzenesulfonamide, 4-{[4-(2,6-dimethylpyridin-3-yl)oxypyridin-2-yl]amino}-N-(3-methoxypropyl)-benzenesulfonamide, 4-{[4-(2,6-dimethylpyridin-3-yl)oxypyridin-2-yl]amino}-N-(2-methoxyethyl)benzenesulfonamide, 3-{[4-(2,6-dimethylpyridin-3-yl)oxypyridin-2-yl]amino}-N-phenylbenzenesulfonamide, 4-{[4-(2,6-dimethylpyridin-3-yl)oxypyridin-2-yl]amino}-N-(2-hydroxyethyl)-N-methylbenzenesulfonamide, N-cyclobutyl-4-{[4-(2,6-dimethylpyridin-3-yl)oxypyridin-2-yl]amino}benzene-sulfonamide, 4-{[4-(2,6-dimethylpyridin-3-yl)oxypyridin-2-yl]amino}-N-pentylbenzenesulfonamide, 4-(2,6-dimethylpyridin-3-yl)oxy-N-(4-piperidin-1-ylsulfonyl-phenyl)pyridin-2-amine, 4-{[4-(2,6-dimethylpyridin-3-yl)oxypyridin-2-yl]amino}-N,N-diethylbenzenesulfonamide, 4-{[4-(2,6-dimethylpyridin-3-yl)oxypyridin-2-yl]amino}-N,N-dimethylbenzenesulfonamide, 4-{[4-(2,6-dimethylpyridin-3-yl)oxypyridin-2-yl]amino}-N-(1-methoxypropan-2-yl)benzenesulfonamide, 4-(2,6-dimethylpyridin-3-yl)oxy-N-(3-morpholin-4-ylsulfonylphenyl)pyridin-2-amine, 4-(2,6-dimethylpyridin-3-yl)oxy-N-(2,2-dioxo-1,3-dihydro-2-benzothiophen-5-yl)pyridin-2-amine, 4-{[4-(2,6-dimethylpyridin-3-yl)oxypyridin-2-yl]amino}-N,N-dimethylbenzamide, N-(4-{[4-(2,6-dimethylpyridin-3-yl)oxypyridin-2-yl]amino}phenyl)-N-methylacetamide, N-(4-{[4-(2,6-dimethylpyridin-3-yl)oxypyridin-2-yl]amino}phenyl)acetamide, 4-(2,6-dimethylpyridin-3-yl)oxy-N-(4-methylsulfonylphenyl)-pyridin-2-amine, 3-{[4-(2,6-dimethylpyridin-3-yl)oxypyridin-2-yl]amino}benzonitrile, 3-{[4-(2,6-dimethylpyridin-3-yl)oxypyridin-2-yl]amino}phenol, N-(5-{[4-(2,6-dimethylpyridin-3-yl)oxypyridin-2-yl]amino}-2-methylphenyl)acetamide, N-(3-{[4-(2,6-dimethylpyridin-3-yl)oxypyridin-2-yl]amino}phenyl)methanesulfonamide, 1-(4-{[4-(2,6-dimethylpyridin-3-yl)oxypyridin-2-yl]amino}phenoxy)-3-(propan-2-ylamino)propan-2-ol, 4-[4-(2,6-dimethylpyridin-3-yloxy)pyridin-2-ylamino]-N-( 1-methylpiperidin-4-yl)benzamide, 4-[4-(2,6-dimethylpyridin-3-yloxy)pyridin-2-ylamino]-N-(2-morpholinoethyl)benzenesulfonamide, 4-{[4-(2,6-dimethylpyridin-3-yl)oxypyridin-2-yl]amino}-N-[2-(1,1-dioxo-1,4-thiazinan-4-yl)ethyl]-benzenesulfonamide, 4-{[4-(2,6-dimethylpyridin-3-yl)oxypyridin-2-yl]amino}-N-[2-(4-methylpiperazin-1-yl)ethyl]benzenesulfonamide, 4-{[4-(2,6-dimethylpyridin-3-yl)oxypyridin-2-yl]amino}-N-(2-piperidin-1-ylethyl)benzenesulfonamide, 4-{[4-(2,6-dimethylpyridin-3-yl)oxypyridin-2-yl]amino}-N-(2-imidazol-1-ylethyl)benzene-sulfonamide, 4-{[4-(2,6-dimethylpyridin-3-yl)oxypyridin-2-yl]amino}-N-(2-pyrazol-1-ylethyl)benzenesulfonamide, 4-{[4-(2,6-dimethylpyridin-3-yl)oxypyridin-2-yl]amino}-N-{2-[(3S)-3-fluoropyrrolidin-1-yl]ethyl}benzenesulfonamide, (S)-{3-[4-(2,6-dimethylpyridin-3-yloxy)pyridin-2-ylamino]phenyl}-[3-(methylsulfonyl)pyrrolidin-1-yl]methanone, (R)-{3-[4-(2,6-dimethylpyridin-3-yloxy)pyridin-2-ylamino]phenyl}-[3-(methylsulfonyl)pyrrolidin-1-yl]methanone, 4-{[4-(2,6-dimethylpyridin-3-yl)oxypyridin-2-yl]amino}-N-(2-pyrazol-1-ylethyl)benzamide, 4-{[4-(2,6-dimethylpyridin-3-yl)oxypyridin-2-yl]amino}-N-[2-(2-oxopyrrolidin-1-yl)ethyl]benzamide, 4-{[4-(2,6-dimethylpyridin-3-yl)oxypyridin-2-yl]amino}-N-[2-(dimethylsulfamoylamino)ethyl]benzamide, methyl 3-{[4-(2,6-dimethylpyridin-3-yl)oxypyridin-2-yl]-amino}benzoate, 2-(4-{[4-(6-methyl-2-pyridin-2-ylpyridin-3-yl)oxypyridin-2-yl]-amino}phenyl)acetonitrile, 4-(6-methyl-2-pyridin-2-ylpyridin-3-yl)oxy-N-pyridin-2-yl-pyridin-2-amine, 4-{[4-(6-methyl-2-pyridin-2-ylpyridin-3-yl)oxypyridin-2-yl]-amino}benzamide, (4-{[4-(6-methyl-2-pyridin-2-ylpyridin-3-yl)oxypyridin-2-yl]-amino}phenyl)methanol, 2-(4-{[4-(6-methyl-2-phenylpyridin-3-yl)oxypyridin-2-yl]-amino}phenyl)acetonitrile, 4-{[4-(6-methyl-2-phenylpyridin-3-yl)oxypyridin-2-yl]amino}benzenesulfonamide, 4-(6-methyl-2-phenylpyridin-3-yl)oxy-N-pyridin-2-ylpyridin-2-amine, 4-{[4-(6-methyl-2-phenylpyridin-3-yl)oxypyridin-2-yl]amino}benzamide, (4-{[4-(6-methyl-2-phenylpyridin-3-yl)oxypyridin-2-yl]amino}phenyl)methanol, 4-(2,6-dimethylpyridin-3-yl)oxy-N-pyridin-2-ylpyridin-2-amine, (4-{[4-(2,6-dimethylpyridin-3-yl)oxypyridin-2-yl]amino}phenyl)methanol, 2-[4-( {4-[2-(4-fluorophenyl)-6-methylpyridin-3-yl]oxypyridin-2-yl}amino)phenyl]acetonitrile, 4-( {4-[2-(4-fluorophenyl)-6-methylpyridin-3-yl]oxypyridin-2-yl}amino)benzenesulfonamide, 4-( {4-[2-(4-fluorophenyl)-6-methylpyridin-3-yl]oxypyridin-2-yl}-amino)benzamide, [4-( {4-[2-(4-fluorophenyl)-6-methylpyridin-3-yl]oxypyridin-2-yl}-amino)phenyl]-methanol, 2-[4-({4-[2-(furan-2-yl)-6-methylpyridin-3-yl]oxypyridin-2-yl}-amino)phenyl]acetonitrile, 4-({4-[2-(furan-2-yl)-6-methylpyridin-3-yl]oxypyridin-2-yl}-amino)benzenesulfonamide, 4-[2-(furan-2-yl)-6-methylpyridin-3-yl]oxy-N-pyridin-2-yl-pyridin-2-amine, 4-({4-[2-(furan-2-yl)-6-methylpyridin-3-yl]oxypyridin-2-yl}-amino)benzamide, [4-({4-[2-(furan-2-yl)-6-methylpyridin-3-yl]oxypyridin-2-yl}amino)phenyl]methanol, 4-(6-methyl-2-(pyrrolidin-1-yl)pyridin-3-yloxy)-N-(3,4,5-trimethoxyphenyl)pyridin-2-amine, 4-[2-(furan-2-yl)-6-methylpyridin-3-yloxy]-N-(3,4,5-trimethoxyphenyl)pyridin-2-amine, 4-(6-methyl-2-propylpyridin-3-yloxy)-N-(3,4,5-trimethoxyphenyl)pyridin-2-amine, 4-[6-methyl-2-(oxolan-2-yl)pyridin-3-yl]oxy-N-(3,4,5-trimethoxyphenyl)pyridin-2-amine, 4-[2-(4-methoxyphenyl)-6-methylpyridin-3-yl-oxy]-N-(3,4,5-trimethoxyphenyl)pyridin-2-amine,4-(6′-methoxy-6-methyl-2,3′-bipyridin-3-yloxy)-N-(3,4,5-trimethoxyphenyl)pyridin-2-amine, 4-[6-methyl-2-(quinolin-4-yl)pyridin-3-yloxy]-N-(3,4,5-trimethoxyphenyl)pyridin-2-amine, 4-[2-(4-fluorophenyl)-6-methylpyridin-3-yloxy]-N-(3,4,5-trimethoxyphenyl)pyridin-2-amine, 4-(6-methyl-2,3′-bipyridin-3-yloxy)-N-(3,4,5-trimethoxyphenyl)pyridin-2-amine, 4-(6-methyl-2-pyrimidin-5-ylpyridin-3-yl)oxy-N-(3,4,5-trimethoxyphenyl)pyridin-2-amine, (3-{[4-(2,6-dimethylpyridin-3-yl)oxypyridin-2-yl]amino}phenyl)methanol, 4-(2,6-dimethylpyridin-3-yl)oxy-N-{3-[(4-methylsulfonyl-piperazin-1-yl)methyl]phenyl}pyridin-2-amine, 4-(2,6-dimethylpyridin-3-yl)oxy-N-[3-(piperazin-1-ylmethyl)-phenyl]pyridin-2-amine, N-(2-adamantyl)-4-[(3-{[4-(2,6-dimethylpyridin-3-yl)oxypyridin-2-yl]amino}phenyl)-methyl]piperazine-1-carboxamide, N-cyclohexyl-4-{3-[4-(2,6-dimethylpyridin-3-yloxy)pyridin-2-ylamino]benzyl}piperazine-1-carboxamide, 4-[(3-{[4-(2,6-dimethylpyridin-3-yl)oxypyridin-2-yl]amino}phenyl)methyl]-N-(4-methoxyphenyl)piperazine-1-carboxamide, N-(3-chloro-4-fluorophenyl)-4-[(3-{[4-(2,6-dimethylpyridin-3-yl)oxypyridin-2-yl]amino}phenyl)methyl]piperazine-1-carboxamide, 4-[(3-{[4-(2,6-dimethylpyridin-3-yl)oxypyridin-2-yl]amino}phenyl)-methyl]-N-pentylpiperazine-1-carboxamide, 4-(2,6-dimethylpyridin-3-yl)oxy-N-[3-( {4-[2-(1,2,4-triazol-1-yl)-pyridin-3-yl]sulfonylpiperazin-1-yl}methyl)phenyl]-pyridin-2-amine, N-(3-{[4-(cyclopropylcarbonyl)piperazin-1-yl]methyl}phenyl)-4-[(2,6-dimethylpyridin-3-yl)oxy]pyridin-2-amine, {4-[(3-{[4-(2,6-dimethylpyridin-3-yl)oxypyridin-2-yl]-amino}phenyl)methyl]piperazin-1-yl}-(1-methylpyrrol-3-yl)methanone, {4-[(3-{[4-(2,6-dimethylpyridin-3-yl)oxypyridin-2-yl]amino}-phenyl)methyl]piperazin-1-yl}-(oxan-4-yl)methanone, 3-{4-[(3-{[4-(2,6-dimethylpyridin-3-yl)oxypyridin-2-yl]amino}phenyl)methyl]piperazin-1-yl}-3-oxopropanamide, N-cyclopropyl-2-{4-[(3-{[4-(2,6-dimethylpyridin-3-yl)oxypyridin-2-yl]amino}phenyl)methyl]piperazin-1-yl}acetamide, 3-amino-1-{4-[(3-{[4-(2,6-dimethylpyridin-3-yl)oxypyridin-2-yl]-amino}phenyl)methyl]piperazin-1-yl}propan-1-one, 2-{4-[3-({4-[(2,6-dimethylpyridin-3-yl)oxy]pyridin-2-yl}amino)-benzyl]-piperazin-1-yl}propanamide, 2-{4-[3-({4-[(2,6-dimethylpyridin-3-yl)oxy]pyridin-2-yl}amino)-benzyl]piperazin-1-yl}-N-methylacetamide, 1-(2-{4-[(3-{[4-(2,6-dimethylpyridin-3-yl)oxypyridin-2-yl]-amino}phenyl)methyl]piperazin-1-yl}ethyl)-imidazolidin-2-one, 4-(2,6-dimethylpyridin-3-yl)oxy-N-{3-[(4-methylpiperazin-1-yl)-methyl]phenyl}pyridin-2-amine, N-cyclopropyl-3-{[4-(2,6-dimethylpyridin-3-yl)oxypyridin-2-yl]-amino}benzamide, N-(2-chlorophenyl)-4-(5,6-dimethyl-2-pyridin-2-ylpyridin-3-yl)-oxypyridin-2-amine, tert-butyl N-[2-(4-{[4-(5,6-dimethyl-2-pyridin-2-ylpyridin-3-yl)-oxypyridin-2-yl]amino}phenoxy)ethyl]carbamate, tert-butyl N-[3-(4-{[4-(5,6-dimethyl-2-pyridin-2-ylpyridin-3-yl)oxypyridin-2-yl]amino}-phenoxy)propyl]carbamate, tert-butyl N-{3-[(4-{[4-(5,6-dimethyl-2-pyridin-2-ylpyridin-3-yl)-oxypyridin-2-yl]amino}benzoyl)amino]propyl}carbamate, tert-butyl N-{2-[(4-{[4-(5,6-dimethyl-2-pyridin-2-ylpyridin-3-yl)oxypyridin-2-yl]amino}-benzoyl)amino]ethyl}carbamate, N-(3-aminopropyl)-4-{[4-(5,6-dimethyl-2-pyridin-2-ylpyridin-3-yl)oxypyridin-2-yl]amino}benzamide, N-(2-aminoethyl)-4-{[4-(5,6-dimethyl-2-pyridin-2-ylpyridin-3-yl)oxypyridin-2-yl]amino}benzamide, 3-{[4-(5,6-dimethyl-2-pyridin-2-ylpyridin-3-yl)oxypyridin-2-yl]-amino}phenol, methyl 3-{[4-(5,6-dimethyl-2-pyridin-2-ylpyridin-3-yl)oxypyridin-2-yl]amino}benzoate, 3-{[4-(5,6-dimethyl-2-pyridin-2-ylpyridin-3-yl)oxypyridin-2-yl]-amino}benzoic acid, N-(2-chloroethyl)-4-[4-(2,6-dimethylpyridin-3-yloxy)pyridin-2-ylamino]benzene-sulfonamide, lithium 4-{[4-(2,6-dimethylpyridin-3-yl)oxypyridin-2-yl]-amino}-benzoate, ethyl 4-{[4-(2,6-dimethylpyridin-3-yl)oxypyridin-2-yl]amino}benzoate, tert-butyl 4-[(3-{[4-(2,6-dimethylpyridin-3-yl)oxypyridin-2-yl]-amino}phenyl)-methyl]piperazine-1-carboxylate, tert-butyl N-(3-{4-[(3-{[4-(2,6-dimethylpyridin-3-yl)oxypyridin-2-yl]amino}phenyl)methyl]piperazin-1-yl}-3-oxopropyl)carbamate, N-[3-(chloromethyl)phenyl]-4-(2,6-dimethylpyridin-3-yl)-oxypyridin-2-amine, sodium 3-{[4-(2,6-dimethylpyridin-3-yl)oxypyridin-2-yl]amino}benzoate, 4-{[4-(5,6-dimethyl-2-pyridin-2-ylpyridin-3-yl)oxypyridin-2-yl]amino}phenol, 4-{[4-(5,6-dimethyl-2-pyridin-2-ylpyridin-3-yl)oxypyridin-2-yl]amino}benzoic acid, ethyl 4-{[4-(5,6-dimethyl-2-pyridin-2-ylpyridin-3-yl)oxypyridin-2-yl]amino}benzoate, 4-{[4-(2,6-dimethylpyridin-3-yl)oxypyridin-2-yl]amino}-N-[2-(1,1-dioxo-1,4-thiazinan-4-yl)ethyl]benzamide, 4-{[4-(2,6-dimethylpyridin-3-yl)oxypyridin-2-yl]amino}-N-(2-pyrrol-1-ylethyl)benzamide, 4-{[4-(2,6-dimethylpyridin-3-yl)oxypyridin-2-yl]amino}-N-(2-piperidin-1-ylethyl)benzamide, 4-{[4-(2,6-dimethylpyridin-3-yl)oxypyridin-2-yl]amino}-N-[2-(8-oxa-3-azabicyclo[3.2.1]octan-3-yl)ethyl]-benzamide, N-[2-(4,4-difluoropiperidin-1-yl)ethyl]-4-{[4-(2,6-dimethyl-pyridin-3-yl)oxypyridin-2-yl]amino}benzamide, 4-{[4-(2,6-dimethylpyridin-3-yl)oxypyridin-2-yl]amino}-N-(2-morpholin-4-ylethyl)benzamide and 2-(3-{2-[(3,4,5-trimethoxyphenyl)-amino]pyridin-4-yl}oxypyridin-2-yl)benzamide;

or a pharmaceutically acceptable salt thereof.

In a further embodiment there is a subset of compounds of formula (I) selected from: 4-(5,6-dimethyl-2-pyridin-2-yl-pyridin-3-yl)oxy-N-pyridin-2-yl-pyridin-2-amine, 4-{[4-(5,6-dimethyl-2-pyridin-2-yl-pyridin-3-yl)oxypyridin-2-yl]amino}benzene-sulfonamide, (4-{[4-(5,6-dimethyl-2-pyridin-2-yl-pyridin-3-yl)-oxypyridin-2-yl]amino}phenyl)methanol, 4-({4-[(2,6-dimethylpyridin-3-yl)oxy]pyridin-2-yl}amino)-benzenesulfonamide, 4-{[4-(6-ethyl-2-pyridin-2-yl-pyridin-3-yl)oxypyridin-2-yl]-amino}benzenesulfonamide, (3-{[4-(2,6-dimethylpyridin-3-yl)oxypyridin-2-yl]amino}phenyl)-(3-methylsulfonylpyrrolidin-1-yl)methanone, N-(2-amino-2-oxoethyl)-3-{[4-(2,6-dimethylpyridin-3-yl)-oxypyridin-2-yl]amino}benzamide, 2-(4-{[4-(5,6-dimethyl-2-pyridin-2-ylpyridin-3-yl)oxypyridin-2-yl]-amino}phenyl)acetonitrile, 4-{[4-(5,6-dimethyl-2-pyridin-2-ylpyridin-3-yl)oxypyridin-2-yl]-amino}benzamide, 3-{[4-(5,6-dimethyl-2-pyridin-2-ylpyridin-3-yl)oxypyridin-2-yl]-amino}benzenesulfonamide, N-(3-{[4-(2,6-dimethylpyridin-3-yl)oxypyridin-2-yl]amino}phenyl)methanesulfonamide, 4-(6-methyl-2-propyl-pyridin-3-yloxy)-N-(3,4,5-trimethoxy-phenyl)pyridin-2-amine, 4-(2,6-dimethyl-pyridin-3-yl)oxy-N-{3-[(4-methylsulfonyl-piperazin-1-yl)methyl]phenyl}pyridin-2-amine, 4-(2,6-dimethylpyridin-3-yl)oxy-N-[3-(piperazin-1-ylmethyl)phenyl]pyridin-2-amine, N-cyclopropyl-2-{4-[(3-{[4-(2,6-dimethylpyridin-3-yl)oxypyridin-2-yl]amino}phenyl)methyl]piperazin-1-yl}acetamide, 2-{4-[3-({4-[(2,6-dimethyl-pyridin-3-yl)oxy]pyridin-2-yl}amino)benzyl]piperazin-1-yl}propanamide and 3-{[4-(5,6-dimethyl-2-pyridin-2-ylpyridin-3-yl)oxypyridin-2-yl]amino}phenol;

or a pharmaceutically acceptable salt thereof

Another aspect of the present invention provides a process for preparing a compound of formula (I) or a pharmaceutically acceptable salt thereof, which process comprises:

### Process a)

reacting a pyridine of formula (II):

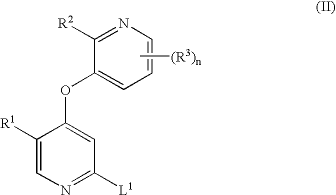

with an compound of formula (III):

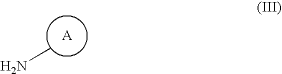

wherein L¹ is a displaceable group, and wherein values of R¹, R², R³, n and A are as defined hereinbefore;

### Process b)

reacting a compound of formula (IV):

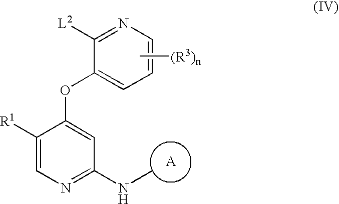

with a boron species of formula (V):

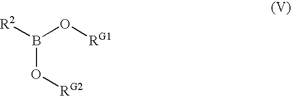

wherein L² is a displaceable group, R^{G1} and R^{G2} are each independently selected from hydrogen, C₁₋₆alkyl, or else R^{G1} and R^{G2} together with the O—B—O to which they are attached form a ring containing a C₂₋₈alkyl group or a C₆₋₈aryl group; and wherein values of R¹, R², R³, n and A are as defined hereinbefore;

### Process c)

reacting a compound of formula (VI):

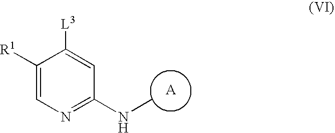

wherein L3 is a displaceable group;

with a compound of formula (VII):

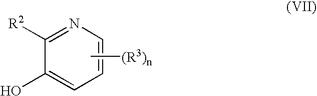

wherein values of R¹, R², R³, n and A are as defined hereinbefore; or

### Process d)

Reacting a compound of formula (VIII):

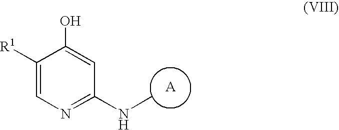

with a compound of formula (IX):

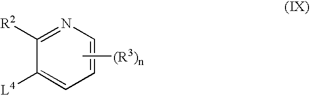

wherein L⁴ is a displaceable group and wherein the values of R¹, R², R³, n and A are as defined hereinbefore;

and optionally removing any protecting groups to provide a compound of formula (I) and optionally, thereafter, carrying out one or both of the following steps:
i) converting a compound of formula (I) into another compound of formula (I);
ii) forming a pharmaceutically acceptable salt.
   Further information relating to the above processes is provided below.

### Process a)

Suitable values for L¹ are for example halo, for example chloro or bromo.

Pyridines of formula (II) and compounds of formula (III) may be reacted together: in the presence of a suitable solvent, for example an ethereal solvent such as 1,4-dioxane or tetrahydrofuran, or an aromatic hydrocarbon solvent such as toluene or xylene, or an dipolar aprotic solvent such as N,N-dimethylacetamide, N,N-dimethylformamide or N-methylpyrrolidinone;

in the presence of a suitable base, for example an inorganic base such as caesium carbonate, or an organic base such as sodium tert-butoxide;

optionally in the presence of a metal catalyst such as a palladium catalyst or precatalyst, for example palladium(II)acetate or palladium(0)dibenzylideneacetone, together with a phosphine ligand such as BINAP (2,2′-diphenylphosphino-1,1′-binaphthyl), dppp (1,3-bis(diphenylphosphino)propane) or (tri-o-tolylphosphine);

using conventional or microwave-assisted heating.

Pyridines of the formula (II) and compounds of formula (III) can be reacted together, for example using N,N-dimethylacetamide as solvent, using a Pd(II)-based catalyst, caesium carbonate as base, using a temperature of approximately 120-150° C. for approximately 30-60 minutes, using a microwave.

Pyridines of the formula (II) and compounds of formula (III) are commercially available compounds, or they are well-known in the literature, or they are prepared by standard processes known in the art, or their syntheses are described hereinafter.

Pyridines of formula (II) may be prepared by reacting a 2,4-dihalopyridine with the requisite 3-hydroxypyridine under basic conditions. Where the leaving group at the 4-position of the pyridine is chloro, the reaction can be performed using N,N-dimethylacetamide as the solvent, sodium hydride as the base, using a temperature of approximately 50-100° C. and a reaction time of approximately 30-60 minutes. Where the leaving group at the 4-position of the pyridine is iodo, the reaction can be performed using N,N-dimethylacetamide as the solvent, caesium carbonate as the base, with the assistance of copper(I)iodide catalyst, using a temperature of approximately 150° C. for approximately 30 minutes in a microwave.

### Process b)

Suitable values of L² are halo, for example chloro.

Suitable boron species are well known in the chemical art and include boronic acids, their corresponding trimeric anhydrides which may interconvert with the boronic acid during reaction, neopentyl esters, pinacol esters, and catechol esters.

Compounds of formula (IV) and boron species of formula (V) may be reacted together in the presence of a suitable solvent, for example an ethereal solvent such as 1,4-dioxane, using a palladium catalyst or precatalyst, for example Pd(PPh₃)₄, with a suitable base, such as an inorganic carbonate base, for example sodium carbonate, optionally in the presence of a small amount of water, by conventional or microwave assisted heating, for example using a temperature of 130° C. in a sealed microwave tube for 1 h.

Compounds of formula (IV) can be synthesised using standard reactions known in the chemical art, and such a synthesis is described within the Examples and Methods of this document. Boron species of formula (V) can be synthesised reactions that are well known in the chemical art, or they are commercially available.

### Processes c and d)

Suitable values of L³ and L⁴ include halo, for example fluoro, chloro, bromo and iodo.

Compounds of the formula (VI) and compounds of the formula (VII) can be reacted together using Ullman ether-forming conditions. Similarly, compounds of formula (VIII) can be coupled to compounds of formula (IX) using Ullman ether-forming conditions. For example involving the use of a copper salt such as copper iodide or copper oxide, an inorganic base such as Cs₂CO₃ or K₂CO₃, in a polar aprotic solvent such as N,N-dimethylacetamide at elevated temperature, for example 150° C.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess anti-cancer activity which is believed to arise from ALK5 inhibitory activity of the compounds. These properties may be assessed, for example, using the procedures set out below:

### ALK5 Enzyme Assay 1

This assay measures the ability of a compound to bind to and inhibit the activity of ALK5 protein in vitro. 6His tagged ALK5 protein (amino acids 162-503) expressed in insect cells was purified and stored at −80° C. in aliquots. Assay measurements were performed in assay buffer comprising 60 mM HEPES pH 7.4, 3 mM MgCl₂, 3 mM MnCl₂, 3 μM Na orthovanadate, 1.2 mM DTT and 0.05% PEG₂₀₀₀. Test compounds were prepared by dilution in 100% DMSO to give the appropriate dose range and dispensed into a 96 well assay plate as a 10× concentrate. Reactions were performed in the presence of 200 μg/mL casein (Sigma-Aldrich C4765-10ML), 200 ng/mL ALK5, 0.5 μM ATP (Sigma-Aldrich A7699), 1 μCi/mL γ-33P ATP. Following addition of casein, ATP and compound, reactions were initiated by adding ALK5 enzyme and reactions run for 90 minutes. After incubation reactions were terminated by addition of 20 μL per well of 50% orthophosphoric acid. 20 minutes after addition of 70% v/v TCA, precipitated protein was transferred to GF/C 96 well plates using a Tomtec harvester with 1.5% orthophosphoric acid. Plates were then dried, 25 μL Microscint 20 (Packard Bioscience 6013621) added to each well and counted on a Packard Topcount. All reactions were performed under linear kinetics.

### ALK5 Enzyme Assay 2

The ability of a compound to bind and inhibit ALK5 can be measured by its capacity to displace a probe molecule from recombinant ALK5 in an in vitro assay. Provided the probe molecule is derivatised with a fluorescent probe or is fluorescent itself, the binding affinity can be measured using Fluorescence Polarisation (FP). 6His tagged human ALK5 (residues162-503) was grown and purified from insect cells and stored at −80° C. in aliquots. Assay measurements were performed in 50 mM HEPES pH7.4, 10 mM MgCl₂, 1 mM DTT, 0.01% CHAPS in a final assay volume of 12 μl. Briefly 6 μl of ALK5 protein, final concentration of 3 μg/mL, was added to each well of a 384 well assay plate containing 12 nL of each concentration of test compound dissolved in DMSO. 6 μL of a solution containing an ALK5 FP probe ((2Z)-3-(6-{4-[2-({4-[(6-chloro[1,3]dioxolo[4,5-b]pyridin-7-yl)amino]-5-(1-methylethoxy)quinazolin-7-yl}oxy)ethyl]piperazin-1-yl}-6-oxohexyl)-2-[(2E,4E)-5-(3-ethyl-1,1-dimethyl-6,8-disulfo-1H-benzo[e]indolium-2-yl)penta-2,4-dien-1-ylidene]-1,1-dimethyl-6-sulfo-2,3-dihydro-1H-benzo[e]indole-8-sulfonate), final concentration 50 nM, and ATP, final concentration 1 μM. Following incubation for 30 minutes at room temperature, fluorescence polarisation was measured on a BMG Labtech PHERAstar plate reader using a 650/690 nm optic module. mP values were then used to calculate IC₅₀ values for each compound tested.

### Cell Assay

Ligand mediated translocation of R-Smads (Smads 1,2,3,5,8) is a well-documented phenomenon in a variety of cell types. (Derynck, R. and Zhang, Y., 2003, Nature, 425, 577-584, Shi, Y. and Massague, J., 2003, Cell, 113, 685-700). Specifically TGFβ1 and TGFβ3 cause phosphorylation and nuclear translocation of Smad2 and Smad3 transcription factors. Thus compound inhibition of TGFβsignalling can be estimated by measuring cellular distribution of Smad2 or 3 under activated TGFβpathway. The Smad2 Redistribution™ Assay (Fisher BioImage ApS) was used to assess in vitro cellular activity of compounds of interest. The breast adenocarcinoma MDA-MB-468 parent cell line was used to create a recombinant cell line, MDA-MB-468 PS213 I GScl4C8 containing Smad2 fused to the C-terminus of enhanced green fluorescent protein (Smad2-EGFP). This enables monitoring of cellular distribution of Smad2 using high throughput fluorescence microscopy.

This assay measures the ability of compounds to inhibit TGFβ1 mediated smad2 nuclear translocation. Compounds dose ranges were created by diluting in 100% DMSO and then further into assay media (RPMI1640, 1% FCS, 10 mM HEPES pH 7.4). Cells were plated in 96 well Packard View plates at 8×10³ per well in 100 μL RPMI1640, 10% FCS, 0.5 mg/mL geneticin and grown for 48 hrs. Following addition of 10 μL of diluted compound and 50 μL TGFβ1 (Calbiochem 616455) to a final concentration of 3 ng/mL, cell were incubated for 90 minutes. Media was removed and cells fixed in 50 μL 4% v/v formaldehyde solution for 10 minutes. After removal of fix, cells were washed and permeabilised in 100 μL of PBS containing 0.5% Triton™ X-100 and 1 μM Hoechst 33258. Plates were then read on an ArrayScan Vti instrument to determine nuclear to cytoplasmic staining. Dose responses were estimated using proprietary software.

IC₅₀ values for compounds of the invention when tested in one or more of the above assays are typically less than 10 μM.

The compounds of formula (I) have activity as pharmaceuticals, in particular as modulators or inhibitors of ALK5 activity, and may be used in the treatment of proliferative, hyperproliferative and metastatic diseases/conditions, examples of which include the following cancers:
(1) carcinoma, including that of the bladder, brain, breast, colon, kidney, liver, lung, ovary, pancreas, prostate, stomach, cervix, colon, thyroid and skin;
(2) hematopoietic tumors of lymphoid lineage, including acute lymphocytic leukaemia, B-cell lymphoma and Burketts lymphoma;
(3) hematopoietic tumours of myeloid lineage, including acute and chronic myelogenous leukaemias and promyelocytic leukaemia;
(4) tumours of mesenchymal origin, including fibrosarcoma and rhabdomyosarcoma; and
(5) other tumours, including melanoma, seminoma, tetratocarcinoma, neuroblastoma and glioma.

In one embodiment the compounds of the invention are useful in the treatment of tumours of the bladder, breast and prostate and multiple myeloma.

Thus, the present invention provides a compound of formula (I), or a pharmaceutically-acceptable salt thereof, as herein defined for use in therapy.

According to a further aspect of the present invention there is provided a compound of the formula (I), or a pharmaceutically acceptable salt thereof, as defined hereinbefore for use in a method of treatment of the human or animal body by therapy.

In a further aspect, the present invention provides the use of a compound of formula (I), or a pharmaceutically acceptable salt, as herein defined in the manufacture of a medicament for use in therapy.

In this aspect, the present invention provides a compound of formula (I), or a pharmaceutically acceptable salt thereof, as herein defined for use in therapy.

In the context of the present specification, the term “therapy” also includes “prophylaxis” unless there are specific indications to the contrary. The terms “therapeutic” and “therapeutically” should be construed accordingly.

The invention also provides a method of treating cancer which comprises administering to a patient in need thereof a therapeutically effective amount of a compound of formula (I), or a pharmaceutically acceptable salt thereof, as herein defined.

We have found that the compounds defined in the present invention, or a pharmaceutically acceptable salt thereof, are effective anti-cancer agents which property is believed to arise from modulating or inhibiting activity of the ALK family, for example ALK5. Accordingly the compounds of the present invention are expected to be useful in the treatment of diseases or medical conditions mediated alone or in part by ALK5, i.e. the compounds may be used to produce an ALK inhibitory effect in a warm-blooded animal in need of such treatment.

Thus the compounds of the present invention provide a method for treating cancer characterised by inhibition of ALK family members, for example ALK5, i.e. the compounds may be used to produce an anti-cancer effect mediated alone or in part by the inhibition of ALK5.

Such a compound of the invention is expected to possess a wide range of anti-cancer properties as activating mutations in ALK5 have been observed in many human cancers, including but not limited to breast, bladder, prostrate and multiple myeloma. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In one embodiment compounds of the invention are expected to slow advantageously either the growth or metastatic capacity or both of primary and recurrent solid tumours of, for example, the skin, colon, thyroid, lungs and ovaries. More particularly such compounds of the invention, or a pharmaceutically acceptable salt thereof, are expected to inhibit the growth and spread (metastasis) of those tumours which are associated with ALK5, especially those tumours which are significantly dependent on ALK5 for their growth and spread (metastasis), including for example, certain tumours of the bladder, lung, prostrate and breast.

Thus according to this aspect of the invention there is provided a compound of the formula (I), or a pharmaceutically acceptable salt thereof, as defined herein for use as a medicament.

According to a further aspect of the invention there is provided the use of a compound of the formula (I), or a pharmaceutically acceptable salt thereof, as defined herein in the manufacture of a medicament for the production of a ALK5 inhibitory effect in a warm-blooded animal such as man.

According to this aspect of the invention there is provided a compound of formula (I), or a pharmaceutically acceptable salt thereof, as defined herein for use in the production of an ALK5 inhibitory effect in a warm-blooded animal such as man.

According to this aspect of the invention there is provided the use of a compound of the formula (I), or a pharmaceutically acceptable salt thereof, as defined herein in the manufacture of a medicament for the production of an anti-cancer effect in a warm-blooded animal such as man.

According to this aspect of the invention there is provided a compound of the formula (I), or a pharmaceutically acceptable salt thereof, as defined herein for use in the production of an anti-cancer effect in a warm-blooded animal such as man.

According to a further feature of the invention, there is provided the use of a compound of the formula (I), or a pharmaceutically acceptable salt thereof, as defined herein in the manufacture of a medicament for the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to this feature of the invention, there is provided a compound of the formula (I), or a pharmaceutically acceptable salt thereof, as defined herein for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined herein in the production of a ALK5 inhibitory effect in a warm-blooded animal such as man.

According to this aspect of the invention there is provided the use of a compound of the formula (I), or a pharmaceutically acceptable salt thereof, as defined herein in the production of an anti-cancer effect in a warm-blooded animal such as man.

According to a further feature of the invention, there is provided the use of a compound of the formula (I), or a pharmaceutically acceptable salt thereof, as defined herein in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a ALK5 inhibitory effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined herein.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the production of a ALK5 inhibitory effect in a warm-blooded animal such as man.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries in a warm-blooded animal such as man.

The compounds of formula (I) and pharmaceutically acceptable salts thereof may be used on their own but will generally be administered in the form of a pharmaceutical composition in which the formula (I) compound or salt (active ingredient) is in association with a pharmaceutically acceptable adjuvant, diluent or carrier. Depending on the mode of administration, the pharmaceutical composition may comprise from 0.01 to 99% w (per cent by weight), from 0.05 to 80% w, from 0.10 to 70% w, and or even from 0.10 to 50 % w, of active ingredient, all percentages by weight being based on total composition.

The present invention also provides a pharmaceutical composition comprising a compound of formula (I), or a pharmaceutically acceptable salt thereof, as herein defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt thereof, as herein defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propylp-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The size of the dose for therapeutic purposes of a compound of the invention will naturally vary according to the nature and severity of the conditions, the age and sex of the animal or patient and the route of administration, according to well known principles of medicine.

In general, a compound of the invention will be administered so that a daily dose in the range, for example, from 0.1 mg to 1000 mg active ingredient per kg body weight is received, given if required in divided doses. However the daily dose will necessarily be varied depending upon the host treated, the particular route of administration, and the severity of the illness being treated. Accordingly the optimum dosage may be determined by the practitioner who is treating any particular patient. In general lower doses will be administered when a parenteral route is employed. Thus, for example, for intravenous administration, a dose in the range, for example, from 0.1 mg to 30 mg active ingredient per kg body weight will generally be used. Similarly, for administration by inhalation, a dose in the range, for example, from 0.1 mg to 25 mg active ingredient per kg body weight will generally be used. Oral administration is however preferred. For example, a formulation intended for oral administration to humans will generally contain, for example, from 0.1 mg to 2 g of active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZDO530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell anergy, approaches using transfected immune cells such as cytokine transfected dendritic cells, approaches using cytokine transfected tumour cell lines and approaches using anti idiotypic antibodies.

According to this aspect of the invention there is provided a pharmaceutical product comprising a compound of formula (I) as defined hereinbefore and an additional anti-tumour substance as defined hereinbefore for the conjoint treatment of cancer.

In the above other pharmaceutical composition, process, method, use and medicament manufacture features, the alternative embodiments of the compounds of the invention described herein also apply.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high performance liquid chromatography (HPLC); the reaction times that are given are not necessarily the minimum attainable;

(iii) when necessary, organic solutions were dried over anhydrous MgSO₄, work-up procedures were carried out using traditional layer separating techniques, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of formula (I) were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Avance DPX300 spectrometer operating at a field strength of 300 MHz, or a Bruker Avance DRX400 operating at 400 MHz. Unless otherwise stated, NMR spectra were obtained at 300 MHz in d⁶-DMSO. The following abbreviations have been used: s, singlet; d, doublet; dd, doublet of doublets; ddd, doublet of doublet of doublets; t, triplet; q, quartet; m, multiplet; br, broad;

(vi) unless stated otherwise compounds containing any asymmetric carbon and/or sulphur atom were not resolved;

(vii) intermediates were not necessarily fully purified but their structures and purity were assessed by TLC, analytical HPLC and/or NMR analysis;

(viii) unless otherwise stated, column chromatography (by the flash procedure, FCC) and medium pressure liquid chromatography (MPLC) were performed on Merck Kieselgel silica (Art. 9385) or on Silicycle cartridges (40-63 microns silica, 12 to 120 g weight) using an Isco Combi Flash Companion system.

(ix) Preparative HPLC was performed on C18 reversed-phase silica, for example on a Waters ‘Xterra’ or ‘XBridge’ preparative reversed-phase column (5 μm silica, 19 mm diameter, 100 mm length) or on a Phenomenex “Gemini” or ‘AXIA’ preparative reversed-phase column (5 μm silica, 110A, 21.1 mm diameter, 100 mm length) using decreasingly polar mixtures as eluent, for example [containing 1% formic acid or 1% aqueous NH₄OH (d=0.88)] as solvent A and acetonitrile as solvent B; either of the following preparative HPLC methods were used:

Method A: a solvent gradient over 9.5 minutes, at 25 mL per minute, from a 85:15 mixture of solvents A and B respectively to a 5:95 mixture of solvents A and B.

Method B: a solvent gradient over 9.5 minutes, at 25 mL per minute, from a 60:40 mixture of solvents A and B respectively to a 5:95 mixture of solvents A and B.

(x) the following analytical HPLC methods were used; in general, reversed-phase silica was used with a flow rate of about 1 mL per minute and detection was by Electrospray Mass Spectrometry and by UV absorbance at a wavelength of 254 nm; for each method Solvent A was water and Solvent B was acetonitrile; the following columns and solvent mixtures were used:

Analytical HPLC was performed on C18 reversed-phase silica, on a Phenomenex “Gemini” preparative reversed-phase column (5 μm silica, 110 A, 2 mm diameter, 50 mm length) using decreasingly polar mixtures as eluent, for example decreasingly polar mixtures of water (containing 0.1% formic acid or 0.1% ammonia) as solvent A and acetonitrile as solvent B; the following analytical HPLC method was used:

A solvent gradient over 4 minutes, at approximately 1 mL per minute, from a 95:5 mixture of solvents A and B respectively to a 5:95 mixture of solvents A and B.

(xi) where reactions refer to the use of a microwave, it was a Smith Synthesizer Microwave that was used;

(xii) the following abbreviations have been used:

| | | | |
|---|---|---|---|
| DMSO | dimethylsulfoxide | FCC | flash column chromatography |
| Pd(OAc)₂ | palladium (II) acetate | MeCN | acetonitrile |
| DIPEA | diisopropylethylamine | NEt₃ | triethylamine |
| TFA | trifluoroacetic acid | EtOH | ethanol |
| MeOH | methanol | mins. | minutes |
| CAA | cell assay activity (μM) | EAA | activity (μM) using Enzyme |
| | | | Assay 1 |
| Pd₂(dba)₃ | tris(dibenzylideneacetone)- | EAA2 | activity (μM) using Enzyme |
| | dipalladium(0) | | Assay 2 |
| DMF | N,N-dimethylformamide | m/z | mass spectrometry peak(s) |
| HBTU | O-Benzotriazole-N,N,N′,N′- | HATU | 2-(1H-7-Azabenzotriazol-1-yl)--1,1,3,3- |
| | tetramethyluronium- | | tetramethyl uronium |
| | hexafluorophosphate | | hexafluorophosphate |
| DMA | N,N-dimethylacetamide | xantphos | 4,5-bis(diphenylphosphino)-9,9- |
| DCM | dichloromethane | | dimethylxanthene |
| THF | tetrahydrofuran | r.t. | room temperature |
| EtOAc | ethyl acetate | Pd(PPh₃)₄ | tetrakis(triphenylphosphine)- |
| h | hour(s) | | palladium(0) |

### Example 1

### 4-[2-(6-Methylpyridin-2-yl)pyridin-3-yl]oxy-N-(3,4,5-trimethoxy-phenyl)pyridin-2-amine

3,4,5-Trimethoxyaniline (70 mg), xantphos (26 mg), Pd(OAc)₂ (15 mg), Cs₂CO₃ (247 mg) and 3-(2-chloropyridin-4-yl)oxy-2-(6-methylpyridin-2-yl)pyridine (Method 1, 114 mg) were combined in 1,4-dioxane (2 mL). The reaction was heated at 120° C. for 30 minutes by microwave. After cooling mixture was partitioned between EtOAc and water and the phases were separated. The aqueous portion was extracted with EtOAc. The combined organic layers were washed with brine, dried (MgSO₄) and concentrated in vacuo. Purification by FCC using a gradient of 0-70% MeOH in DCM provided the title compound (70 mg, 41%) as a white solid after trituration using a mixture of EtOAc, iso-hexane and diethyl ether; ¹H NMR: 2.32 (3H, s), 3.59 (3H, s), 3.70 (6H, s), 6.08 (1H, d), 6.33-6.35 (0.1H, m), 6.94 (2H, s), 7.21 (1H, d), 7.59-7.62 (1H, m), 7.65 (1H, d), 7.73-7.77 (1H, m), 7.80 (1H, dd), 7.99 (1H, d), 8.60 (1H, dd), 8.78 (1H, s); m/z: MH⁺ 445.56; EAA: 0.0783; EAA2: 0.08898.

### Example 2

### 4-[5,6-Dimethyl-2-(6-methylpyridin-2-yl)pyridin-3-yl]oxy-N-(3,4,5-trimethoxy-phenyl)pyridin-2-amine

3,4,5-Trimethoxyaniline (51 mg), xantphos (19 mg), Pd(OAc)₂ (15 mg), Cs₂CO₃ (182 mg) and 5-(2-chloropyridin-4-yl)oxy-2,3-dimethyl-6-(6-methylpyridin-2-yl)pyridine (Method 5, 90 mg) were combined in 1,4-dioxane (2 mL). The mixture was heated to 120° C. for 30 mins. by microwave. After cooling mixture was partitioned between EtOAc and water and the phases were separated. The aqueous portion was extracted with EtOAc. The combined organic portions were washed with brine, dried (MgSO₄) and concentrated in vacuo. Purification by FCC using a gradient of 0-70% MeOH in DCM afforded the title compound (100 mg, 76%) as a white solid after trituration with a mixture of EtOAc, iso-hexane and diethyl ether; ¹H NMR: 2.29 (3H, s), 2.34 (3H, s), 2.52 (3H, s), 3.57 (3H, s), 3.70 (6H, s), 6.04 (1H, d), 6.32 (1H, dd), 6.95 (2H, s), 7.15 (1H, d), 7.55 (1H, s), 7.62 (1H, d), 7.67-7.72 (1H, m), 7.96 (1H, d), 8.74 (1H, s); m/z: MH⁺ 473.53; EAA: 0.0337; EAA2: 0.07232.

### Example 3

### 4-[6-Ethyl-2-(6-methylpyridin-2-yl)pyridin-3-yl]oxy-N-(3,4,5-trimethoxy-phenyl)pyridin-2-amine

3,4,5-Trimethoxyaniline (56 mg), xantphos (22 mg), Pd(OAc)₂ (15 mg), Cs₂CO₃ (202 mg) and 3-(2-chloropyridin-4-yl)oxy-6-ethyl-2-(6-methylpyridin-2-yl)pyridine (Method 6, 100 mg) were combined in 1,4-dioxane (2 mL). The mixture was heated to 120° C. for 30 mins. in a microwave. After cooling the mixture was diluted with water and EtOAc and the phases were separated. The aqueous portion was extracted with EtOAc. The combined organic portions were washed with brine, dried (MgSO₄) and concentrated in vacuo. Part-purification by FCC using a gradient of 0-70% MeOH in DCM followed by further purification using basic reverse phase HPLC afforded the title compound (64 mg, 44%) as a white solid after trituration with a mixture of EtOAc, hexanes and diethyl ether; ¹H NMR: 1.32 (3H, t), 2.32 (3H, s), 2.87 (2H, q), 3.58 (3H, s), 3.70 (6H, s), 6.06 (1H, d), 6.33 (1H, dd), 6.94 (2H, s), 7.20 (1H, d), 7.47 (1H, d), 7.64 (1H, d), 7.70-7.76 (2H, m), 7.98 (1H, d), 8.76 (1H, d); m/z: MH⁺ 473.52; EAA: 0.0274; EAA2: 0.04587.

### Example 4

### {4-[2-(4,5-Dimethyl-thiazol-2-yl)-5,6-dimethyl-pyridin-3-yloxy]-pyridin-2-yl}-(3,4,5-trimethoxy-phenyl)-amine

NaH (240 mg, 60% dispersion in mineral oil, 6 mmol) and 2,4-dichloropyridine (666 mg, 4.5 mmol) were added sequentially to a solution of 2-(4,5-dimethyl-1,3-thiazol-2-yl)-5,6-dimethyl-pyridin-3-ol (prepared according to WO2004/018430, 703 mg, 3 mmol) in DMF (8 mL) and the resulting mixture was heated to reflux for 1 h. The mixture was diluted with EtOAc and water, the phases separated, and the aqueous phase was extracted twice with EtOAc. The combined organic layers were washed with brine, then dried (Na₂SO₄) and concentrated in vacuo. The resulting crude mixture was partially purified by FCC, eluting with 20% EtOAc/hexane, to obtain impure intermediate 3-(2-chloropyridin-4-yl)oxy-2-(4,5-dimethyl-1,3-thiazol-2-yl)-5,6-dimethyl-pyridine (407 mg). A portion of this intermediate (139 mg) was combined with 3,4,5-trimethoxyaniline (147 mg, 0.8 mmol), xantphos (28 mg, 0.05 mmol), Cs₂CO₃ (260 mg, 0.8 mmol) and Pd(OAc)₂ (ca. 5 mg) in a microwave tube and the tube was purged with nitrogen. DMA (3 mL) was added and the mixture was heated in a microwave at 150° C. for 15 mins. The mixture was diluted with EtOAc and filtered through diatomaceous earth. The diatomaceous earth was washed with EtOAc and the combined organic portions were concentrated in vacuo. The residue was purified by FCC, eluting with 50-100% EtOAc in hexane, followed by recrystallisation from MeOH/CHCl₃ to obtain the title compound as a white solid; (45 mg, 23%); ¹H NMR: (400 MHz, CDCl₃) 2.30 (3H, s), 2.31 (3H, s), 2.34 (3H, s), 2.58 (3H, s), 3.77 (6H, s), 3.80 (3H, s), 6.27 (1H, d), 6.36-6.38 (2H, m), 6.46 (2H, s), 7.23 (1H, s), 8.04 (1H, d); m/z: MH⁺ 493.45; EAA: 0.00951; EAA2: 0.05604

### Example 5

### 4-[6-Methyl-2-(1-methylpyrazol-4-yl)pyridin-3-yl]oxy-N-(3,4,5-trimethoxy-phenyl)pyridin-2-amine

3,4,5-Trimethoxyaniline (90 mg), xantphos (34 mg), Pd(OAc)₂ (10 mg), Cs₂CO₃ (325 mg) and 3-(2-chloropyridin-4-yl)oxy-6-methyl-2-(1-methylpyrazol-4-yl)pyridine (Method 10, 148 mg) were combined in 1,4-dioxane (2 mL). The mixture was heated to 120° C. for 30 minutes in a microwave. After cooling the mixture was diluted with water and EtOAc and the phases were separated. The aqueous portion was extracted with EtOAc. The combined organic portions were washed with brine, dried (MgSO₄) and concentrated in vacuo. Purification by FCC using a gradient of 0-100% EtOAc in iso-hexane then 0-20% MeOH in DCM afforded the title compound as a white foam; (7 mg, 6%); ¹H NMR: (300 MHz, CDCl₃) 2.57 (3H, s), 3.77 (6H, s), 3.81 (3H, s), 3.89 (3H, s), 6.27 (1H, d), 6.36 (1H, dd), 6.45 (2H, s), 6.72 (1H, s), 6.99 (1H, d), 7.26 (1H, d), 7.89 (1H, s), 8.01 (1H, s), 8.04 (1H, d); m/z: MH⁺ 448.53; EAA: 0.0153; EAA2: 0.01687.

### Example 6

### 4-Pyridin-3-yloxy-N-(3,4,5-trimethoxyphenyl)pyridin-2-amine

3,4,5-Trimethoxyaniline (66 mg), xantphos (25 mg), Pd(OAc)₂ (15 mg), Cs₂CO₃ (234 mg) and 2-chloro-4-pyridin-3-yloxy-pyridine (Method 12, 75 mg) were combined in 1,4-dioxane (2.5 mL). The mixture was heated to 150° C. for 40 mins. in a microwave. After cooling the mixture was diluted with water and EtOAc, and the phases were separated. The aqueous portion was extracted with EtOAc. The combined organic portions were washed with brine, dried (MgSO₄) and concentrated in vacuo. Purification by acidic reverse phase HPLC afforded the title compound as a solid; (20 mg, 16%); ¹H NMR: 3.60 (3H, s), 3.72 (6H, s), 6.20 (1H, d), 6.46 (1H, dd), 6.96 (2H, s), 7.54 (1H, dd), 7.68-7.72 (1H, m), 8.09 (1H, d), 8.51-8.53 (2H, m), 8.90 (1H, s); m/z: MH⁺ 353.98; EAA: 5.502; EAA2: 1.971.

The procedure described above was repeated using the appropriate 4-(2-chloro-pyridin-4-yl)oxyheterocycle and 3,4,5-trimethoxyaniline and using DMA in place of 1,4-dioxane to provide the Examples of the table below. The first example in the table was purified by FCC. The remaining examples were purified by acidic reverse phase chromatography. The necessary starting materials were prepared by procedures analogous to those of the numbered Methods that are indicated for each entry.

| Ex. | Name and Data |
|---|---|
| 7 | 4-(2,6-Dimethylpyridin-3-yl)oxy-N-(3,4,5-trimethoxyphenyl)-pyridin-2-amine |
| | ¹H NMR: 2.29 (3H, s), 2.48 (3H, s), 3.60 (3H, s), 3.72 (6H, s), 6.02 (1H, |
| | dd), 6.80 (1H, d), 6.96-6.96 (2H, m), 7.21 (1H, d), 7.48 (1H, d), |
| | 8.06 (1H, d), 8.85 (1H, s); m/z: 382.7 MH⁺; EAA: 0.0206; EAA2: 0.03485. (Method 13). |
| 8 | 4-(6-Methylpyridin-3-yl)oxy-N-(3,4,5-trimethoxyphenyl)-pyridin-2-amine |
| | ¹H NMR: 2.49 (3H, s), 3.60 (3H, s), 3.72 (6H, s), 6.15 (1H, d), 6.44 (1H, |
| | dd), 6.97 (2H, s), 7.38 (1H, d), 7.58 (1H, dd), 8.07 (1H, d), 8.37 (1H, d), |
| | 8.87 (1H, s); m/z: 367.8 MH⁺; EAA: 0.174; EAA2: 0.294; (Method 14). |
| 9 | 4-(2-Methylpyridin-3-yl)oxy-N-(3,4,5-trimethoxyphenyl)-pyridin-2-amine |
| | ¹H NMR: 2.35 (3H, s), 3.60 (3H, s), 3.72 (6H, s), 6.06 (1H, d), 6.43 (1H, |
| | dd), 6.95 (2H, s), 7.35-7.40 (1H, m), 7.61 (1H, dd), 8.08 (1H, d), |
| | 8.42 (1H, dd), 8.86 (1H, s); m/z: 368.1 MH⁺; EAA: 0.114; EAA2: 0.1865; (Method 15). |

### Example 10

### 4-(5,6-Dimethyl-2-pyridin-2-yl-pyridin-3-yl)oxy-N-(4-fluorophenyl)pyridin-2-amine

4-Fluoroaniline (30 mg), xantphos (15 mg), Pd(OAc)₂ (4 mg), Cs₂CO₃ (133 mg) and 5-(2-chloropyridin-4-yl)oxy-2,3-dimethyl-6-pyridin-2-yl-pyridine (Method 16, 62 mg) were dissolved in DMA or dioxane. The mixture was heated to 150° C. for 15 minutes in a microwave. After cooling the mixture was purified directly from solution by reverse phase basic HPLC, to afford the title compound as a solid; (13 mg, 17%); ¹H NMR: 2.36 (3H, s), 2.53 (3H, s), 6.05 (1H, d), 6.35 (1H, dd), 7.05 (2H, dd), 7.32 (1H, ddd), 7.57 (1H, s), 7.60 (2H, ddd), 7.78-7.87 (2H, m), 7.95 (1H, d), 8.50 (1H, d), 8.88 (1H, s); m/z: MH⁺ 387.22; EAA: 0.0170; EAA2: 0.08828.

The procedure described above was repeated using the appropriate aniline with 5-(2-chloropyridin-4-yl)oxy-2,3-dimethyl-6-pyridin-2-yl-pyridine (Method 16), to provide Examples 11 to 28 below:

### Example 11

### 4-(5,6-Dimethyl-2-pyridin-2-yl-pyridin-3-yl)oxy-N-(2-morpholin-4-yl-phenyl)pyridin-2-amine

¹H NMR: 2.34 (3H, s), 2.5 (3H, under DMSO), 2.77 (4H, t), 3.71 (4H, t), 6.25 (1H, dd), 6.41 (1H, d), 6.92-7.03 (2H, m), 7.08 (1H, d), 7.32 (1H, t), 7.53 (1H, s), 7.76 (1H, d), 7.81-7.85 (3H, m), 7.92 (1H, d), 8.50 (1H, d); m/z: 454.4 M⁺; EAA: 1.502; EAA2: 0.5155.

### Example 12

### N-(3-Chlorophenyl)-4-(5,6-dimethyl-2-pyridin-2-yl-pyridin-3-yl)-oxy-pyridin-2-amine

¹H NMR: 2.36 (3H, s), 2.54 (3H, s), 6.10 (1H, d), 6.42 (1H, dd), 6.87 (1H, d), 7.22 (1H, t), 7.30-7.35 (1H, m), 7.38 (1H, d), 7.58 (1H, s), 7.79-7.87 (2H, m), 7.95 (1H, s), 8.03 (1H, d), 8.49 (1H, d), 9.10 (1H, s); m/z: 403.2 M⁺; EAA: 0.00730; EAA2: 0.04176.

### Example 13

### 4-(5,6-Dimethyl-2-pyridin-2-yl-pyridin-3-yl)oxy-N-(4-methylsulfonyl-phenyl)-pyridin-2-amine

¹H NMR: 2.36 (3H, s), 2.54 (3H, s), 3.11 (3H, s), 6.20 (1H, d), 6.49-6.51 (1H, m), 7.30-7.34 (1H, m), 7.60 (1H, s), 7.74 (2H, d), 7.80-7.87 (4H, m), 8.07 (1H, d), 8.49 (1H, d), 9.46 (1H, s); m/z: 447.3 M⁺; EAA: 0.00853; EAA2: 0.01027.

### Example 14

### 4-(5,6-Dimethyl-2-pyridin-2-yl-pyridin-3-yl)oxy-N-pyridin-2-yl-pyridin-2-amine

¹H NMR: 2.35 (3H, s), 2.54 (3H, s), 6.33 (1H, dd), 6.84 (1H, ddd), 7.28-7.33 (2H, m), 7.55 (1H, s), 7.59-7.69 (2H, m), 7.77-7.86 (2H, m), 8.01 (1H, d), 8.13-8.15 (1H, m), 8.48-8.51 (1H, m), 9.54 (1H, s); m/z: 370.2 M⁻; EAA: 0.0255; EAA: 0.008555.

### Example 15

### 4-(5,6-Dimethyl-2-pyridin-2-yl-pyridin-3-yl)oxy-N-pyridin-3-yl-pyridin-2-amine

¹H NMR: 2.36 (3H, s), 2.54 (3H, s), 6.12 (1H, d), 6.42 (1H, dd), 7.23 (1H, dd), 7.33 (1H, ddd), 7.58 (1H, s), 7.79-7.88 (2H, m), 8.01 (1H, d), 8.06 (1H, dd), 8.15 (1H, ddd), 8.50 (1H, dd), 8.70 (1H, d), 9.07 (1H, s); m/z: 370.2 M⁺; EAA: 0.0129; EAA2: 0.01465.

### Example 16

### N-(5-{[4-(5,6-Dimethyl-2-pyridin-2-yl-pyridin-3-yl)oxypyridin-2-yl]amino}-2-methyl-phenyl)acetamide

¹H NMR: 2.03 (3H, s), 2.09 (3H, s), 2.35 (3H, s), 2.5 (3H, under DMSO), 6.09 (1H, s), 6.31 (1H, d), 7.01 (1H, d), 7.33 (2H, dd), 7.56 (1H, s), 7.61 (1H, s), 7.77-7.87 (2H, m), 7.94 (1H, d), 8.51 (1H, d), 8.79 (1H, s), 9.18 (1H, s); m/z: 440.3 M⁺; EAA: 0.0128; EAA2: 0.006015.

### Example 17

### 4-(5,6-Dimethyl-2-pyridin-2-yl-pyridin-3-yl)oxy-N-(2-methoxy-5-methylsulfonyl-phenyl)pyridin-2-amine

¹H NMR: 2.42 (3H, s), 2.60 (3H, s), 3.16 (3H, s), 3.97 (3H, s), 6.47 (1H, dd), 6.64 (1H, d), 7.23 (1H, d), 7.38 (1H, ddd), 7.49 (1H, dd), 7.61 (1H, s), 7.84-7.93 (2H, m), 8.08 (1H, d), 8.48 (1H, s), 8.55 (1H, d), 9.02 (1H, d); m/z: 477.2 M⁺; EAA: 0.0147; EAA2: 0.05141.

### Example 18

### 4-(5,6-Dimethyl-2-pyridin-2-yl-pyridin-3-yl)oxy-N-(3-methoxy-phenyl)-pyridin-2-amine

¹H NMR: 2.36 (3H, s), 2.53 (3H, s), 3.70 (3H, s), 6.10 (1H, d), 6.36 (1H, dd), 6.42-6.45 (1H, m), 7.10 (2H, d), 7.30-7.35 (2H, m), 7.57 (1H, s), 7.78-7.88 (2H, m), 7.98 (1H, d), 8.50 (1H, d), 8.86 (1H, s); m/z: 399.3 M⁺; EAA: 0.00389; EAA2: 0.004374.

### Example 19

### 4-(5,6-Dimethyl-2-pyridin-2-yl-pyridin-3-y1)oxy-N-(2-methoxy-phenyl)-pyridin-2-amine

¹H NMR: 2.23 (3H, s), 2.5 (3H, under DMSO), 3.67 (3H, s), 6.18 (1H, dd), 6.29 (1H, d), 6.70-6.85 (3H, m), 7.18-7.22 (1H, m), 7.40 (1H, s), 7.64-7.74 (2H, m), 7.81 (1H, d), 7.92 (1H, s), 8.01-8.05 (1H, m), 8.39 (1H, d); m/z: 399.3 M⁺; EAA: 0.0128; EAA2: 0.002156.

### Example 20

### 4-(5,6-Dimethyl-2-pyridin-2-yl-pyridin-3-yl)oxy-N-[4-(4-methyl-piperazin-1-yl)phenyl]pyridin-2-amine

¹H NMR: 2.21 (3H, s), 2.35 (3H, s), 2.44 (4H, t), 2.5 (3H, under DMSO), 3.02 (4H, t), 6.00 (1H, d), 6.25 (1H, dd), 6.82 (2H, d), 7.33 (1H, ddd), 7.38 (2H, d), 7.54 (1H, s), 7.77 (1H, d), 7.84 (1H, td), 7.90 (1H, d), 8.51 (1H, d), 8.58 (1H, s); m/z: 467.4 M⁺; EAA: 0.0104; EAA2: 0.0166.

### Example 21

### 4-(5,6-Dimethyl-2-pyridin-2-yl-pyridin-3-yl)oxy-N-(3,5-dimorpholin-4-ylphenyl)pyridin-2-amine

¹H NMR: 2.34 (3H, s), 2.5 (3H, under DMSO), 2.99 (8H, t), 3.69 (8H, t), 6.05 (1H, s), 6.10 (1H, d), 6.31 (1H, dd), 6.63 (2H, d), 7.32 (1H, ddd), 7.54 (1H, s), 7.76-7.87 (2H, m), 7.95 (1H, d), 8.48-8.51 (1H, m), 8.61 (1H, s); m/z: 539.4 M⁺; EAA: 0.0772; EAA2: 0.125.

### Example 22

### 4-(5,6-Dimethyl-2-pyridin-2-yl-pyridin-3-yl)oxy-N-(3-morpholin-4-ylphenyl)pyridin-2-amine

¹H NMR: 2.35 (3H, s), 2.5 (3H, under DMSO), 3.02 (4H, t), 3.72 (4H, t), 6.09 (1H, d), 6.33 (1H, dd), 6.45-6.49 (1H, m), 7.01-7.09 (2H, m), 7.17 (1H, s), 7.30-7.35 (1H, m), 7.56 (1H, s), 7.76-7.88 (2H, m), 7.96 (1H, d), 8.50 (1H, d), 8.74 (1H, s); m/z: 454.3 M⁺; EAA: 0.0140; EAA2: 0.006131.

### Example 23

### 4-(5,6-Dimethyl-2-pyridin-2-yl-pyridin-3-yl)oxy-N-(4-morpholin-4-ylphenyl)pyridin-2-amine

¹H NMR: 2.35 (3H, s), 2.5 (3H, under DMSO), 3.00 (4H, t), 3.72 (4H, t), 6.00 (1H, d), 6.26 (1H, dd), 6.84 (2H, d), 7.33 (1H, ddd), 7.41 (2H, d), 7.55 (1H, s), 7.77 (1H, d), 7.85 (1H, td), 7.90 (1H, d), 8.51 (1H, d), 8.60 (1H, s); m/z: 454.3 M⁺; EAA: 0.00619; EAA2: 0.05784.

### Example 24

### 4-(5,6-Dimethyl-2-pyridin-2-yl-pyridin-3-yl)oxy-N-phenyl-pyridin-2-amine

¹H NMR: 2.36 (3H, s), 2.53 (3H, s), 6.10 (1H, d), 6.35 (1H, dd), 6.85 (1H, t), 7.17-7.24 (2H, m), 7.32 (1H, ddd), 7.57 (2H, s), 7.60 (1H, s), 7.77-7.88 (2H, m), 7.97 (1H, d), 8.51 (1H, d), 8.86 (1H, s); m/z: 369.2 M⁺; EAA: 0.00417; EAA: 0.004169.

### Example 25

### 4-(5,6-Dimethyl-2-pyridin-2-yl-pyridin-3-yl)oxy-N-(4-methoxy-phenyl)-pyridin-2-amine

¹H NMR: 2.35 (3H, s), 2.5 (3H, under DMSO), 3.70 (3H, s), 6.01 (1H, d), 6.27 (1H, dd), 6.81 (2H, d), 7.33 (1H, t), 7.45 (2H, d), 7.55 (1H, s), 7.76-7.88 (2H, m), 7.91 (1H, d), 8.51 (1H, d), 8.65 (1H, s); m/z: 399.7 M⁺; EAA: 0.00167; EAA2: 0.02175.

### Example 26

### 4-{[4-(5,6-Dimethyl-2-pyridin-2-yl-pyridin-3-yl)oxypyridin-2-yl]-amino}benzenesulfonamide

¹H NMR: 2.36 (3H, s), 2.54 (3H, s), 6.18 (1H, d), 6.46 (1H, dd), 7.10 (2H, s), 7.32 (1H, ddd), 7.59 (1H, s), 7.66 (2H, d), 7.75-7.88 (4H, m), 8.05 (1H, d), 8.49 (1H, d), 9.34 (1H, s); m/z: 448.7 M⁻; EAA: 0.0219; EAA2: 0.01244.

### Example 27

### 4-{[4-(5,6-Dimethyl-2-pyridin-2-yl-pyridin-3-yl)oxypyridin-2-yl]-amino}-N,N-dimethyl-benzamide

¹H NMR: 2.36 (3H, s), 2.54 (3H, s), 2.95 (6H, s), 6.14 (1H, d), 6.41 (1H, dd), 7.28-7.34 (3H, m), 7.58 (1H, s), 7.66 (2H, d), 7.79-7.87 (2H, m), 8.01 (1H, d), 8.50 (1H, d), 9.10 (1H, s); m/z: 440.6 MH⁺; EAA: 0.00706; EAA2: 0.0124.

### Example 28

### 4-(5,6-Dimethyl-2-pyridin-2-yl-pyridin-3-yl)oxy-N-(3,4,5-tri-methoxy-phenyl)pyridin-2-amine

¹H NMR: (CDCl₃): 2.34 (3H, s), 2.61 (3H, s), 3.78 (6H, s), 3.81 (3H, s), 6.21-6.26 (2H, m), 6.32 (1H, s), 6.49 (2H, s), 7.17-7.22 (1H, m), 7.64-7.70 (1H, m), 7.76-7.79 (1H, m), 7.97 (1H, d), 8.63-8.66 (1H, m); m/z: 459.4 MH⁺; EAA: 0.0202; EAA2: 0.02658.

### Example 29

### N-(3-Fluorophenyl)-4-(5,6-dimethyl-2-pyridin-2-yl-pyridin-3-yl)-oxy-pyridin-2-amine

A mixture of Pd₂(dba)₃ (0.012 g), 5-(2-chloropyridin-4-yl)oxy-2,3-dimethyl-6-pyridin-2-yl-pyridine (Method 16, 0.1 g), 3-fluoroaniline (0.062 g), xantphos (0.022 g), Cs₂CO₃ (0.314 g) and DMA (5 mL) was heated to 150° C. for 1 h by microwave in a sealed tube. Upon cooling the mixture was concentrated in vacuo and the residue was partitioned between DCM and water. The aqueous portion was extracted twice with DCM. The combined organic portions were filtered through a phase-separating filtercup and stirred with polystyrene-based thiophenol resin at r.t. for 3 h. The resin was removed by filtration and the filtrate was concentrated in vacuo. The residue was purified by reverse-phase HPLC, eluting with 5-95% MeCN in water containing 1% concentrated aqueous ammonia solution. The appropriate fractions were concentrated in vacuo to give the title compound as an off-white solid, (11 mg, 9%); ¹H NMR: 2.37 (3H, s), 2.54 (3H, s), 6.11 (1H, d), 6.43 (1H, dd), 6.64 (1H, dddd), 7.22 (2H, m), 7.33 (1H, ddd), 7.59 (1H, s), 7.78 (1H, ddd), 7.82 (1H, ddd), 7.86 (1H, dd), 8.03 (1H, d), 8.50 (1H, dd), 9.13 (1H, s); m/z: MH⁺ 387.5; EAA: 0.0166; EAA2: 0.01841.

The procedure described above was repeated using the appropriate aniline with 5-(2-chloropyridin-4-yl)oxy-2,3-dimethyl-6-pyridin-2-yl-pyridine (prepared according to Method 16). The examples described below were thus obtained:

| Ex. | Name and Data |
|---|---|
| 30 | N-(4-Chlorophenyl)-4-(5,6-dimethyl-2-pyridin-2-yl-pyridin-3-yl)oxy- |
| | pyridin-2-amine |
| | m/z: 403.5 MH⁺; EAA: 1.67; EAA2: 0.8942. |
| 31 | N-(2,5-Difluorophenyl)-4-(5,6-dimethyl-2-pyridin-2-yl-pyridin-3-yl)- |
| | oxy-pyridin-2-amine |
| | m/z: 405.5MH⁺; EAA: 0.0274; EAA2: 0.0312. |
| 32 | 4-(5,6-Dimethyl-2-pyridin-2-yl-pyridin-3-yl)oxy-N-(2-ethylpyrazol-3- |
| | yl)-pyridin-2-amine |
| | m/z: 387.6 MH⁺; EAA: 0.0425; EAA2: 0.03391. |
| 33 | (4-{[4-(5,6-Dimethyl-2-pyridin-2-yl-pyridin-3-yl)-oxypyridin-2- |
| | yl]amino}phenyl)methanol |
| | m/z: 399.5 MH⁺; EAA: 0.0172; EAA2: 0.01674. |
| 34 | 4-{[4-(5,6-Dimethyl-2-pyridin-2-yl-pyridin-3-yl)-oxypyridin-2- |
| | yl]amino}benzonitrile |
| | m/z: 394.5 MH⁺; EAA: 0.0215; EAA2: 0.02547. |
| 35 | N-[4-(5,6-Dimethyl-2-pyridin-2-yl-pyridin-3-yl)-oxypyridin-2-yl]-1H- |
| | indol-5-amine |
| | m/z: 408.6 MH⁺; EAA: 0.205; EAA2: 0.463. |
| 36 | 4-(5,6-Dimethyl-2-pyridin-2-yl-pyridin-3-yl)oxy-N-pyridin-4-yl- |
| | pyridin-2-amine |
| | m/z: 370.6 MH⁺; EAA: 0.0632; EAA2: 0.05147. |
| 37 | N-(2,4-Difluorophenyl)-4-(5,6-dimethyl-2-pyridin-2-yl-pyridin-3- |
| | yl)oxy-pyridin-2-amine |
| | m/z: 405.6 MH⁺; EAA: 0.0360; EAA2: 0.02155. |
| 38 | (3-{[4-(5,6-Dimethyl-2-pyridin-2-yl-pyridin-3-yl)-oxypyridin-2- |
| | yl]amino}phenyl)methanol |
| | m/z: 399.6 MH⁺; EAA: 0.00977; EAA2: 0.003777. |
| 39 | 4-(5,6-Dimethyl-2-pyridin-2-yl-pyridin-3-yl)oxy-N-(1- |
| | methylpyrazol-3-yl)-pyridin-2-amine |
| | m/z: 373.6 MH⁺; EAA: 0.0935; EAA: 0.09347. |
| 40 | N-[4-(5,6-Dimethyl-2-pyridin-2-yl-pyridin-3-yl)-oxypyridin-2-yl]-1H- |
| | indol-6-amine |
| | ¹H NMR: 2.38 (3H, s), 2.55 (3H, s), 4.95 (1H, s), 6.48 (1H, d), |
| | 6.53 (1H, dd), 6.62 (1H, dd), 7.11 (1H, d), 7.25 (1H, d), 7.31 |
| | (1H, ddd), 7.56 (1H, d), 7.56 (1H, s), 7.65 (1H, s), 7.85 (2H, m), |
| | 8.30 (1H, d), 8.48 (1H, ddd); m/z: 408.6 MH⁺; |
| | EAA: 1.14; EAA: 1.115. |

### Example 41

### 3-{[4-(5,6-Dimethyl-2-pyridin-2-yl-pyridin-3-yl)oxypyridin-2-yl]-amino}-benzonitrile

A mixture of Pd₂(dba)₃ (0.06 g), 5-(2-chloropyridin-4-yl)oxy-2,3-dimethyl-6-pyridin-2-yl-pyridine (Method 16, 0.05 g), m-aminobenzonitrile (0.028 g), xantphos (0.011 g), Cs₂CO₃ (0.157 g) and DMA (5 mL) was heated to 150° C. for 1 h by microwave in a sealed tube. After cooling, the mixture was concentrated in vacuo and the residue was purified by FCC using a gradient of 0-2% (10:1 MeOH/concentrated aqueous ammonia solution) in DCM to afford the title compound (21 mg, 33.3%) as a beige solid after trituration with diethyl ether; ¹H NMR: 2.37 (3H, s), 2.61 (3H, s), 6.13 (1H, d), 6.38 (1H, dd), 6.58 (1H, s), 7.19 (1H, dd), 7.22 (1H, ddd), 7.29 (1H, s), 7.33 (1H, dd), 7.46 (1H, ddd), 7.68 (1H, ddd), 7.80 (1H, dd), 7.85 (1H, dd), 8.02 (1H, d), 8.63 (1H, dd); m/z: MH⁺ 394.4; EAA: 0.0141; EAA2: 0.01235.

### Example 42

### 4-{4-[(2,6-Dimethylpyridin-3-yl)oxy]pyridin-2-yl}amino)-benzenesulfonamide

A mixture of 3-(2-chloropyridin-4-yl)oxy-2,6-dimethyl-pyridine (Method 13, 0.200 g, 0.85 mmol), sulfanilamide (0.191 g), Cs₂CO₃ (0.417 g), Pd(OAc)₂ (0.013 g), xantphos (49 mg) and DMA (2 mL) was heated to 150° C. for 10 minutes by microwave in a sealed tube. After cooling the crude product was semi-purified by ion exchange chromatography, using an SCX column eluting with 7M NH₃/MeOH. Further purification by preparative HPLC using decreasingly polar mixtures of water (containing 0.1% TFA) and MeCN as eluent afforded the title compound (0.17 g, 54%); ¹H NMR: 2.30 (3H, s), 2.49 (3H, s), 6.13 (1H, d), 6.52-6.56 (1H, m), 7.10 (2H, s), 7.23 (1H, d), 7.50 (1H, d), 7.68 (2H, d), 7.79 (2H, d), 8.14 (1H, d), 9.38 (1H, s); m/z: MH⁺ 371; EAA: 0.0431; EAA2: 0.07167.

### Example 43

### 4-{[4-(6-Methyl-2-pyridin-2-yl-pyridin-3-yl)oxypyridin-2-yl]-amino}benzene-sulfonamide

A mixture of sulfanilamide (0.069 g), 3-(2-chloropyridin-4-yl)oxy-6-methyl-2-pyridin-2-yl-pyridine (Method 17, 0.120 g), Cs₂CO₃ (0.197 g), Pd(OAc)₂ (6.33 mg), xantphos (0.023 g) and DMF (4 mL) was heated to 150° C. for 10 minutes by microwave in a sealed tube. After cooling, the crude product was semi-purified by ion exchange chromatography, using an SCX column eluting with 7M NH₃/MeOH and appropriate fractions were evaporated to dryness to afford crude product which was further purified by FCC eluting with a gradient of 0-10% EtOH in DCM to afford the title compound; (84 mg, 48%); ¹H NMR: 2.70 (3H, s), 6.27 (1H, d), 6.57-6.54 (1H, m), 7.19 (2H, s), 7.48-7.42 (1H, m), 7.58 (1H, d), 7.75 (2H, d), 7.81 (1H, d), 7.86 (2H, d), 7.92-7.87 (1H, m), 7.99-7.93 (1H, m), 8.15 (1H, d), 8.63-8.59 (1H, m), 9.42 (1H, s); m/z: MH⁺ 434; EAA: 0.0162; EAA2: 0.007278.

### Example 44

### 4-{[4-(6-Ethyl-2-pyridin-2-yl-pyridin-3-yl)oxypyridin-2-yl]-amino}benzene-sulfonamide

A mixture of sulfanilamide (0.085 g), 3-(2-chloropyridin-4-yl)oxy-6-ethyl-2-pyridin-2-yl-pyridine (Method 19, 0.153 g), Cs₂CO₃ (0.240 g), Pd(OAc)₂ (0.077g), xantphos (0.028 g) and DMA (4 mL) was heated to 150° C. for 10 minutes by microwave in a sealed tube. The crude product was semi-purified by ion exchange chromatography, using an SCX column eluting with 7M NH₃/MeOH and appropriate fractions were evaporated to dryness to afford semi-purified product. FCC, using a gradient of 0-10% MeOH in DCM afforded the title compound; (0.101 g, 46%); ¹H NMR: 1.43 (3H, t), 2.98 (2H, q), 6.28 (1H, d), 6.57-6.53 (1H, m), 7.18 (2H, s), 7.47-7.42 (1H, m), 7.59 (1H, d), 7.75 (2H, d), 7.82 (1H, d), 7.86 (2H, d), 7.94-7.90 (1H, m), 7.98-7.94 (1H, m), 8.15 (1H, d), 8.62-8.59 (1H, m), 9.42 (1H, s); m/z: MH⁺ 448; EAA: 0.0161; EAA2: 0.01652.

### Example 45

### 4-(6-Methyl-2-phenyl-pyridin-3-yl)oxy-N-(3,4,5-trimethoxy-phenyl)pyridin-2-amine

A mixture of 3,4,5-trimethoxyaniline (130 mg), 3-(2-chloropyridin-4-yl)oxy-6-methyl-2-phenyl-pyridine (Method 23, 0.14 g), xantphos (0.33 g), Pd₂(dba)₃ (0.017 g), Cs₂CO₃ (0.46 g) and DMA (5 mL) was heated to 130° C. for 30 minutes by microwave in a sealed tube. After cooling the mixture was filtered. The filtrate was diluted with EtOAc (20 mL), and washed with saturated brine (15 mL). The organic portion was dried (MgSO₄) and concentrated in vacuo. Part-purification by FCC using a gradient of 0-30% EtOAc in DCM followed by a further purification by FCC using a gradient of 10-50% EtOAc in iso-hexane afforded the title compound (0.12 g, 57%); ¹H NMR: 2.56 (3H, s), 3.71 (6H, s), 3.74 (3H, s), 6.17 (1H, d), 6.18-6.21 (1H, m), 6.32 (1H, s), 6.39 (2H, s), 7.07 (1H, d), 7.23-7.31 (4H, m), 7.70-7.74 (2H, m), 7.92-7.93 (1H, m); m/z: MH⁺ 444.54; EAA: 0.00537; EAA2: 0.007992.

### Example 46

### 4-[6-Methyl-2-(4-methylthiophen-3-yl)pyridin-3-yl]oxy-N-(3,4,5-trimethoxyphenyl)pyridin-2-amine

A mixture of 3,4,5-trimethoxyaniline (0.125 g), 3-(2-chloropyridin-4-yloxy)-6-methyl-2-(4-methylthiophen-3-yl)pyridine (Method 24, 0.18 g), xantphos (0.04 g), Pd₂(dba)₃ (0.02 g), Cs₂CO₃ (0.55g) and DMA (5mL) was heated to 130° C. for 30 minutes by microwave in a sealed tube. After cooling the mixture was filtered. The filtrate was diluted with EtOAc (20 mL) and washed with saturated brine (15 mL). The organic portion was dried (MgSO₄) and concentrated in vacuo. Part-purification by FCC using a gradient of 0-30% EtOAc in DCM afforded material that was further purified by FCC using a gradient of 10-50% EtOAc in iso-hexane to afford the title compound (0.135 g, 51%) as a beige solid; ¹H NMR: 2.17 (3H, s), 2.53 (3H, s), 3.72 (6H, s), 3.74 (3H, s), 6.10-6.14 (2H, m), 6.30 (1H, s), 6.40 (2H, s), 6.83-6.85 (1H, m), 7.05 (1H, s), 7.07 (1H, s), 7.27-7.31 (2H, m), 7.91 (1H, d); m/z: MH⁺ 464.46; EAA: 0.0153; EAA2: 0.01746.

### Example 47

### 4-(6-Methyl-2-pyridin-2-yl-pyridin-3-yl)oxy-N-(3,4,5-trimethoxy-phenyl)pyridin-2-amine

A mixture of 3-(2-chloropyridin-4-yl)oxy-6-methyl-2-pyridin-2-yl-pyridine (Method 17, 0.125 g), 3,4,5-trimethoxyaniline (0.092 g), xantphos (0.029 g), Pd₂(dba)₃ (0.015 g), Cs₂CO₃ (0.41 g) and DMA (5mL) was heated to 130° C. for 30 minutes by microwave in a sealed tube. After cooling, the mixture was filtered. The filtrate was diluted with EtOAc (20 mL) and washed with saturated brine (15 mL). The organic portion was dried (MgSO₄) and concentrated in vacuo. Purification by FCC using a gradient of 0-5% methanolic ammonia (7M) in DCM afforded the title compound; (26 mg, 14%); ¹H NMR: 2.61 (3H, s), 3.72 (6H, s), 3.74 (3H s), 6.15 (2H, d), 6.19 (2H, q), 6.27 (1H, s), 6.41 (2H, s), 7.13-7.18 (2H, m), 7.35 (1H, d), 7.60-7.64 (1H, m), 7.70 (1H, d), 7.90 (1H, d), 8.59 (1H, d); m/z: MH⁺ 445.49; EAA: 0.0248; EAA2: 0.01084.

### Example 48

### 4-(6-Methyl-2-pyrazin-2-yl-pyridin-3-yl)oxy-N-(3,4,5-trimethoxy-phenyl)pyridin-2-amine

A mixture of 3,4,5-trimethoxyaniline (66 mg), 2-{3-[(2-chloropyridin-4-yl)oxy]-6-methylpyridin-2-yl}pyrazine (Method 25, 0.09 g) and xantphos (21 mg) Pd₂(dba)₃ (11 mg), Cs₂CO₃ (72 mg) and DMA (5 mL) was heated to 130° C. for 30 minutes by microwave in a sealed tube. After cooling the mixture was diluted with EtOAc (20 mL) and washed with saturated brine (15 mL). The organic portion was dried (MgSO₄) and concentrated in vacuo. Purification by FCC using a gradient of 0-5% methanolic ammonia in DCM afforded the title compound (23 mg, 17%) as a beige solid after trituration with diethyl ether; ¹H NMR: 2.62 (3H, s), 3.73 (6H, s), 3.74 (1H, s), 6.17-6.20 (2H, m), 6.30 (1H, s), 6.41 (2H, s), 7.23 (1H, d), 7.39 (1H, d), 7.93 (1H, d), 8.45 (1H, d), 8.54-8.56 (1H, m), 9.03 (1H, d); /z: MH⁺ 446.50; EAA: 0.0751; EAA2: 0.04857.

### Example 49

### 4-(2-Phenylpyridin-3-yl)oxy-N-(3,4,5-trimethoxyphenyl)pyridin-2-amine

A mixture of Pd(PPh₃)₄ (5 mg), 4-(2-chloropyridin-3-yl)oxy-N-(3,4,5-trimethoxyphenyl)pyridin-2-amine (Example 53, 35 mg), phenylboronic acid (22 mg), Na₂CO₃ (29 mg), 1,4-dioxane (1 mL) and water (0.1 mL) was heated at 80° C. in a microwave for 1.5 h in a sealed tube. After cooling the mixture was diluted with in EtOAc and water and the phases were separated. The aqueous portion was extracted with EtOAc. The combined organic portions were dried (Na₂SO₄) and concentrated in vacuo. Part-purification by FCC using 3-10% MeOH in DCM, followed by further purification by FCC using 50% EtOAc in hexane, followed by further purification by basic HPLC afforded the title compound (9 mg, 22%) as a gum; ¹H NMR: 3.78 (6H, s), 3.81 (3H, s), 6.25-6.29 (2H, m), 6.47 (2H, s), 6.50 (1H, s), 7.26-7.42 (4H, m), 7.47 (1H, dd), 7.80-7.84 (2H, m), 8.02 (1H, d), 8.60 (1H, dd); m/z: MH⁺ 430.58; EAA: 0.00782; EAA2: 0.008042.

The following examples were prepared in a similar fashion using 4-(2-chloropyridin-3-yl)oxy-N-(3,4,5-trimethoxyphenyl)pyridin-2-amine (which was prepared as described in Example 53).

| Ex. | Name and Data |
|---|---|
| 50 | 2-(3-{2-[(3,4,5-Trimethoxyphenyl)amino]pyridin-4-yl}-oxypyridin-2- |
| | yl)benzonitrile |
| | ¹H NMR: 3.81 (6H, s), 3.81 (3H, s), 6.25-6.30 (2H, m), 6.42 (1H, s), |
| | 6.54 (2H, s), 7.39-7.63 (5H, m), 7.73 (1H, dt), 8.03 (1H, d), 8.63 (1H, dd); m/z: 455.3 |
| | MH⁺; EAA: 0.1256; EAA2: 0.0308. |
| 51 | N-[4-(3-{2-[(3,4,5-Trimethoxyphenyl)amino]pyridin-4-yl}-oxypyridin- |
| | 2-yl)phenyl]acetamide |
| | ¹H NMR: 2.02 (3H, s), 3.62 (3H, s), 3.68 (6H, s), 6.01 (1H, d), 6.22 (1H, |
| | dd), 6.62 (2H, s), 7.37 (1H, dd), 7.41-7.66 (7H, m), 7.82 (1H, d), 8.45 (1H, |
| | dd); m/z: 487.3 MH⁺; EAA: 0.230; EAA2: 0.02739. |
| 52 | 4-[2-(4-Fluorophenyl)pyridin-3-yl]oxy-N-(3,4,5-trimethoxy- |
| | phenyl)pyridin-2-amine |
| | ¹H NMR: 3.59 (3H, s), 3.71 (6H, s), 6.09 (1H, d), 6.40-6.42 (1H, m), |
| | 6.93-6.94 (2H, m), 7.23-7.29 (2H, m), 7.52-7.56 (1H, m), 7.77-7.80 (1H, m), |
| | 7.85-7.90 (2H, m), 8.03 (1H, d), 8.64-8.66 (1H, m), 8.84 (1H, s); m/z: |
| | 447.5 MH⁺; EAA: 0.0156; EAA2: 0.03286. |

### Example 53

### 4-(2-Chloropyridin-3-yl)oxy-N-(3,4,5-trimethoxyphenyl)pyridin-2-amine

Pd(OAc)₂ (56 mg) was added to a mixture of 3,4,5-trimethoxyaniline (500 mg, 2.73 mmol), 2-chloro-4-(2-chloropyridin-3-yl)oxy-pyridine (Method 26, 0.6 g), xantphos (0.273 g) and Cs₂CO₃ (1.50 g) in DMA (12 mL) in a microwave tube. The mixture was heated in a microwave at 100° C. for 15 mins. After cooling the mixture was diluted with EtOAc and water and the phases were separated. The aqueous portion was extracted with EtOAc. The combined organic portions were washed with saturated brine, dried (Na₂SO₄) and concentrated in vacuo. Part-purification by FCC using 50% EtOAc in hexane, followed by further purification by FCC using 6% MeOH in DCM afforded the title compound (0.2 g, 21%) as a white solid; ¹H NMR: 3.81 (6H, s), 3.81 (3H, s), 6.26 (1H, d), 6.31 (1H, dd), 6.44 (1H, s), 6.50 (2H, s), 7.30 (1H, dd), 7.48 (1H, dd), 8.10 (1H, d), 8.30 (1H, dd); m/z: MH⁺ 388.39; EAA: 0.481; EAA2: 0.2486.

### Example 54

### 4-(2-Chloro-6-methyl-pyridin-3-yl)oxy-N-(3,4,5-trimethoxyphenyl)-pyridin-2-amine

A mixture of 2-chloro-3-(2-chloropyridin-4-yl)oxy-6-methyl-pyridine (Method 27, 0.66 g), 3,4,5-trimethoxyaniline (0.5 g), xantphos (0.45 g), Pd(OAc)₂ (0.059 g), Cs₂CO₃ (1.69 g) in DMA (30 mL) was heated in a microwave at 90° C. for 10 minutes. After cooling the mixture was diluted with EtOAc and filtered through diatomaceous earth. The diatomaceous earth was washed with further EtOAc. The combined organic portions were washed with water and with saturated brine, then dried (Na₂SO₄) and concentrated in vacuo. Part-purification by FCC using 0-10% MeOH in DCM followed by further purification by FCC using 30-70% EtOAc in hexane afforded the title compound (0.232 g, 11%) as a white solid; ¹H NMR: 2.56 (3H, s), 3.76-3.82 (9H, m), 6.24 (1H, d), 6.30 (1H, dd), 6.43-6.45 (1H, m), 6.49 (2H, s), 7.13 (1H, d), 7.37 (1H, d), 8.08 (1H, d).

### Example 55

### 4-[2-(3-Chlorophenyl)-6-methyl-pyridin-3-yl]oxy-N-(3,4,5-trimethoxyphenyl)-pyridin-2-amine

A mixture of Pd(PPh₃)₄ (8 mg), 4-(2-chloro-6-methyl-pyridin-3-yl)oxy-N-(3,4,5-trimethoxyphenyl)-pyridin-2-amine (Example 54, 58 mg), (3-chlorophenyl)boronic acid (45 mg), Na₂CO₃ (46 mg), dioxane (1.2 mL) and water (0.24 mL) heated in a microwave at 130° C. for 1 h in a sealed tube. After cooling water and DCM were added and the mixture was stirred for 5 minutes, then the organic phase was isolated by separation through a PTFE filter. The organic portion was concentrated in vacuo and purified by basic HPLC to afford the title compound (11 mg, 16%); ¹H NMR: 2.63 (3H, s), 3.79 (6H, s), 3.81 (3H, s), 6.22-6.26 (2H, m), 6.42 (1H, br s), 6.46 (2H, s), 7.16 (1H, d), 7.26-7.31 (3H, m), 7.36 (1H, d), 7.69-7.72 (1H, m), 7.84-7.86 (1H, m), 8.00 (1H, d); m/z: MH⁺ 477.90; EAA: 0.00929; EAA2: 0.03455.

The following examples were prepared in an identical fashion using 4-(2-chloro-6-methyl-pyridin-3-yl)oxy-N-(3,4,5-trimethoxyphenyl)pyridin-2-amine (which was prepared as described in Example 54).

| Ex. | Name and Data |
|---|---|
| 56 | 4-[2-(3-Fluoro-phenyl)-6-methyl-pyridin-3-yl]oxy-N-(3,4,5-trimethoxy- |
| | phenyl)pyridin-2-amine |
| | ¹H NMR: 2.63 (3H, s), 3.79 (6H, s), 3.82 (3H, s), 6.23-6.26 (2H, m), |
| | 6.42 (1H, s), 6.46 (2H, s), 7.03-7.04 (1H, m), 7.16 (1H, d), 7.29-7.38 (2H, m), |
| | 7.55-7.60 (1H, m), 7.61-7.64 (1H, m), 7.99-8.01 (1H, m); m/z: 461.9 MH⁺; |
| | EAA: 0.00379; EAA2: 0.07949. |
| 57 | 4-[6-Methyl-2-(3-methylphenyl)pyridin-3-yl]oxy-N-(3,4,5- |
| | trimethoxyphenyl)-pyridin-2-amine |
| | ¹H NMR: 2.35 (3H, s), 2.63 (3H, s), 3.78 (6H, s), 3.81 (3H, s), |
| | 6.22-6.23 (1H, m), 6.26-6.29 (1H, s), 6.45 (3H, s), 7.11-7.15 (2H, m), 7.21 (1H, d), |
| | 7.34 (1H, d), 7.55 (1H, d), 7.60 (1H, s), 7.99 (1H, d); m/z: 458.0 MH⁺; CAA: |
| | 0.0904; EAA: 0.01538. |
| 58 | 4-[2-(3-Methoxy-phenyl)-6-methyl-pyridin-3-yl]oxy-N-(3,4,5- |
| | trimethoxy-phenyl)pyridin-2-amine |
| | ¹H NMR: 2.63 (3H, s), 3.77 (3H, s), 3.78 (6H, s), 3.81 (3H, s), |
| | 6.23-6.29 (2H, m), 6.43 (1H, s), 6.46 (2H, s), 6.86-6.90 (1H, m), 7.14 (1H, d), |
| | 7.24-7.40 (4H, m), 7.99 (1H, d); m/z: 474.0 MH⁺; EAA: 0.0187; EAA2: 0.1354. |

### Example 59

### N-Cyclopropyl-3-{[4-(2,6-dimethylpyridin-3-yl)oxypyridin-2-yl]-amino}-N-(1-methylpiperidin-4-yl)benzamide

Sodium 3-[4-(2,6-dimethylpyridin-3-yloxy)pyridin-2-ylamino]benzoate (Example 210, 0.100 g) was added to N-hydroxybenzotriazole (0.064 g) in THF (3.0 mL). The resulting solution was treated with N-cyclopropyl-1-methyl-piperidin-4-amine (2 eq) and silica-supported dicyclohexylcarbodiimide (DCC) (1.12 mmol/g, 0.625 g). The mixture was heated to 110° C. for 30 mins by microwave After cooling to r.t. the mixture was diluted with THF (2 mL). The mixture was loaded onto a 1 g cartridge of silica-supported carbonate and the cartridge was eluted with DCM (5 mL). Further purification by basic reverse phase HPLC afforded the title compound (0.010 g, 10%); ¹H NMR: 0.46-0.54 (2H, m), 0.55-0.66 (2H, m), 1.85-1.94 (2H, m), 2.04-2.22 (5H, m), 2.32 (3H, s), 2.39 (3H, s), 2.54 (3H, s), 2.93-3.02 (2H, m), 4.13-4.25 (1H, m), 6.21-6.27 (2H, m), 6.76 (1H, s), 7.02 (1H, d), 7.08-7.12 (1H, m), 7.22 (1H, d), 7.30 (1H, d), 7.37-7.44 (2H, m), 8.04 (1H, d); m/z: MH⁺ 472; EAA2: 0.02139.

The procedure described above was repeated by reacting sodium 3-[4-(2,6-dimethylpyridin-3-yloxy)pyridin-2-ylamino]benzoate (Example 210) with the appropriate amine (or if the HCl salt of the amine was used then DIPEA (3 eq) was also added). This provided the Examples listed below:

### Example 60

### N-Cyclopropyl-3-{[4-(2,6-dimethylpyridin-3-yl)oxypyridin-2-yl]-amino}-N-(oxan-4-yl)benzamide

¹H NMR: 0.47-0.54 (2H, m), 0.57-0.67 (2H, m), 1.79-1.87 (2H, m), 2.09-2.25 (2H, m), 2.38 (3H, s), 2.51-2.60 (1H, m), 2.53 (3H, s), 3.44-3.55 (2H, m), 4.01-4.09 (2H, m), 4.34-4.47 (1H, m), 6.22-6.25 (2H, m), 7.02 (1H, d), 7.08-7.14 (1H, m), 7.22 (1H, d), 7.27 (1H, d), 7.30 (1H, d), 7.39-7.47 (2H, m), 8.03-8.06 (1H, m); m/z: 459 MH⁺; EAA2: 0.02204.

### Example 61

### Azetidin-1-yl-(3-{[4-(2,6-dimethylpyridin-3-yl)oxypyridin-2-yl]-amino}phenyl)methanone

¹H NMR: 2.26-2.37 (2H, m), 2.38 (3H, s), 2.53 (3H, s), 4.15-4.24 (2H, m), 4.25-4.35 (2H, m), 6.24 (1H, d), 6.25-6.28 (1H, m), 7.03 (1H, d), 7.20-7.25 (2H, m), 7.27 (1H, d), 7.32 (1H, d), 7.47-7.52 (1H, m), 7.68-7.71 (1H, m), 8.07 (1H, d); m/z: 375 MH⁺; EAA2: 0.0227.

### Example 62

### 3-{[4-(2,6-Dimethylpyridin-3-yl)oxypyridin-2-yl]amino}-N,N-dimethylbenzamide

¹H NMR: 2.39 (3H, s), 2.54 (3H, s), 2.98 (3H, s), 3.09 (3H, s), 6.24-6.27 (2H, m), 7.00-7.06 (2H, m), 7.09 (1H, s), 7.23 (1H, d), 7.27-7.34 (1H, m), 7.37-7.44 (2H, m), 8.05 (1H, d); m/z: 363 MH⁻; EAA2: 0.0535.

### Example 63

### 3-{[4-(2,6-Dimethylpyridin-3-yl)oxypyridin-2-yl]amino}-N-(1H-imidazol-2-ylmethyl)benzamide

¹H NMR: 2.37 (3H, s), 2.52 (3H, s), 4.64 (2H, d), 4.84 (2H, s), 6.21-6.26 (2H, m), 6.96 (2H, s), 7.01 (1H, d), 7.21 (1H, d), 7.28 (1H, d), 7.46 (1H, d), 7.54-7.59 (1H, m), 7.83-7.87 (1H, m), 8.01 (1H, d), 8.54-8.60 (1H, m); m/z: 415 MH⁺; EAA2: 0.008235.

### Example 64

### 3-{[4-(2,6-Dimethylpyridin-3-yl)oxypyridin-2-yl]amino}-N-(pyrazin-2-ylmethyl)benzamide

¹H NMR: 2.39 (3H, s), 2.53 (3H, s), 4.81 (2H, d), 6.23-6.30 (2H, m), 7.01-7.10 (1H, m), 7.03 (1H, d), 7.24 (1H, d), 7.35 (2H, t), 7.43-7.47 (1H, m), 7.53-7.58 (1H, m), 7.85-7.88 (1H, m), 8.06 (1H, d), 8.50-8.54 (2H, m), 8.68 (1H, d); m/z: 427 MH⁺ EAA2: 0.04152.

### Example 65

### N-[1-(3-{[4-(2,6-Dimethylpyridin-3-yl)oxypyridin-2-yl]-amino}benzoyl)pyrrolidin-3-yl]acetamide

¹H NMR: 1.84-2.00 (2H, m), 2.20 (3H, s), 2.37 (3H, s), 2.54 (3H, s), 3.31-3.37 (0.5H, m), 3.45-3.57 (1.5H, m), 3.61-3.85 (2H, m), 4.37-4.46 (0.5H, m), 4.50-4.61 (0.5H, m), 6.19 (1H, d), 6.24-6.32 (1H, m), 6.54 (0.5H, d), 6.92-6.94 (0.5H, m), 7.01-7.08 (1H, m), 7.03 (1H, d), 7.23 (1H, d), 7.23-7.56 (4H, m), 8.03 (1H, d); m/z: 446 MH⁺; EAA2: 0.04571.

### Example 66

### (3-{[4-(2,6-Dimethylpyridin-3-yl)oxypyridin-2-yl]amino}phenyl)-(3-methylsulfonylpyrrolidin-1-yl)methanone

¹H NMR: 2.26 (2H, s), 2.38 (3H, s), 2.53 (3H, s), 2.84-2.99 (3H, m), 3.53-4.16 (5H, m), 6.20 (1H, d), 6.26-6.30 (1H, m), 7.03 (1H, d), 7.09 (1H, d), 7.24 (1H, d), 7.31 (1H, d), 7.42 (1H, s), 7.50 (1H, d), 7.58 (1H, s), 8.06 (1H, d); m/z: 467 MH⁺; EAA2: 0.01845.

### Example 67

### N-(2-Amino-2-oxoethyl)-3-{[4-(2,6-dimethylpyridin-3-yl)-oxypyridin-2-yl]amino}benzamide

¹H NMR: 2.35 (3H, s), 2.50 (3H, s), 4.05 (2H, d), 6.14 (1H, s), 6.20-6.25 (2H, m), 6.74 (1H, s), 6.99 (1H, d), 7.20 (1H, d), 7.25 (1H, d), 7.37 (1H, d), 7.53-7.65 (3H, m), 7.71 (1H, s), 7.98 (1H, d); m/z: 392 MH⁺; EAA2: 0.05397.

### Example 68

### 1-(3-{[4-(2,6-Dimethylpyridin-3-yl)oxypyridin-2-yl]amino}-benzoyl)piperidine-3-carboxamide

¹H NMR: 1.37-1.73 (2H, m), 1.75-2.08 (2H, m), 2.21 (2H, s), 2.38 (3H, s), 2.54 (3H, s), 2.73-2.88 (0.5H, m), 3.05-3.34 (1H, m), 3.45-3.64 (1H, m), 3.84-4.02 (0.5H, m), 4.13-4.34 (1H, m), 5.62-5.81 (1H, m), 6.21 (1H, s), 6.26-6.31 (1H, m), 6.85-6.99 (1H, m), 7.04 (1H, d), 7.23 (1H, d), 7.25-7.66 (4H, m), 8.04 (1H, d); m/z: 446 MH⁺; EAA2: 0.008952.

### Example 69

### 2-[4-(3-{[4-(2,6-Dimethylpyridin-3-yl)oxypyridin-2-yl]amino}-benzoyl)piperazin-1-yl]acetamide

¹H NMR: 2.01-2.09 (2H, m), 2.50-2.67 (4H, m), 2.54 (3H, s), 3.06 (3H, s), 3.41-3.88 (4H, m), 5.77-5.83 (1H, m), 6.24 (1H, d), 6.26-6.29 (1H, m), 6.91-6.97 (1H, m), 6.99-7.04 (1H, m), 7.06 (1H, s), 7.12 (1H, s), 7.23 (1H, d), 7.30 (1H, t), 7.38-7.43 (1H, m), 7.44-7.48 (1H, m), 8.05 (1H, d); /z: 461 MH⁺; EAA2: 0.08273.

### Example 70

### 3-{[4-(2,6-Dimethylpyridin-3-yl)oxypyridin-2-yl]amino}-N-(1-methylpiperidin-4-yl)benzamide

¹H NMR: 2.26-2.37 (2H, m), 2.38 (3H, s), 2.53 (3H, s), 4.15-4.24 (2H, m), 4.25-4.35 (2H, m), 6.24 (1H, d), 6.25-6.28 (1H, m), 7.03 (1H, d), 7.20-7.25 (2H, m), 7.27 (1H, d), 7.32 (1H, d), 7.47-7.52 (1H, m), 7.68-7.71 (1H, m), 8.07 (1H, d); m/z: 375 MH⁺; EAA2: 0.03227.

### Example 71

### [3-{[4-(2,6-Dimethylpyridin-3-yl)oxypyridin-2-yl]amino}phenyl]-(4-methylpiperazin-1-yl)methanone

¹H NMR: 2.00 (4H, s), 2.39 (3H, s), 2.51 (3H, s), 2.56 (3H, s), 2.68 (4H, s), 6.24-6.25 (1H, m), 6.26-6.30 (1H, m), 6.95 (1H, s), 7.04-7.08 (2H, m), 7.24 (2H, d), 7.36-7.38 (1H, m), 7.48 (1H, s), 8.04 (1H, d); m/z: 418 MH⁺; EAA2: 0.09596.

### Example 72

### N-Cyclopropyl-3-{[4-(5,6-dimethyl-2-pyridin-2-ylpyridin-3-yl)-oxypyridin-2-yl]amino}benzamide

5-(2-Chloropyridin-4-yl)oxy-2,3-dimethyl-6-pyridin-2-yl-pyridine (Method 16, 0.125 g), 3-amino-N-cyclopropylbenzamide (0.070 g), Cs₂CO₃ (0.195 g), Pd(OAc)₂ (6.29 mg) and xantphos (0.023 g) were suspended in DMF (4 mL) and sealed into a microwave tube. The mixture was heated to 150° C. for 15 mins in a microwave and was then cooled to r.t. The crude mixture was part-purified by ion exchange chromatography using an SCX column and eluting with 7M NH₃/MeOH. Appropriate fractions were concentrated to afford a brown oil. Further purification by FCC using a gradient of 0-8% EtOH in DCM. Concentration in vacuo provided the title compound (0.062 g, 34%) as a white solid; ¹H NMR: 8.66-8.62 (1H, m), 7.94 (1H, d), 7.75-7.64 (3H, m), 7.45-7.41 (1H, m), 7.35-7.25 (3H, m), 7.24-7.18 (1H, m), 7.12 (1H, s), 6.75 (1H, s), 6.30-6.26 (1H, m), 6.10 (1H, d), 2.96-2.87 (1H, m), 2.58 (3H, s), 2.34 (3H, s), 0.87-0.79 (2H, m), 0.72-0.66 (2H, m); m/z: MH⁺ 452; EAA2: 0.01075.

The procedure described above (Example 72) was repeated using the appropriate amine and 5-(2-chloropyridin-4-yl)oxy-2,3-dimethyl-6-pyridin-2-yl-pyridine (Method 16) to provide the Examples listed below:

### Example 73

### N-(3-{[4-(5,6-Dimethyl-2-pyridin-2-ylpyridin-3-yl)oxypyridin-2-yl]-amino}-4-methoxyphenyl)acetamide

¹H NMR: 2.25 (3H, s), 2.26 (3H, s), 2.50 (3H, s), 3.71 (3H, s), 6.02 (1H, d), 6.35-6.31 (1H, m), 6.68 (3H, d), 7.09 (1H, d), 7.19-7.15 (1H, m), 7.68-7.59 (2H, m), 7.77-7.72 (1H, m), 7.88 (1H, d), 8.63-8.59 (1H, m), 8.32 (1H, s), 8.06-8.03 (1H, m), 7.47-7.39 (2H, m), 7.30 (1H, d), 7.27 (1H, d); m/z: 456 MH⁺; EAA: 0.4544.

### Example 74

### 2-(4-{[4-(5,6-Dimethyl-2-pyridin-2-ylpyridin-3-yl)oxypyridin-2-yl]-amino}phenyl)acetonitrile

¹H NMR: 2.35 (3H, s), 2.61 (3H, s), 3.68 (2H, s), 7.18-7.31 (6H, m), 6.22 (1H, d), 6.26-6.30 (1H, m), 6.57 (1H, s), 7.65-7.72 (1H, m), 7.76-7.81 (1H, m), 7.98 (1H, d), 8.63-8.67 (1H, m); m/z: 408 MH⁺; EAA2: 0.00488.

### Example 75

### 1-(4-{[4-(5,6-Dimethyl-2-pyridin-2-ylpyridin-3-yl)oxypyridin-2-yl]-amino}phenoxy)-3-(propan-2-ylamino)propan-2-ol

¹H NMR: 1.12 (6H, d), 2.33 (3H, s), 2.60 (3H, s), 2.72-2.80 (1H, m), 2.84-2.96 (2H, m), 3.91-4.00 (2H, m), 4.01-4.11 (1H, m), 4.70 (1H, s), 6.11-6.18 (2H, m), 6.49 (1H, s), 6.85 (2H, d), 7.12 (2H, d), 7.17-7.25 (2H, m), 7.64-7.71 (1H, m), 7.72-7.77 (1H, m), 7.91 (1H, d), 8.62-8.68 (1H, m); m/z: 500 MH⁺; EAA2: 0.01363.

### Example 76

### N-(3-{[4-(5,6-Dimethyl-2-pyridin-2-ylpyridin-3-yl)oxypyridin-2-yl]-amino}phenyl)methanesulfonamide

¹H NMR: 2.33 (3H, s), 2.59 (3H, s), 3.05 (3H, s), 6.12 (1H, d), 6.34-6.37 (1H, m), 6.80-6.84 (1H, m), 6.93-6.98 (1H, m), 7.00-7.04 (1H, m), 7.16 (1H, d), 7.20-7.27 (4H, m), 7.65-7.72 (1H, m), 7.76-7.80 (1H, m), 7.95 (1H, d), 8.68-8.71 (1H, m); m/z: 462 MH⁺; EAA2: 0.01483.

### Example 77

### 4-(5,6-Dimethyl-2-pyridin-2-ylpyridin-3-yl)oxy-N-[3-(1.3-oxazol-5-yl)phenyl]pyridin-2-amine

¹H NMR: 2.42 (3H, s), 2.59 (3H, s), 6.18 (1H, d), 6.43-6.47 (1H, m), 7.26-7.30 (1H, m), 7.34-7.41 (2H, m), 7.62-7.66 (3H, m), 7.83-7.94 (2H, m), 8.06-8.10 (2H, m), 8.48 (1H, s), 8.54-8.58 (1H, m), 9.11 (1H, s); m/z: 436 MH⁻; EAA2: 0.008625.

### Example 78

### 4-(5,6-Dimethyl-2-pyridin-2-ylpyridin-3-yl)oxy-N-{3-[(4-methyl-piperazin-1-yl)methyl]phenyl}pyridin-2-amine

¹H NMR: 2.27 (3H, s), 2.34 (3H, s), 2.38-2.55 (8H, m), 2.60 (3H, s), 3.46 (2H, s), 6.20-6.24 (1H, m), 6.30 (1H, d), 6.70 (1H, s), 6.97 (1H, d), 7.13-7.25 (4H, m), 7.25-7.28 (1H, m), 7.64-7.71 (1H, m), 7.76-7.80 (1H, m), 7.96 (1H, d), 8.64-8.69 (1H, m); m/z: 481 MH⁺; EAA2: 0.03843.

### Example 79

### 4-(5,6-Dimethyl-2-pyridin-2-ylpyridin-3-yl)oxy-N-[3-(2-morpholin-4-ylethoxy)phenyl]pyridin-2-amine

¹H NMR: 2.34 (3H, s), 2.54-2.58 (4H, m), 2.60 (3H, s), 2.77 (2H, t), 3.69-3.75 (4H, m), 4.06 (2H, t), 6.21-6.25 (1H, m), 6.32 (1H, d), 6.53-6.58 (1H, m), 6.75-6.80 (1H, m), 6.86 (1H, s), 6.90-6.92 (1H, m), 7.14 (1H, d), 7.17-7.22 (1H, m), 7.26 (1H, s), 7.64-7.71 (1H, m), 7.75-7.80 (1H, m), 7.96 (1H, d), 8.62-8.66 (1H, m); m/z: 498 MH⁺; EAA2: 0.03962.

### Example 80

### 1-(4-{[4-(5,6-Dimethyl-2-pyridin-2-ylpyridin-3-yl)oxypyridin-2-yl]-amino}phenyl)-N-methylmethanesulfonamide

¹H NMR: 2.35 (3H, s), 2.61 (3H, s), 2.74 (3H, d), 4.19 (2H, s), 4.37 (1H, d), 6.20 (1H, d), 6.28-6.32 (1H, m), 6.86 (1H, s), 7.18-7.23 (1H, m), 7.23-7.30 (5H, m), 7.65-7.72 (1H, m), 7.77-7.82 (1H, m), 7.96 (1H, d), 8.62-8.66 (1H, m); m/z: 476 MH⁺; EAA2: 0.01695.

### Example 81

### 4-{[4-(5,6-Dimethyl-2-pyridin-2-ylpyridin-3-yl)oxypyridin-2-yl]-amino}benzamide

¹H NMR: 2.42 (3H, s), 2.59 (3H, s), 6.21 (1H, d), 6.47-6.51 (1H, m), 7.11 (1H, s), 7.35-7.41 (1H, m), 7.64 (1H, s), 7.71 (2H, d), 7.80 (2H, d), 7.68-7.83 (1H, m), 7.84-7.94 (2H, m), 8.09 (1H, d), 8.53-8.57 (1H, m), 9.22 (1H, s); m/z: 412 MH⁺; EAA2: 0.01107.

### Example 82

### Methyl 3-(2-anilinopyridin-4-yl)oxy-6-methylpyridine-2-carboxylate

Methyl 3-[(2-chloropyridin-4-yl)oxy]-6-methylpyridine-2-carboxylate (Method 30, 0.982 g), aniline (0.417 mL), Cs₂CO₃ (1.722 g), Pd(OAc)₂ (0.055 g) and xantphos (0.204 g) were suspended in DMA (20 mL) and sealed into a microwave tube. The mixture was heated to 160° C. for 20 minutes by microwave and was then cooled to r.t. The mixture was evaporated to dryness and redissolved in EtOAc (150 mL), and washed sequentially with water (25 mL) and saturated brine (25 mL). The organic layer was dried (MgSO₄), filtered and concentrated in vacuo to afford crude material that was triturated with DCM to provide a solid which was collected by filtration and dried in vacuo to afford the title compound (0.130 g, 11%) as a white solid. The remaining crude product was purified by FCC eluting with 1:1 EtOAc-isohexane to afford the title compound; (0.267 g, 22%); ¹H NMR: 2.56 (3H, s), 3.76 (3H, s), 6.12 (1H, d), 6.40-6.44 (1H, m), 6.84-6.91 (1H, m), 7.22 (1H, d), 7.24 (1H, d), 7.58-7.65 (3H, m), 7.76 (1H, d), 8.06 (1H, d), 8.94 (1H, s); m/z: 336 MH⁺.

### Example 83

### N-(4-{[4-(5,6-Dimethyl-2-pyridin-2-ylpyridin-3-yl)oxypyridin-2-yl]-amino}phenyl)-N-methylacetamide

The procedure described above (Example 72) was repeated using N-(4-aminophenyl)-N-methylacetamide and 5-(2-chloropyridin-4-yl)oxy-2,3-dimethyl-6-pyridin-2-yl-pyridine (Method 16) to provide the title compound: ¹H NMR: 1.87 (3H, s), 2.36 (3H, s), 2.62 (3H, s), 3.23 (3H, s), 6.20 (1H, d), 6.27-6.33 (1H, m), 6.47 (1H, s), 7.08 (2H, d), 7.18-7.24 (1H, m), 7.28 (1H, s), 7.35 (2H, d), 7.65-7.72 (1H, m), 7.77-7.82 (1H, m), 7.99 (1H, d), 8.62-8.67 (1H, m); m/z: 440 MH⁺; EAA2: 0.02587.

### Example 84

### 3-{[4-(5,6-Dimethyl-2-pyridin-2-ylpyridin-3-yl)oxypyridin-2-yl]-amino}-5-methanesulfonamidobenzamide

The procedure described above (Example 72) was repeated using 3-amino-5-[(methylsulfonyl)amino]benzamide and 5-(2-chloropyridin-4-yl)oxy-2,3-dimethyl-6-pyridin-2-yl-pyridine (Method 16) to provide the title compound: ¹H NMR: 2.32 (3H, s), 2.55 (3H, s), 3.01 (3H, s), 6.01 (1H, d), 6.07 (1H, s), 6.36 (1H, d), 7.16-7.25 (3H, m), 7.27 (1H, s), 7.35 (1H, s), 7.42 (1H, s), 7.63-7.76 (3H, m), 7.91 (1H, d), 8.64 (1H, d); m/z: 505 MH⁺; EAA2: 0.01602.

### Example 85

### Sodium 3-(2-Anilinopyridin-4-yl)oxy-6-methylpyridine-2-carboxylate

NaOH (1M in water, 2.368 mL) was added in one portion to methyl 3-(2-anilino-pyridin-4-yl)oxy-6-methylpyridine-2-carboxylate (Example 82, 0.397 g) in THF (3 mL) and MeOH (3 mL) at 40° C. The mixture was evaporated to dryness and redissolved in EtOAc (25 mL) and water (20 mL). The aqueous layer was extracted with EtOAc (4×30 mL). The organic layer was dried (MgSO₄), filtered and concentrated in vacuo to afford the title compound (0.272 g, 72%). The aqueous layer was then purified by preparative HPLC (Waters XBridge Prep C18 OBD column, 5 μm silica, 21 mm diameter, 150 mm length), using decreasingly polar mixtures of water (containing 1% NH₃) and MeCN as eluents to provide more of the title compound (0.069 g, 18%); ¹H NMR: 2.52 (3H, s), 6.28 (1H, s), 6.51 (1H, s), 7.00-7.38 (8H, m), 7.57 (1H, d), 11.02 (1H, s); m/z: 322 MH⁺.

### Example 86

### N-(4-{[4-(5,6-Dimethyl-2-pyridin-2-ylpyridin-3-yl)oxypyridin-2-yl]-amino}phenyl)acetamide

The procedure described above (Example 72) was repeated using N-(4-aminophenyl)-acetamide and 5-(2-chloropyridin-4-yl)oxy-2,3-dimethyl-6-pyridin-2-yl-pyridine (Method 16) to provide the title compound: ¹H NMR: 2.16 (3H, s), 2.35 (3H, s), 2.60 (3H, s), 6.20 (1H, d), 6.22-6.26 (1H, m), 7.08 (1H, s), 7.14 (2H, d), 7.19-7.24 (1H, m), 7.25 (1H, s), 7.31 (1H, s), 7.43 (2H, d), 7.66-7.73 (1H, m), 7.81 (1H, d), 7.89 (1H, d), 8.60-8.65 (1H, m); m/z: 426 MH⁺; EAA2: 0.008415.

### Example 87

### 3-{[4-(5,6-Dimethyl-2-pyridin-2-ylpyridin-3-yl)oxypyridin-2-yl]-amino}-5-(hydroxymethyl)benzenesulfonamide

The procedure described above (Example 72) was repeated using 3-amino-5-(hydroxymethyl)benzenesulfonamide and 5-(2-chloropyridin-4-yl)oxy-2,3-dimethyl-6-pyridin-2-yl-pyridine (Method 16) to provide the title compound: m/z: 478 MH⁺; EAA2: 0.07314.

### Example 88

### 4-[(2,6-Dimethylpyridin-3-yl)oxy]-N-(1-ethyl-1H-pyrazol-5-yl)-pyridin-2-amine

Pd(OAc)₂ (11.5 mg), 3-(2-chloropyridin-4-yl)oxy-2,6-dimethylpyridine (Method 13, 0.118 g), Cs₂CO₃ (0.163 g), xantphos (0.035 g) and 2-ethylpyrazol-3-amine (0.111 g) were dissolved in dimethoxyethane (4 mL) and sealed into a microwave tube. The mixture was heated to 150° C. for 30 mins in the microwave and was then cooled to r.t. The mixture was filtered and then concentrated. Purification by basic reverse phase HPLC afforded the title compound; (0.079 g, 51%); ¹H NMR: 1.38 (3H, t), 2.36 (3H, s), 2.53 (3H, s), 4.07 (2H, q), 5.97 (1H, d), 6.08 (1H, d), 6.17-6.20 (1H, m), 7.01 (1H, d), 7.01 (1H, s), 7.18 (1H, d), 7.49 (1H, d), 7.96 (1H, d); m/z: MH⁺ 310; EAA2: 0.2045.

The procedure described above was repeated using the appropriate amine and 3-(2-chloropyridin-4-yl)oxy-2,6-dimethylpyridine (Method 13) to provide the Examples listed below:

### Example 89

### 4-{[4-(2,6-Dimethylpyridin-3-yl)oxypyridin-2-yl]amino}-benzonitrile

¹H NMR: 2.37 (3H, s), 2.53 (3H, s), 6.17 (1H, d), 6.42-6.46 (1H, m), 7.06 (1H, d), 7.26 (1H, d), 7.51 (1H, s), 7.52-7.61 (4H, m), 8.15 (1H, d); m/z: 317 MH⁻; EAA2: 0.3459.

### Example 90

### 4-(2,6-Dimethylpyridin-3-yl)oxy-N-(4-methoxyphenyl)pyridin-2-amine

¹H NMR: 2.38 (3H, s), 2.53 (3H, s), 3.80 (3H, s), 6.10-6.13 (1H, m), 6.15 (1H, d), 6.65 (1H, s), 6.84-6.90 (1H, m), 6.87 (1H, d), 7.00 (1H, d), 7.16-7.22 (1H, m), 7.18 (1H, s), 7.19 (1H, d), 7.99 (1H, d); m/z: 322 MH⁺; EAA2: 0.1352.

### Example 91

### 2-(4-{[4-(2,6-Dimethylpyridin-3-yl)oxypyridin-2-yl]amino}phenyl)-acetonitrile

¹H NMR: 2.38 (3H, s), 2.54 (3H, s), 3.70 (2H, s), 6.23 (1H, d), 6.25-6.28 (1H, m), 7.02 (1H, s), 7.04 (1H, d), 7.21-7.26 (3H, m), 7.33-7.39 (2H, m), 8.06 (1H, d); m/z: 331 MH⁺; EAA2: 0.02858.

### Example 92

### 4-{[4-(2,6-Dimethylpyridin-3-yl)oxypyridin-2-yl]amino}benzamide

¹H NMR: 2.39 (1.5H, s), 2.41 (1.5H, s), 2.55 (1.5H, s), 2.56 (1.5H, s), 4.07 (1H, s), 5.82 (1H, s), 6.30 (0.5H, d), 6.33-6.36 (0.5H, m), 6.51-6.54 (0.5H, m), 6.66-6.71 (1H, m), 7.03-7.08 (1H, m), 7.13 (0.5H, s), 7.26 (1H, d), 7.40-7.46 (1H, m), 7.70-7.80 (2H, m), 7.93 (0.5H, d), 8.11 (1H, t), 8.57 (0.5H, s); m/z: 335 MH⁺; EAA2: 0.06944.

### Example 93

### 3-(2-Anilinopyridin-4-yl)oxy-N-(cyclopropylmethyl)-6-methyl-pyridine-2-carboxamide

3-(2-Anilinopyridin-4-yl)oxy-6-methylpyridine-2-carboxylic acid (Example 85, 0.068 g) in DMF (2 mL) was treated with cyclopropylmethanamine (0.030 g), HATU (0.097 g) and DIPEA (0.045 mL). The mixture was stirred for 2 h then concentrated. The crude material was purified by basic reverse phase HPLC to afford the title compound (0.030 g, 38%); ¹H NMR: 0.22-0.27 (2H, m), 0.48-0.55 (2H, m), 0.96-1.10 (1H, m), 2.61 (3H, s), 3.23-3.27 (2H, m), 6.20-6.23 (1H, m), 6.39 (1H, d), 6.72 (1H, s), 6.98-7.05 (1H, m), 7.23-7.30 (4H, m), 7.32 (1H, d), 7.40 (1H, d), 7.93 (1H, t), 8.01 (1H, d); m/z: 375 MH⁺; EAA2: 0.1541.

The procedure described above (Example 93) was repeated using the appropriate amine and 3-(2-anilinopyridin-4-yl)oxy-6-methylpyridine-2-carboxylic acid (Example 85) to provide the Examples listed below:

### Example 94

### 3-(2-Anilinopyridin-4-yl)oxy-6-methyl-N-(1H-pyrrol-2-ylmethyl)-pyridine-2-carboxamide

¹H NMR: 2.55 (3H, s), 4.47 (2H, d), 6.05-6.07 (2H, m), 6.20-6.23 (1H, m), 6.39 (1H, d), 6.65-6.68 (1H, m), 6.80 (1H, s), 7.00-7.07 (1H, m), 7.24-7.31 (4H, m), 7.31 (1H, d), 7.39 (1H, d), 8.03 (1H, d), 8.37 (1H, t), 8.96 (1H, s); m/z: 400 MH⁺; EAA2: 0.1191.

### Example 95

### 3-(2-Anilinopyridin-4-yl)oxy-6-methyl-N-phenethylpyridine-2-carboxamide

¹H NMR: 2.55 (3H, s), 2.89 (1H, t), 3.60-3.67 (2H, m), 6.18-6.21 (1H, m), 6.40 (1H, d), 6.66 (1H, s), 6.98-7.05 (1H, m), 7.20-7.32 (1OH, m), 7.22 (1H, d), 7.38 (1H, d), 7.96 (1H, t), 8.03 (1H, d); m/z: 425 MH⁺; EAA2: 0.02094.

### Example 96

### 3-(2-Anilinopyridin-4-yl)oxy-N-benzyl-6-methylpyridine-2-carboxamide

¹H NMR: 2.56 (3H, s), 4.59 (2H, d), 6.21-6.25 (1H, m), 6.41 (1H, d), 6.76 (1H, s), 7.00-7.06 (1H, m), 7.24-7.34 (10H, m), 7.41 (1H, d), 8.02 (1H, d), 8.18 (1H, t); m/z: 411 MH⁺; EAA2: 0.2922.

### Example 97

### 3-(2-Anilinopyridin-4-yl)oxy-N,6-dimethylpyridine-2-carboxamide

¹H NMR: 2.59 (3H, s), 2.95 (3H, d), 6.20-6.24 (1H, m), 6.40 (1H, d), 7.24-7.36 (6H, m), 7.33 (1H, d), 7.40 (1H, d), 7.87 (1H, s), 7.98 (1H, d); m/z: 335 MH⁺; EAA2: 0.3482; IT6: 0.9458.

### Example 98

### Azetidin-1-yl-(3-{[4-(5,6-dimethyl-2-pyridin-2-ylpyridin-3-yl)-oxypyridin-2-yl]amino}phenyl)methanone

A mixture of 3-{[4-(5,6-dimethyl-2-pyridin-2-ylpyridin-3-yl)oxypyridin-2-yl]amino}-benzoic acid (Example 203) (95 mg) and N-hydroxybenzotriazole (53 mg) in THF (4 mL) was added to azetidine (28 mg). Then silica-supported N,N′-dicyclohexylcarbodiimide (700 mg, 1.12 mmol/g) was added and the mixture was shaken for 5 days. The mixture was then heated in a microwave for 30 mins at 130° C. After cooling it was filtered through a 1 g cartridge containing carbonate adsorted onto silica (from Silicycle) eluting with DCM. Further purification by reverse phase basic HPLC afforded the title compound (30 mg, 29%); m/z: MH⁺: 452.3; EAA2: 0.0513.

The procedure described for Example 98 was repeated using the appropriate amine with 3-{[4-(5,6-dimethyl-2-pyridin-2-ylpyridin-3-yl)oxypyridin-2-yl]amino}benzoic acid (Example 203), to provide the Examples listed below:

### Example 99

### 3-{[4-(5,6-Dimethyl-2-pyridin-2-ylpyridin-3-yl)oxypyridin-2-yl]-amino}-N-(pyridin-2-ylmethyl)benzamide

m/z: 503.4 MH⁺; EAA2: 0.007343.

### Example 100

### 3-{[4-(5,6-Dimethyl-2-pyridin-2-ylpyridin-3-yl)oxypyridin-2-yl]-amino}-N-(pyridin-4-ylmethyl)benzamide

m/z: 503.4 MH⁺; EAA2: 0.005761.

### Example 101

### (3-{[4-(5,6-Dimethyl-2-pyridin-2-ylpyridin-3-yl)oxypyridin-2-yl]-amino}phenyl)-(3-methylpiperidin-1-yl)methanone

To 3-{[4-(5,6-dimethyl-2-pyridin-2-ylpyridin-3-yl)oxypyridin-2-yl]amino}benzoic acid (Example 203, 50% pure, 166 mg) was added a solution of HATU (92 mg) in DMF (3 mL), followed by 3-methylpiperidine (42 μL) then DIPEA (45 μL). The mixture was heated in a microwave reactor at 100° C. for 10 mins, then returned to the microwave reactor and heated at 100° C. for 15 mins. Further HATU (50 mg), amine (20 μL) and DIPEA (25 μL) were added and mixture was then heated for a further 15 minutes at 100° C. in the microwave. After cooling the mixture was semi-purified by ion exchange chromatography, using an SCX column eluting with 7M NH₃/MeOH and appropriate fractions were evaporated to dryness. Further purification by reverse phase basic HPLC afforded the title compound as a solid (59.3 mg, 60%); m/z: MH⁺ 493; EAA2: 0.01143.

The procedure described above was repeated using the appropriate amine with 3-{[4-(5,6-dimethyl-2-pyridin-2-ylpyridin-3-yl)oxypyridin-2-yl]amino}benzoic acid (Example 203), to provide the Examples listed below:

### Example 102

### Azepan-1-yl-(3-{[4-(5,6-dimethyl-2-pyridin-2-ylpyridin-3-yl)-oxypyridin-2-yl]amino}phenyl)methanone

m/z: 494 MH⁺; EAA2: 0.01129.

### Example 103

### 3-{[4-(5,6-Dimethyl-2-pyridin-2-ylpyridin-3-yl)oxypyridin-2-yl]-amino}-N-(2-methylbut-3-yn-2-yl)benzamide

m/z: 478 MH⁺; EAA2: 0.03751.

### Example 104

### 3-{[4-(5,6-Dimethyl-2-pyridin-2-ylpyridin-3-yl)oxypyridin-2-yl]-amino}benzenesulfonamide

5-(2-Chloropyridin-4-yl)oxy-2,3-dimethyl-6-pyridin-2-yl-pyridine (Method 16, 220 mg), 3-aminobenzenesulfonamide (182 mg), Pd(OAc)₂ (12.67 mg), xantphos (49.0 mg) and Cs₂CO₃ (460 mg,) were suspended in DMA (4 mL) and sealed into a microwave tube. The mixture was heated to 100° C. for 30 mins in a microwave, then to 130° C. for 30 mins. After cooling to r.t. the crude material was semi-purified by ion exchange chromatography, using an SCX column eluting with 7M NH₃/MeOH. Appropriate fractions were evaporated to dryness. Further purification was achieved by reverse phase basic HPLC. Finally, purification by FCC using a gradient of 1-5% MeOH in DCM afforded the title compound as a solid (10.50 mg, 3.33%); ¹H NMR: 2.4 (3H, s), 2.55 (3H, s), 6.25 (1H, d) 6.35 (1H, d), 7.1 (1H, d), 7.3 (d,1H), 7.4 (4H, s), 7.45 (1H, m), 7.6 (1H, m), 7.95 (1H, m), 8.05 (1H,d); m/z: MH⁺ 448; EAA2: 0.01213.

### Example 105

### 3-{[4-(2,6-Dimethylpyridin-3-yl)oxypyridin-2-yl]amino}benzene-sulfonamide

3-(2-Chloropyridin-4-yloxy)-2,6-dimethylpyridine (Method 13) (200 mg), 3-aminobenzenesulfonamide (220 mg), Pd(OAc)₂ (15.31 mg), xantphos (59.2 mg,) and Cs₂CO₃ (555 mg) were suspended in DMA (4 mL) and sealed into a microwave tube. The mixture was heated to 100° C. for 30 min in a microwave and was then cooled to r.t. The crude material was semi-purified by ion exchange chromatography, using an SCX column eluting with 7M NH₃/MeOH and appropriate fractions were evaporated to dryness. Further purification was achieved by reverse phase basic HPLC. Trituration with DCM to afforded the title compound as a solid (10.00 mg, 3.17%); ¹H NMR: (deuterated methanol) 2.4 (3H, s), 2.6 (3H, s), 6.05 (1H, d), 6.35 (1H, d), 7.25-7.45 (4H, m), 7.75 (2H, m), 7.8 (1H, m) 7.9 (1H, d), 8.7 (1H, d); m/z: MH⁻ 371; EAA2: 0.007641.

### Example 106

### 4-[4-(2,6-Dimethylpyridin-3-yloxy)pyridin-2-ylamino]-N-(pyridin-2-yl)benzenesulfonamide

3-(2-Chloropyridin-4-yloxy)-2,6-dimethylpyridine (Method 13, 100 mg) and 4-amino-N-pyridin-2-ylbenzenesulfonamide (115 mg) were dissolved in 1,4-dioxane (4 mL). The mixture was treated with Cs₂CO₃ (283 mg) and sparged with argon for 5 mins. Pd(OAc)₂ (5 mg) and xantphos (27 mg) were then added and the resulting mixture was heated to 90° C. for 45 mins. DMA (2 mL) was then added and stirring was continued at 90° C. overnight. The reaction was incomplete so the reaction mixture was sealed into a microwave tube and heated to 150° C. for 20 mins in a microwave. The mixture was cooled to r.t. and subjected to ion exchange chromatography, using an SCX-3 cartridge. The cartridge was eluted with MeOH then the desired product was eluted from the column using 2M NH₃/MeOH. The ammoniacal eluant was evaporated to dryness to afford the crude product. Further purification was achieved by preparative HPLC (Waters XTerra C 18 column, 5 μm silica, 19 mm diameter, 100 mm length), using decreasingly polar mixtures of water (containing 1% NH₃) and MeCN as eluents. Appropriate fractions were concentrated to afford the title compound (17 mg, 9%); ¹H NMR: 2.28 (3H, s), 2.48 (3H, s), 6.12 (1H, d), 6.54 (1H, dd), 6.85-6.90 (1H, m), 7.11 (1H, d), 7.22 (1H, d), 7.49 (1H, d), 7.65-7.69 (1H, m), 7.72-7.78 (4H, m), 8.03-8.07 (1H, m), 8.12 (1H, d), 9.39 (1H, s); m/z: MH⁺ 448.0; EAA2: 0.5266.

### Example 107

### N-{4-[4-(2,6-Dimethylpyridin-3-yloxy)pyridin-2-ylamino]phenyl-sulfonyl}acetamide

3-(2-Chloropyridin-4-yloxy)-2,6-dimethylpyridine (Method 13, 100 mg) and sulfacetamide (97 mg) were dissolved in DMA (4 mL). Cs₂CO₃ (283 mg) was then added and mixture was sparged with nitrogen for 5 mins. Pd(OAc)₂ (5 mg) and xantphos (27 mg) were then added and the mixture was sealed into a microwave tube. The reaction was heated to 150° C. for 10 mins in a microwave. After cooling, the mixture was part purified by ion exchange chromatography, using an SCX-3 column (5 g). The column was first eluted through with MeOH before the crude product was eluted using 2M NH₃/MeOH. The ammoniacal solution was evaporated to afford crude product, which was purified by preparative HPLC (Waters XBridge Prep C18 OBD column, 5 μm silica, 21 mm diameter, 100 mm length), using decreasingly polar mixtures of water (containing 1% NH₃) and MeCN as eluents. Fractions containing the desired compound were evaporated to dryness and lyophilised to afford the title compound (50 mg, 28%); ¹H NMR: 1.85 (3H, s), 2.29 (3H, s), 2.49 (3H, s), 6.14 (1H, d), 6.57 (1H, dd), 7.24 (1H, d), 7.52 (1H, d), 7.71-7.80 (4H, m), 8.14 (1H, d), 9.49 (1H, s); m/z: MH⁺ 413.0; EAA2: 0.5924.

Using an analogous procedure to that described above the appropriate aniline was reacted with 3-(2-chloropyridin-4-yloxy)-2,6-dimethylpyridine (Method 13) to provide the Examples listed below: (The anilines were generally obtained from commercial sources, but where unavailable commercially they were synthesised according to the references cited.)

### Example 108

### 4-{[4-(2,6-Dimethylpyridin-3-yl)oxypyridin-2-yl]amino}-N-methylbenzenesulfonamide

¹H NMR: 2.30 (3H, s), 2.38 (3H, d), 2.49 (3H, s), 6.15 (1H, d), 6.56 (1H, dd), 7.15 (1H, q), 7.23 (1H, d), 7.51 (1H, d), 7.61-7.64 (2H, m), 7.81-7.85 (2H, m), 8.15 (1H, d), 9.43 (1H, s); m/z: 385.5 MH⁺; EAA2: 0.1708.

### Example 109

### 4-{[4-(2,6-Dimethylpyridin-3-yl)oxypyridin-2-yl]amino}-N-(3-methoxypropyl)benzenesulfonamide

The aniline starting material [CAS: 27678-19-9] was synthesised according to WO2002/096887. Title compound: ¹H NMR: 1.58 (2H, quintet), 2.30 (3H, s), 2.49 (3H, s), 2.75 (2H, q), 3.15 (3H, s), 3.27 (2H, t), 6.14 (1H, d), 6.56 (1H, dd), 7.23 (1H, d), 7.29 (1H, t), 7.51 (1H, d), 7.61-7.65 (2H, m), 7.80-7.84 (2H, m), 8.14 (1H, d), 9.42 (1H, s); m/z: 443.6 MH⁺; EAA2: 0.269.

### Example 110

### 4-{[4-(2,6-Dimethylpyridin-3-yl)oxypyridin-2-yl]amino}-N-(2-methoxyethyl)benzenesulfonamide

The aniline starting material [CAS: 328062-38-0] was synthesised according to WO2002/096887. Title compound: ¹H NMR: 2.30 (3H, s), 2.49 (3H, s), 2.86 (2H, q), 3.17 (3H, s), 3.28-3.31 (2H, m), 6.14 (1H, d), 6.55 (1H, dd), 7.23 (1H, d), 7.41 (1H, t), 7.50 (1H, d), 7.63-7.66 (2H, m), 7.80-7.83 (2H, m), 8.14 (1H, d), 9.42 (1H, s); m/z: 429.5 MH⁺; EAA2: 0.2031.

### Example 111

### 3-{[4-(2,6-Dimethylpyridin-3-yl)oxypyridin-2-yl]amino}-N-phenylbenzenesulfonamide

¹H NMR: 2.38 (3H, s), 2.56 (3H, s), 6.17 (1H, d), 6.54 (1H, dd), 6.98-7.03 (1H, m), 7.10-7.12 (2H, m), 7.20-7.28 (3H, m), 7.38-7.42 (2H, m), 7.72-7.77 (2H, m), 8.12 (1H, d), 8.22 (1H, t), 9.39 (1H, s), 10.24 (1H, s); m/z: 447.6 MH⁺; EAA2: 0.6212.

### Example 112

### 4-{[4-(2,6-Dimethylpyridin-3-yl)oxypyridin-2-yl]amino}-N-(2-hydroxyethyl)-N-methylbenzenesulfonamide

The aniline starting material is known: [CAS 328072-15-7]. Title compound: ¹H NMR: 2.30 (3H, s), 2.49 (3H, s), 2.68 (3H, s), 2.96 (2H, t), 3.51 (2H, q), 4.73 (1H, t), 6.15 (1H, d), 6.57 (1H, dd), 7.24 (1H, d), 7.51 (1H, d), 7.60-7.63 (2H, m), 7.84-7.88 (2H, m), 8.15 (1H, d), 9.48 (1H, s); m/z: 429.5 MH⁺; EAA2: 0.505.

### Example 113

### N-Cyclobutyl-4-{[4-(2,6-dimethylpyridin-3-yl)oxypyridin-2-yl]-amino}benzenesulfonamide

The aniline starting material [CAS: 477723-16-3] was synthesised according to WO2002/096887. Title compound: ¹H NMR: 1.40-1.52 (2H, m), 1.67-1.77 (2H, m), 1.85-1.93 (2H, m), 2.30 (3H, s), 2.49 (3H, s), 3.53-3.63 (1H, m), 6.14 (1H, d), 6.56 (1H, dd), 7.23 (1H, d), 7.50 (1H, d), 7.60-7.64 (2H, m), 7.66 (1H, d), 7.79-7.82 (2H, m), 8.15 (1H, d), 9.41 (1H, s); m/z: 425.6 MH⁺; EAA2: 0.2875.

### Example 114

### 4-{[4-(2,6-Dimethylpyridin-3-yl)oxypyridin-2-yl]amino}-N-pentylbenzenesulfonamide

The aniline starting material [CAS 58556-64-2] was synthesised according to WO2002/004429. Title compound: ¹H NMR: 0.79-0.82 (3H, m), 1.16-1.21 (4H, m), 1.31-1.38 (2H, m), 2.30 (3H, s), 2.49 (3H, s), 2.69 (2H, q), 6.14 (1H, d), 6.55 (1H, dd), 7.22-7.28 (2H, m), 7.51 (1H, d), 7.61-7.65 (2H, m), 7.80-7.83 (2H, m), 8.14 (1H, d), 9.41 (1H, s); m/z: 441.6 MH⁺; EAA2: 3.014.

### Example 115

### 4-(2,6-Dimethylpyridin-3-yl)oxy-N-(4-piperidin-1-ylsulfonyl-phenyl)pyridin-2-amine

¹H NMR: 1.32-1.38 (2H, m), 1.51-1.56 (4H, m), 2.41 (3H, s), 2.59 (3H, s), 2.84-2.86 (4H, m), 6.30 (1H, d), 6.60 (1H, dd), 7.47 (1H, d), 7.58-7.61 (2H, m), 7.82-7.88 (3H, m), 8.18 (1H, d), 9.61 (1H, s); m/z: 439.6 MH⁺; EAA2: 5.411.

### Example 116

### 4-{[4-(2,6-Dimethylpyridin-3-yl)oxypyridin-2-yl]amino}-N,N-diethylbenzenesulfonamide

¹H NMR: 1.03 (6H, t), 2.30 (3H, s), 2.49 (3H, s), 3.12 (4H, q), 6.14 (1H, d), 6.56 (1H, dd), 7.23 (1H, d), 7.50 (1H, d), 7.62-7.65 (2H, m), 7.82-7.85 (2H, m), 8.15 (1H, d), 9.45 (1H, s); m/z: 427.6 MH⁺; EAA2: 1.419.

### Example 117

### 4-{[4-(2,6-Dimethylpyridin-3-yl)oxypyridin-2-yl]amino}-N,N-dimethylbenzenesulfonamide

¹H NMR: 2.30 (3H, s), 2.49 (3H, s), 2.57 (6H, s), 6.16 (1H, d), 6.57 (1H, dd), 7.24 (1H, d), 7.51 (1H, d), 7.59-7.62 (2H, m), 7.87-7.91 (2H, m), 8.16 (1H, d), 9.50 (1H, s); m/z: 399.5 MH⁺; EAA2: 0.3697.

### Example 118

### 4-{[4-(2,6-Dimethylpyridin-3-yl)oxypyridin-2-yl]amino}-N-(1-methoxypropan-2-yl)benzenesulfonamide

¹H NMR: 0.88 (3H, d), 2.30 (3H, s), 2.49 (3H, s), 3.04-3.08 (1H, m), 3.14 (3H, s), 3.17-3.25 (2H, m), 6.14 (1H, d), 6.56 (1H, dd), 7.23 (1H, d), 7.36 (1H, d), 7.50 (1H, d), 7.64-7.68 (2H, m), 7.80-7.83 (2H, m), 8.15 (1H, d), 9.41 (1H, s); m/z: 443.5 MH⁺; EAA2: 0.155.

### Example 119

### 4-(2,6-Dimethylpyridin-3-yl)oxy-N-(3-morpholin-4-ylsulfonyl-phenyl)pyridin-2-amine

¹H NMR: 2.30 (3H, s), 2.49 (3H, s), 2.88-2.90 (4H, m), 3.62-3.65 (4H, m), 6.09 (1H, d), 6.52-6.54 (1H, m), 7.18-7.21 (1H, m), 7.23 (1H, d), 7.48-7.53 (2H, m), 7.93-7.96 (1H, m), 8.11-8.15 (2H, m), 9.39 (1H, s); m/z: 441.5 MH⁺; EAA2: 0.03189.

### Example 120

### 4-(2,6-Dimethylpyridin-3-yl)oxy-N-(2,2-dioxo-1,3-dihydro-2-benzothiophen-5-yl)pyridin-2-amine

¹H NMR: 2.41 (3H, s), 2.58 (3H, s), 4.42 (2H, s), 4.47 (2H, s), 6.25 (1H, d), 6.58 (1H, dd), 7.30 (1H, d), 7.45 (2H, d), 7.70 (1H, s), 7.81 (1H, d), 8.09 (1H, d), 9.53 (1H, s); m/z: 382.5 MH⁺.

### Example 121

### 4-{[4-(2,6-Dimethylpyridin-3-yl)oxypyridin-2-yl]amino}-N,N-dimethylbenzamide

3-(2-Chloropyridin-4-yl)oxy-2,6-dimethylpyridine (Method 13, 100 mg) and 4-amino-N,N-dimethylbenzamide (77 mg) were dissolved in DMA (4 mL). Cs₂CO₃ (283 mg) was added and the mixture was flushed with nitrogen for 5 mins. Pd(OAc)₂ (5 mg) and xantphos (29 mg) were then added and the mixture was sealed in a microwave tube. The mixture was heated to 150° C. for 30 mins in the microwave. After cooling, the mixture was subjected to ion exchange chromatography, using an SCX-3 column (5 g). The column was first eluted with MeOH then the crude product was eluted from the column using 2M NH₃/MeOH. The ammoniacal solution was evaporated to afford crude material, which was further purified by preparative HPLC (Waters XBridge Prep C18 OBD column, 5 μm silica, 21 mm diameter, 100 mm length), using decreasingly polar mixtures of water (containing 1% NH₃) and MeCN as eluents. Fractions containing the desired compound were evaporated to dryness and lyophilised to afford the title compound (81 mg, 52%); ¹H NMR: 2.30 (3H, s), 2.49 (3H, s), 2.96 (6H, s), 6.10 (1H, s), 6.48-6.50 (1H, m), 7.23 (1H, d), 7.32 (2H, d), 7.50 (1H, d), 7.69 (2H, d), 8.10 (1H, d), 9.17 (1H, s); m/z: MH⁺ 363.5; EAA2: 0.088.

The procedure described above was repeated using 3-(2-chloropyridin-4-yloxy)-2,6-dimethylpyridine (Method 13) in reaction with the appropriate aniline with, to provide the Examples listed below:

### Example 122

### N-(4-{[4-(2,6-Dimethylpyridin-3-yl)oxypyridin-2-yl]amino}-phenyl)-N-methylacetamide

¹H NMR: 1.75 (3H, s), 2.30 (3H, s), 2.49 (3H, s), 3.10 (3H, s), 6.07 (1H, s), 6.46-6.47 (1H, m), 7.17 (2H, d), 7.23 (1H, d), 7.49 (1H, d), 7.69 (2H, d), 8.08 (1H, d), 9.09 (1H, s); m/z: 363.5 MH⁺; EAA2: 0.2147.

### Example 123

### N-(4-{[4-(2,6-Dimethylpyridin-3-yl)oxypyridin-2-yl]amino}-phenyl)acetamide

¹H NMR: 2.00 (3H, s), 2.29 (3H, s), 2.48 (3H, s), 6.02 (1H, s), 6.37-6.39 (1H, m), 7.21 (1H, d), 7.41-7.52 (5H, m), 8.03 (1H, d), 8.84 (1H, s), 9.72 (1H, s); m/z: 349.5 MH⁺; EAA2: 0.03243.

### Example 124

### 4-(2,6-Dimethylpyridin-3-yl)oxy-N-(4-methylsulfonylphenyl)-pyridin-2-amine

m/z: 370.5 MH⁺; EAA2: 0.07696.

### Example 125

### 3-{[4-(2,6-Dimethylpyridin-3-yl)oxypyridin-2-yl]amino}-benzonitrile

¹H NMR: 2.30 (3H, s), 2.49 (3H, s), 6.09 (1H, s), 6.54-6.55 (1H, m), 7.23 (1H, d), 7.29 (1H, d), 7.41-7.45 (1H, m), 7.51 (1H, d), 7.75 (1H, d), 8.15 (1H, d), 8.30 (1H, s), 9.34 (1H, s); m/z: 317.4 MH⁺; EAA2: 0.1919.

### Example 126

### 3-{[4-(2,6-Dimethylpyridin-3-yl)oxypyridin-2-yl]amino}phenol

¹H NMR: 2.30 (3H, s), 2.48 (3H, s), 6.06 (1H, s), 6.27-6.29 (1H, m), 6.39-6.42 (1H, m), 6.93-7.01 (2H, m), 7.21-7.23 (2H, m), 7.48 (1H, d), 8.05 (1H, d), 8.81 (1H, s), 9.14 (1H, s); m/z: 308.4 MH⁺; EAA2: 0.03769.

### Example 127

### N-(5-{[4-(2,6-Dimethylpyridin-3-yl)oxypyridin-2-yl]amino}-2-methylphenyl)acetamide

¹H NMR: 2.04 (3H, s), 2.11 (3H, s), 2.29 (3H, s), 2.48 (3H, s), 6.06 (1H, s), 6.38-6.40 (1H, m), 7.04 (1H, d), 7.21 (1H, d), 7.36-7.39 (1H, m), 7.48 (1H, d), 7.64 (1H, s), 8.03 (1H, d), 8.89 (1H, s), 9.18 (1H, s); m/z: 363.5 MH⁺; EAA2: 0.01449.

### Example 128

### N-(3-{[4-(2,6-Dimethylpyridin-3-yl)oxypyridin-2-yl]amino}-phenyl)methanesulfonamide

¹H NMR: 2.30 (3H, s), 2.48 (3H, s), 2.97 (3H, s), 6.10 (1H, s), 6.43-6.45 (1H, m), 6.72 (1H, d), 7.14-7.18 (1H, m), 7.22 (1H, d), 7.46-7.49 (3H, m), 8.06 (1H, d), 9.02 (1H, s), 9.60 (1H, s); m/z: 385.5 MH⁺; EAA2: 0.03211.

### Example 129

### 1-(4-{[4-(2,6-Dimethylpyridin-3-yl)oxypyridin-2-yl]amino}-phenoxy)-3-(propan-2-ylamino)propan-2-ol

m/z: 423.6 MH⁺; EAA2: 0.103.

### Example 130

### 4-[4-(2,6-Dimethylpyridin-3-yloxy)pyridin-2-ylamino]-N-(1-methylpiperidin-4-yl)benzamide

4-Amino-N-(1-methylpiperidin-4-yl)benzamide (Method 34, 99 mg), 3-(2-chloropyridin-4-yloxy)-2,6-dimethylpyridine (Method 13, 100 mg) and p-toluenesulfonic acid monohydrate (162 mg) were suspended in 4-methyl-2-pentanol (2 mL) and sealed into a microwave tube. The mixture was heated to 160° C. for 45 mins in a microwave and was then cooled to r.t. MeOH (5 mL) and water (2 mL) were added and the mixture was sonicated to give a solution, which was subjected to ion exchange chromatography, using an SCX-3 column (5 g). The column was first eluted with MeOH thene the crude product was eluted from the column using 2M NH₃/MeOH. The ammoniacal solution was evaporated to dryness to afford the crude material that was further purified by preparative HPLC (Waters XTerra C18 column, 5 μm silica, 19 mm diameter, 100 mm length), using decreasingly polar mixtures of water (containing 1% NH₃) and MeCN as eluents. Fractions containing the desired compound were evaporated to dryness and lyophilised to afford the title compound (38.0 mg, 21%); ¹H NMR: 1.52-1.62 (2H, m), 1.71-1.77 (2H, m), 1.89-1.97 (2H, m), 2.16 (3H, s), 2.30 (3H, s), 2.49 (3H, s), 2.73-2.79 (2H, m), 3.65-3.76 (1H, m), 6.12 (1H, d), 6.50 (1H, dd), 7.23 (1H, d), 7.49 (1H, d), 7.67-7.75 (4H, m), 7.93 (1H, d), 8.12 (1H, d), 9.22 (1H, s); m/z: MH⁺ 432.4; EAA2: 0.01891.

### Example 131

### 4-[4-(2,6-Dimethylpyridin-3-yloxy)pyridin-2-ylamino]-N-(2-morpholinoethyl)benzenesulfonamide

3-(2-Chloropyridin-4-yloxy)-2,6-dimethylpyridine (Method 13; 100 mg), 4-amino-N-(2-chloroethyl)benzenesulfonamide (100 mg) and p-toluenesulfonic acid monohydrate (162 mg) were dissolved in 4-methyl-2-pentanol (2 mL) and mixture sealed into a microwave tube. The mixture was heated to 160° C. for 30 mins in a microwave and was then cooled to r.t. The mixture was diluted with MeOH and water and subjected to ion exchange chromatography, using an SCX-3 column (5 g). The column was first eluted with MeOH, before eluting the product using 2M NH₃/MeOH. The ammoniacal eluent was evaporated to dryness to afford crude material, which was dissolved in DMA (2 mL) and treated with morpholine (200 μL). The mixture was sealed into another microwave tube and heated to 120° C. for a total of 40 mins in the microwave. The mixture was then cooled to r.t. and poured onto an SCX-3 (5 g) column. The column was first washed through with MeOH, before products were eluted from the column using 2M NH₃/MeOH. The ammoniacal solution was evaporated to dryness to afford crude product, which was purified by preparative HPLC (Waters XTerra C18 column, 5 μm silica, 19 mm diameter, 100 mm length), firstly using decreasingly polar mixtures of water (containing 1% NH₃) and MeCN as eluents, then re-purified using preparative HPLC (Waters XTerra C18 column, 5 μm silica, 19 mm diameter, 100 mm length), using decreasingly polar mixtures of water (containing 0.1% TFA) and MeCN as eluents. Fractions that contained product were treated with macroporous triethylammonium methylpolystyrene carbonate, filtered and concentrated.

The product was further purified by ion exchange chromatography, using an SCX-3 column. The desired product was eluted from the column using 2M NH₃/MeOH and appropriate fractions were evaporated to dryness, triturated under diethyl ether/isohexane and dried under vacuum at 50° C. for 4 h. to afford the title compound (31.0 mg, 15%) as a white solid; ¹H NMR: 2.25-2.32 (9H, m), 2.49 (3H, s), 2.83 (2H, q), 3.49-3.52 (4H, m), 6.14 (1H, d), 6.56 (1H, dd), 7.20-7.24 (2H, m), 7.51 (1H, d), 7.64-7.67 (2H, m), 7.80-7.84 (2H, m), 8.14 (1H, d), 9.42 (1H, s); m/z: MH⁺ 484.6; EAA2: 0.2563.

### Example 132

### 4-{[4-(2,6-Dimethylpyridin-3-yl)oxypyridin-2-yl]amino}-N-[2-(1,1-dioxo-1,4-thiazinan-4-yl)ethyl]benzenesulfonamide

A solution of N-(2-chloroethyl)-4-[4-(2,6-dimethylpyridin-3-yloxy)pyridin-2-ylamino]benzenesulfonamide (Example 204, 150 mg) in DMA (2 mL) was added to thiomorpholine 1,1-dioxide (272 mg). The contents were sealed into a microwave tube and heated to 120° C. and held at that temperature for 30 mins. The reaction was incomplete so the mixture was heated again to 160° C. and held for a total period of 1 h. After cooling, the mixture was poured onto an SCX-3 (5 g) column. The column was first washed through with MeOH, before products were eluted from the column using 2M NH₃/MeOH. The ammoniacal solution was evaporated to dryness to afford the crude product, which was purified by preparative HPLC (Waters XTerra C18 column, 5 μm silica, 19 mm diameter, 100 mm length), firstly using decreasingly polar mixtures of water (containing 1% NH₃) and MeCN as eluents, followed by using decreasingly polar mixtures of water (containing 0.1% TFA) and MeCN as eluents. Fractions containing product were converted from the acid salt to the free amine using ion exchange chromatography, evaporated to dryness and lyophilised to afford the title compound (23 mg, 12%); ¹H NMR: 2.30 (3H, s), 2.47-2.52 (5H, m), 2.78-2.86 (6H, m), 3.00-3.03 (4H, m), 6.14 (1H, d), 6.56 (1H, dd), 7.22-7.25 (2H, m), 7.50 (1H, d), 7.65-7.68 (2H, m), 7.80-7.84 (2H, m), 8.14 (1H, d), 9.42 (1H, s); m/z: MH⁺ 532.6; EAA2: 0.1416.

Using an analagous procedure to that described above, the appropriate amine was reacted with N-(2-chloroethyl)-4-[4-(2,6-dimethylpyridin-3-yloxy)pyridin-2-ylamino]benzenesulfonamide (Example 204), to provide the Examples listed below:

### Example 133

### 4-{[4-(2,6-Dimethylpyridin-3-yl)oxypyridin-2-yl]amino}-N-[2-(4-methylpiperazin-1-yl)ethyl]benzenesulfonamide

¹H NMR: 2.11 (3H, s), 2.20-2.32 (13H, m), 2.49 (3H, s), 2.78-2.83 (2H, m), 6.14 (1H, d), 6.56 (1H, dd), 7.17 (1H, t), 7.23 (1H, d), 7.50 (1H, d), 7.63-7.67 (2H, m), 7.80-7.84 (2H, m), 8.14 (1H, d), 9.42 (1H, s); m/z: 497.6 MH⁺; EAA2: 0.09323.

### Example 134

### 4-{[4-(2,6-Dimethylpyridin-3-yl)oxypyridin-2-yl]amino}-N-(2-piperidin-1-ylethyl)benzenesulfonamide*

¹H NMR: 1.31-1.42 (2H, m), 1.57-1.70 (4H, m), 1.76-1.83 (2H, m), 2.31 (3H, s), 2.86-2.94 (2H, m), 3.04-3.15 (4H, m), 3.40-3.45 (3H, m), 6.16 (1H, d), 6.58 (1H, dd), 7.26 (1H, d), 7.53 (1H, d), 7.65-7.70 (3H, m), 7.84-7.88 (2H, m), 8.15 (1H, d), 9.08 (1H, s), 9.50 (1H, s); m/z: 482.6 MH⁺; EAA2: 0.1993. *The compound isolated appears to be a salt, presumably the trifluoroacetate.

### Example 135

### 4-{[4-(2,6-Dimethylpyridin-3-yl)oxypyridin-2-yl]amino}-N-(2-imidazol-1-ylethyl)benzenesulfonamide

¹H NMR: 2.30 (3H, s), 2.49 (3H, s), 3.14 (2H, q), 4.18 (2H, t), 6.15 (1H, d), 6.57 (1H, dd), 7.24 (1H, d), 7.47-7.47 (1H, m), 7.50 (1H, d), 7.56-7.57 (1H, m), 7.60-7.65 (3H, m), 7.82-7.84 (2H, m), 8.15 (1H, d), 8.69 (1H, s), 9.46 (1H, s); /z: 465.5 MH⁺; EAA2: 0.1198.

### Example 136

### 4-{[4-(2,6-Dimethylpyridin-3-yl)oxypyridin-2-yl]amino}-N-(2-pyrazol-1-ylethyl)benzenesulfonamide

¹H NMR: 2.30 (3H, s), 2.49 (3H, s), 3.08 (2H, t), 4.14 (2H, t), 6.14 (1H, d), 6.21 (1H, t), 6.56 (1H, dd), 7.23 (1H, d), 7.42 (1H, dd), 7.51 (1H, d), 7.52-7.56 (1H, m), 7.61-7.65 (2H, m), 7.66 (1H, dd), 7.80-7.84 (2H, m), 8.15 (1H, d), 9.44 (1H, s); m/z: 465.5 MH⁺; EAA2: 0.101.

### Example 137

### 4-{[4-(2,6-Dimethylpyridin-3-yl)oxypyridin-2-yl]amino}-N-12-[(3S)-3-fluoropyrrolidin-1-yl]ethyl}benzenesulfonamide

m/z: 486.6 MH⁺; EAA2: 0.163.

### Example 138 and Example 139

### (S)-{3-[4-(2,6-Dimethylpyridin-3-yloxy)pyridin-2-ylamino]phenyl}-[3-(methylsulfonyl)pyrrolidin-1-yl]methanone and (R)-{3-[4-(2,6-Dimethylpyridin-3-yloxy)pyridin-2-ylamino]phenyl}-[3-(methylsulfonyl)pyrrolidin-1-yl]methanone

(±)-{3-[4-(2,6-Dimethylpyridin-3-yloxy)pyridin-2-ylamino]phenyl}-[3-(methylsulfonyl)pyrrolidin-1-yl]methanone (Example 66, 50 mg) was purified by preparative chiral-HPLC on a Chiralpak IA column, eluting isocratically with 50% EtOH in isohexane (modified with Et₃N and acetic acid). The fractions containing the desired compounds were evaporated to dryness and lyophilised to afford, in order of elution:

the title compound isomer 1 (17 mg) as a white solid; ¹H NMR: 2.23-2.33 (5H, m), 2.47 (3H, s), 2.95-3.08 (3H, m), 3.47-4.04 (5H, m), 6.05 (1H, d), 6.45 (1H, dd), 6.95-7.01 (1H, m), 7.21 (1H, d), 7.26-7.31 (1H, m), 7.48 (1H, d), 7.60-7.65 (1H, m), 7.86-7.93 (1H, m), 8.07 (1H, d), 9.11 (1H, s); m/z: MH⁺ 467.5; EAA2: 0.01429.

and the title compound isomer 2 (15 mg) as an offwhite solid; ¹H NMR: (500 MHz) 2.23-2.33 (5H, m), 2.47 (3H, s), 2.95-3.08 (3H, m), 3.47-4.04 (5H, m), 6.05 (1H, d), 6.46 (1H, dd), 6.94-7.01 (1H, m), 7.21 (1H, d), 7.26-7.31 (1H, m), 7.48 (1H, d), 7.60-7.65 (1H, m), 7.86-7.93 (1H, m), 8.07 (1H, d), 9.11 (1H, s); m/z: MH⁺ 467.5; EAA2: 0.04955.

### Example 140

### 4-{[4-(2,6-Dimethylpyridin-3-yl)oxypyridin-2-yl]amino}-N-(2-pyrazol-1-ylethyl)benzamide

HBTU (144 mg) was added to lithium 4-[4-(2,6-dimethylpyridin-3-yloxy)pyridin-2-ylamino]benzoate (Example 205, 100 mg), 2-(1H-pyrazol-1-yl)-ethanamine (35.7 mg) and NEt₃ (0.086 mL) in DMF (2 mL) and the resulting solution was stirred at r.t. for 18 h. The mixture was subjected to ion exchange chromatography using an SCX-3 column (5 g). The column was first eluted with MeOH before the crude product was eluted from the column using 2M NH₃/MeOH. The ammoniacal solution was evaporated to dryness to afford the crude product, which was purified by preparative HPLC (Waters XTerra C18 column, 5 μm silica, 19 mm diameter, 100 mm length) using decreasingly polar mixtures of water (containing 1% NH₃) and MeCN as eluents. Fractions containing the desired compound were evaporated to dryness to afford the title compound (34.0 mg, 27%); ¹H NMR: 2.30 (3H, s), 2.49 (3H, s), 3.59-3.63 (2H, m), 4.28 (2H, t), 6.12 (1H, s), 6.22 (1H, s), 6.50-6.52 (1H, m), 7.23 (1H, d), 7.45 (1H, s), 7.50 (1H, d), 7.68-7.69 (1H, m), 7.70 (4H, s), 8.12 (1H, d), 8.31 (1H, t), 9.24 (1H, s); m/z: MH⁺ 429.6; EAA2: 0.02078.

### Example 141

### 4-{[4-(2,6-dimethylpyridin-3-yl)oxypyridin-2-yl]amino}-N-[2-(2-oxopyrrolidin-1-yl)ethyl]benzamide

HBTU (144 mg) was added to lithium 4-[4-(2,6-dimethylpyridin-3-yloxy)pyridin-2-ylamino]benzoate (Example 205,100 mg), 1-(2-aminoethyl)-pyrrolidin-2-one (41.2 mg) and NEt₃ (0.086 mL) in DMF (2 mL) and the resulting solution was stirred at r.t. for 72 h. The mixture was subjected to ion exchange chromatography using an SCX-3 column (5 g). The column was first eluted with MeOH, then the crude product was eluted from the column using 2M NH₃/MeOH. The ammoniacal solution was evaporated to dryness to afford the crude product, which was purified by preparative HPLC (Waters XTerra C18 column, 5 μm silica, 19 mm diameter, 100 mm length) using decreasingly polar mixtures of water (containing 1% NH₃) and MeCN as eluents. Fractions containing the desired compound were evaporated to dryness to afford the title compound (35.0 mg, 27%); ¹H NMR: 1.87-1.94 (2H, m), 2.18 (2H, t), 2.30 (3H, s), 2.49 (3H, s), 3.32-3.42 (6H, m), 6.12 (1H, s), 6.50-6.52 (1H, m), 7.23 (1H, d), 7.50 (1H, d), 7.70 (4H, s), 8.12 (1H, d), 8.24-8.26 (1H, m), 9.23 (1H, s); m/z: MH⁺ 446.6; EAA2: 0.0693.

### Example 142

### 4-{[4-(2,6-Dimethylpyridin-3-yl)oxypyridin-2-yl]amino}-N-[2-(dimethylsulfamoylamino)ethyl]benzamide

HBTU (144 mg) was added to lithium 4-[4-(2,6-dimethylpyridin-3-yloxy)pyridin-2-ylamino]benzoate (Example 205,100 mg), N′-(2-aminoethyl)-N,N-dimethylsulfamide (50.3 mg) and NEt₃ (0.086 mL) in DMF (2 mL) and the resulting solution was stirred at r.t. for 72 h. The mixture was then subjected to ion exchange chromatography using an SCX-3 column (5 g). The column was first eluted with MeOH before the crude product was eluted from the column using 2M NH₃/MeOH. The ammoniacal solution was evaporated to dryness to afford the crude product, which was purified by preparative HPLC (Waters XTerra C18 column, 5 μm silica, 19 mm diameter, 100 mm length) using decreasingly polar mixtures of water (containing 1% NH₃) and MeCN as eluents. Fractions containing the desired compound were evaporated to dryness to afford the title compound (33.0 mg, 23%); ¹H NMR: 2.30 (3H, s), 2.49 (3H, s), 2.67 (6H, s), 3.04-3.09 (2H, m), 3.32-3.37 (2H, m), 6.12 (1H, s), 6.50-6.52 (1H, m), 7.22-7.27 (2H, m), 7.50 (1H, d), 7.70 (2H, d), 7.74 (2H, d), 8.13 (1H, d), 8.23 (1H, t), 9.24 (1H, s); m/z: MH⁺ 485.5; EAA2: 0.05056.

### Example 143

### Methyl 3-{[4-(2,6-dimethylpyridin-3-yl)oxypyridin-2-yl]-amino}benzoate

Pd(OAc)₂ (0.149 g) was added to 3-(2-chloropyridin-4-yloxy)-2,6-dimethylpyridine (Method 13, 3.10 g), methyl 3-aminobenzoate (2 g ), Cs₂CO₃ (8.62 g) and xantphos (0.764 g) in dioxane (80 mL) at r.t. under nitrogen. The resulting orange mixture was stirred at 100° C. for 5 h. The mixture was then diluted with EtOAc (100 mL), and the resulting solution was washed with water (100 mL) and then saturated brine (100 mL). The organic portion was dried (MgSO₄), filtered and concentrated in vacuo to afford crude product as a brown gum. Purification by FCC using a gradient of 0 to 75% EtOAc in DCM afforded the title compound (2.95 g, 63.8%) as a white solid; ¹H NMR: 2.30 (3H, s), 2.49 (3H, s), 3.85 (3H, s), 6.09 (1H, s), 6.48 (1H, d), 7.23 (1H, d), 7.35-7.39 (1H, m), 7.46 (1H, d), 7.49 (1H, d), 7.94 (1H, d), 8.11 (1H, d), 8.26 (1H, s), 9.20 (1H, s); m/z: MH⁺ 350.5.

### Examples 144-147

The procedure described above for Example 43 was repeated using the appropriate aniline with 3-(2-chloropyridin-4-yl)oxy-6-methyl-2-pyridin-2-yl-pyridine (prepared according to Method 17). The Examples described below were thus obtained:

### Example 144

### 2-(4-{[4-(6-Methyl-2-pyridin-2-ylpyridin-3-yl)oxypyridin-2-yl]-amino}phenyl)acetonitrile

m/z: 394.37 MH⁺; EAA2: 0.02757.

### Example 145

### 4-(6-Methyl-2-pyridin-2-ylpyridin-3-yl)oxy-N-pyridin-2-yl-pyridin-2-amine

m/z: 356.38 MH⁺ EAA2: 0.008254.

### Example 146

### 4-{[4-(6-Methyl-2-pyridin-2-ylpyridin-3-yl)oxypyridin-2-yl]-amino}benzamide

m/z: 396.35 MH⁻; EAA2: 0.01524.

### Example 147

### (4-{[4-(6-Methyl-2-pyridin-2-ylpyridin-3-yl)oxypyridin-2-yl]-amino}phenyl)methanol

m/z: 385.4 MH⁺; EAA2: 0.009209.

### Examples 148-152

The procedure described above for Example 45 was repeated using the appropriate aniline with 3-(2-chloropyridin-4-yl)oxy-6-methyl-2-phenyl-pyridine (prepared according to Method 23). The examples described below were thus obtained:

### Example 148

### 2-(4-{[4-(6-Methyl-2-phenylpyridin-3-yl)oxypyridin-2-yl]-amino}phenyl)acetonitrile

m/z: 391.38 MH⁻; EAA2: 0.03391.

### Example 149

### 4-{[4-(6-Methyl-2-phenylpyridin-3-yl)oxypyridin-2-yl]amino}-benzenesulfonamide

m/z: 433.32 MH⁺; EAA2: 0.01397.

### Example 150

### 4-(6-Methyl-2-phenylpyridin-3-yl)oxy-N-pyridin-2-ylpyridin-2-amine

m/z: 355.39 MH⁺; EAA2: 0.004702.

### Example 151

### 4-{[4-(6-Methyl-2-phenylpyridin-3-yl)oxypyridin-2-yl]amino}-benzamide

m/z: 397.4 MH⁺; EAA2: 0.01369.

### Example 152

### (4-{[4-(6-Methyl-2-phenylpyridin-3-yl)oxypyridin-2-yl]amino}-phenyl)methanol

m/z: 384.42 MH⁺; EAA2: 0.007051.

### Examples 153-154

The procedure described above for Example 42 was repeated using the appropriate amino compound with 3-(2-chloropyridin-4-yl)oxy-2,6-dimethyl-pyridine (prepared according to Method 13). The examples described below were thus obtained:

### Example 153

### 4-(2,6-Dimethylpyridin-3-yl)oxy-N-pyridin-2-ylpyridin-2-amine

m/z: 293.38 MH⁺; EAA2: 0.02379.

### Example 154

### (4-{[4-(2,6-Dimethylpyridin-3-yl)oxypyridin-2-yl]amino}phenyl)-methanol

m/z: 322.38 MH⁺ EAA2: 0.01974.

### Examples 155-158

The procedure described above for Example 42 was repeated using the appropriate aniline with 3-(2-chloropyridin-4-yloxy)-2-(4-fluorophenyl)-6-methylpyridine (prepared according to Method 38). The examples described below were thus obtained:

### Example 155

### 2-[4-({4-[2-(4-Fluorophenyl)-6-methylpyridin-3-yl]oxypyridin-2-yl}amino)phenyl]acetonitrile

m/z: 409.39 MH⁻; EAA2: 0.08163.

### Example 156

### 4-({4-[2-(4-Fluorophenyl)-6-methylpyridin-3-yl]oxypyridin-2-yl}amino)benzenesulfonamide

m/z: 451.33 MH⁺; EAA2: 0.0454.

### Example 157

### 4-({4-[2-(4-Fluorophenyl)-6-methylpyridin-3-yl]oxypyridin-2-yl}-amino)benzamide

m/z: 415.39 MH⁺; EAA2: 0.04742.

### Example 158

### [4-({4-[2-(4-Fluorophenyl)-6-methylpyridin-3-yl]oxypyridin-2-yl}-amino)phenyl]methanol

m/z: 402.41; MH⁺; Retention time: 2.12 min. EAA2: 0.03152.

### Examples 159-163

The procedure described above for Example 42 was repeated using the appropriate aniline with 3-(2-chloropyridin-4-yloxy)-2-(furan-2-yl)-6-methylpyridine (prepared according to Method 39). The examples described below were thus obtained:

### Example 159

### 2-[4-({4-[2-(Furan-2-yl)-6-methylpyridin-3-yl]oxypyridin-2-yl}-amino)phenyl]acetonitrile

m/z: 383.43 MH⁺; EAA2: 0.002987.

### Example 160

### 4-({4-[2-(Furan-2-yl)-6-methylpyridin-3-yl]oxypyridin-2-yl}-amino)benzenesulfonamide

m/z: 423.37 MH⁺; EAA2: 0.01275.

### Example 161

### 4-[2-(Furan-2-yl)-6-methylpyridin-3-yl]oxy-N-pyridin-2-yl-pyridin-2-amine

m/z: 345.42 MH⁺; EAA2: 0.008547.

### Example 162

### 4-({4-[2-(Furan-2-yl)-6-methylpyridin-3-yl]oxypyridin-2-yl}-amino)benzamide

m/z: 387.43 MH⁺; EAA2: 0.02043.

### Example 163

### [4-({4-[2-(Furan-2-yl)-6-methylpyridin-3-yl]oxypyridin-2-yl}-amino)phenyl]methanol

m/z: 374.44 MH⁺; EAA2: 0.005277.

### Example 164

### 4-(6-Methyl-2-(pyrrolidin-1-yl)pyridin-3-yloxy)-N-(3,4,5-trimethoxyphenyl)pyridin-2-amine

4-Fluoro-N-(3,4,5-trimethoxyphenyl)pyridin-2-amine (Method 40, 74.9 mg, 0.27 mmol), 6-methyl-2-(pyrrolidin-1-yl)pyridin-3-ol (Method 41, 48 mg, 0.27 mmol) and K₂CO₃ (112 mg, 0.81 mmol) were suspended in DMF (3 mL) and sealed into a microwave tube. The mixture was heated to 180° C. for 15 mins in a microwave and was then cooled to r.t. The mixture was filtered and concentrated. The crude product was purified by preparative HPLC (Waters XBridge Prep C18 OBD column, 5 μm silica, 19 mm diameter, 100 mm length), using decreasingly polar mixtures of water (containing 1% NH₃) and MeCN as eluents. Fractions containing the desired compound were evaporated to dryness to afford the title compound (31.0 mg, 26.4%) as a white solid. ¹H NMR: 1.76-1.80 (4H, m), 2.34 (3H, s), 3.41-3.45 (4H, m), 3.59 (3H, s), 3.71 (6H, s), 6.04 (1H, d), 6.37-6.39 (1H, m), 6.53 (1H, d), 6.96 (2H, s), 7.22 (1H, d), 8.03 (1H, d), 8.85 (1H, s); m/z: MH⁺ 437.52; EAA2: 0.02752.

### Example 165

### 4-[2-(Furan-2-yl)-6-methylpyridin-3-yloxy]-N-(3,4,5-trimethoxy-phenyl)pyridin-2-amine

3-(2-Chloropyridin-4-yloxy)-2-(furan-2-yl)-6-methylpyridine (Method 39, 100 mg, 0.35 mmol), 3,4,5-trimethoxyaniline (77 mg, 0.42 mmol), Cs₂CO₃ (170 mg, 0.52 mmol), Pd(OAc)₂ (5.48 mg, 0.02 mmol) and xantphos (20.18 mg, 0.03 mmol) were suspended in DMA (2 mL) and sealed into a microwave tube. The mixture was heated to 150° C. for 15 mins in a microwave and was then cooled to r.t. The mixture was filtered and concentrated. The crude product was purified by preparative HPLC (Waters XBridge Prep C18 OBD column, 5 μm silica, 19 mm diameter, 100 mm length), using decreasingly polar mixtures of water (containing 1% NH₃) and MeCN as eluents. Fractions containing the desired compound were evaporated to dryness to afford the title compound (36.0 mg, 23.81%) as a white solid; ¹H NMR: 2.58 (3H, s), 3.70 (6H, s), 3.74 (3H, s), 6.23 (1H, s), 6.32 (1H, d), 6.37-6.41 (5H, m), 6.88 (1H, d), 7.00 (1H, d), 7.25 (3H, d), 7.50 (1H, s), 8.01 (1H, s); m/z: MH⁺ 434.11; EAA2: 0.01261.

### Example 166

### 4-(6-Methyl-2-propylpyridin-3-yloxy)-N-(3,4,5-trimethoxy-phenyl)pyridin-2-amine

4-Fluoro-N-(3,4,5-trimethoxyphenyl)pyridin-2-amine (Method 40, 100 mg, 0.36 mmol), 6-methyl-2-propylpyridin-3-ol (Method 43, 54.3 mg, 0.36 mmol) and K₂CO₃ (149 mg, 1.08 mmol) were suspended in DMF (3 mL) and sealed into a microwave tube. The mixture was heated to 180° C. for 15 mins in a microwave and was then cooled to r.t. The mixture was filtered and concentrated. The crude product was purified by preparative HPLC (Waters XBridge Prep C18 OBD column, 5 μm silica, 19 mm diameter, 100 mm length), using decreasingly polar mixtures of water (containing 1% NH₃) and MeCN as eluents. Fractions containing the desired compound were evaporated to dryness to afford the title compound (69.0 mg, 47%) as a white solid. ¹H NMR: 0.85 (3H, t), 1.56-1.67 (2H, m), 2.47 (3H, s), 2.57-2.62 (2H, m), 3.72 (6H, s), 3.74 (3H, s), 6.17 (1H, d), 6.20-6.23 (1H, m), 6.41 (3H, s), 6.93 (1H, d), 7.13 (1H, d), 7.98 (1H, d); m/z: MH⁺ 410.56; EAA2: 0.5972.

### Example 167

### 4-[6-Methyl-2-(oxolan-2-yl)pyridin-3-yl]oxy-N-(3,4,5-trimethoxy-phenyl)pyridin-2-amine

4-Fluoro-N-(3,4,5-trimethoxyphenyl)pyridin-2-amine (Method 40, 100 mg, 0.36 mmol), 6-methyl-2-(tetrahydrofuran-2-yl)pyridin-3-ol (Method 46, 77 mg, 0.43 mmol) and K₂CO₃ (149 mg, 1.08 mmol) were suspended in DMF (3 mL) and sealed into a microwave tube. The mixture was heated to 180° C. for 15 mins in the microwave and was then cooled to r.t. The mixture was filtered and concentrated. The crude product was purified by preparative HPLC (Waters XBridge Prep C18 OBD column, 5 μm silica, 19 mm diameter, 100 mm length), using decreasingly polar mixtures of water (containing 1% NH₃) and MeCN as eluents. Fractions containing the desired compound were evaporated to dryness to afford the title compound (20 mg, 12%) as a white solid. ¹H NMR: 1.87-2.00 (1H, m), 2.07-2.20 (3H, m), 2.56 (3H, s), 3.79 (6H, s), 3.81 (3H, s), 3.83-3.88 (1H, m), 4.03-4.10 (1H, m), 5.07 (1H, t), 6.26 (1H, d), 6.29-6.32 (1H, m), 6.45 (1H, s), 6.50 (2H, s), 7.07 (1H, d), 7.22 (1H, d), 8.05 (1H, d); m/z: MH⁺ 438.17; EAA2: 0.05683.

### Examples 168-172

The procedure described above for Example 164 was repeated using the appropriate alcohol with 4-fluoro-N-(3,4,5-trimethoxyphenyl)pyridin-2-amine (Method 40). The examples described below were thus obtained:

| | | Method |
|---|---|---|
| | | to make |
| Ex. | Compound name and Data | phenol |
| 168 | 4-[2-(4-Methoxyphenyl)-6-methylpyridin-3-yl-oxy]-N-(3,4,5- | 50 |
| | trimethoxyphenyl)pyridin-2-amine | |
| | ¹H NMR: (CDCl₃) 2.53 (3H, s), 3.69 (6H, s), 3.72 (3H, s), 3.73 (3H, s), | |
| | 6.16-6.21 (2H, m), 6.38 (2H, s), 6.71 (1H, s), 6.78-6.83 (2H, m), | |
| | 7.00 (1H, d), 7.24 (1H, d), 7.69-7.74 (2H, m), 7.92 (1H, d); m/z: 474.55 | |
| | MH⁺; EAA2: 0.02282. | |
| 169 | 4-(6′-Methoxy-6-methyl-2,3′-bipyridin-3-yloxy)-N-(3,4,5- | 52 |
| | trimethoxyphenyl)pyridin-2-amine | |
| | ¹H NMR: (CDCl₃) 2.55 (3H, s), 3.72 (6H, s), 3.74 (3H, s), 3.87 (3H, s), | |
| | 6.18 (1H, s), 6.19-6.20 (1H, m), 6.40 (3H, s), 6.67-6.70 (1H, m), | |
| | 7.05 (1H, d), 7.27 (1H, d), 7.93-7.95 (1H, m), 8.02-8.06 (1H, m), | |
| | 8.60-8.61 (1H, m); m/z: 475.55 MH⁺; EAA2: 0.07514. | |
| 170 | 4-[6-Methyl-2-(quinolin-4-yl)pyridin-3-yloxy]-N-(3,4,5- | 54 |
| | trimethoxyphenyl)pyridin-2-amine | |
| | ¹H NMR: (CDCl₃) 2.68 (3H, s), 3.77 (6H, s), 3.81 (3H, s), | |
| | 6.06-6.10 (2H, m), 6.34 (1H, s), 6.40 (2H, s), 7.31 (1H, d), 7.51 (1H, d), | |
| | 7.55-7.65 (2H, m), 7.73-7.77 (1H, m), 7.85-7.87 (1H, m), 7.95-7.98 (1H, m), | |
| | 8.53 (1H, s), 9.21 (1H, s); m/z: 495.59 MH⁺; EAA: 0.6145. | |
| 171 | 4-[2-(4-Fluorophenyl)-6-methylpyridin-3-yloxy]-N-(3,4,5- | 56 |
| | trimethoxyphenyl)pyridin-2-amine | |
| | ¹H NMR: (CDCl₃) 2.55 (3H, s), 3.71 (6H, s), 3.74 (3H, s), | |
| | 6.14-6.19 (2H, m), 6.39 (3H, s), 6.94-7.01 (2H, m), 7.06 (1H, d), 7.28 (1H, d), | |
| | 7.71-7.78 (2H, m), 7.93 (1H, d); m/z: 462.15 MH⁺; EAA2: 0.02395. | |
| 172 | 4-(6-Methyl-2,3′-bipyridin-3-yloxy)-N-(3,4,5-trimethoxyphenyl)- | 48 |
| | pyridin-2-amine | |
| | ¹H NMR: (CDCl₃) 2.57 (3H, s), 3.72 (6H, s), 3.74 (3H, s), | |
| | 6.16-6.19 (2H, m), 6.40 (3H, s), 7.12 (1H, d), 7.22-7.26 (1H, m), 7.32 (1H, d), | |
| | 7.93-7.95 (1H, m), 8.06-8.10 (1H, m), 8.49-8.51 (1H, m), | |
| | 9.00-9.01 (1H, m); m/z: 445.52 MH⁺; EAA2: 0.06101. | |

### Example 173

### 4-(6-Methyl-2-pyrimidin-5-ylpyridin-3-yl)oxy-N-(3,4,5-trimethoxyphenyl)pyridin-2-amine

5-[3-(2-Chloropyridin-4-yloxy)-6-methylpyridin-2-yl]pyrimidine (Method 58, 100 mg, 0.33 mmol), 3,4,5-trimethoxyaniline (73.6 mg, 0.40 mmol), Cs₂CO₃ (164 mg, 0.50 mmol), Pd(OAc)₂ (5.26 mg, 0.02 mmol) and xantphos (19.37 mg, 0.03 mmol) were suspended in DMA (4 mL), thoroughly degassed using nitrogen and sealed in a microwave vial. The mixture was heated to 130° C. for 15 mins in the microwave and was then cooled to r.t. The crude mixture was filtered and purified by ion exchange chromatography, using an SCX column. The desired product was eluted from the column using 0.35M NH₃/MeOH and pure fractions were evaporated to dryness to afford the crude product as a brown gum. Purification by preparative HPLC (Waters XBridge Prep C18 OBD column, 5 μm silica, 19 mm diameter, 100 mm length), eluting with decreasingly polar mixtures of water (containing 1% NH₃) and MeCN afforded the title compound; (72 mg, 48%) as a white solid. ¹HNMR: (CDCl₃) 2.58 (3H, s), 3.74 (6H, s), 3.75 (3H, s), 6.15-6.18 (1H, m), 6.21 (1H, d), 6.42 (2H, s), 6.44 (1H, s), 7.16 (1H, d), 7.34 (1H, d), 7.96 (1H, d), 9.10 (1H, s), 9.17 (2H, s); m/z: MH⁺ 446.53; EAA2: 0.09659.

### Example 174

### (3-{[4-(2,6-Dimethylpyridin-3-yl)oxypyridin-2-yl]amino}phenyl)-methanol

3-(2-Chloropyridin-4-yloxy)-2,6-dimethylpyridine (Method 13, 3.81 g, 16.24 mmol), (3-aminophenyl)methanol (3 g, 24.36 mmol), Cs₂CO₃ (7.94 g, 24.36 mmol), Pd(OAc)₂ (0.255 g, 1.14 mmol) and xantphos (0.940 g, 1.62 mmol) were suspended in DMA (54 mL) and the mixture was thoroughly degassed with nitrogen then sealed into 3 microwave tubes. The mixture was heated to 140° C. for 15 mins in the microwave and was then cooled to r.t. The crude material was part-purified by ion exchange chromatography, using an SCX column, eluting with 0.35M NH₃/MeOH to afford a brown gum. Further purification by FCC using a gradient of 10-90% EtOAc in DCM afforded the title compound; (0.535 g, 10.25%) as a white solid. ¹H NMR: (CDCl₃) 2.32 (3H, s), 2.47 (3H, s), 4.59 (2H, s), 6.16-6.18 (1H, m), 6.19 (1H, s), 6.59 (1H, s), 6.92-6.97 (2H, m), 7.06-7.09 (1H, m), 7.14 (1H, s), 7.17 (1H, d), 7.20-7.23 (2H, m), 7.96-7.99 (1H, m); m/z: MH⁺ 322.51; EAA2: 0.01131.

### Example 175

### 4-(2,6-Dimethylpyridin-3-yl)oxy-N-{3-[(4-methylsulfonyl-piperazin-1-yl)methyl]phenyl}pyridin-2-amine

Methanesulfonyl chloride (0.012 mL, 0.15 mmol) was added dropwise to 4-(2,6-dimethylpyridin-3-yloxy)-N-[3-(piperazin-1-ylmethyl)phenyl]pyridin-2-amine (Example 176, 60 mg, 0.15 mmol) and DIPEA (0.027 mL, 0.15 mmol) in DCM (4 mL) at 10° C. over a period of 5 mins under nitrogen. The resulting solution was stirred at r.t. for 24 h. After this time the mixture was concentrated. The crude material was purified by preparative HPLC (Waters XBridge Prep C18 OBD column, 5 μm silica, 19 mm diameter, 100 mm length), eluting with decreasingly polar mixtures of water (containing 1% NH₃) and MeCN to afford the title compound; (58.0 mg, 81%) as a white solid. ¹H NMR: (CDCl₃) 2.33 (3H, s), 2.48 (3H, s), 2.48-2.51 (4H, m), 2.70 (3H, s), 3.18 (4H, t), 3.44 (2H, s), 6.14-6.17 (1H, m), 6.22 (1H, d), 6.42 (1H, s), 6.89-6.93 (1H, m), 6.96 (1H, d), 7.14-7.17 (4H, m), 7.98 (1H, d); m/z: MH⁺ 468.56; EAA: 0.01 148; EAA2: 0.006108.

### Example 176

### 4-(2,6-Dimethylpyridin-3-yl)oxy-N-[3-(Piperazin-1-ylmethyl)-phenyl]pyridin-2-amine

Hydrogen chloride (4M solution in dioxane, 4.08 mL, 16.34 mmol) was added dropwise to tert-butyl 4-{3-[4-(2,6-dimethylpyridin-3-yloxy)pyridin-2-ylamino]benzyl}piperazine-1-carboxylate (Example 207, 1.600 g, 3.27 mmol) in DCM (35 mL) cooled to 10° C. over a period of 5 minutes. The resulting solution was stirred at r.t. for 60 mins. After this time, the precipitate was filtered off and washed thoroughly with diethyl ether then dried. The crude product was purified by ion exchange chromatography, using an SCX column. The desired product was eluted from the column using 0.35M NH₃/MeOH and pure fractions were evaporated to dryness to afford the title product (1.2 g, 94%) as a white solid. m/z: MH⁺ 390.72; EAA2: 0.008287.

### Example 177

### N-(2-Adamantyl)-4-[(3-{[4-(2,6-dimethylpyridin-3-yl)oxypyridin-2-yl]amino}phenyl)methyl]piperazine-1-carboxamide

1-Adamantyl isocyanate (27.3 mg, 0.15 mmol) was added to 4-(2,6-dimethylpyridin-3-yloxy)-N-[3-(piperazin-1-ylmethyl)phenyl]pyridin-2-amine (Example 176, 60 mg, 0.15 mmol) in DCM (4 mL) at 25° C. under nitrogen. The resulting solution was stirred at r.t. for 24 h, then concentrated. The crude product was purified by preparative HPLC (Waters XBridge Prep C18 OBD column, 5 μm silica, 19 mm diameter, 100 mm length), using decreasingly polar mixtures of water (containing 1% NH₃) and MeCN as eluents. Fractions containing the desired compound were evaporated to dryness to afford the title compound (57 mg, 65%) as a beige solid. ¹H NMR: (CDCl₃) 1.60 (6H, t), 1.90 (6H, d), 1.99 (3H, s), 2.33 (3H, s), 2.35 (4H, t), 2.48 (3H, s), 3.24 (4H, t), 3.40 (2H, s), 4.11 (1H, s), 6.14-6.16 (1H, m), 6.22 (1H, d), 6.44 (1H, s), 6.92 (1H, d), 6.96 (1H, d), 7.11-7.17 (4H, m), 7.98 (1H, d); m/z: MH⁺ 567.64; EAA2: 0.04694.

### Examples 178-181

The procedure described above for Example 177 was repeated using the appropriate isocyanate with 4-(2,6-dimethylpyridin-3-yl)oxy-N-[3-(piperazin-1-ylmethyl)phenyl]-pyridin-2-amine (Example 176). The examples described below were thus obtained:

| Ex. | Name and Data |
|---|---|
| 178 | N-Cyclohexyl-4-{3-[4-(2,6-dimethylpyridin-3-yloxy)pyridin-2- |
| | ylamino]benzyl}piperazine-1-carboxamide |
| | ¹H NMR: (CDCl₃) 1.02-1.20 (3H, m), 1.26-1.44 (2H, m), 1.63-1.73 (3H, |
| | m), 1.92-1.97 (2H, m), 2.40-2.44 (7H, m), 2.54 (3H, s), 3.34 (4H, t), |
| | 3.47 (2H, s), 3.58-3.70 (1H, m), 4.28 (1H, d), 6.21-6.24 (1H, m), 6.29 (1H, d), |
| | 6.51 (1H, s), 6.97-7.04 (2H, m), 7.18-7.23 (4H, m), 8.05 (1H, d); m/z: |
| | MH⁺ 515.68; EAA2: 0.02374. |
| 179 | 4-[(3-{[4-(2,6-Dimethylpyridin-3-yl)oxypyridin-2-yl]- |
| | amino}phenyl)methyl]-N-(4-methoxyphenyl)piperazine-1-carboxamide |
| | ¹H NMR: (CDCl₃) 2.34 (3H, s), 2.38 (4H, t), 2.47 (3H, s), 3.40 (4H, t), |
| | 3.41 (2H, s), 3.71 (3H, s), 6.18-6.21 (2H, m), 6.36 (1H, s), 6.48 (1H, s), |
| | 6.75-6.78 (2H, m), 6.92-6.97 (2H, m), 7.12-7.22 (6H, m), 8.00 (1H, d); |
| | m/z: MH⁺ 538.66; EAA2: 0.02334. |
| 180 | N-(3-Chloro-4-fluorophenyl)-4-[(3-{[4-(2,6-dimethylpyridin-3-yl)oxypyridin-2- |
| | yl]amino}phenyl)methyl]piperazine-1-carboxamide |
| | ¹H NMR: (CDCl₃) 2.34-2.37 (7H, m), 2.46 (3H, s), 3.40 (4H, t), |
| | 3.40 (2H, s), 6.20 (1H, d), 6.22-6.25 (1H, m), 6.50 (1H, s), 6.69 (1H, s), |
| | 6.92-6.99 (3H, m), 7.07-7.23 (5H, m), 7.48-7.51 (1H, m), 8.01 (1H, d); m/z: MH⁺ 561.60; |
| | EAA2: 0.0896. |
| 181 | 4-[(3-{[4-(2,6-Dimethylpyridin-3-yl)oxypyridin-2-yl]amino}- |
| | phenyl)methyl]-N-pentylpiperazine-1-carboxamide |
| | ¹H NMR: (CDCl₃) 0.82 (3H, t), 1.17-1.29 (4H, m), 1.38-1.48 (2H, m), |
| | 2.33 (3H, s), 2.35 (4H, t), 2.47 (3H, s), 3.11-3.18 (2H, m), 3.28 (4H, t), |
| | 3.40 (2H, s), 4.37 (1H, t), 6.15-6.17 (1H, m), 6.22 (1H, d), 6.48 (1H, s), |
| | 6.92 (1H, d), 6.96 (1H, d), 7.09-7.21 (4H, m), 7.98 (1H, d); m/z: MH⁺ |
| | 503.25; EAA2: 0.01589. |

### Example 182

### 4-(2,6-Dimethylpyridin-3-yl)oxy-N-[3-({4-[2-(1,2,4-triazol-1-yl)-pyridin-3-yl]sulfonylpiperazin-1-yl}methyl)phenyl]pyridin-2-amine

4-(2,6-Dimethylpyridin-3-yloxy)-N-[3-(piperazin-1-ylmethyl)phenyl]pyridin-2-amine (Example 176, 60 mg, 0.15 mmol) was added to 2-(1H-1,2,4-triazol-1-yl)pyridine-3-sulfonyl chloride (41.5 mg, 0.17 mmol) and DIPEA (0.027 mL, 0.15 mmol) in DCM (4 mL) at 25° C. over a period of 2 mins under nitrogen. The resulting solution was stirred at r.t. for 24 h. After this time the mixture was concentrated. The crude material was purified by preparative HPLC (Waters XBridge Prep C18 OBD column, 5 μm silica, 19 mm diameter, 100 mm length), eluting with decreasingly polar mixtures of water (containing 1% NH₃) and MeCN to afford the title compound (70 mg, 76%) as a yellow solid; ¹H NMR: (CDCl₃) 2.30 (4H, t), 2.32 (3H, s), 2.47 (3H, s), 2.96 (4H, t), 3.35 (2H, s), 6.13-6.19 (2H, m), 6.40 (1H, s), 6.82-6.86 (1H, m), 6.96 (1H, d), 7.08 (1H, s), 7.12-7.16 (3H, m), 7.54-7.58 (1H, m), 7.98 (1H, d), 8.07 (1H, s), 8.43-8.46 (1H, m), 8.65 (1H, s), 8.71-8.73 (1H, m); m/z: MH⁺ 598.57; EAA2: 0.01825.

### Example 183

### N-(3-{[4-(cyclopropylcarbonyl)piperazin-1-yl]methyl}phenyl)-4-[(2,6-dimethylpyridin-3-yl)oxy]pyridin-2-amine

Cyclopropanecarbonyl chloride (17.71 mg, 0.17 mmol) was added dropwise to DIPEA (0.035 mL, 0.20 mmol) and 4-(2,6-dimethylpyridin-3-yloxy)-N-[3-(piperazin-1-ylmethyl)phenyl]pyridin-2-amine (Example 176, 60 mg, 0.15 mmol) in DCM (4 mL) cooled to 10° C. over a period of 1 min. under nitrogen. The resulting solution was stirred at r.t. for 20 h and was then concentrated. The crude material was purified by preparative HPLC (Waters XBridge Prep C18 OBD column, 5 μm silica, 19 mm diameter, 100 mm length), eluting with decreasingly polar mixtures of water (containing 1% NH₃) and MeCN to afford the title compound (38.0 mg, 53.9%) as a white solid. ¹H NMR: (CDCl₃) 0.64-0.70 (2H, m), 0.88-0.93 (2H, m), 1.60-1.69 (1H, m), 2.33 (3H, s), 2.39 (4H, s), 2.48 (3H, s), 3.42 (2H, s), 3.59 (4H, s), 6.15 (1H, d), 6.47 (1H, s), 6.47 (1H, s), 6.91-6.97 (2H, m), 7.13-7.19 (4H, m), 7.99 (1H, d); m/z: MH⁺ 458.62; EAA2: 0.01272.

### Examples 185-187

The procedure described above for Example 183 was repeated using the appropriate acid chloride with 4-(2,6-dimethylpyridin-3-yl)oxy-N-[3-(piperazin-1-ylmethyl)phenyl]pyridin-2-amine (Example 176). The examples described below were thus obtained:

| Ex. | Name and Data |
|---|---|
| 185 | {4-[(3-{[4-(2,6-Dimethylpyridin-3-yl)oxypyridin-2-yl]-amino}phenyl)- |
| | methyl]piperazin-1-yl}-(1-methylpyrrol-3-yl)methanone |
| | ¹H NMR: (CDCl₃) 2.32 (3H, s), 2.40 (4H, t), 2.47 (3H, s), 3.42 (2H, s), |
| | 3.57 (3H, s), 3.68 (4H, t), 6.13-6.18 (2H, m), 6.23 (1H, d), 6.44-6.45 (2H, |
| | m), 6.89-6.94 (2H, m), 6.96 (1H, d), 7.12-7.21 (4H, m), 7.98 (1H, d); m/z: MH⁺ 515.68; |
| | EAA: 0.0112. |
| 186 | {4-[(3-{[4-(2,6-Dimethylpyridin-3-yl)oxypyridin-2-yl]amino}- |
| | phenyl)methyl]piperazin-1-yl}-(oxan-4-yl)methanone |
| | ¹H NMR: (CDCl₃) 1.50-1.54 (2H, m), 1.77-1.91 (2H, m), 2.32 (3H, s), |
| | 2.37 (4H, t), 2.48 (3H, s), 2.59-2.69 (1H, m), 3.31-3.40 (2H, m), |
| | 3.41 (4H, s), 3.56 (2H, s), 3.91-3.96 (2H, m), 6.14-6.16 (1H, m), 6.22 (1H, d), |
| | 6.48 (1H, s), 6.91 (1H, d), 6.96 (1H, d), 7.12-7.17 (4H, m), 7.98 (1H, d); |
| | m/z: MH⁺ 502.71; EAA2: 0.0238. |
| 187 | 3-{4-[(3-{[4-(2,6-Dimethylpyridin-3-yl)oxypyridin-2- |
| | yl]amino}phenyl)methyl]piperazin-1-yl}-3-oxopropanamide |
| | ¹H NMR: (CDCl₃) 2.32 (3H, s), 2.37-2.40 (4H, m), 2.47 (3H, s), |
| | 3.26 (2H, s), 3.41 (2H, s), 3.47 (2H, t), 3.58 (2H, t), 5.56 (1H, s), 6.15 (1H, d), |
| | 6.69 (1H, s), 6.69 (1H, s), 6.90 (1H, d), 6.96 (1H, d), 7.12-7.18 (4H, m), |
| | 7.40 (1H, s), 7.98 (1H, d); m/z: MH⁺ 515.68; EAA2: 0.02325. |

### Example 188

### N-cyclopropyl-2-{4-[(3-{[4-(2,6-dimethylpyridin-3-yl)oxypyridin-2-yl]amino}phenyl)methyl]piperazin-1-yl}acetamide

4-(2,6-Dimethylpyridin-3-yloxy)-N-[3-(piperazin-1-ylmethyl)phenyl]pyridin-2-amine (Example 176, 60 mg, 0.15 mmol), 2-chloro-N-cyclopropylacetamide (20.58 mg, 0.15 mmol) and N,N-diethylmethylamine (0.019 mL, 0.15 mmol) were dissolved in DCM (4 mL) and sealed into a microwave tube. The mixture was heated to 110° C. for 45 mins in a microwave and was then cooled to r.t. The mixture was concentrated. The crude material was purified by preparative HPLC (Waters XBridge Prep C18 OBD column, 5 μm silica, 19 mm diameter, 100 mm length), eluting with decreasingly polar mixtures of water (containing 1% NH₃) and MeCN to afford the title compound (33.0 mg, 44.0%) as a white solid. ¹H NMR: (CDCl₃) 0.46-0.52 (2H, m), 0.75-0.82 (2H, m), 2.40 (3H, s), 2.48-2.53 (8H, m), 2.55 (3H, s), 2.68-2.76 (1H, m), 2.97 (2H, s), 3.47-3.49 (2H, m), 6.20-6.23 (1H, m), 6.29 (1H, d), 6.51 (1H, s), 6.96-6.99 (1H, m), 7.02-7.04 (1H, m), 7.13 (1H, s), 7.20-7.25 (4H, m), 8.05 (1H, d); m/z: MH⁺ 487.66; EAA2: 0.006908.

### Example 189

### 3-Amino-1-{4-[(3-{[4-(2,6-dimethylpyridin-3-yl)oxypyridin-2-yl]-amino}phenyl)methyl]piperazin-1-yl}propan-1-one

Hydrogen chloride (4M solution in dioxane, 0.054 mL, 0.21 mmol) was added to tert-butyl 3-(4-{3-[4-(2,6-dimethylpyridin-3-yloxy)pyridin-2-ylamino]benzyl}piperazin-1-yl)-3-oxopropylcarbamate (Example 208, 30.0 mg, 0.05 mmol) in DCM (5 mL) at 25° C. over a period of 20 seconds under nitrogen. The resulting solution was stirred at r.t. for 24 h. After this time the mixture was diluted with MeOH (10 mL) and purified by ion exchange chromatography, using an SCX column and eluting with 0.35M NH₃/MeOH to afford the title compound (22.00 mg, 89%) as a white solid. ¹H NMR: (CDCl₃) 2.27 (3H, s), 2.32 (2H, s), 2.34-2.37 (4H, m), 2.42 (2H, t), 2.47 (3H, s), 2.96 (2H, t), 3.36-3.40 (2H, m), 3.38 (2H, t), 3.40 (2H, s), 6.13-6.16 (1H, m), 6.22 (1H, d), 6.70 (1H, s), 6.89-6.92 (1H, m), 6.96 (1H, d), 7.12-7.18 (4H, m), 7.98 (1H, d); m/z: MH⁺ 461.64; EAA2: 0.02618.

### Example 190

### 2-14-[3-({4-[(2,6-dimethylpyridin-3-yl)oxylpyridin-2-yl}amino)-benzyl]piperazin-1-yl}propanamide

2-Bromopropanamide (21.46 mg, 0.14 mmol) was added to 4-(2,6-dimethylpyridin-3-yloxy)-N-[3-(piperazin-1-ylmethyl)phenyl]pyridin-2-amine (Example 176, 50 mg, 0.13 mmol) and DIPEA (0.067 mL, 0.39 mmol) in dioxane (2.5 mL) at 25° C. under nitrogen. The resulting solution heated in a microwave vial to 100° C. for 15 mins. After this time the mixture was concentrated and purified by preparative HPLC (Waters XBridge Prep C18 OBD column, 5 μm silica, 19 mm diameter, 100 mm length), using decreasingly polar mixtures of water (containing 1% NH₃) and MeCN as eluents to afford the title compound; (42.0 mg, 71.0%) as a white solid. ¹H NMR: (CDCl₃) 1.16 (3H, d), 2.33 (3H, s), 2.43 (4H, s), 2.48 (3H, s), 2.52-2.58 (4H, m), 2.96-3.03 (1H, m), 3.41 (2H, s), 5.21 (1H, s), 6.13-6.15 (1H, m), 6.23-6.24 (1H, m), 6.46 (1H, s), 6.90-6.97 (2H, m), 7.00 (1H, s), 7.12-7.18 (4H, m), 7.98 (1H, d); m/z: MH⁺ 461.74; EAA2: 0.03225.

### Example 191

### 2-{4-[3-({4-[(2,6-dimethylpyridin-3-yl)oxylpyridin-2-yl]amino)-benzyl]piperazin-1-yl}-N-methylacetamide

4-(2,6-Dimethylpyridin-3-yloxy)-N-[3-(piperazin-1-ylmethyl)phenyl]pyridin-2-amine (Example 176, 50 mg, 0.13 mmol), 2-chloro-N-methylacetamide (15.19 mg, 0.14 mmol) and DIPEA (0.067 mL, 0.39 mmol) were dissolved in dioxane (2.5 mL) and sealed into a microwave tube. The mixture was heated to 1 50° C. for 10 mins in a microwave and was then cooled to r.t. The solution was concentrated and purified by preparative HPLC (Waters XBridge Prep C18 OBD column, 5 μm silica, 19 mm diameter, 100 mm length), eluting with decreasingly polar mixtures of water (containing 1% NH₃) and MeCN, to afford the title compound (42.0 mg, 71.0%) as a white solid. ¹H NMR: (CDCl₃) 2.33 (3H, s), 2.41-2.42 (4H, m), 2.46-2.50 (7H, m), 2.77 (3H, d), 2.93 (2H, s), 3.41 (2H, s), 6.13-6.16 (1H, m), 6.22 (1H, d), 6.47 (1H, s), 6.90-6.92 (1H, m), 6.96 (1H, d), 7.05 (1H, s), 7.12-7.18 (4H, m), 7.98 (1H, d); m/z: MH⁺ 461.77; EAA2: 0.02125.

### Example 192

### 1-(2-{4-[(3-{[4-(2,6-Dimethylpyridin-3-yl)oxypyridin-2-yl]-amino}phenyl)methyl]piperazin-1-yl}ethyl)imidazolidin-2-one

4-(2,6-Dimethylpyridin-3-yloxy)-N-[3-(piperazin-1-ylmethyl)phenyl]pyridin-2-amine (Example 176, 50 mg, 0.13 mmol), 1-(2-chloroethyl)imidazolidin-2-one (19.07 mg, 0.13 mmol) and DIPEA (0.067 mL, 0.39 mmol) were dissolved in dioxane (2.5 mL) and sealed into a microwave tube. The mixture was heated to 150° C. for 60 mins in a microwave and was then cooled to r.t. After this time the reaction mixture was concentrated and purified by preparative HPLC (Waters XBridge Prep C18 OBD column, 5 μm silica, 19 mm diameter, 100 mm length), eluting with decreasingly polar mixtures of water (containing 1% NH₃) and MeCN, to afford the title compound (30 mg, 47%) as a white solid. ¹H NMR: (CDCl₃) 2.06 (1H, s), 2.32 (4H, s), 2.40-2.44 (7H, m), 2.47 (4H, s), 3.24 (2H, t), 3.29-3.45 (6H, m), 4.54 (1H, s), 6.11-6.14 (1H, m), 6.23 (1H, s), 6.80 (1H, s), 6.90 (1H, d), 6.95 (1H, d), 7.11-7.16 (4H, m), 7.97 (1H, d); m/z: MH⁺ 502.83; EAA2: 0.0155.

### Example 193

### 4-(2,6-Dimethylpyridin-3-yl)oxy-N-{3-[(4-methylpiperazin-1-yl)-methyl]phenyl}pyridin-2-amine

N-[3-(Chloromethyl)phenyl]-4-(2,6-dimethylpyridin-3-yloxy)pyridin-2-amine (Example 209, 70 mg, 0.16 mmol), 1-methylpiperazine (0.037 mL, 0.33 mmol) and N,N-diethylmethylamine (0.060 mL, 0.49 mmol) were dissolved in DCM (3 mL) and sealed into a microwave tube. The mixture was heated to 100° C. for 60 mins in a microwave and was then cooled to r.t. and concentrated. The mixture was purified by preparative HPLC (Waters XBridge Prep C18 OBD column, 5 μm silica, 19 mm diameter, 100 mm length), eluting with decreasingly polar mixtures of water (containing 1% NH₃) and MeCN, to afford the title compound (29.0 mg, 43.6%) as a white solid. ¹H NMR: (CDCl₃) 2.21 (3H, s), 2.33 (3H, s), 2.39 (4H, s), 2.48 (3H, s), 3.40 (2H, s), 6.12-6.15 (1H, m), 6.24 (1H, d), 6.91-6.97 (2H, m), 7.11-7.18 (9H, m), 7.98 (1H, d); m/z: MH⁺ 404.51; EAA2: 0.004273.

### Example 194A

### N-Cyclopropyl-3-{[4-(2,6-dimethylpyridin-3-yl)oxypyridin-2-yl]-amino}benzamide

Cyclopropylamine (0.050 mL, 0.72 mmol) was added to 3-[4-(2,6-dimethylpyridin-3-yloxy)pyridin-2-ylamino]benzoic acid (Example 210, 120 mg, 0.36 mmol), DIPEA (50.9 mg, 0.39 mmol) and HBTU (150 mg, 0.39 mmol) in DMF (3 mL) at 25° C. over a period of 1 min. The resulting solution was stirred at r.t. for 24 h. After this time the solution was concentrated and purified by preparative HPLC (Waters XBridge Prep C18 OBD column, 5 μm silica, 19 mm diameter, 100 mm length), using decreasingly polar mixtures of water (containing 1% NH₃) and MeCN as eluents to afford the title compound (92 mg, 69%) as a white solid. ¹H NMR: (CDCl₃) 0.51-0.56 (2H, m), 0.75-0.81 (2H, m), 2.31 (3H, s), 2.46 (3H, s), 2.77-2.85 (1H, m), 6.16 (1H, d), 6.19-6.21 (1H, m), 6.27 (1H, s), 6.96 (1H, d), 6.97 (1H, s), 7.15-7.27 (3H, m), 7.46-7.50 (1H, m), 7.70-7.70 (1H, m), 8.00 (1H, d); m/z: MH⁺ 375.39; EAA2: 0.04574.

### Example 194B

### N-(2-Chlorophenyl)-4-(5,6-dimethyl-2-pyridin-2-ylpyridin-3-yl)-oxypyridin-2-amine

The title compound was prepared according to the method described in Example 29 using 2-chloroaniline. m/z: MH⁺ 403.5; EAA: 2.798.

### Example 195

### tert-Butyl N-[2-(4-{[4-(5,6-dimethyl-2-pyridin-2-ylpyridin-3-yl)-oxypyridin-2-yl]amino}phenoxy)ethyl]carbamate

Diisopropylazodicarboxylate (0.350 mL, 1.78 mmol) was added dropwise to a mixture of tert-butyl N-(2-hydroxyethyl)carbamate (0.055 mL, 0.36 mmol), 4-{[4-(5,6-dimethyl-2-pyridin-2-ylpyridin-3-yl)oxypyridin-2-yl]amino}phenol (Example 212, 91 mg, 0.24 mmol) and triphenylphosphine (155 mg, 0.59 mmol) in THF (15 mL) at 22° C. over a period of 5 mins. The resulting solution was stirred at 22° C. for 2 h. The reaction was incomplete and further triphenylphosphine (155 mg, 0.59 mmol) and diisopropyl azodicarboxylate (0.117 mL, 0.59 mmol) were added then the solution was stirred at 22° C. for a further 16 h. The reaction was incomplete and further triphenylphosphine (155 mg, 0.59 mmol) and diisopropyl azodicarboxylate (0.117 mL, 0.59 mmol) were added and the solution was stirred at 22° C. for a further 24 h. The mixture was concentrated and then purified by ion exchange chromatography, using an SCX column, eluting with 7M NH₃ in MeOH. Appropriate fractions were concentrated in vacuo. The residue was purified by FCC using an elution gradient of 50 to 80% EtOAc in isohexane. Clean fractions were concentrated in vacuo and the resulting residue was triturated with diethyl ether to afford the title compound as a white solid, (12 mg, 10%); ¹H NMR: (CDCl₃): 1.46 (9H, s), 2.60 (3H, s), 2.60 (3H, s), 3.52 (2H, dt), 4.00 (2H, t), 4.99 (1H, br s), 6.14 (1H, d), 6.17 (1H, dd), 6.30 (1H, s), 6.84 (2H, d), 7.14 (2H, d), 7.21 (1H, ddd), 7.24 (1H, s), 7.67 (1H, ddd), 7.76 (1H, dd), 7.92 (1H, d), 8.67 (1H, dd); m/z: MH⁺ 528.3; EAA2: 0.01211.

### Example 196

### tert-Butyl N-[3-(4-{[4-(5,6-dimethyl-2-pyridin-2-ylpyridin-3-yl)-oxypyridin-2-yl]amino}phenoxy)propyl]carbamate

The title compound was prepared using the general method described in Example 195 using tert-butyl N-(3-hydroxypropyl)carbamate, in 13% yield as a white solid; ¹H NMR: (CDCl₃) 1.44 (9H, s), 1.97 (2H, tt), 2.34 (3H, s), 2.60 (3H, s), 3.33 (2H, dt), 4.00 (2H, t), 4.75 (1H, br.s), 6.14 (1H, d), 6.16 (1H, dd), 6.29 (1H, s), 6.84 (2H, d), 7.13 (2H, d), 7.21 (1H, ddd), 7.24 (1H, s), 7.67 (1H, ddd), 7.75 (1H, dd), 7.92 (1H, d), 8.67 (1H, dd); m/z: MH⁺ 542.3; EAA2: 0.009025.

### Example 197

### tert-Butyl N-{3-[(4-{[4-(5,6-dimethyl-2-pyridin-2-ylpyridin-3-yl)-oxypyridin-2-yl]amino}benzoyl)amino]propyl}carbamate

HBTU (127 mg, 0.33 mmol) was added dropwise as a solution in DMA (5 mL) to 4-{[4-(5,6-dimethyl-2-pyridin-2-ylpyridin-3-yl)oxypyridin-2-yl]amino}benzoic acid (Example 213, 92 mg, 0.22 mmol) and tert-butyl N-(3-aminopropyl)carbamate (0.047 mL, 0.27 mmol) in DMA (10 mL). The resulting solution was stirred at 22° C. for 20 h. The mixture was concentrated in vacuo and the residue partitioned between EtOAc (30 mL) and water (30 mL). The phases were separated and the aqueous portion was extracted with EtOAc (30 mL). The combined organic portions were washed with water (4×30 mL), brine (30 mL) and was then dried (MgSO₄), filtered and concentrated in vacuo. The residue was purified by FCC using an elution gradient of 0-2% [10:1 MeOH/conc. NH₃ (aq)] in EtOAc. Clean fractions were evaporated to dryness to afford the title compound as a white dry film, (85 mg, 67%); ¹H NMR: (CDCl₃) 1.46 (9H, s), 1.70 (2H, tt), 2.36 (3H, s), 2.61 (3H, s), 3.24 (2H, dt), 3.50 (2H, t), 4.90 (1H, br.s), 6.28 (1H, d), 6.33 (1H, dd), 6.64 (1H, s), 7.06 (1H, br s), 7.20 (1H, ddd), 7.28 (1H, s), 7.33 (2H, d), 7.69 (1H, ddd), 7.77 (2H, d), 7.81 (1H, d), 8.02 (1H, d), 8.65 (1H, dd); m/z: MH⁺ 569.6; EAA2: 0.006097.

### Example 198

### tert-Butyl N-{2-[(4-{[4-(5,6-dimethyl-2-pyridin-2-ylpyridin-3-yl)oxypyridin-2-yl]amino}benzoyl)amino]ethyl}carbamate

This compound was prepared in a similar manner as in Example 197 using tert-butyl N-(2-aminoethyl)carbamate. The title compound was obtained in 65% yield as a white solid after trituration with diethyl ether; ¹H NMR: (CDCl₃) 1.42 (9H, s), 2.36 (3H, s), 2.62 (3H, s), 3.39 (2H, dt), 3.54 (2H, dt), 4.97 (1H, br s), 6.27 (1H, d), 6.33 (1H, dd), 6.98 (1H, br.s), 7.20 (1H, ddd), 7.28 (1H, s), 7.32 (2H, d), 7.69 (1H, ddd), 7.74 (2H, d), 7.80 (1H, dd), 8.02 (1H, d), 8.65 (1H, dd); m/z: MH⁺ 555.6; EAA2: 0.0147.

### Example 199

### N-(3-Aminopropyl)-4-{[4-(5,6-dimethyl-2-pyridin-2-ylpyridin-3-yl)oxypyridin-2-yl]amino}benzamide

tert-Butyl N-[3-(4-{[4-(5,6-dimethyl-2-pyridin-2-ylpyridin-3-yl)oxypyridin-2-yl]amino}phenoxy)propyl]carbamate (Example 197, 42 mg, 0.074 mmol) was dissolved in TFA (3 mL) and the mixture was stirred for 2 h. The mixture was concentrated and then purified by ion exchange chromatography using an SCX column. The column was eluted with 30% MeOH in DCM to remove impurities, then with 30% (3M NH₃ in MeOH) in DCM to elute the product. Appropriate fractions were concentrated in vacuo to give the title compound as a white solid, (32 mg, 97%); ¹H NMR: (CDCl₃) 1.73 (2H, tt), 2.35 (3H, s), 2.60 (3H, s), 2.90 (2H, t), 3.56 (2H, dt), 6.26 (1H, d), 6.32 (1H, dd), 6.80 (1H, s), 7.20 (1H, ddd), 7.27 (1H, s), 7.32 (2H, d), 7.53 (1H, br t), 7.68 (1H, ddd), 7.71 (2H, d), 7.79 (1H, dd), 8.01 (1H, d), 8.64 (1H, dd); m/z: MH⁺ 469.3; EAA2: 0.00145.

### Example 200

### N-(2-Aminoethyl)-4-{[4-(5,6-dimethyl-2-pyridin-2-ylpyridin-3-yl)oxypyridin-2-yl]amino}benzamide

This compound was prepared in a similar manner to Example 199, using tert-butyl N-{2-[(4-{[4-(5,6-dimethyl-2-pyridin-2-ylpyridin-3-yl)oxypyridin-2-yl]amino}benzoyl)-amino]ethyl}carbamate (Example 198). The compound was isolated in a 90% yield as a white solid; ¹H NMR: (CDCl₃) 2.36 (3H, s), 2.62 (3H, s), 2.94 (2H, t), 3.49 (2H, dt), 6.25 (1H, d), 6.34 (1H, dd), 6.60 (2H, m), 7.20 (1H, ddd), 7.28 (1H, s), 7.33 (2H, d), 7.69 (1H, ddd), 7.72 (2H, d), 7.80 (1H, dd), 8.02 (1H, d), 8.65 (1H, dd); m/z: MH⁻ 455.3; EAA2: 0.004845.

### Example 201

### 3-{[4-(5,6-Dimethyl-2-pyridin-2-ylpyridin-3-yl)oxypyridin-2-yl]-amino}phenol

3-Aminophenol (52.5 mg, 0.48 mmol), 5-(2-chloropyridin-4-yl)oxy-2,3-dimethyl-6-pyridin-2-yl-pyridine (Method 16, 100 mg, 0.32 mmol), Cs₂CO₃ (314 mg, 0.96 mmol) and xantphos (22.27 mg, 0.04 mmol) were suspended in 1,4-dioxane (5 mL). The mixture was purged with nitrogen and Pd₂(dba)₃ (11.75 mg, 0.01 mmol) was added. The mixture was purged with nitrogen and sealed into a microwave tube. The mixture was heated to 150° C. for 60 mins in a microwave and was then cooled to r.t. The mixture was filtered and the residue was washed with DCM. The combined filtrates were concentrated then purified by FCC eluting with EtOAc to afford the title compound (105 mg, 85%) as a light brown gum; ¹H NMR: (CDCl₃) 2.34 (3H, s), 2.60 (3H, s), 6.12 (1H, d), 6.37 (1H, ddd), 6.39 (1H, d), 6.50 (1H, ddd), 6.50 (1H, s), 6.62 (1H, dd), 7.07 (1H, dd), 7.27 (1H, s), 7.28 (4H, ddd), 7.71 (1H, ddd), 7.83 (1H, dd), 7.92 (1H, d), 8.72 (1H, ddd); m/z: MH⁺ 385.3; EAA2: 0.003929.

### Example 202

### Methyl 3-{[4-(5,6-dimethyl-2-pyridin-2-ylpyridin-3-yl)oxypyridin-2-yl]amino}benzoate

5-(2-Chloropyridin-4-yl)oxy-2,3-dimethyl-6-pyridin-2-yl-pyridine (Method 16, 1.894 g), methyl-3-aminobenzoate (1.377 g), xantphos (0.422 g), Pd(OAc)₂ (0.109 g) and Cs₂CO₃ (3.96 g) were suspended in DMA (15 mL) and sealed into a microwave tube. The mixture was heated to 150° C. for 45 mins in a microwave. The crude material was purified by ion exchange chromatography, using an SCX column. The desired product eluted from the column in both fractions and so all fractions were combined and evaporated to a small volume. A solid precipitated out, this was filtered and washed with ether to afford the title compound; (1.620 g, 62.5%); ¹H NMR: 2.35 (3H, s), 2.45 (3H, s), 3.8 (3H, s), 6.1 (1H, d), 6.4 (1H, dd), 7.3 (2H, m); EAA2: 0.0279.

### Example 203

### 3-{[4-(5,6-Dimethyl-2-pyridin-2-ylpyridin-3-yl)oxypyridin-2-yl]-amino}benzoic acid

Methyl 3-{[4-(5,6-dimethyl-2-pyridin-2-ylpyridin-3-yl)oxypyridin-2-yl]amino}-benzoate (Example 202, 1.62 g) and NaOH solution (11.4 mL, 1M) were suspended in MeOH (7 mL) and sealed into a microwave tube. The mixture was heated to 120° C. for 5 mins in a microwave. After cooling the aqueous layer was acidified with 2M HCl solution. Upon standing, precipitation occurred to provide the title compound. (0.857 g, 54.7%).

### Example 204

### N-(2-Chloroethyl)-4-[4-(2,6-dimethylpyridin-3-yloxy)pyridin-2-ylamino]benzenesulfonamide

3-(2-Chloropyridin-4-yloxy)-2,6-dimethylpyridine (Method 13, 913 mg), 4-amino-N-(2-chloroethyl)benzenesulfonamide (964 mg) and p-toluenesulfonic acid monohydrate (1.52 g) were dissolved in 4-methyl-2-pentanol (20 mL) and heated to 130° C. over 15 mins. The mixture was then heated at 130° C., under nitrogen for 6 h. The mixture was then cooled and evaporated to dryness. The residue was partitioned between water and diethyl ether/EtOAc (containing a little MeOH). The aqueous layer was separated and was adjusted carefully to pH 8 by addition of an aqueous solution of saturated NaHCO₃. The mixture was then extracted with DCM (×2) and the combined organics were dried (MgSO₄), filtered and evaporated to afford the title compound (1.21 g, 72%), which was used without further purification; m/z: MH⁺ 432.9.

### Example 205

### Lithium 4-{[4-(2,6-dimethylpyridin-3-yl)oxypyridin-2-yl]-amino}benzoate

Lithium hydroxide monohydrate (12.1 mg) in water (1 mL) was added to ethyl 4-[4-(2,6-dimethylpyridin-3-yloxy)pyridin-2-ylamino]benzoate (Example 206, 105 mg) in EtOH (5 mL) at 20° C. under air. The resulting pale yellow solution was stirred at 50° C. for 24 h. Solvent was removed by evaporation. The resulting residue was diluted with water (20 mL) and extracted with EtOAc (2×20 mL). The aqueous solution was evaporated to dryness to afford the title compound as a dry film; (60 mg, 62%); ¹H NMR: 2.30 (3H, s), 2.48 (3H, s), 6.10 (1H, s), 6.40-6.42 (1H, m), 7.21 (1H, d), 7.47-7.51 (3H, m), 7.74 (2H, d), 8.07 (1H, d), 8.98 (1H, s); m/z: MH⁺ 336.5; EAA: 0.1504.

### Example 206

### Ethyl 4-{[4-(2,6-dimethylpyridin-3-yl)oxypyridin-2-yl]amino}-benzoate

Pd(OAc)₂ (0.130 g) was added to 3-(2-chloropyridin-4-yloxy)-2,6-dimethylpyridine (Method 13, 2.71 g), ethyl 4-aminobenzoate (2.098 g), Cs₂CO₃ (7.52 g) and xantphos (0.570 g) in dioxane (80 mL) at 20° C. under nitrogen. The resulting orange mixture was stirred at 100° C. for 5 h. The mixture was diluted with EtOAc (100 mL), and washed sequentially with water (100 mL) then saturated brine (100 mL). The organic portion was dried (MgSO₄), filtered and concentrated in vacuo to afford crude material as a brown gum. Purification by FCC eluting with a gradient of 0-75% EtOAc in DCM afforded the title compound; (3.40 g, 81%); as a white solid; ¹H NMR: 1.38 (3H, t), 2.40 (3H, s), 2.55 (3H, s), 4.35 (2H, q), 6.31 (1H, s), 6.33-6.35 (1H, m), 6.85 (1H, s), 7.05 (1H, d), 7.24 (1H, d), 7.40 (2H, d), 7.98 (2H, d), 8.13 (1H, d); m/z: MH⁺ 364.2.

### Example 207

### tert-Butyl 4-[(3-{[4-(2,6-dimethylpyridin-3-yl)oxypyridin-2-yl]-amino}phenyl)methyl]piperazine-1-carboxylate

tert-Butyl 4-(3-aminobenzyl)piperazine-1-carboxylate (2.5 g, 8.58 mmol), 3-(2-chloropyridin-4-yloxy)-2,6-dimethylpyridine (Method 13, 1.918 g, 8.17 mmol), Cs₂CO₃ (3.99 g, 12.26 mmol), Pd(OAc)₂ (0.128 g, 0.57 mmol) and xantphos (0.473 g, 0.82 mmol) were suspended in DMA (35 mL). The mixture was then thoroughly degassed using nitrogen and heated in a microwave for 15 mins. Then the mixture was cooled and filtered. Purification by ion exchange chromatography, using an SCX column and eluting with 0.35M NH₃/MeOH afforded the title compound; (1.63 g, 41%) as a white solid. ¹H NMR: (CDCl₃) 1.45 (9H, s), 2.37-2.40 (7H, m), 2.54 (3H, s), 3.41-3.44 (4H, m), 3.46 (2H, s), 6.20-6.23 (1H, m), 6.29 (1H, s), 6.70 (1H, s), 6.97-7.04 (2H, m), 7.18-7.27 (4H, m), 8.05 (1H, d); m/z: MH⁺ 490.80; EAA2: 0.03844.

### Example 208

### tert-Butyl N-(3-{4-[(3-{[4-(2,6-dimethylpyridin-3-yl)oxypyridin-2-yl]amino}phenyl)methyl]piperazin-1-yl}-3-oxopropyl)carbamate

4-(2,6-Dimethylpyridin-3-yloxy)-N-(3-(piperazin-1-ylmethyl)phenyl)pyridin-2-amine (Example 176, 60 mg, 0.15 mmol) was added portionwise to 3-(tert-butoxycarbonyl-amino)propanoic acid (32.1 mg, 0.17 mmol), HBTU (64.4 mg, 0.17 mmol) and DIPEA (0.053 mL, 0.31 mmol) in DMF (2 mL) at 25° C. over a period of 2 mins under nitrogen. The resulting solution was stirred at r.t. for 24 h. Then the mixture was concentrated. Purification by preparative HPLC (Waters XBridge Prep C18 OBD column, 5 μm silica, 19 mm diameter, 100 mm length), using decreasingly polar mixtures of water (containing 1% NH₃) and MeCN as eluents afforded the title compound; (52 mg, 60%) as a white solid. ¹H NMR: (CDCl₃) 1.36 (9H, s), 2.33 (3H, s), 2.36 (4H, t), 2.42 (2H, t), 2.48 (3H, s), 3.31-3.37 (4H, m), 3.40-3.42 (2H, m), 3.55 (2H, t), 5.21 (1H, s), 6.14-6.16 (1H, m), 6.22 (1H, d), 6.45 (1H, s), 6.89-6.92 (1H, m), 6.96 (1H, d), 7.14-7.17 (4H, m), 7.98 (1H, d); m/z: MH⁺ 561.66; EAA2: 0.02038.

### Example 209

### N-[3-(Chloromethyl)phenyl]-4-(2,6-dimethylpyridin-3-yl)-oxypyridin-2-amine

Methanesulfonyl chloride (0.077 mL, 1.00 mmol) was added dropwise to a cooled (5° C.) solution of {3-[4-(2,6-dimethylpyridin-3-yloxy)pyridin-2-ylamino]phenyl}-methanol (Example 174, 320 mg, 1.00 mmol) and DIPEA (0.172 mL, 1.00 mmol) in DCM (20 mL) over a period of 5 mins under nitrogen. The mixture was then stirred at r.t. for 3 days. Then the mixture was concentrated in vacuo and purified by FCC using a gradient of 10-70% EtOAc in DCM to afford the title compound; (263 mg, 78%) as a yellow solid. ¹H NMR: (CDCl₃) 2.32 (3H, s), 2.47 (3H, s), 4.47 (2H, s), 6.19-6.22 (2H, m), 6.62 (1H, s), 6.95 (1H, s), 6.98 (1H, s), 7.14-7.18 (3H, m), 7.27 (1H, s), 8.00 (1H, d); m/z: MH⁺ 340.46.

### Example 210

### Sodium 3-{[4-(2,6-dimethylpyridin-3-yl)oxypyridin-2-yl]amino}benzoate

NaOH (12.88 mL, 12.88 mmol) and methyl 3-[4-(2,6-dimethylpyridin-3-yloxy)pyridin-2-ylamino]benzoate (Example 143, 1.5 g, 4.29 mmol) were suspended in MeOH (5 mL) and the mixture was sealed into a microwave tube. The mixture was heated to 120° C. for 5 mins in a microwave. After cooling a white precipitate was filtered off, washed with cold water and then dried under vacuum to afford the title compound; (1.420 g, 93%); ¹H NMR: 2.30 (3H, s), 2.47 (3H, s), 6.13 (1H, d), 6.33-6.36 (1H, m), 7.10-7.15 (1H, m), 7.20 (1H, d), 7.39-7.43 (1H, m), 7.47 (1H, d), 7.72-7.75 (1H, m), 7.94 (1H, s), 8.04 (1H, d), 8.93 (1H, s); m/z: MH⁺ 336.49; EAA2: 0.03922.

### Example 212

### 4-{[4-(5,6-Dimethyl-2-pyridin-2-ylpyridin-3-yl)oxypyridin-2-yl]-amino}phenol

4-Aminophenol (210 mg, 1.92 mmol), 5-(2-chloropyridin-4-yl)oxy-2,3-dimethyl-6-pyridin-2-yl-pyridine (Method 16, 400 mg, 1.28 mmol), Cs₂CO₃ (1.25 g, 3.85 mmol) and xantphos (89 mg, 0.15 mmol) were suspended in 1,4-dioxane (5 mL). The mixture was purged with nitrogen and Pd₂(dba)₃ (47.0 mg, 0.05 mmol) was added. The mixture was purged with nitrogen and sealed into a microwave tube. The mixture was heated to 150° C. for 60 mins in a microwave and was then cooled to r.t. The mixture was filtered and the residue was washed with DCM and EtOAc. The combined filtrates were concentrated in vacuo and the residue was triturated with DCM. The resulting crystalline precipitate was collected by filtration, washed with DCM and dried to afford the title compound as a pale green solid (188 mg, 38%); ¹H NMR: 2.35 (3H, s), 2.53 (3H, s), 5.98 (1H, d), 6.24 (1H, dd), 6.65 (2H, d), 7.30 (2H, d), 7.33 (1H, ddd), 7.54 (1H, s), 7.78 (1H, d), 7.85 (1H, ddd), 7.89 (1H, d), 8.50 (1H, s), 8.52 (1H, dd), 8.92 (1H, s); m/z: MH⁺ 385.5.

### Example 213

### 4-{[4-(5,6-Dimethyl-2-pyridin-2-ylpyridin-3-yl)oxypyridin-2-yl]-amino}benzoic acid

KOH (62.4 mg, 1.11 mmol) was added as a solution in water (5 mL) to ethyl 4-{[4-(5,6-dimethyl-2-pyridin-2-ylpyridin-3-yl)oxypyridin-2-yl]amino}benzoate (Example 214, 196 mg, 0.44 mmol) in MeOH (5 mL). The resulting solution was stirred at 90° C. (reflux) for 20 h. After cooling to r.t., hydrochloric acid (1M, 1.11 mL, 1.11 mmol) was added. The mixture was evaporated to dryness to provide the title compound as an off-white solid; m/z: MH⁺ 413.5.

### Example 214

### Ethyl 4-{[4-(5,6-Dimethyl-2-pyridin-2-ylpyridin-3-yl)oxypyridin-2-yl]amino}benzoate

Ethyl 4-aminobenzoate (397 mg, 2.41 mmol), 5-(2-chloropyridin-4-yl)oxy-2,3-dimethyl-6-pyridin-2-yl-pyridine (Method 16, 500 mg, 1.60 mmol), Cs₂CO₃ (1.57 g, 4.81 mmol) and xantphos (111 mg, 0.19 mmol) were suspended in 1,4-dioxane (15 mL). The mixture was purged with nitrogen and Pd₂(dba)₃ (59 mg, 0.06 mmol) was added. The mixture was purged with nitrogen and sealed into a microwave tube. The mixture was heated to 150° C. for 60 mins in a microwave and was then cooled to r.t. The mixture was then filtered and the residue was washed with DCM. The combined filtrates were concentrated in vacuo and the residue was purified by FCC using a gradient of 40-70% EtOAc in isohexane to afford the title compound as a white solid; (202 mg, 28.6%); ¹H NMR: (CDCl₃) 1.38 (3H, t), 2.36 (3H, s), 2.62 (3H, s), 4.34 (2H, q), 6.28 (1H, d), 6.35 (1H, dd), 6.68 (1H, s), 7.20 (1H, ddd), 7.28 (1H, s), 7.34 (2H, d), 7.69 (1H, ddd), 7.80 (1H, dd), 7.95 (2H, d), 8.64 (1H, dd); m/z: MH⁺ 441.4.

### Example 215

### 4-{[4-(2,6-Dimethylpyridin-3-yl)oxypyridin-2-yl]amino}-N-[2-(1,1-dioxo-1,4-thiazinan-4-yl)ethyl]benzamide

NEt₃ (0.086 mL, 0.61 mmol) was added in one portion to lithium 4-[4-(2,6-dimethylpyridin-3-yloxy)pyridin-2-ylamino]benzoate (Example 205, 100 mg, 0.29 mmol), N-(2-aminoethyl)thiomorpholine-1,1-dioxide (52.1 mg, 0.29 mmol) and HBTU (144 mg, 0.38 mmol) in DMA (2 mL) at 20° C. under air. The resulting mixture was stirred at 20° C. for 2 h. Purification by preparative HPLC, using decreasingly polar mixtures of water (containing 1% NH₃) and MeCN as eluents, afforded the title compound; (96 mg, 66%) as a white solid; ¹H NMR: (400 MHz) 2.30 (3H, s), 2.49 (3H, s), 2.66 (2H, t), 2.95-2.98 (4H, m), 3.07-3.09 (4H, m), 3.34-3.38 (2H, m), 6.12 (1H, s), 6.50-6.52 (1H, m), 7.23 (1H, d), 7.50 (1H, d), 7.69-7.74 (4H, m), 8.11-8.14 (2H, m), 9.24 (1H, s); m/z: MH⁺ 496.05; EAA2: 0.03623.

### Example 216

### 4-{[4-(2,6-Dimethylpyridin-3-yl)oxypyridin-2-yl]amino}-N-(2-pyrrol-1-ylethyl)benzamide

NEt₃ (0.086 mL, 0.62 mmol) was added in one portion to lithium 4-[4-(2,6-dimethylpyridin-3-yloxy)pyridin-2-ylamino]benzoate (Example 205, 101 mg, 0.30 mmol), 2-(1H-pyrrol-1-yl)ethanamine (34.9 mg, 0.32 mmol) and HBTU (145 mg, 0.38 mmol) in DMA (2 mL) at 20° C. under air. The resulting mixture was stirred at 20° C. for 18 h. Purification by preparative HPLC, using decreasingly polar mixtures of water (containing 1% NH₃) and MeCN as eluents afforded the title compound; (11 mg, 9%) as a colourless gum; m/z: MH⁺ 428.10; EAA2: 0.1085.

### Example 217

### 4-{[4-(2,6-Dimethylpyridin-3-yl)oxypyridin-2-yl]amino}-N-(2-piperidin-1-ylethyl)benzamide

NEt₃ (0.086 mL, 0.62 mmol) was added in one portion to lithium 4-[4-(2,6-dimethylpyridin-3-yloxy)pyridin-2-ylamino]benzoate (Example 205, 101 mg, 0.30 mmol), 2-(piperidin-1-yl)ethanamine (39.4 mg, 0.31 mmol) and HBTU (145 mg, 0.38 mmol) in DMA (2 mL) at 20° C. under air. The resulting mixture was stirred at 20° C. for 18 h. Purification by preparative HPLC using decreasingly polar mixtures of water (containing 1% NH₃) and MeCN as eluents afforded the title compound; (18 mg, 14%) as a white solid; ¹H NMR: (400 MHz) 1.34-1.40 (2H, m), 1.47-1.52 (4H, m), 2.30 (3H, s), 2.34-2.43 (6H, m), 2.49 (3H, s), 3.32-3.37 (2H, m), 6.12 (1H, s), 6.50 (1H, d), 7.23 (1H, d), 7.50 (1H, d), 7.68-7.74 (4H, m), 8.08 (1H, t), 8.12 (1H, d), 9.23 (1H, s); m/z: MH⁺ 446.30; EAA2: 0.01209.

### Example 218

### 4-{[4-(2,6-Dimethylpyridin-3-yl)oxypyridin-2-yl]amino}-N-[2-(8-oxa-3-azabicyclo[3.2.1]octan-3-yl)ethyl]benzamide

NEt₃ (0.086 mL, 0.61 mmol) was added in one portion to lithium 4-[4-(2,6-dimethylpyridin-3-yloxy)pyridin-2-ylamino]benzoate (Example 205, 100 mg, 0.29 mmol), 2-(8-oxa-3-azabicyclo[3.2.1]octan-3-yl)ethanamine (47.9mg, 0.31 mmol) and HBTU (144 mg, 0.38 mmol) in DMA (2 mL) at 20° C. under air. The resulting mixture was stirred at 20° C. for 18 h. Purification by preparative HPLC eluting with decreasingly polar mixtures of water (containing 1% NH₃) and MeCN afforded the title compound; (61 mg, 44%) as a white solid; ¹H NMR: (400MHz) 1.64-1.67 (2H, m), 1.80-1.84 (2H, m), 2.20 (2H, d), 2.30 (3H, s), 2.41 (2H, t), 2.49 (3H, s), 2.61 (2H, d), 3.27-3.35 (2H, m), 4.19 (2H, s), 6.12 (1H, s), 6.51 (1H, d), 7.23 (1H, d), 7.50 (1H, d), 7.71 (4H, s), 8.02 (1H, t), 8.12 (1H, d), 9.23 (1H, s); m/z: MH⁺ 474.17; EAA2: 0.04951.

### Example 219

### N-[2-(4,4-Difluoropiperidin-1-yl)ethyl]-4-{[4-(2,6-dimethyl-pyridin-3-yl)oxypyridin-2-yl]amino}benzamide

NEt₃ (0.086 mL, 0.61 mmol) was added in one portion to lithium 4-[4-(2,6-dimethylpyridin-3-yloxy)pyridin-2-ylamino)benzoate (Example 205, 100 mg, 0.29 mmol), 2-(4,4-difluoropiperidin-1-yl)ethanamine (50.4 mg, 0.31 mmol) and HBTU (144 mg, 0.38 mmol) in DMA (2 mL) at 20° C. under air. The resulting mixture was stirred at 20° C. for 18 h. Purification by preparative HPLC eluting with decreasingly polar mixtures of water (containing 1% NH₃) and MeCN afforded the title compound; (24 mg, 17%) as a colourless gum; ¹H NMR: (400 MHz) 1.10 (2H, t), 1.90-1.99 (2H, m), 2.30 (3H, s), 2.49 (3H, s), 2.53-2.57 (6H, m), 3.34-3.39 (2H, m), 6.09-6.12 (1H, m), 6.48-6.52 (1H, m), 7.22-7.25 (2H, m), 7.50 (1H, d), 7.67-7.74 (3H, m), 8.09-8.13 (2H, m), 9.23 (1H, s); m/z: MH⁺ 482.10; EAA2: 0.1081.

### Example 220

### 4-{[4-(2,6-Dimethylpyridin-3-yl)oxypyridin-2-yl]amino}-N-(2-morpholin-4-ylethyl)benzamide

NEt₃ (0.086 mL, 0.61 mmol) was added in one portion to lithium 4-[4-(2,6-dimethylpyridin-3-yloxy)pyridin-2-ylamino)benzoate (Example 205, 100 mg, 0.29 mmol), 2-morpholinoethylamine (40 mg, 0.31 mmol) and HBTU (144 mg, 0.38 mmol) in DMA (2 mL) at 20° C. under air. The resulting mixture was stirred at 20° C. for 18 h. The crude product was purified by preparative HPLC using decreasingly polar mixtures of water (containing 1% NH₃) and MeCN as eluents. Fractions containing the desired compound were evaporated to dryness to afford the product (90 mg, 69%) as a colourless gum; ¹H NMR: (400 MHz) 2.30 (3H, s), 2.40-2.47 (6H, m), 2.49 (3H, s), 3.34-3.39 (2H, m), 3.56-3.59 (4H, m), 6.12 (1H, s), 6.49-6.52 (1H, m), 7.23 (1H, d), 7.50 (1H, d), 7.68-7.78 (4H, m), 8.10-8.13 (2H, m), 9.23 (1H, s); m/z: MH⁺ 448.14; EAA2: 0.06751.

### Example 221

### 2-(3-{2-[(3,4,5-Trimethoxyphenyl)amino]pyridin-4-yl}oxypyridin-2-yl)benzamide

Pd(PPh₃)₄ (5 mg) was added to a mixture of 4-(2-chloropyridin-3-yl)oxy-N-(3,4,5-trimethoxyphenyl)pyridin-2-amine (Example 53), 39 mg, 0.1 mmol), (2-cyanophenyl)boronic acid (30 mg, 0.2 mmol) and Na₂CO₃ (32 mg, 0.3 mmol) in 1,4-dioxane (1 mL) and water (0.1 mL) in a microwave tube. The tube was flushed with nitrogen and the mixture was heated in a microwave at 100° C. for 10 mins, then 120° C. for 10 mins, then additional water was added (0.1 mL) and the reaction mixture was heated at 120° C. for 1 h. The mixture was dissolved in MeOH and the organic phase was decanted, filtered and concentrated in vacuo. Purification by basic HPLC provided the title compound; (30.5 mg, 65%) as a white solid; ¹H NMR: (CDCl₃) 3.78 (s, 6H), 3.79 (s, 3H), 6.25-6.30 (m, 2H), 6.56 (s, 2H), 6.68 (s, 1H), 7.29-7.49 (m, 5H), 7.63 (dd, 1H), 7.96 (d, 1H), 8.51 (dd, 1H); m/z: MH⁺ 473.36; EAA2: 3.022.

### Method 1

### 3-(2-Chloropyridin-4-yl)oxy-2-(6-methylpyridin-2-yl)pyridine

NaH (60% dispersion in mineral oil) (0.091 g) and 2,4-dichloropyridine (0.2 g) were added to a solution of 2-(6-methylpyridin-2-yl)pyridin-3-ol (Method 2, 0.25 g) in DMF (2 mL) the resulting mixture was heated to 125° C. for 2 h. The reaction was quenched with water. The resulting mixture was partitioned between EtOAc and water and the phases were separated. The aqueous portion was extracted with EtOAc. The combined organic portions were washed with brine, dried (MgSO₄) and concentrated in vacuo. Purification by FCC using a gradient of 0-4% MeOH in DCM afforded the title compound (0.12 g, 31%) as a beige solid after trituration with a mixture of EtOAc, iso-hexane and diethyl ether; ¹H NMR: 2.18 (3H, s), 6.84-6.87 (1H, m), 6.93 (1H, d), 7.19-7.22 (1H, m), 7.61-7.66 (1H, m), 7.73-7.80 (2H, m), 7.89 (1H, d), 8.22 (1H, d), 8.68-8.71 (1H, m); m/z: MH⁺ 298.43.

### Method 2

### 2-(6-Methylpyridin-2-yl)pyridin-3-ol

2-Furyl-(6-methylpyridin-2-yl)methanone (Method 3, 1.4 g) and NH₄OH (35% solution, 20 mL) were combined and heated to 150° C. for 6 h in a microwave. The mixture was then concentrated in vacuo and purified by FCC eluting with a gradient of 0-20% MeOH in DCM to afford the title compound as a yellow solid (1.17 g, 78%); ¹H NMR: 2.60 (3H, s), 7.36-7.37 (2H, m), 7.42 (1H, d), 7.97-8.02 (1H, m), 8.20-8.21 (1H, m), 8.34 (1H, d), 14.44 (1H, s); m/z: MH⁺ 187.5.

### Method 3

### 2-Furyl-(6-methylpyridin-2-yl)methanone

MnO₂ (3 g) was added to a solution of 2-furyl-(6-methylpyridin-2-yl)methanol (Method 4, 2 g) in DCM (50 mL). The resulting mixture was stirred for 16 h at r.t. The mixture was diluted by DCM and filtered through diatomaceous earth. The filtrate was concentrated in vacuo. Purification by FCC using a gradient of 0-40% EtOAc in iso-hexane afforded the title compound as a yellow solid (1.57 g, 79%); ¹H NMR: 2.67 (3H, s), 6.85 (1H, dd), 7.60 (1H, d), 7.91 (1H, d), 7.96-8.01 (1H, m), 8.11-8.12 (1H, dd), 8.17-8.18 (1H, m); m/z: MH⁺ 188.49.

### Method 4

### 2-Furyl-(6-methylpyridin-2-yl)methanol

n-Butyllithium (2.5M solution in hexanes, 21 mL) was added dropwise to a solution of 2-bromo-6-methyl-pyridine (5 mL) in THF (80 mL) at −78° C. The mixture was stirred at −78° C. for 10 minutes, then furan-2-carbaldehyde (4.37 mL) was added. The mixture was stirred at −78° C. for 30 minutes, then at r.t. for 4 h. The reaction was quenched with saturated aqueous NH₄Cl. The resulting mixture was partitioned between EtOAc and water, and the phases separated. The aqueous portion was extracted with EtOAc. The combined organic portions were washed with brine, dried (MgSO₄) and concentrated in vacuo. Purification by FCC using a gradient of 0-100% EtOAc in iso-hexane afforded the title compound as a yellow solid (4.39 g, 44%); ¹H NMR: 2.41 (3H, s), 5.61 (1H, d), 6.03 (1H, d), 6.14 (1H, d), 6.34-6.36 (1H, m), 7.13 (1H, d), 7.36 (1H, d), 7.52-7.53 (1H, m), 7.66-7.71 (1H, m); m/z: MH⁺ 190.49.

### Method 5

### 5-(2-Chloropyridin-4-yl)oxy-2,3-dimethyl-6-(6-methylpyridin-2-yl)pyridine

NaH (60% dispersion in mineral oil) (91 mg) and 2,4-dichloropyridine (0.2 g) was added to a solution of 5,6-dimethyl-2-(6-methylpyridin-2-yl)pyridin-3-ol (prepared according to WO2005080377, 0.21 g) in DMF (2 mL) the resulting mixture was heated to 130° C. for 2 h. The reaction was then quenched with water. The resulting mixture was partitioned between EtOAc and water and the phases were separated. The aqueous portion was extracted with EtOAc. The combined organic portions were washed with brine, dried (MgSO₄) and concentrated in vacuo. Purification by FCC using a gradient of 0-4% MeOH in DCM afforded the title compound (0.1 g, 23%) as a beige solid after trituration with a mixture of EtOAc, iso-hexane and diethyl ether; ¹H NMR: 2.14 (3H, s), 2.35 (3H, s), 2.53 (3H, s), 6.83 (1H, d), 6.88 (1H, d), 7.13-7.17 (1H, m), 7.61 (1H, s), 7.72 (1H, d), 7.73 (1H, s), 8.18 (1H, d); m/z: MH⁺ 326.48.

### Method 6

### 3-(2-Chloropyridin-4-yl)oxy-6-ethyl-2-(6-methylpyridin-2-yl)pyridine

NaH (60% dispersion in mineral oil) (91 mg) and 2,4-dichloropyridine (0.2 g) were added to a solution of 6-ethyl-2-(6-methylpyridin-2-yl)pyridin-3-ol (Method 7, 0.214 g) in DMF (2 mL) and the resulting mixture was heated to 130° C. for 1 h. The mixture was then allowed to cool to r.t. and was concentrated in vacuo. Purification by FCC eluting with 0-4% MeOH in DCM afforded the title compound as an oil; (0.10 g, 23%); ¹H NMR: 1.32 (3H, t), 2.18 (3H, s), 2.89 (2H, q), 6.84 (1H, dd), 6.90 (1H, d), 7.18-7.22 (1H, m), 7.49 (1H, d), 7.73-7.79 (3H, m), 8.21 (1H, d); m/z: MH⁺ 326.45.

### Method 7

### 6-Ethyl-2-(6-methylpyridin-2-yl)pyridin-3-ol

(5-Ethyl-2-furyl)-(6-methylpyridin-2-yl)methanone (Method 8, 0.8 g) and NH₄OH (35% solution, 10 mL) were combined and heated to 140° C. for 4 h in a microwave. The mixture was then concentrated in vacuo and purified by FCC eluting with a gradient of 0-10% MeOH in DCM to afford the title compound as a yellow solid; (0.523 g, 66%); ¹H NMR: 1.27 (3H, t), 2.59 (3H, s), 2.75 (2H, q), 7.23 (1H, d), 7.28 (1H, d), 7.39 (1H, d), 7.95-8.00 (1H, m), 8.36 (1H, d), 14.13 (1H, s); m/z: MH⁺ 215.5.

### Method 8

### (5-Ethyl-2-furyl)-(6-methylpyridin-2-yl)methanone

MnO₂ (3.86 g) was added to a solution of (5-ethyl-2-furyl)-(6-methylpyridin-2-yl)-methanol (Method 9, 1.55 g) in DCM (50 mL). The resulting mixture was stirred for 32 h at r.t. The mixture was then diluted by DCM and filtered through diatomaceous earth. The filtrate was concentrated in vacuo to afford the title compound as a yellow oil; (1.53 g, 99%); ¹H NMR: 1.25 (3H, t), 2.60 (3H, s), 2.78 (2H, q), 6.47 (1H, d), 7.52 (1H, d), 7.82 (1H, d), 7.89-7.94 (1H, m), 7.99 (1H, d); m/z: MH⁺ 216.53.

### Method 9

### (5-Ethyl-2-furyl)-(6-methylpyridin-2-yl)methanol

n-Butyllithium (2.5M solution in hexanes, 5.8 mL), was added dropwise to a solution of 2-bromo-6-methyl-pyridine (2.1 g) in THF (30 mL) at −78° C., under nitrogen. The mixture was stirred at −78° C. for 10 minutes then 5-ethylfuran-2-carbaldehyde (1.78 mL) was added. The mixture was stirred at −78° C. for 0.5 h, then at r.t. for 0.5 h. The reaction was quenched with saturated aqueous NH₄Cl. The resulting mixture was partitioned between EtOAc and water and the phases were separated. The aqueous portion was extracted with EtOAc. The combined organic portions were washed with brine, dried (MgSO₄) and concentrated in vacuo. Purification by FCC using a gradient of 0-70% EtOAc in iso-hexane afforded the title compound as a yellow oil; (1.57 g, 60%); ¹H NMR: 1.11 (3H, t), 2.42 (3H, s), 2.54 (2H, q), 5.53 (1H, d), 5.93 (1H, d), 5.95-5.96 (2H, m), 7.13 (1H, d), 7.35 (1H, d), 7.66-7.71 (1H, m); m/z: MH⁺ 218.51.

### Method 10

### 3-(2-Chloropyridin-4-yl)oxy-6-methyl-2-(1-methylpyrazol-4-yl)-pyridine

NaH (60% dispersion in mineral oil, 80 mg) and 2,4-dichloropyridine (0.15 g) were added to a solution of 6-methyl-2-(1-methylpyrazol-4-yl)pyridin-3-ol (Method 11, 0.16 g), in DMF (3 mL). The mixture was heated to 100° C. for 1 h. The reaction was quenched with water. The resulting mixture was partitioned between EtOAc and water and the phases were separated. The aqueous portion was extracted with EtOAc. The combined organic portions were washed with brine, dried (MgSO₄) and concentrated in vacuo. Purification by FCC using a gradient of 0-70% EtOAc in iso-hexane afforded the title compound as a solid; (0.214 g, 88%); ¹H NMR: 2.53 (3H, s), 3.84 (3H, s), 6.96 (1H, dd), 7.11 (1H, d), 7.20 (1H, d), 7.60 (1H, d), 7.87 (1H, s), 8.15 (1H, s), 8.28 (1H, d); m/z: MH⁺ 301.46.

### Method 11

### 6-Methyl-2-(1-methylpyrazol-4-yl)pyridin-3-ol

2-Iodo-6-methyl-pyridin-3-ol (0.79 g), 1-methyl-4-(4,4,5,5-tetramethyl-1,3,2-dioxaborolan-2-yl)pyrazole (0.7 g), Cs₂CO₃ (2.74 g) and Pd(PPh₃)₄ (0.195 g) were combined with 1,4-dioxane (8 mL) and water (2 mL) and the mixture was heated to 100° C. for 0.75 h. The mixture was then concentrated in vacuo and the resulting aqueous residue was taken to pH 7 with 10% aqueous HCl. The resulting mixture was partitioned between water and EtOAc and the phases were separated. The aqueous portion was extracted with EtOAc and the combined organic portions were dried (MgSO₄) and concentrated in vacuo. Purification by FCC using a gradient of 0-15% MeOH in DCM afforded the title compound as a yellow solid; (0.313 g, 49%); ¹H NMR: 2.37 (3H, s), 3.88 (3H, s), 6.87 (1H, d), 7.12 (1H, d), 7.99 (1H, s), 8.20 (1H, s), 9.87 (1H, s); m/z: MH⁺ 190.52.

### Method 12

### 2-Chloro-4-pyridin-3-yloxy-pyridine

A mixture of CuT (0.1 14 g), 2-chloro-4-iodo pyridine (0.718 g), 3-hydroxy pyridine (0.289 g), Cs₂CO₃ and DMF (12 mL) was heated in a microwave to 150° C. for 40 mins. After cooling the mixture was diluted with water and EtOAc. The phases were separated, and the aqueous phase was extracted with EtOAc. The combined organic portions were dried (MgSO₄) and concentrated in vacuo. Purification by FCC eluting with 0-100% EtOAc in iso-hexane afforded the title compound as a solid; (0.478 g, 77%); ¹H NMR: 7.01 (1H, dd), 7.11 (1H, d), 7.55 (1H, dd), 7.74 (1H, d), 8.33 (1H, d), 8.55 (2H, d); m/z: MH⁺ 207.32.

### Method 13

### 3-(2-Chloropyridin-4-yl)oxy-2,6-dimethyl-pyridine

To a suspension of NaH (60% dispersion in mineral oil, 0.136 g) in DMF (5 mL) was added sequentially a solution of 2,6-dimethylpyridin-3-ol (0.209 g), in DMF (2 mL) and 2,4-dichloropyridine (0.3 g) in DMF (3 mL). After stirring for 20 minutes at r.t., the mixture was heated to 100° C. for 2 h. The reaction was then quenched with water. The resulting mixture was partitioned between EtOAc and water. The layers were separated, and the aqueous phase was extracted with EtOAc. The combined organic portions were washed with brine, dried (MgSO₄) and concentrated in vacuo. Purification by FCC using a gradient of 0-70% EtOAc in iso-hexane afforded the title compound as a yellow solid; (0.212 g, 53%); ¹H NMR: 2.28 (3H, s), 2.48 (3H, s), 6.90 (1H, dd), 6.99 (1H, d), 7.22 (1H, d), 7.52 (1H, d), 8.29 (1H, d); m/z: MH⁺ 235.65.

### Method 14

### 2-Chloro-4-(6-methylpyridin-3-yl)oxy-pyridine

This compound was prepared according to the method described for 3-(2-chloro-pyridin-4-yl)oxy-2,6-dimethyl-pyridine (Method 13) using 2,4-dichloropyridine and 6-methylpyridin-3-ol, to afford the title compound as a colourless oil; (0.441 g, 67%); ¹H NMR: (300 MHz, CDCl₃) 2.61 (3H, s), 6.79 (1H, dd), 6.82 (1H, d), 7.25 (1H, d), 7.34 (1H, dd), 8.26 (1H, d), 8.35 (1H, d); m/z: MH⁺ 221.36.

### Method 15

### 2-Chloro-4-(2-methylpyridin-3-yl)oxy-pyridine

This compound was prepared according to the method described for 3-(2-chloropyridin-4-yl)oxy-2,6-dimethyl-pyridine (Method 13) using 2,4-dichloropyridine and 2-methylpyridin-3-ol, to afford the title compound as a cream solid; (0.54 g, 82%); ¹H NMR: 2.34 (3H, s), 6.92 (1H, dd), 7.03 (1H, d), 7.36-7.40 (1H, m), 7.64 (1H, dd), 8.31 (1H, d), 8.44 (1H, dd); m/z: MH⁺ 221.36.

### Method 16

### 5-(2-Chloropyridin-4-yl)oxy-2,3-dimethyl-6-pyridin-2-yl-pyridine

NaH (69 mg, 1.72 mmol, 60% dispersion in mineral oil) and 2,4-dichloropyridine were sequentially added to a solution of 5,6-dimethyl-2-pyridin-2-yl-pyridin-3-ol (prepared according to WO2005080377, 173 mg, 0.86 mmol) in DMF (2 mL), and the resulting mixture was heated to reflux for 40 minutes and was then allowed to cool to r.t. The mixture was quenched with water, taken up in EtOAc and the phases separated. The aqueous phase was extracted three times with EtOAc. The combined organic portions were washed with water then with brine, dried (MgSO₄) and concentrated in vacuo. The resulting residue was part-purified by FCC using a gradient of 0-4% MeOH in DCM, then further purified by FCC eluting with 3% MeOH in DCM to obtain impure title compound as a white solid; (136 mg, 51% if pure). This material was used in subsequent steps without further purification; ¹H NMR: (300 MHz, CDCl₃) 2.38 (3H, s), 2.63 (3H, s), 6.68-6.71 (1H, m), 6.73 (1H, d), 7.17-7.22 (1H, m), 7.28 (1H, s), 7.67-7.73 (1H, m), 7.82-7.86 (1H, m), 8.13 (1H, d), 8.55-8.58 (1H, m); m/z: MH⁺ 312.44.

### Method 17

### 3-(2-Chloropyridin-4-yl)oxy-6-methyl-2-pyridin-2-yl-pyridine

A mixture of 3-(2-chloropyridin-4-yl)oxy-2-iodo-6-methylpyridine (Method 18, 0.3 g), 2-pyridylzinc bromide (0.232 g) and Pd(PPh₃)₄ (0.1 g), THF (2 mL) and DMA (1 mL) was heated at 150° C. for 3 h in a microwave in a sealed tube. After cooling, the mixture was diluted with EtOAc (20 mL) and washed with saturated brine (15 mL). The phases were separated and the organic layer was dried (MgSO₄) and concentrated in vacuo. Purification by FCC using a gradient of 10-50% EtOAc in iso-hexane afforded the title compound (0.145 g, 56%) as a beige solid; m/z: MH⁺ 298.15.

### Method 18

### 3-(2-Chloropyridin-4-yl)oxy-2-iodo-6-methyl-pyridine

NaH (0.286 g) was added to a solution of 2-iodo-6-methylpyridin-3-ol (2.80 g), in DMF (20 mL), at r.t. over a period of 5 minutes. The resulting suspension was stirred for 10 minutes at r.t. 2,4-Dichloropyridine (1.47 g) was then added portionwise and the mixture was heated to 140° C. over a period of 20 minutes. The resulting solution was stirred at 140° C. for 2 h. The mixture was then concentrated in vacuo. The residue was redissolved in DCM (150 mL) and washed with water (50 mL). The organic portion was dried (MgSO₄) and concentrated in vacuo. Purification by FCC using a gradient of 0-5% EtOAc in DCM afforded the title compound (3.43 g, 100%) as a beige solid; ¹H NMR: 2.61 (3H, s), 6.74-6.79 (2H, m), 7.18 (1H, d), 7.24 (1H, d), 8.28 (1H, d); m/z: MH⁺ 347.13.

### Method 19

### 3-(2-Chloropyridin-4-yl)oxy-6-ethyl-2-pyridin-2-yl-pyridine

NaH (0.140 g) was added portionwise to 6-ethyl-2-pyridin-2-yl-pyridin-3-ol (see Method 20, 0.280 g) in DMF (5 mL) at 20° C. over a period of 5 minutes and the resulting suspension was stirred at r.t. for 0.5 h. 2-Chloro-4-fluoropyridine (0.239 g) was added to the mixture and the resulting solution was stirred at r.t. for 1 h. The mixture was heated to 120° C. for 20 minutes in a microwave. After cooling the crude material was semi-purified by ion exchange chromatography, using an SCX column eluting with 7M NH₃/MeOH and appropriate fractions were evaporated to dryness to afford part-purified material. Further purification by FCC using a gradient of 30-60% EtOAc in iso-hexane afforded the title compound; (0.213 g, 48%); ¹H NMR: (CDCl₃) 1.40 (3H, t), 2.98 (2H, q), 6.72-6.68 (1H, m), 6.75 (1H, d), 7.25-7.19 (1H, m), 7.32 (1H, d), 7.46 (1H, d), 7.77-7.69 (1H, m), 7.90-7.86 (1H, m), 8.14 (1H, d), 8.58-8.53 (1H, m); m/z: MH⁺ 312.

### Method 20

### 6-Ethyl-2-pyridin-2-yl-pyridin-3-ol

Water (3 mL), (5-ethyl-2-furyl)-pyridin-2-yl-methanone (see Method 21, 1.05 g) and 0.880 ammonia (3 mL) were heated together in a sealed microwave tube at 150° C. for 3 h. After cooling, the mixture was then concentrated in vacuo and purified by FCC using a gradient of 0-6% EtOAc in iso-hexane to afford the title compound (0.45 g, 43%); m/z: MH⁺ 201.

### Method 21

### (5-Ethyl-2-furyl)-pyridin-2-yl-methanone

MnO₂ (0.898 g) was added in one portion to (5-ethyl-2-furyl)-pyridin-2-yl-methanol (see Method 22, 1.05 g) in DCM at 20° C. After 1 h the reaction mixture was filtered through diatomaceous earth. Further MnO₂ (0.898 g) was added in one portion and the suspension was stirred at 20° C. for a further 18 h. The reaction mixture was filtered through diatomaceous earth and concentrated in vacuo to afford the title compound; (1.00 g, 100%); m/z: MH⁺ 202.

### Method 22

### (5-Ethyl-2-furyl)-pyridin-2-yl-methanol

n-Butyllithium (9.67 mL, 2.5M in hexanes, 24.2 mmol) was added dropwise over a period of 10 minutes to a solution of 2-bromopyridine (3.18 g) in THF (20 mL) at −78° C. The resulting solution was stirred at −78° C. for 10 minutes. 5-Ethylfuran-2-carbaldehyde (2.00 g) was added in one portion to the mixture and the resulting solution was stirred at −78 ° C. for 1 h, then allowed to warm to r.t. overnight. The reaction was quenched using sat. aq. NH₄Cl. The resulting mixture was partitioned between EtOAc and water and the phases were separated. The aqueous portion was extracted three times with EtOAc, and the combined organic portions were washed with brine, dried (MgSO₄) and concentrated in vacuo. Purification by FCC using a gradient of 0-100% EtOAc in iso-hexane afforded the title compound (1.050 g, 25%); ¹H NMR: (CDCl₃) 1.17 (3H, t), 2.60 (2H, q), 4.99 (1H, s), 5.75 (1H, s), 5.89-5.92 (1H, m), 6.10 (1H, d), 7.21-7.25 (1H, m), 7.28 (1H, d), 7.65-7.72 (1H, m), 8.56-8.61 (1H, m); m/z: MH⁻ 204.

### Method 23

### 3-(2-Chloropyridin-4-yl)oxy-6-methyl-2-phenyl-pyridine

A mixture of phenylboronic acid (0.106 g), 3-(2-chloropyridin-4-yl)oxy-2-iodo-6-methylpyridine (Method 18, 0.25 g), Cs₂CO₃ (0.144 g), Pd(PPh₃)₄ (0.042 g), dioxane (2 mL) and water (0.5 mL) was heated to 100° C. for 15 minutes by microwave in a sealed tube. After cooling, the mixture was diluted with EtOAc (20 mL) and washed with saturated brine (15 mL). The organic portion was dried (MgSO₄) and concentrated in vacuo. Purification by FCC using a gradient of 0-30% EtOAc in DCM afforded the title compound (0.165 g, 77%); ¹H NMR: 2.60 (3H, s), 6.59-6.61 (1H, m), 6.65 (1H, s), 7.13 (1H, d), 7.24-7.32 (4H, m), 7.66-7.71 (2H, m), 8.08 (1H, d); m/z: MH⁺ 297.4 and 299.2.

### Method 24

### 3-(2-Chloropyridin-4-yl)oxy-6-methyl-2-(4-methylthiophen-3-yl)-pyridine

A mixture of (4-methylthiophen-3-yl)boronic acid (0.123 g), 3-(2-chloropyridin-4-yl)oxy-2-iodo-6-methyl-pyridine (Method 18, 0.25 g), Cs₂CO₃ (590 mg), Pd(PPh₃)₄ (25 mg), dioxane (10 mL) and water (2 mL) was heated to 100° C. for 20 minutes by microwave in a sealed tube. After cooling, the mixture was diluted with EtOAc (30 mL) and washed with saturated brine (15 mL). The organic portion was dried (MgSO₄) and concentrated in vacuo. Purification by FCC using a gradient of 0-30% EtOAc in DCM afforded the title compound; (0.202 g, 88%); ¹H NMR: 2.19 (3H, s), 2.57 (3H, s), 6.54-6.58 (2H, m), 6.84-6.86 (1H, m), 7.13 (1H, d), 7.26 (1H, d), 7.32 (1H, d), 8.06 (1H, d); m/z: MH⁺ 317.43.

### Method 25

### 2-{3-[(2-Chloropyridin-4-yl)oxy]-6-methylpyridin-2-yl}pyrazine

A mixture of 3-(2-chloropyridin-4-yl)oxy-2-iodo-6-methylpyridine (Method 18, 0.3 g), 2-tributylstannylpyrazine (0.38 g), 1,1′-bis(diphenylphosphino)ferrocene (58 mg), Pd₂(dba)₃ (32 mg), THF (2 mL) and DMA (1 mL) was heated to 140° C. for 30 minutes by microwave in a sealed tube. After cooling, the mixture was filtered. The filtrate was diluted with EtOAc (20 mL) and washed with saturated brine (15 mL). The organic portion was dried (MgSO₄) and concentrated in vacuo. Purification by FCC using a gradient of 10-50% EtOAc in iso-hexane afforded the title compound (95 mg, 36%) as a beige solid; m/z: MH⁺ 299.13.

### Method 26

### 2-Chloro-4-(2-chloropyridin-3-yl)oxy-pyridine

A suspension of 2-chloro-4-iodopyridine (2.88 g), 2-chloro-3-hydroxypyridine (1.30 g), Cs₂CO₃ (6.51 g) and copper iodide (0.38 g) in DMA (24 mL) was heated in a microwave at 120° C. for 1 h. The mixture was diluted with EtOAc and filtered through diatomaceous earth. The diatomaceous earth was washed with additional EtOAc and the combined organic portions were concentrated in vacuo. Purification by FCC using 20% EtOAc in hexane afforded the title compound (604 mg, 25% if pure) as a foam; ¹H NMR: 6.76 (1H, dd), 6.79-6.81 (1H, m), 7.38 (1H, dd), 7.54 (1H, dd), 8.30 (1H, d), 8.39 (1H, dd); m/z: MH⁺ 241.36.

### Method 27

### 2-Chloro-3-(2-chloropyridin-4-yl)oxy-6-methyl-pyridine

A suspension of 2-chloro-3-hydroxy-6-methylpyridine (1.20 g), 2-chloro-4-iodopyridine (2.35 g), copper iodide (0.317 g) and Cs₂CO₃ in DMF (20 mL) was heated in a microwave at 130° C. for 1 h. After cooling, the mixture was diluted with EtOAc and filtered through diatomaceous earth. The diatomaceous earth was washed with additional EtOAc and the combined organic portions were washed with water and then saturated brine. The solution was dried (Na₂SO₄) concentrated in vacuo. Purification by FCC using 20% EtOAc in hexane afforded the title compound (0.882 g, 42%); ¹H NMR: 2.60 (3H, s), 6.74-6.78 (2H, m), 7.20 (1H, dd), 7.41-7.44 (1H, m), 8.27 (1H, d).

### Method 30

### Methyl 3-[(2-chloropyridin-4-yl)oxy]-6-methylpyridine-2-carboxylate

K₂CO₃ (4.56 g, 33 mmol), 2-chloro-4-fluoropyridine (2.17 g) and methyl 3-hydroxy-6-methylpyridine-2-carboxylate (Method 31, 2.76 g) were suspended in DMF (2 mL) and the mixture was heated to 120° C. for 1.5 h and then cooled. The mixture was then diluted with DCM (100 mL) and washed with water (50 mL). The organic layer was dried (MgSO₄), filtered and concentrated in vacuo. Purification by FCC using a gradient of 5-50% EtOAc in isohexane afforded the title compound (0.982 g, 21.36%); m/z: 279 MH⁺.

### Method 31

### Methyl 3-hydroxy-6-methylpyridine-2-carboxylate

2-Iodo-6-methylpyridin-3-ol (0.402 g), bis(triphenylphosphine)palladium(II) chloride (1.2 g) and NEt₃ (0.262 mL) in MeOH (50 mL) were stirred under an atmosphere of carbon monoxide at 10 bar and 100° C. for 3 h. The mixture was then filtered and concentrated in vacuo to afford the title compound (0.286, 100%); m/z: 168 MH⁺.

### Method 34

### 4-Amino-N-(1-methylpiperidin-4-yl)benzamide

To a solution of 4-aminobenzoic acid (4.0 g) in DMF (100 mL) was added 4-amino-1-methylpiperidine (3.55 g). To the resultant paste was added DIPEA(15 mL). The mixture was then treated portionwise (4-5 portions) with HATU (13 g) [Caution! EXOTHERM:- Portions were added at a rate that ensured an internal reaction temperature of <30° C.]. The solution was then stirred at r.t. for 20 h. The solvents were removed, in vacuo, and the residue was partitioned between saturated aqueous NaHCO₃ solution (250 mL) and DCM (250 mL). The organic layer was separated and the aqueous portion was re-extracted with further DCM (250 mL). The aqueous portions were then filtered to remove insoluble material and the pH was adjusted to >12 by addition of 1M NaOH (aq). The basic mixture was then extracted with EtOAc (2×300 mL), the organic extracts were washed with brine, dried (MgSO₄), filtered and evaporated to afford the title compound (2.35 g, 35%) as an opaque yellow syrup, which solidified on standing; ¹H NMR: 1.59 (m, 2H), 1.77 (m, 2H), 2.17 (m, 2H), 2.29 (s, 3H), 2.87 (m, 2H), 3.74 (m, 1H), 5.54 (s, 2H), 6.53 (m, 2H), 7.57 (m, 2H), 7.72 (m, 1H); m/z: MH⁺ 234.5.

### Method 38

### 3-(2-Chloropyridin-4-yloxy)-2-(4-fluorophenyl)-6-methylpyridine

NaH (160 mg, 4.00 mmol) was added portionwise to 2-(4-fluorophenyl)-6-methylpyridin-3-ol (Method 56, 325 mg, 1.60 mmol) in DMF (5 mL) at 20° C. over a period of 5 mins under nitrogen. The resulting suspension was stirred at r.t. for 30 mins. 2-Chloro-4-fluoropyridine (210 mg, 1.60 mmol) was added to the mixture and the resulting solution was stirred at r.t. for 4 h. The mixture was quenched with saturated NH₄Cl (2 mL), diluted with water. The resulting precipitate was collected by filtration, washed with water (25 mL) and dried under vacuum to afford the title product; (349 mg, 69.3%); as a beige gum which was used without further purification. m/z: MH⁺ 315.57.

### Method 39

### 3-(2-Chloropyridin-4-yloxy)-2-(furan-2-yl)-6-methylpyridine

3-(2-Chloropyridin-4-yloxy)-2-iodo-6-methylpyridine (Method 18, 750 mg, 2.16 mmol), tributyl(furan-2-yl)stannane (850 mg, 2.38 mmol) and Pd(PPh₃)₄ (125 mg, 0.1 1 mmol) were dissolved in THF (10 mL) and sealed into a microwave tube. The mixture was heated to 140° C. for 30 mins in a microwave and was then cooled to r.t. Then the mixture was poured onto a pre-equilibrated (MeOH) 50 g SCX-2 cartridge and flushed thoroughly with MeOH followed by 2% NH₄OH in MeOH. Concentration of appropriate fractions afforded the title product; (478 mg, 77%) as a brown solid. m/z MH⁺ 287.44

### Method 40

### 4-Fluoro-N-(3,4,5-trimethoxyphenyl)pyridin-2-amine

2-Chloro-4-fluoropyridine (1.00 g, 7.60 mmol), Cs₂CO₃ (3.72 g, 11.40 mmol), Pd(OAc)₂ (0.119 g, 0.53 mmol) and xantphos (0.440 g, 0.76 mmol) were suspended in DMA (2 mL) and sealed into a microwave tube. The mixture was heated to 130° C. for 15 mins in a microwave and was then cooled to r.t. The crude product was purified by ion exchange chromatography, using an SCX column. The desired product was eluted from the column using 7M NH₃/MeOH and fractions were evaporated to dryness to afford a white solid. Further purification by FCC using a gradient of 20-60% EtOAc in isohexane afforded the title compound (1.120 g, 52.9%) as a white solid; ¹H NMR: 3.85 (6H, s), 6.44-6.51 (2H, m), 6.55 (2H, s), 6.60 (1H, s), 8.09-8.17 (1H, m); m/z: MH⁺ 279.

### Method 41

### 6-Methyl-2-(pyrrolidin-1-yl)pyridin-3-ol

3-(Benzyloxy)-6-methyl-2-(pyrrolidin-1-yl)pyridine (Method 42, 75 mg, 0.28 mmol) and palladium (14.87 mg, 0.01 mmol) in EtOH was stirred at r.t. under an atmosphere of hydrogen at 1 atm for 90 mins. Then the mixture was filtered and concentrated afford the title product as a grey solid (48 mg, 96%). ¹H NMR: 1.86 (4H, m), 2.31 (3H, s), 3.16-3.60 (4H, m), 6.29-6.51 (1H, m), 6.84-6.94 (1H, m); m/z: MH⁺ 179.41.

### Method 42

### 3-(Benzyloxy)-6-methyl-2-(pyrrolidin-1-yl)pyridine

Methyllithium (0.658 mL of a 1.6M solution in hexanes, 1.05 mmol) was added dropwise to a cooled (0° C.) solution of 3-(benzyloxy)-6-iodo-2-(pyrrolidin-1-yl)pyridine (200 mg, 0.53 mmol) in THF (5 mL) over a period of 5 mins. The resulting solution was stirred at r.t. for 90 mins. Then the reaction was quenched with saturated NH₄Cl (5 mL) and the mixture was partitioned between EtOAc (15 mL) and water (25 mL). The organic layer was separated and was washed with water (15 mL). The organics were concentrated in vacuo. Purification by preparative HPLC (Waters XBridge Prep C18 OBD column, 5 μm silica, 19 mm diameter, 100 mm length), using decreasingly polar mixtures of water (containing 1% NH₃) and MeCN as eluents afforded the title compound; (93 mg, 66%) as a colourless oil. ¹H NMR: 1.77-1.81 (4H, m), 2.27 (3H, s), 3.52-3.57 (4H, m), 4.89 (2H, s), 6.28 (1H, d), 6.82 (1H, d), 7.23-7.36 (5H, m); m/z: MH⁺ 269.49.

### Method 43

### 6-Methyl-2-propylpyridin-3-ol

3-(Benzyloxy)-6-methyl-2-propylpyridine (Method 44, 660mg, 2.73 mmol) and palladium (146 mg, 0.14 mmol) in EtOH (12 mL) was stirred at r.t. under an atmosphere of hydrogen at 1 atm for 90 mins. Then the mixture was filtered and concentrated to dryness to provide the title compound (407 mg, 98%) as a white solid. ¹H NMR: 0.89 (2H, t), 1.59-1.71 (2H, m), 2.37 (3H, s), 2.71-2.76 (2H, m), 6.77 (1H, d), 6.94 (1H, d), 8.30 (1H, br s); m/z: MH⁺ 152.34.

### Method 44

### 3-(Benzyloxy)-6-methyl-2-propylpyridine

3-(Benzyloxy)-2-iodo-6-methylpyridine (Method 45, 2 g, 6.15 mmol), propylzinc bromide (25.8 mL, 12.92 mmol) and Pd(PPh₃)₄ (0.355 g, 0.31 mmol) were dissolved in THF (10 mL) and sealed into 2 microwave tubes. The mixtures were heated to 150° C. for 60 mins in a microwave reactor and were then cooled to r.t. Then a few drops of 2M HCl were added and the mixture was poured onto a pre-equilibrated (MeOH) 50 g SCX-2 cartridge and flushed thoroughly with MeOH followed by 2% NH₄OH in MeOH. Appropriate fractions were concentrated in vacuo. Further purification by FCC using a gradient of 0-20% EtOH in DCM afforded the title compound; (0.760 g, 51.2%) as a colourless oil. ¹H NMR: 0.91 (3H, t), 1.59-1.72 (2H, m), 2.38 (3H, s), 2.73-2.79 (2H, m), 4.97 (2H, s), 6.80 (1H, d), 6.96 (1H, d), 7.21-7.35 (5H, m); m/z: MH⁺ 242.58.

### Method 45

### 3-(Benzyloxy)-2-iodo-6-methylpyridine

A solution of 2-iodo-6-methylpyridin-3-ol (3 g, 12.76 mmol) in DMF (15 mL) was added dropwise to a cooled (10° C.), stirred suspension of NaH (0.562 g, 14.04 mmol) in DMF (15 mL) over a period of 10 mins under nitrogen. The resulting solution was stirred at r.t. for 15 mins. Benzyl bromide (2.402 g, 14.04 mmol) was then added dropwise and the mixture was stirred for 2 h. Then MeOH (5 mL) was added and the mixture was stirred at r.t. for a further 30 mins. Saturated NH₄Cl (3 mL) was added and the mixture was then filtered and concentrated. Purification by FCC using a gradient of 10-100% EtOAc in isohexane provided the title compound; (3.61 g, 87%) as colourless oil that solidified on standing. ¹H NMR: 2.37 (3H, s), 5.03 (2H, s), 6.80-6.88 (2H, m), 7.23-7.39 (5H, m); m/z: MH⁺ 326.41.

### Method 46

### 6-Methyl-2-(tetrahydrofuran-2-yl)pyridin-3-ol

A mixture of 3-(benzyloxy)-2-(furan-2-yl)-6-methylpyridine (Method 47, 1.85 g, 6.97 mmol) and palladium (0.371 g, 0.35 mmol) in EtOH (30 mL) was stirred at r.t. under an atmosphere of H₂ at 1 atm for 3 h. Then the mixture was filtered and the filtrate was concentrated to afford the title compound; (1.200 g, 96%); ¹H NMR: 1.90-2.00 (2H, m), 2.07-2.19 (1H, m), 2.35 (3H, s), 2.37-2.46 (2H, m), 3.88-4.08 (2H, m), 5.11 (1H, t), 6.85 (1H, d), 6.97 (1H, d); m/z: MH⁺ 180.51.

### Method 47

### 3-(Benzyloxy)-2-(furan-2-yl)-6-methylpyridine

3-(Benzyloxy)-2-iodo-6-methylpyridine (Method 45, 2.5 g, 7.69 mmol), tributyl(furan-2-yl)stannane (3.02 g, 8.46 mmol) and Pd(PPh₃)₄ (0.444 g, 0.38 mmol) were dissolved in THF (15 mL) and sealed into a microwave tube. The mixture was heated to 150° C. for 45 mins in a microwave and was then cooled to r.t. Then the mixture was poured onto a pre-equilibrated (MeOH) 50g SCX-2 cartridge which was flushed thoroughly with MeOH followed by 2% NH₄OH in MeOH. Appropriate fractions were concentrated. Further purification by FCC using a gradient of 10-80% EtOAc in DCM afforded the title compound; (1.900 g, 93%) as a white solid; m/z: MH⁻ 266.52.

### Method 48

### 6-Methyl-2-pyridin-3-ylpyridin-3-ol

The title compound was prepared from 6-methyl-3-benzyloxy-2-pyridin-3-ylpyridine (Method 49) using the method described in Method 46. ¹H NMR: (CDCl₃) 2.50 (3H, s), 6.97 (1H, d), 7.19 (1H, d), 7.40-7.44 (1H, m), 8.40-8.44 (2H, m), 8.47-8.49 (1H, m), 11.67 (1H, br s); m/z: MH⁺ 187.48.

### Method 49

### 6-Methyl-3-benzyloxy-2-pyridin-3-ylpyridine

3-(Benzyloxy)-2-iodo-6-methylpyridine (Method 45, 1.75 g, 5.38 mmol), 3-(4,4,5,5-tetramethyl-1,3,2-dioxaborolan-2-yl)pyridine (1.656 g, 8.07 mmol), (1,1′-bis-(diphenylphosphino)ferrocene)-dichloropalladium(II) (0.221 g, 0.27 mmol) and Cs₂CO₃ (3.51 g, 10.76 mmol) were dissolved in dioxane (15 mL) and the mixture was sealed into a microwave tube. The mixture was then heated to 150° C. for 30 mins in a microwave and was then cooled to r.t. Then the mixture was poured onto a pre-equilibrated (MeOH) 50 g SCX-2 cartridge which was then flushed thoroughly with MeOH followed by 2% NH₄OH in MeOH. Appropriate fractions were concentrated. Further purification by FCC using a gradient of 10-70% EtOAc in DCM afforded the title compound; (1.3 g, 87%) as a yellow solid. ¹HNMR: 2.48 (3H, s), 5.03 (2H, s), 7.00 (2H, d), 7.17-7.20 (1H, m), 7.24-7.28 (5H, m), 8.16-8.20 (1H, m), 8.50-8.52 (1H, m), 9.14 (1H, d); m/z: MH⁺ 277.54.

### Method 50

### 2-(4-Methoxyphenyl)-6-methylpyridin-3-ol

The title compound was prepared from 2-(4-methoxyphenyl)-6-methyl-3-benzyloxy-pyridine (Method 51) using the method described in Method 46. ¹H NMR: (CDCl₃) 2.44 (3H, s), 3.78 (3H, s), 5.51 (1H, s), 6.89-6.96 (3H, m), 7.05 (1H, d), 7.56-7.61 (2H, m); m/z: MH⁺ 216.12.

### Method 51

### 2-(4-Methoxyphenyl)-6-methyl-3-benzyloxypyridine

The title compound was prepared from 3-(benzyloxy)-2-iodo-6-methylpyridine (Method 45) and p-methoxyphenylboronic acid using the method described in Method 49. ¹H NMR: 2.54 (3H, s), 3.89 (3H, s), 5.27 (2H, s), 7.06 (2H, d), 7.22 (1H, d), 7.39-7.54 (4H, m), 7.59 (2H, d), 8.01 (2H, d); m/z: MH⁺ 306.19.

### Method 52

### 2-(6-Methoxypyridin-3-yl)-6-methylpyridin-3-ol

The title compound was prepared from 2-(6-methoxypyridin-3-yl)-6-methyl-3-benzyloxypyridine (Method 53) using the method described in Method 46. ¹H NMR: (CDCl₃) 2.41 (3H, s), 3.86 (3H, s), 6.68-6.71 (1H, m), 6.83 (1H, d), 6.96 (1H, d), 7.97-8.01 (1H, m), 8.58-8.59 (1H, m); m/z: MH⁺ 217.12.

### Method 53

### 2-(6-Methoxypyridin-3-yl)-6-methyl-3-benzyloxypyridine

The title compound was prepared from 3-(benzyloxy)-2-iodo-6-methylpyridine (Method 45) and 6-methoxypyridin-3-ylboronic acid using the method described in Method 49. ¹H NMR: 2.55 (3H, s), 3.99 (3H, s), 5.30 (2H, s), 6.96 (1H, d), 7.28 (1H, d), 7.40-7.55 (5H, m), 7.64 (1H, d), 8.31-8.36 (1H, m), 8.85 (1H, s); m/z: MH⁺ 307.

### Method 54

### 2-Isoquinolin-4-yl-6-methylpyridin-3-ol

The title compound was prepared from 4-(6-methyl-3-benzyloxypyridin-2-yl)-isoquinoline (Method 55) using the method described in Method 46. ¹H NMR: (CDCl₃) 2.52 (3H, s), 7.13 (1H, d), 7.31 (1H, d), 7.58-7.78 (4H, m), 8.40 (1H, s), 8.59 (1H, s); m/z: MH⁺ 237.05.

### Method 55

### 4-(6-Methyl-3-benzyloxypyridin-2-yl)isoquinoline

The title compound was prepared from 3-(benzyloxy)-2-iodo-6-methylpyridine (Method 45) and isoquinolin-4-ylboronic acid using the method described in Method 49; m/z: 327 MH⁺.

### Method 56

### 2-(4-Fluorophenyl)-6-methylpyridin-3-ol

The title compound was prepared from 2-(4-fluorophenyl)-6-methyl-3-benzyloxy-pyridine (Method 57) using the method described in Method 46. ¹H NMR: 2.41 (3H, s), 7.05 (1H, d), 7.19-7.26 (3H, m), 8.05-8.10 (2H, m), 9.92 (1H, s); m/z: MH⁺ 204.48.

### Method 57

### 2-(4-Fluorophenyl)-6-methyl-3-benzyloxypyridine

The title compound was prepared from 3-(benzyloxy)-2-iodo-6-methylpyridine (Method 45) and p-fluorophenylboronic acid using the method described in Method 49. ¹H NMR: (CDCl₃) 2.47 (3H, s), 5.00 (2H, s), 6.95 (1H, d), 6.98-7.05 (3H, m), 7.15 (1H, d), 7.23-7.31 (6H, m), 7.85-7.91 (2H, m); m/z: MH⁻ 294.54.

### Method 58

### 5-[3-(2-Chloropyridin-4-yloxy)-6-methylpyridin-2-yl]pyrimidine

3-(2-Chloropyridin-4-yloxy)-2-iodo-6-methylpyridine (Method 18, 300 mg, 0.87 mmol), 5-(tributylstannyl)pyrimidine (352 mg, 0.95 mmol) and Pd(PPh₃)₄ (50.0 mg, 0.04 mmol) were dissolved in THF (5 mL) and sealed into a microwave tube. The mixture was heated to 140° C. for 30 mins in a microwave and was then cooled to r.t. Then the mixture was poured onto a pre-equilibrated (MeOH) 50 g SCX-2 cartridge which was then flushed thoroughly with MeOH followed by 2% NH₄OH in MeOH. The product containing fractions were combined and concentrated to afford the title compound which was used without further purification; (157 mg, 60.7%); ¹H NMR: (CDCl₃) 2.69 (3H, s), 6.74-6.79 (2H, m), 7.31 (1H, d), 7.43 (1H, d), 7.64-7.71 (1H, m), 8.24 (1H, d), 9.23 (2H, s); m/z: MH⁺ 299.40.

1. A compound of formula (I): 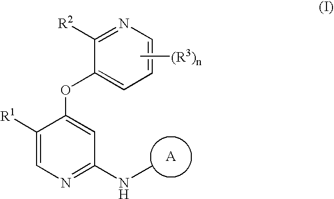
wherein:
R¹ is hydrogen, halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R³)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;
R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R³)-1-(R⁹)ureido, cyclopropyl-R¹⁰—, azetidin-1-yl-R¹¹— and (C₁₋₃alkyl)-S(O)ₐ— wherein a is 0 to 2; wherein said group may be independently optionally substituted on carbon by one or more R¹²;
R² is hydrogen, halo, cyano, nitro, mercapto, sulfo, hydroxy, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N,N—(C₁₋₆alkyl)₂amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;
R²¹ is halo, cyano, nitro, mercapto, sulfo, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R²³)amino, N—(C₁₋₆alkoxycarbonyl)-N—(R²⁴)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁵)amino, 3,3-(R²⁶)(R²⁷)-1-(R²⁸)ureido, carbocyclyl-R²⁹—, heterocyclyl-R³⁰— and (C₁₋₆alkyl)-S(O)ₐ— wherein a is 0 to 2; wherein said group may be optionally substituted on carbon by one or more R³¹; and wherein if said heterocyclyl contains an —NH— moiety that nitrogen may be optionally substituted by R³²;
n is 0 to 3; wherein the values of R³ may be the same or different;
Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;
R³³ is independently halo, cyano, nitro, mercapto, sulfo, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R³⁷)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;
R³⁴ is carbamoyl, sulfamoyl or a group selected from C₁₋₆alkyl, C₂₋₆alkenyl, C₂₋₆alkynyl, C₁₋₆alkoxycarbonyl, C₁₋₆alkanoyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R⁴⁵—, heterocyclyl-R⁴⁶— and (C₁₋₆alkyl)-S(O)ₐ— wherein a is 1 to 2; wherein said group may be optionally substituted on carbon by one or more R⁴⁷; and wherein if said heterocyclyl contains an —NH— moiety that nitrogen may be optionally substituted by R⁴⁸;
R⁴⁵ and R⁴⁶ are independently selected from a direct bond, —C(O)—, —C(═NH)—, —N(R¹⁰¹)—C(═NH)—, —N(R⁴⁹)C(O)—, —N(R⁵⁰)SO₂—, —O—C(O)— and —S(O)ₐ— wherein a is 1 or 2;
R⁴³ and R⁴⁷ are independently halo, cyano, nitro, mercapto, sulfo, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;
R²² and R³² are independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;
R⁴⁴, R⁴⁸ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R ²- and heterocyclyl-R⁸³—; wherein R⁴⁴, R⁴⁸ and R⁶⁰ are each optionally independently substituted on carbon by one or more R⁸⁴; and wherein if said heterocyclyl contains an —NH— moiety that nitrogen may be optionally substituted by R⁸⁵;
R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷—, carbocyclyl-R⁸⁸— and (C₁₋₆alkyl)-S(O)ₐ— wherein a is 0 to 2; and wherein if said heterocyclyl contains an —NH— moiety that nitrogen may be optionally substituted by R⁸⁹;
R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;
R⁸⁵ and R⁸⁹ are each independently selected from C₁₋₆alkyl, C₁₋₆alkanoyl; C₁₋₆alkylsulfonyl;
R¹⁹ and R²⁰ are independently selected from a direct bond, —CH(R⁶¹)—, —CH(OR⁶²)—, —C(R⁶³)═C(R⁶⁴)—, ethynylene, —O—, —C(O)—, —N(R⁶⁶)C(O)—, —N(R⁶⁹)SO₂— and —S(O)ₐ— wherein a is 0 to 2;
R¹⁰, R¹¹, R²⁹, R³⁰, R⁴¹, R⁴², R⁵⁷ and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;
R¹², R³¹ and R⁵⁹ are independently selected from fluoro, chloro, cyano, nitro, hydroxy, trifluoromethoxy, trifluoromethyl, amino, carboxy, sulfo, carbamoyl, mercapto, sulfamoyl, carbamimidoyl, carbamimidoylamino, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;
R⁴, R⁵, R⁶, R⁷, R⁸, R⁹, R²³, R²⁴, R²⁵, R²⁶, R²⁷, R²⁸, R³⁵, R³⁶, R³⁷, R³⁸, R³⁹, R⁴⁰, R⁴⁹, R⁵⁰, R⁵¹, R⁵², R⁵³, R⁵⁴, R⁵⁵, R⁵⁶, R⁶¹, R⁶², R⁶³, R⁶⁴, R⁶⁶, R⁶⁹, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl;
or a pharmaceutically acceptable salt thereof,
wherein the compound of formula (I) is other than:
3-(2-{[4-(1-acetylpiperidin-4-yl)-1,3-thiazol-2-yl]amino}pyridin-4-yl)-oxypyridine-4-carbonitrile;
N-(4-methyl-1,3-thiazol-2-yl)-4-pyridin-3-yloxypyridin-2-amine;
ethyl 5-{5-bromo-2-[(3-{1-[(2-methylpropan-2-yl)oxycarbonyl]piperidin-4-yl}-1,2,4-thiadiazol-5-yl)amino]pyridin-4-yl}oxy-4,6-dimethylpyridine-3-carboxylate;
5-{5-bromo-2-[(3-piperidin-4-yl-1,2,4-thiadiazol-5-yl)amino]pyridin-4-yl]oxy-N-(2-dimethylaminoethyl}-4,6-dimethylpyridine-3-carboxamide;
tert-butyl 4-[5-({5-bromo-4-[5-(2-dimethylaminoethylcarbamoyl)-2,4-dimethylpyridin-3-yl]oxypyridin-2-yl}amino)-1,2,4-thiadiazol-3-yl]piperidine-1-carboxylate; or
5-{5-bromo-2-[(3-{1-[(2-methylpropan-2-yl)oxycarbonyl]piperidin-4-yl}-1,2,4-thiadiazol-5-yl)amino]pyridin-4-yl}oxy-4,6-dimethylpyridine-3-carboxylic acid; or a pharmaceutically acceptable salt thereof.

2. The compound of formula (I), or a pharmaceutically acceptable salt thereof, as claimed in claim 1, wherein R³ are each independently halo, nitro, cyano, mercapto, sulfo, hydroxy, carbamoyl, sulfamoyl, amino, carboxy or a group selected from C₁₋₃alkyl, C₂₋₃alkenyl, C₂₋₃alkynyl, C₁₋₃alkoxy, C₁₋₃alkoxycarbonyl, C₁₋₃alkanoyl, C₁₋₃alkanoyloxy, C₁₋₃alkylsulfonyloxy, N—(C₁₋₃alkyl)amino, N,N—(C₁₋₃alkyl)₂amino, N—(C₁₋₃alkanoyl)-N—(R⁴)amino, N—(C₁₋₃alkoxycarbonyl)-N—(R⁵)amino, N—(C₁₋₃alkyl)carbamoyl, N,N—(C₁₋₃alkyl)₂carbamoyl, N—(C₁₋₃alkyl)sulfamoyl, N,N—(C₁₋₃alkyl)₂sulfamoyl, N—[(C₁₋₃alkyl)sulfonyl]-N—(R⁶)amino, 3,3-(R⁷)(R⁸)-1-(R⁹)ureido, cyclopropyl-R¹⁰—, azetidin-1-yl-R¹¹— and (C₁₋₃alkyl)-S(O)ₐ— wherein a is 0 to 2.

3. The compound of formula (I), or a pharmaceutically acceptable salt thereof, as claimed in claim 1, wherein the compound of formula (I) is a compound of formula (IA): 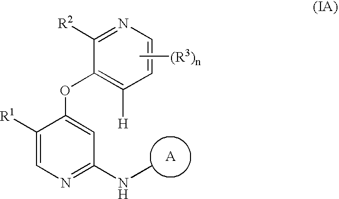
wherein:
R² is halo, cyano, nitro, mercapto, sulfo, hydroxy, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N,N—(C₁₋₆alkyl)₂amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, carbocyclyl-R¹⁹—, heterocyclyl-R²⁰— and (C₁₋₆alkyl)-S(O)ₐ— wherein a is 0 to 2; wherein said group may be independently optionally substituted on carbon by one or more R²¹; and wherein if said heterocyclyl contains an —NH— moiety that nitrogen may be optionally substituted by R²²;
n is 0 to 2; wherein the values of R³ may be the same or different;
and when n is 1 or 2, R² is hydrogen or a value of R² as defined above within this claim;
and R¹ and Ring A are as defined in claim 1.

4. The compound of formula (I), or a pharmaceutically acceptable salt thereof, as claimed in claim 3 wherein R¹ is hydrogen.

5. The compound of formula (I), or a pharmaceutically acceptable salt thereof, as claimed in claim 3, wherein:
R² is halo, carboxy or a group selected from C₁₋₆alkyl, C₁₋₆alkoxycarbonyl, N—(C₁₋₆alkyl)carbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;
R²¹ is halo, cyano, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, carbocyclyl-, heterocyclyl- and (C₁₋₆alkyl)-S(O)ₐ— wherein a is 0 to 2; and
R²² is C₁₋₆alkyl.

6. The compound of formula (I), or a pharmaceutically acceptable salt thereof, as claimed in claim 3 wherein:
Ring A is a carbocyclic group or a heterocyclic group, wherein said heterocyclic group or carbocyclic group may be optionally substituted on one or more carbons by R³³; and wherein if said heterocyclic group contains an —NH— moiety that nitrogen may be optionally substituted by R³⁴;
R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R³⁷)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;
R³⁴ is carbamoyl, sulfamoyl or a group selected from C₁₋₆alkyl, C₂₋₆alkenyl, C₂₋₆alkynyl, C₁₋₆alkoxycarbonyl and C₁₋₆alkanoyl;
R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R^{5 1})amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;
R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;
R⁸⁴ is selected from halo, hydroxy, cyano, carbamimidoyl, C₁₋₆alkyl, C₂₋₆alkenyl, C₂₋₆alkynyl, C₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷— and carbocyclyl-R⁸⁸—;
R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;
R⁸⁵ is selected from C₁₋₆alkyl, C₁₋₆alkanoyl and C₁₋₆alkylsulfonyl;
R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;
R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino; and
R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl.

7. The compound of formula (I), or a pharmaceutically acceptable salt thereof, as claimed in claim 3 wherein R³ is independently C₁₋₃alkyl and n is 1 or 2 wherein the values of R³ may be the same or different.

8. The compound of formula (I), or a pharmaceutically acceptable salt thereof, as claimed in claim 1, wherein the compound of formula (I) is a compound of formula (IB): 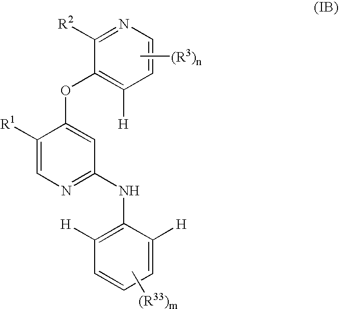
wherein:
m is 0 to 3; wherein the values of R³³ may be the same or different;
R¹ is hydrogen;
R³ is independently methyl or ethyl;
n is 0 to 2; wherein the values of R³ may be the same or different;
R² is halo, carboxy or a group selected from C₁₋₆alkyl, C₁₋₆alkoxycarbonyl, N—(C₁₋₆alkyl)carbamoyl, phenyl, pyridinyl, pyrazolyl, thiazolyl, thienyl, pyrazinyl, furanyl, quinolinyl, pyrimidinyl, tetrahydrofuranyl and pyrrolidinyl; wherein said group may be independently optionally substituted on carbon by one or more R²¹; and wherein if said pyrazolyl contains an —NH— moiety that nitrogen may be optionally substituted by R²²;
R²¹ is halo, cyano, hydroxy, amino, carboxy, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, carbocyclyl-, heterocyclyl- and (C₁₋₆alkyl)-S(O)ₐ— wherein a is 0 to 2;
R²² is C₁₋₆alkyl;
R³³ is independently halo, cyano, hydroxy, carboxy, carbamimidoyl, amino, carbamoyl, sulfamoyl or a group selected from C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁵)amino, N—(C₁₋₆alkoxycarbonyl)-N—(R³⁶)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—(C₁₋₆alkanoyl)-N—(R⁷⁵)-sulfamoyl, N—[(C₁₋₆alkyl)sulfonyl]-N—(R³⁷)amino, 3,3-(R³⁸)(R³⁹)-1-(R⁴⁰)ureido, (R⁷⁶)(R⁷⁷)N—S(O)₂—N(R⁷⁸)—, N—(C₁₋₆alkyl)carbamimidoyl, N,N—(C₁₋₆alkyl)₂carbamimidoyl, N—[N′,N′—(R⁹⁸)(R⁹⁹)carbamimidoyl]-N—(R¹⁰⁰)amino, carbocyclyl-R⁴¹—, heterocyclyl-R⁴²— and (C₁₋₆alkyl)-S(O)ₐ— wherein a is 0 to 2; wherein said group may be optionally substituted on carbon by one or more R⁴³; and wherein if said heterocyclyl contains an —NH— moiety that nitrogen may be optionally substituted by R⁴⁴;
R⁴³ is halo, cyano, hydroxy, amino, carbamimidoyl, carboxy, carbamoyl, sulfamoyl, or a group selected from C₁₋₆alkyl, C₂₋₆alkenyl, C₂₋₆alkynyl, C₁₋₆alkoxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R⁵¹)amino, N—(C₁₋₆alkoxycarbonyl)-N—(R⁵²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁵³)amino, 3,3-(R⁵⁴)(R⁵⁵)-1-(R⁵⁶)ureido, N—(C₁₋₆alkanoyl)-N—(R⁹⁵)-sulfamoyl, (R⁷⁹)(R⁸⁰)N—S(O)₂—N(R⁸¹)—, N—(C₁₋₆alkyl)carbamimidoyl, N,N—(C₁₋₆alkyl)₂carbamimidoyl, N—[N′,N′—(R¹⁰²)(R¹⁰³)carbamimidoyl]-N—(R¹⁰⁴)amino, carbocyclyl-R⁵⁷—, heterocyclyl-R⁵⁸— and (C₁₋₆alkyl)-S(O)ₐ— wherein a is 0 to 2; wherein said group may each be optionally independently substituted on carbon by one or more R⁵⁹; and wherein if said heterocyclyl contains an —NH— moiety that nitrogen may be optionally substituted by R⁶⁰;
R⁴⁴ and R⁶⁰ are independently selected from carbamimidoyl, C₁₋₆alkyl, C₂₋₆alkenyl, C₂₋₆alkynyl, C₁₋₆alkanoyl, C₁₋₆alkylsulfonyl, N—(C₁₋₆alkyl)sulfamoyl, N,N—(C₁₋₆alkyl)₂sulfamoyl, C₁₋₆alkoxycarbonyl, carbamoyl, sulfamoyl, N—(C₁₋₆alkyl)carbamoyl, N,N—(C₁₋₆alkyl)₂carbamoyl, N—(C₁₋₆alkyl)carbamimidoyl, N,N—(C₁₋₆alkyl)₂carbamimidoyl, carbocyclyl-R⁸²— and heterocyclyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;
R⁸⁴ is selected from halo, hydroxy, cyano, carbamimidoyl, C₁₋₆alkyl, C₂₋₆alkenyl, C₂₋₆alkynyl, CI₁₋₆alkoxy, amino, N—(C₁₋₆alkyl)amino, N,N—(C₁₋₆alkyl)₂amino, carbamoyl, sulfamoyl, N—(C₁₋₆alkyl)carbamoyl, N,N—(C₁₋₆alkyl)₂carbamoyl, C₁₋₆alkoxycarbonyl, N—(C₁₋₆alkoxycarbonyl)-N—(R⁸⁶)-amino, (R⁹⁶)(R⁹⁷)N—S(O)₂—N(R⁹⁸)—, 3,3-(R⁹²)(R⁹³)-1-(R⁹⁴)ureido, N—(C₁₋₆alkyl)carbamimidoyl, N,N—(C₁₋₆alkyl)₂carbamimidoyl, N—[N′,N′—(R¹⁰⁵)(R¹⁰⁶)carbamimidoyl]-N—(R¹⁰⁷)amino, heterocyclyl-R⁸⁷— and carbocyclyl-R⁸⁸—;
R⁸², R⁸³, R⁸⁷ and R⁸⁸ are each independently selected from a direct bond, —C(O)—, —C(═NH)—, —N(R¹⁰⁸)—C(═NH)—, —C(═NH)—N(R¹⁰⁹)—, —N(R⁹⁰)C(O)—, —N(R⁹¹)SO₂—, —O—C(O)— and —S(O)ₐ— wherein a is 1 or 2;
R⁸⁵ is selected from C₁₋₆alkyl, C₁₋₆alkanoyl and C₁₋₆alkylsulfonyl;
R⁴¹, R⁴² and R⁵⁸ are independently selected from a direct bond, —O—, —N(R⁷⁰)—, —C(O)—, —C(═NH)—, —N(R¹¹⁰)—C(═NH)—, —C(═NH)—N(R¹¹¹)—, —N(R⁷¹)C(O)—, —C(O)N(R⁷²)—, —SO₂N(R⁷³)—, —N(R⁷⁴)SO₂— and —S(O)ₐ— wherein a is 0 to 2;
R⁵⁹ is selected from fluoro, chloro, cyano, hydroxy, trifluoromethoxy, trifluoromethyl, amino, carboxy, carbamoyl, sulfamoyl, carbamimidoyl and carbamimidoylamino; and
R³⁵, R³⁶, R³⁷, R³⁸, R³⁹, R⁴⁰, R⁵¹, R⁵², R⁵³, R⁵⁴, R⁵⁵, R⁵⁶, R⁷⁰, R⁷¹, R⁷², R⁷³, R⁷⁴, R⁷⁵, R⁷⁶, R⁷⁷, R⁷⁸, R⁷⁹, R⁸⁰, R⁸¹, R⁸⁶, R⁹⁰, R⁹¹, R⁹², R⁹³, R⁹⁴, R⁹⁵, R⁹⁶, R⁹⁷, R⁹⁸, R⁹⁹, R¹⁰⁰, R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰ and R¹¹¹ are independently selected from hydrogen, C₁₋₃alkyl and cyclopropyl.

9. The compound of formula (I), or a pharmaceutically acceptable salt thereof, as claimed in claim 8 wherein m is 1 to 3; and the values of R³³ may be the same or different.

10. The compound of formula (I), or a pharmaceutically acceptable salt thereof, as claimed in claims 8 wherein n is 1 or 2; and the values of R³ may be the same or different.

11. The compound of formula (I), or a pharmaceutically acceptable salt thereof, as claimed in claim 1, wherein the compound of formula (I) is a compound of formula (IA), as depicted above, wherein:
R¹ is hydrogen;
R³ is methyl or ethyl and n is 1 or 2 wherein the values of R³ may be the same or different;
R² is hydrogen, chloro or a group selected from methyl, phenyl, pyridinyl, thiazolyl, thienyl, pyrazinyl and pyrazolyl; wherein said group may be optionally substituted on carbon by one or more R²¹; and wherein if said pyrazolyl contains an —NH— moiety that nitrogen may be optionally substituted by R²²;
R²¹ is chloro, fluoro, cyano, or a group selected from methyl, methoxy and acetylamino;
R²² is methyl;
Ring A is phenyl or a heterocyclic group selected from pyridinyl, pyrazolyl, indolyl and 2,2-dioxido-1,3-dihydro-2-benzothienyl; wherein said phenyl and heterocyclic group may be optionally substituted on one or more carbons by R³³; and wherein if said heterocyclic group contains an —NH— moiety that nitrogen may be optionally substituted by R³⁴;
R³³ is independently fluoro, chloro, cyano, hydroxy, carboxy, carbamoyl, sulfamoyl or a group selected from methyl, methoxy, ethoxy, propoxy, methoxycarbonyl, ethoxycarbonyl, N-acetyl-N—(R³⁵)amino, methylcarbamoyl, ethylcarbamoyl, propylcarbamoyl, isopropylcarbamoyl, dimethylcarbamoyl, methylsulfamoyl, ethylsulfamoyl, propylsulfamoyl, isopropylsulfamoyl, pentylsulfamoyl, N-ethyl-N-methyl-sulfamoyl, N,N-diethylsulfamoyl, N,N-dimethylsulfamoyl, N-(acetyl)sulfamoyl, N-(methylsulfonyl)amino, cyclopropyl-R⁴¹—, cyclobutyl-R⁴¹—, phenyl-R⁴¹—, morpholinyl-R⁴²—, piperazinyl-R⁴²— piperdinyl-R⁴²—, tetrahydropyranyl-R⁴²—, azetidinyl-R⁴²—, pyrrolidinyl-R⁴²—, oxazolyl-R⁴²—, azepanyl-R⁴²—, pyridinyl-R⁴²— 2-oxopyrrolidinyl-R⁴²— and methylsulfonyl; wherein said group may be optionally substituted on carbon by one or more R⁴³; and wherein if said heterocyclyl contains an —NH— moiety that nitrogen may be optionally substituted by R⁴⁴;
R³⁴ is methyl or ethyl;
R⁴³ is chloro, cyano, hydroxy, amino, carbamoyl, or a group selected from methyl, ethynyl, methoxy, isopropylamino, acetylamino, methylsulfamoyl, N-(tert-butoxycarbonyl)amino, 3,3-dimethyl-2,2-dioxido-2λ⁶-diazathianyl, pyrazinyl-R⁵⁸—, piperazinyl-R⁵⁸—, morpholinyl-R⁵⁸—, pyridinyl-R⁵⁸— 1,1-dioxidothiomorpholinyl-R⁵⁸—, piperidinyl-R⁵⁸—, imidazolyl-R⁵⁸—, pyrazolyl-R⁵⁸—, pyrrolidinyl-R⁵⁸—, pyrrolyl-R⁵⁸—, 8-oxa-3-azabicyclo[3.2.1]octanyl-R⁵⁸— and methylsulfonyl; wherein said group may each be optionally independently substituted on carbon by one or more R⁵⁹; and wherein if said heterocyclyl contains an —NH— moiety that nitrogen may be optionally substituted by R⁶⁰;
R⁴⁴ and R⁶⁰ are independently selected from methyl, ethyl, ethanoyl, propanoyl, methylsulfonyl, tert-butoxycarbonyl, pentylcarbamoyl, adamantyl-R⁸²—, cyclohexyl-R⁸²—, phenyl-R⁸²—, cyclopropyl-R⁸²—, pyridinyl-R⁸³—, pyrrolyl-R⁸³— and tetrahydropyranyl-R⁸³—; wherein R⁴⁴ and R⁶⁰ are each optionally independently substituted on carbon by one or more R⁸⁴; and wherein if said heterocyclyl contains an —NH— moiety that nitrogen may be optionally substituted by R⁸⁵;
R⁸⁴ is selected from chloro, fluoro, methoxy, amino, carbamoyl, methylcarbamoyl, N-(tert-butoxycarbonyl)amino, triazolyl-R⁸⁷—, 2-oxoimidazolidinyl-R⁸⁷— and cyclopropyl-R⁸⁸—;
R⁸², R⁸³, R⁸⁷ and R⁸⁸ are each independently selected from a direct bond, —C(O)—, —NH—C(O)—, and —S(O)₂—;
R⁸⁵ is methyl;
R⁴¹, R⁴² and R⁵⁸ are each independently selected from a direct bond, —C(O)—, —N(R⁷¹)C(O)—, —NH—SO₂— and —S(O)₂—;
R⁵⁹ is fluoro;
R³⁵ is hydrogen or methyl; and
R⁷¹ is hydrogen or cyclopropyl.

12. A compound of formula (I), or a pharmaceutically acceptable salt thereof, as claimed in claim 1, selected from:
4-(5,6-dimethyl-2-pyridin-2-yl-pyridin-3-yl)oxy-N-pyridin-2-yl-pyridin-2-amine, 4-{[4-(5,6-dimethyl-2-pyridin-2-yl-pyridin-3-yl)oxypyridin-2-yl]amino}benzene-sulfonamide, (4-{[4-(5,6-dimethyl-2-pyridin-2-yl-pyridin-3-yl)-oxypyridin-2-yl]amino}phenyl)methanol, 4-( {4-[(2,6-dimethylpyridin-3-yl)oxy]pyridin-2-yl}amino)-benzenesulfonamide, 4-{[4-(6-ethyl-2-pyridin-2-yl-pyridin-3-yl)oxypyridin-2-yl]-amino}benzenesulfonamide, (3-{[4-(2,6-dimethylpyridin-3-yl)oxypyridin-2-yl]amino}phenyl)-(3-methylsulfonylpyrrolidin-1-yl)methanone, N-(2-amino-2-oxoethyl)-3-{[4-(2,6-dimethylpyridin-3-yl)-oxypyridin-2-yl]amino}benzamide, 2-(4-{[4-(5,6-dimethyl-2-pyridin-2-ylpyridin-3-yl)oxypyridin-2-yl]-amino}phenyl)acetonitrile, 4-{[4-(5,6-dimethyl-2-pyridin-2-ylpyridin-3-yl)oxypyridin-2-yl]-amino}benzamide, 3-{[4-(5,6-dimethyl-2-pyridin-2-ylpyridin-3-yl)oxypyridin-2-yl]-amino}benzenesulfonamide, N-(3-{[4-(2,6-dimethylpyridin-3-yl)oxypyridin-2-yl]amino}phenyl)methanesulfonamide, 4-(6-methyl-2-propyl-pyridin-3-yloxy)-N-(3,4,5-trimethoxy-phenyl)pyridin-2-amine, 4-(2,6-dimethyl-pyridin-3-yl)oxy-N-{3-[(4-methylsulfonyl-piperazin-1-yl)methyl]phenyl}pyridin-2-amine, 4-(2,6-dimethylpyridin-3-yl)oxy-N-[3-(piperazin-1-ylmethyl)phenyl]pyridin-2-amine, N-cyclopropyl-2-{4-[(3-{[4-(2,6-dimethylpyridin-3-yl)oxypyridin-2-yl]amino}phenyl)methyl]piperazin-1-yl}acetamide, 2-{4-[3-({4-[(2,6-dimethyl-pyridin-3-yl)oxy]pyridin-2-yl}amino)benzyl]piperazin-1-yl}propanamide and 3-{[4-(5,6-dimethyl-2-pyridin-2-ylpyridin-3-yl)oxypyridin-2-yl]amino}phenol.

13. A pharmaceutical composition comprising a compound of formula (I), or a pharmaceutically acceptable salt thereof, as claimed in claims 1, in association with a pharmaceutically acceptable adjuvant, diluent or carrier.

14-16. (canceled)

17. A method of treating cancer which comprises administering to a patient in need thereof a therapeutically effective amount of a compound of formula (I) as in claim 1, or a pharmaceutically acceptable salt thereof.
